(19) United States
(12) Patent Application Publication
(10) Pub. No.: US 2025/0235456 A1
(43) Pub. Date: Jul. 24, 2025

(54) METHODS OF USE FOR AZA-QUINAZOLINE COMPOUNDS

(71) Applicant: Iambic Therapeutics, Inc., San Diego, CA (US)

(72) Inventors: William VERNIER, Vista, CA (US); Quynh Nhu NGUYEN, Redwood City, CA (US); Nomaan REZAYEE, San Diego, CA (US); Laurent GOMEZ, San Diego, CA (US); Chao ZHANG, San Diego, CA (US); Thomas Francis MILLER, III, La Jolla, CA (US); Frederick Roy MANBY, Bristol (GB); Angus VOICE, Bristol (GB); Chunmei ZHAO, San Diego, CA (US); Chang ZHAO, San Diego, CA (US)

(21) Appl. No.: 19/025,976

(22) Filed: Jan. 16, 2025

Related U.S. Application Data
(63) Continuation of application No. PCT/US2025/011907, filed on Jan. 16, 2025.
(60) Provisional application No. 63/621,996, filed on Jan. 17, 2024.

(51) Int. Cl. A61K 31/519; A61K 31/5386; A61K 45/06; A61P 35/00; A61P 35/04

(52) U.S. Cl. CPC A61K 31/519; A61K 31/5386; A61K 45/06; A61P 35/00; A61P 35/04

(57) ABSTRACT

Disclosed is a method of treating a disease or condition comprising administering to a subject in need thereof a therapeutically effective amount of a compound having the structure of Formula (I0), or a pharmaceutically acceptable salt or solvate thereof, in combination with one or more additional therapeutic agents or therapies, wherein Formula (I0) is:

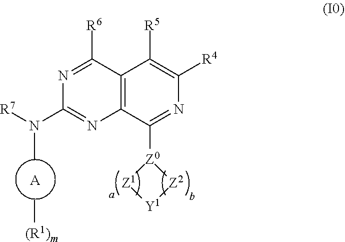

wherein, A, Z⁰, Z¹, Z², Y¹, a, b, m, R¹, R⁴, R⁵, R⁶, R⁷ are as described in the specification.

### CROSS REFERENCE

This application is a continuation of International Application No. PCT/US2025/011907, filed Jan. 16, 2025, which claims benefit of U.S. Provisional Application No. 63/621,996, filed January, which is incorporated herein by reference in its entirety.

### BACKGROUND OF THE INVENTION

Division and proliferation of mammalian cells mediated by the cell cycle is an important and fundamental biological process, which controls production and generation of cells with critical biological functions. Cell cycle is a highly regulated process and responds to a complex set of cell signals within the cell and externally. The complex network of cell signaling, including components promoting and suppressing cancer, plays a key role controlling the cell cycle. Gain-of-function of tumor-promoting components or loss-of-function of tumor-suppressing products can lead to unregulated cell cycle and subsequently tumorigenesis.

Cyclins and cyclin-dependent kinases (CDKs) are crucial for driving and controlling cell cycle transitions and cell division (34176404). Cyclin is a family of proteins whose expression levels vary at different stages in the cell cycle. Cyclins bind and activate CDKs during different stages of cell cycle, of which the progression is tightly synchronized involving sequential activation of several cyclin-CDK complexes. Of more than 20 CDKs discovered so far, CDK1, 2, 4, 6 have been reported to play a direct role in cell cycle progression. CDK4-cyclin D and CDK6-cyclin D complexes are essential for entry in G1 phase of cell cycle. CDK2-cyclin E complex regulates progression from G1 into S phase, while CDK2-cyclin A is required during S phase. CDK1-cyclin A complex promotes entry into M phase, and mitosis is further regulated by CDK1-cyclin B complex. Progressive phosphorylation of retinoblastoma (Rb) by CDK4-cyclin D, CDK6-cyclin D and CDK2-cyclin E releases the GI transcription factor, E2F, and promotes S-phase entry. Activation of CDK2-cyclin A during early S-phase promotes phosphorylation of endogenous substrates that permit DNA replication and inactivation of E2F, for S-phase completion.

Dysregulation of cell-cycle machinery is a hallmark of cancer, leading to overactivation of CDKs and uncontrolled cell division and proliferation. Genetic alterations of the genes encoding cyclin D, CDK4/6, and CDK4/6-inhibiting proteins (such as p21, p27) all contribute to tumorigenesis. Cyclin E, the regulatory cyclin for CDK2, is frequently overexpressed in cancer. Since tumor development is closely related to gene mutation and deregulation of CDK and its regulators, CDK inhibitors are useful for anticancer therapy. CDK inhibitors have been developed as cancer therapy since the early 90s, with multiple FDA-approved drugs (Palbociclib, ribociclib and abemaciclib). However, these early generation CDK inhibitors on the market have poor selectivity and high toxicity (such as myelosuppression), leading to adverse effects limiting clinical dosing level for further patient benefit. There remains an unmet medical need to develop methods of use for novel CDK inhibitors with better selectivity and less side effects for normal cells.

### SUMMARY OF THE INVENTION

The present disclosure generally relates methods of use for substituted quinolinone amide compounds or salts of Formula (I0), (I), (IA), (IAA), (IB), (IBB), (IC), (ICC), (ID), (IDD), (IE), and (IEE) and pharmaceutical compositions thereof. The substituted quinolinone amide compounds or salts of Formula (I0), (I), (IA), (IAA), (IB), (IBB), (IC), (ICC), (ID), (IDD), (IE), and (IEE) disclosed herein may be used for the treatment of abnormal cell growth, such as cancer, in a subject in need thereof.

In some aspects, methods of treating cancer may comprise administering a compound or pharmaceutically acceptable salt of any one of Formula (I0), (I), (IA), (IAA), (IB), (IBB), (IC), (ICC), (ID), (IDD), (IE), and (IEE) in combination with one or more additional therapeutic agents or therapies to in an individual in need thereof.

In certain aspects, the disclosure provides a method of treating a disease or condition comprising administering to a subject in need thereof a therapeutically effective amount of a compound having the structure of Formula (I0), or a pharmaceutically acceptable salt or solvate thereof, in combination with one or more additional therapeutic agents or therapies, wherein Formula (I0) is:

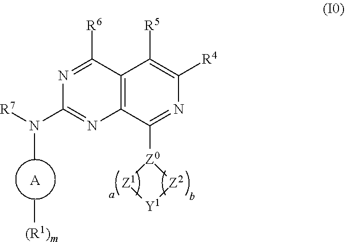

wherein,
   A is a ring selected from optionally substituted carbocycle, optionally substituted 4- to 8-membered heterocycle, optionally substituted tetrahydro-triazolopyrazine, and optionally substituted isoindoline;
   Z⁰ is —C(H)— or nitrogen;
   each of Z¹, Z², and Y¹ are independently selected from —C(R²)₂—, —C(O)—, —NR³—, —N(C(O)R²)—, —NS(O₂)R², —O—, —S—, —S(O)—, and —S(O)₂—;
   each of a and b are independently selected from 1, 2, 3, and 4;
   each R¹ is independently selected from halogen, —CN, —NO₂, optionally substituted alkyl, optionally substituted alkenyl, optionally substituted alkynyl, optionally substituted carbocycle, and optionally substituted heterocycle;
   m is selected from 0 to 5;
   each R² is independently selected from hydrogen, halogen, —CN, —OH, optionally substituted alkoxy, optionally substituted alkyl, optionally substituted cycloalkyl, optionally substituted —O-cycloalkyl, and optionally substituted heterocycloalkyl, or two R² substituents come together to form an optionally substituted heterocycle or an optionally substituted carbocycle, or R² and R³ substituents come together to form an optionally substituted heterocycle;
   each R³ is independently selected from hydrogen, optionally substituted alkyl, optionally substituted C₃₋₄ carbocycle, and optionally substituted 3- to 4-membered heterocycloalkyl;
   R⁴ is selected from hydrogen, halogen, —CN, optionally substituted C₁₋₄ alkyl, optionally substituted C₃₋₄ carbocycle, and optionally substituted 3- to 4-membered heterocycloalkyl;
   each of R⁵, R⁶, is independently selected from hydrogen, halogen, —CN, optionally substituted C₁₋₄ alkyl, optionally substituted C₃₋₄ carbocycle, and optionally substituted 3- to 4-membered heterocycloalkyl; and
   R⁷ is selected from hydrogen and optionally substituted C₁₋₄ alkyl, and
   wherein if A is optionally substituted phenyl, m is from 1 to 5 and at least one R¹ is a heterocycloalkyl,
   wherein if A is optionally substituted pyridine, optionally substituted pyridazine, or optionally substituted pyrimidine, R⁴ is selected from hydrogen, halogen, and —CN,
   wherein if A is an optionally substituted piperidine sulfonamide, then either (i) Y¹ is —C(R²)₂— and the two R² substituents come together to form a ring selected from an optionally substituted heterocycle and an optionally substituted carbocycle, or (ii) R⁴ is selected from hydrogen, halogen, methyl, and —CN, and
   wherein if A-R¹ is

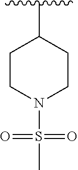 [SMILES: *C1CCN(S(C)(=O)=O)CC1]

and Z⁰ is CH, then R⁴ is methyl or cyclopropyl.

In certain aspects, the disclosure provides a pharmaceutical composition comprising a compound described herein and a pharmaceutically acceptable excipient.

In certain aspects, the disclosure provides a method of treating cancer comprising administering to a subject in need thereof a compound or pharmaceutical composition described herein. In certain aspects, the disclosure provides a method of inhibiting a cyclin dependent kinase (CDK) in a cell with a compound or pharmaceutically acceptable salt or the pharmaceutical composition described herein.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Basic functions for cell regulation, cell division, and cell proliferation are controlled by cyclin-dependent kinases (CDKs) activated by regulatory subunits such as cyclins. CDK inhibitors are useful in the treatment of cancer due to CDKs role in cell regulation. It has been shown that increased activity or transient abnormal activation of CDKs leads to the development of tumors; development of tumors are often associated with changes in the CDKs or regulators of CDKs.

CDKs bind to cyclin, which a regulatory protein, and without cyclin, it has little kinase activity. The cyclin-CDK complex is an active kinase typically modulated by phosphorylation and other binding proteins. There are currently 21 CDKs and 5 CDK-like genes that are known in the human genome. While many of the CDKs have been linked to transcription, CDK2, CDK4, and CDK6 are associated with the cell cycle. CDK2 is associated with DNA replication in higher eukaryotes whereas CDK4 and CDK6 are associated with various growth-regulatory signals.

CDK2 overexpression is associated with abnormal regulation of the cell cycle. Cyclin E, the cyclin partner of CDK2, binds to CDK2 to form an active kinase complex. The CDK2-cyclin E complex is important in the regulation of the G1/S transition, centrosome replication, and histone biosynthesis. Progressive phosphorylation can release the G1 transcription factor E2F and promote entry into the S phase. Another cyclin partner of CDK2, cyclin A, can bind and activate CDK2 during the initial phase of the S phase, and promote endogenous substrate phosphorylation, which allows DNA replication and E2F inactivation to complete the S phase.

CDK4 and CDK6 are also associated with the cell cycle. CDK4 and CDK6 inhibitors can arrest the cell cycle form the G1 to S phase by blocking phosphorylation of Rb protein and inhibiting proliferation of Rb-positive tumor cells. Besides cell cycle activity, CDK4 and CDK6 inhibitors can also suppress tumor growth through other mechanisms including, but not limited to inducing senescence, promoting anti-tumor immune responses, regulation of cell metabolism, and enhancing cytostasis caused by signaling pathway inhibitors.

### Definitions

Unless defined otherwise, all technical and scientific terms used herein have the same meaning as is commonly understood by one of skill in the art to which this invention belongs.

As used in the specification and claims, the singular form “a”, “an” and “the” includes plural references unless the context clearly dictates otherwise.

As used in the specification and appended claims, unless specified to the contrary, the following terms have the meaning indicated below.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Hydrazino” refers to the ═N—NH₂ radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₅ alkyl). In other embodiments, an alkyl comprises one to five carbon atoms (e.g., C₁-C₅ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈ alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond.

“Heteroalkyl” refers to an alkyl group, as defined above, having from one or more carbon atoms replaced with a heteroatom, such as wherein the heteroatom is individually selected from N, O and S at each replacement location. Additional heteroatoms can also be useful, including, but not limited to, B, Al, Si and P. The heteroatoms can also be oxidized, such as, but not limited to, —S(O)— and —S(O)₂— For example, heteroalkyl can include ethers, thioethers and alkyl-amines.

Heteroalkyl consisting of the stated number of carbon atoms and may include one or more heteroatoms selected from the group consisting of O, N, Si and S, wherein the nitrogen heteroatom may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Two heteroatoms may be consecutive, such as, for example, —CH₂NHOCH₃ and —CH₂OSi(CH₃)₃. Heteroalkyl can include any stated number of carbon atoms as defined herein and in the definition of alkyl.

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like.

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₈ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈ alkylene). In other embodiments, an alkylene comprises two to five carbon atoms (e.g., C₂-C₅ alkylene). In other embodiments, an alkylene comprises three to five carbon atoms (e.g., C₃-C₅ alkylene).

“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. In certain embodiments, an alkenylene comprises two to eight carbon atoms (e.g., C₂-C₈ alkenylene). In other embodiments, an alkenylene comprises two to five carbon atoms (e.g., C₂-C₅ alkenylene). In other embodiments, an alkenylene comprises two to four carbon atoms (e.g., C₂-C₄ alkenylene). In other embodiments, an alkenylene comprises two to three carbon atoms (e.g., C₂-C₃ alkenylene). In other embodiments, an alkenylene comprises five to eight carbon atoms (e.g., C₅-C₈ alkenylene). In other embodiments, an alkenylene comprises two to five carbon atoms (e.g., C₂-C₅ alkenylene). In other embodiments, an alkenylene comprises three to five carbon atoms (e.g., C₃-C₅ alkenylene).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. In certain embodiments, an alkynylene comprises two to eight carbon atoms (e.g., C₂-C₅ alkynylene). In other embodiments, an alkynylene comprises two to five carbon atoms (e.g., C₂-C₅ alkynylene). In other embodiments, an alkynylene comprises two to four carbon atoms (e.g., C₂-C₄ alkynylene). In other embodiments, an alkynylene comprises two to three carbon atoms (e.g., C₂-C₃ alkynylene). In other embodiments, an alkynylene comprises two carbon atoms (e.g., C₂ alkylene). In other embodiments, an alkynylene comprises five to eight carbon atoms (e.g., C₅-C₅ alkynylene). In other embodiments, an alkynylene comprises three to five carbon atoms (e.g., C₃-C₅ alkynylene).

“Heteroalkylene” refers to a straight or branched divalent heteroalkyl chain linking the rest of the molecule to a radical group, consisting of heteroatoms such as N, O and S Additional heteroatoms can also be useful, including, but not limited to, B, Al, Si and P. The heteroalkylene chain is attached to the rest of the molecule through a single bond and to the radical group through a single bond. In certain embodiments, a heteroalkylene comprises one heteroatom. In certain embodiments, a heteroalkylene comprises two heteroatoms. In certain embodiments, a heteroalkylene comprises three heteroatoms. In certain embodiments, a heteroalkylene comprises four heteroatoms. In certain embodiments, a heteroalkylene comprises five heteroatoms. In certain embodiments, the heteroatoms can be N, O, S, Si, or P, or a combination thereof in certain embodiments, the heteroatoms can be N, O, or S, or a combination thereof. In certain embodies, the heteroatoms can be N, O, or a combination thereof.

The term “C_{x-y}” or “Cₓ-C_{y}” when used in conjunction with a chemical moiety, such as alkyl, alkenyl, or alkynyl is meant to include groups that contain from x to y carbons in the chain. For example, the term “C₁₋₆alkyl” refers to substituted or unsubstituted saturated hydrocarbon groups, including straight-chain alkyl and branched-chain alkyl groups that contain from 1 to 6 carbons.

The terms “C_{x-y}alkenyl” and “C_{x-y}alkynyl” refer to substituted or unsubstituted unsaturated aliphatic groups analogous in length and possible substitution to the alkyls described above, but that contain at least one double or triple bond, respectively.

The term “carbocycle” as used herein refers to a saturated, unsaturated or aromatic ring in which each atom of the ring is carbon. Carbocycle includes 3- to 10-membered monocyclic rings, 5- to 12-membered bicyclic rings, 5- to 12-membered spiro bicycles, and 5- to 12-membered bridged rings. Each ring of a bicyclic carbocycle may be selected from saturated, unsaturated, and aromatic rings. In an exemplary embodiment, an aromatic ring, e.g., phenyl, may be fused to a saturated or unsaturated ring, e.g., cyclohexane, cyclopentane, or cyclohexene.

A bicyclic carbocycle includes any combination of saturated, unsaturated and aromatic bicyclic rings, as valence permits. A bicyclic carbocycle further includes spiro bicyclic rings such as spiropentane. A bicyclic carbocycle includes any combination of ring sizes such as 3-3 spiro ring systems, 4-4 spiro ring systems, 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. Exemplary carbocycles include cyclopentyl, cyclohexyl, cyclohexenyl, adamantyl, phenyl, indanyl, naphthyl, and bicyclo[1.1.1]pentanyl.

The term “aryl” refers to an aromatic monocyclic or aromatic multicyclic hydrocarbon ring system. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2)π-electron system in accordance with the Hückel theory. The ring system from which aryl groups are derived include, but are not limited to, groups such as benzene, fluorene, indane, indene, tetralin and naphthalene.

The term “cycloalkyl” refers to a saturated ring in which each atom of the ring is carbon. Cycloalkyl may include monocyclic and polycyclic rings such as 3- to 10-membered monocyclic rings, 5- to 12-membered bicyclic rings, 5- to 12-membered spiro bicycles, and 5- to 12-membered bridged rings. In certain embodiments, a cycloalkyl comprises three to ten carbon atoms. In other embodiments, a cycloalkyl comprises five to seven carbon atoms. The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, spiropentane, norbornyl (i.e., bicyclo[2.2.1]heptanyl), decalinyl, 7,7 dimethyl bicyclo[2.2.1]heptanyl, bicyclo[1.1.1]pentanyl, and the like.

The term “cycloalkenyl” refers to a saturated ring in which each atom of the ring is carbon and there is at least one double bond between two ring carbons. Cycloalkenyl may include monocyclic and polycyclic rings such as 3- to 10-membered monocyclic rings, 6- to 12-membered bicyclic rings, and 5- to 12-membered bridged rings. In other embodiments, a cycloalkenyl comprises five to seven carbon atoms. The cycloalkenyl may be attached to the rest of the molecule by a single bond. Examples of monocyclic cycloalkenyls include, e.g., cyclopentenyl, cyclohexenyl, cycloheptenyl, and cyclooctenyl.

The term “halo” or, alternatively, “halogen” or “halide,” means fluoro, chloro, bromo or iodo. In some embodiments, halo is fluoro, chloro, or bromo.

The term “haloalkyl” refers to an alkyl radical, as defined above, that is substituted by one or more halo radicals, for example, trifluoromethyl, dichloromethyl, bromomethyl, 2,2,2-trifluoroethyl, 1-chloromethyl-2-fluoroethyl, and the like. In some embodiments, the alkyl part of the haloalkyl radical is optionally further substituted as described herein.

The term “heterocycle” as used herein refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, 5- to 12-membered spiro bicycles, and 5- to 12-membered bridged rings. A monocylic heterocycle includes any saturated, unsaturated, and aromatic rings as valence permits. A monocyclic heterocycle includes but is not limited to, oxetane, azetidine, furan, tetrahydrofuran, pyrrole, pyrrolidine, pyran, piperidine, piperazine, imidazole, thiazole, morpholine, pyridine, and pyrimidine. A bicyclic heterocycle includes any combination of saturated, unsaturated and aromatic bicyclic rings, as valence permits. In an exemplary embodiment, an aromatic ring, e.g., pyridyl, may be fused to a saturated or unsaturated ring, e.g., cyclohexane, cyclopentane, morpholine, piperidine or cyclohexene. A bicyclic heterocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. Examples of fused ring systems include, but are not limited to, isoindoline, isoquinoline, tetrahydroisoquinoline, 3-azabicyclo[3.1.0]hexane and 6-oxa-3-azabicyclo[3.1.1]heptane. A bicyclic heterocycle further includes spiro bicyclic rings, e.g., 5 to 12-membered spiro bicycles, such as but not limited to 2-azaspiro[3.3]heptane, 5-azaspiro[2.4]heptane, 2-oxa-6-azaspiro[3.3]heptane, 2,6-diazaspiro[3.3]heptane, 1-thia-6-azaspiro[3.3]heptane, 6-azaspiro[3.4]octane, 2,6-diazaspiro[3.4]octane, 2-thia-6-azaspiro[3.4]octane, 4-oxa-7-azaspiro[2.5]octane, 2-azaspiro[4.4]nonane, 2,7-diazaspiro[4.4]nonane, 2-oxa-6-azaspiro[3.5]nonane, 7-oxa-2-azaspiro[3.5]nonane, 2-azaspiro[4.5]decane, 2,8-diazaspiro[4.5]decane, 8-oxa-2-azaspiro[4.5]decane, and 2-oxa-7-azaspiro[4.5]decane.

The term “heteroaryl” refers to a radical derived from a 5 to 18 membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is aromatic, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, benzimidazolyl, 1,3-benzodioxolyl, benzofuranyl, benzoxazolyl, benzo[d]thiazolyl, benzodioxinyl, benzopyranyl, benzopyranonyl, benzofuranyl, benzofuranonyl, benzothienyl (benzothiophenyl), benzotriazolyl, furanyl, isothiazolyl, imidazolyl, indazolyl, indolyl, indazolyl, isoindolyl, indolinyl, isoindolinyl, pyrrolyl, pyrazolyl, pyridinyl, pyridopyrimidinyl, pyrazinyl, pyrimidinyl, pyridazinyl, pyrrolyl, quinazolinyl, quinolinyl, isoquinolinyl, tetrahydroquinolinyl, and thiophenyl (i.e. thienyl).

The term “heterocycloalkyl” refers to a saturated ring with carbon atoms and at least one heteroatom. Exemplary heteroatoms include N, O, Si, P, B, and S atoms. Heterocycloalkyl may include monocyclic and polycyclic rings such as 3- to 10-membered monocyclic rings, 6- to 12-membered bicyclic rings, 5- to 12-membered spiro bicycles, and 5- to 12-membered bridged rings. The heteroatoms in the heterocycloalkyl radical are optionally oxidized. One or more nitrogen atoms, if present, are optionally quaternized. The heterocycloalkyl is attached to the rest of the molecule through any atom of the heterocycloalkyl, valence permitting, such as any carbon or nitrogen atoms of the heterocycloalkyl. Examples of heterocycloalkyl radicals include, but are not limited to, azetidinyl, dioxolanyl, decahydroisoquinolyl, imidazolinyl, imidazolidinyl, isothiazolidinyl, isoxazolidinyl, morpholinyl, octahydroindolyl, octahydroisoindolyl, oxazolidinyl, oxetanyl, piperidinyl, piperazinyl, pyrrolidinyl, pyrazolidinyl, quinuclidinyl, thiazolidinyl, tetrahydrofuryl, trithianyl, tetrahydropyranyl, thiomorpholinyl, 3-azabicyclo[3.1.0]hexane, 2-azaspiro[3.3]heptane, 5-azaspiro[2.4]heptane, 2-oxa-6-azaspiro[3.3]heptane, 2,6-diazaspiro[3.3]heptane, 6-oxa-3-azabicyclo[3.1.1]heptane, 1-thia-6-azaspiro[3.3]heptane, 6-azaspiro[3.4]octane, 2,6-diazaspiro[3.4]octane, 2-thia-6-azaspiro[3.4]octane, 4-oxa-7-azaspiro[2.5]octane, 2-azaspiro[4.4]nonane, 2,7-diazaspiro[4.4]nonane, 2-oxa-6-azaspiro[3.5]nonane, 7-oxa-2-azaspiro[3.5]nonane, 2-azaspiro[4.5]decane, 2,8-diazaspiro[4.5]decane, 8-oxa-2-azaspiro[4.5]decane, 2-oxa-7-azaspiro[4.5]decane, and 1,1-dioxo-thiomorpholinyl.

The term “heterocycloalkenyl” refers to an unsaturated ring with carbon atoms and at least one heteroatom and there is at least one double bond between two ring carbons.

Heterocycloalkenyl does not include heteroaryl rings. Exemplary heteroatoms include N, O, Si, P, B, and S atoms. Heterocycloalkenyl may include monocyclic and polycyclic rings such as 3- to 10-membered monocyclic rings, 6- to 12-membered bicyclic rings, and 5- to 12-membered bridged rings. In other embodiments, a heterocycloalkenyl comprises five to seven ring atoms.

The heterocycloalkenyl may be attached to the rest of the molecule by a single bond. Examples of monocyclic cycloalkenyls include, e.g., pyrroline (dihydropyrrole), pyrazoline (dihydropyrazole), imidazoline (dihydroimidazole), triazoline (dihydrotriazole), dihydrofuran, dihydrothiophene, oxazoline (dihydrooxazole), isoxazoline (dihydroisoxazole), thiazoline (dihydrothiazole), isothiazoline (dihydroisothiazole), oxadiazoline (dihydrooxadiazole), thiadiazoline (dihydrothiadiazole), dihydropyridine, tetrahydropyridine, dihydropyridazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine.

The term “substituted” refers to moieties having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, spirocyclic and non-spirocyclic, aromatic and non-aromatic substituents of organic compounds. The permissible substituents can be one or more and the same or different for appropriate organic compounds.

In some embodiments, each substituent may individually include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2), and —R^{b}—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl, any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)OR^{a} (where t is 1 or 2) and —R^{b}—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R^{b}—N(R^{a})C(O)OR^{a}, —R^{b}—N(R^{a})C(O)R^{a}, —R^{b}—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

Double bonds to oxygen atoms, such as oxo groups, are represented herein as both “═O” and “(O)”. Double bonds to nitrogen atoms are represented as both “═NR” and “(NR)”. Double bonds to sulfur atoms are represented as both “═S” and “(S)”.

The phrases “parenteral administration” and “administered parenterally” as used herein means modes of administration other than enteral and topical administration, usually by injection, and includes, without limitation, intravenous, intramuscular, intra-arterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

As used herein, “treatment” or “treating” refers to an approach for obtaining beneficial or desired results with respect to a disease, disorder, or medical condition including but not limited to a therapeutic benefit and/or a prophylactic benefit. A therapeutic benefit can include, for example, the eradication or amelioration of the underlying disorder being treated. Also, a therapeutic benefit can include, for example, the eradication or amelioration of one or more of the physiological symptoms associated with the underlying disorder such that an improvement is observed in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made. Treatment via administration of a compound described herein does not require the involvement of a medical professional.

TABLE
of Abbreviations

| | |
|---|---|
| ACN, MeCN | Acetonitrile |
| ° C. | Degree Celsius |
| boc | tert-butoxycarbonyl |
| Cmpd | Compound |
| DCM | Dichloromethane |
| DIEA or | Diisopropylethylamine |
| DIPEA | |
| DMF | Dimethylformamide |
| DMSO | Dimethylsulfoxide |
| EA, ETOAc, | Ethyl acetate |
| ETAC | |
| EDC, EDAC, | 1-Ethyl-3-(3- |
| EDCI | dimethylaminopropyl) |
| | carbodiimide |
| ESI | Electrospray |
| | ionization |
| HATU | Hexafluorophosphate |
| | Azabenzotriazole |
| | Tetramethyl Uronium |
| HOBt | Hydroxybenzotriazole |
| HPLC | High Performance |
| | Liquid Chromatography |
| IC₅₀ | Half minimal (50%) |
| | inhibitory concentration |
| LCMS | Liquid Chromatography |
| | Mass Spectrometry |
| [M + H+]+ | Mass peak plus hydrogen |
| or (MH)+ | |
| [M − H−]− | Mass peak minus hydrogen |
| or (MH)− | |
| Me | Methyl |
| MeOH | Methanol |
| MS | Mass spectrometry |
| N | Normal |
| PE | Petroleum ether |
| PMB | (4-methoxyphenyl) |
| | methanamine or para- |
| | methoxy benzyl |
| Prep-TLC | Preparative thin layer |
| | chromatography |
| PyBroP | bromotri(pyrrolidino) |
| | phosphonium hexafluorophosphate |
| RP | Reverse phase |
| RP-HPLC | Reverse phase high pressure liquid |
| | chromatography |
| RT or rt | Room temperature |
| TLC | thin-layer chromatography |
| THF | Tetrahydrofuran |
| TFA | Trifluoroacetic acid |

### Combination Therapies

The combination therapies contemplated in this disclosure include, for example, co-administering a disclosed compound and an additional therapeutic agent or therapy, as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually hours, days, weeks, months or years depending upon the combination selected). Combination therapy is intended to embrace administration of multiple therapeutic agents in a sequential manner, that is, wherein each therapeutic agent is administered at a different time, as well as administration of these therapeutic agents, or at least two of the therapeutic agents, in a substantially simultaneous manner.

Substantially simultaneous administration is accomplished, for example, by administering to the subject a single formulation or composition, (e.g., a tablet or capsule having a fixed ratio of each therapeutic agent or in multiple, single formulations (e.g., capsules) for each of the therapeutic agents. Sequential or substantially simultaneous administration of each therapeutic agent is affected by any appropriate route including, but not limited to, oral routes, intravenous routes, intramuscular routes, and direct absorption through mucous membrane tissues. The therapeutic agents are administered by the same route or by different routes. For example, a first therapeutic agent of the combination selected is administered by intravenous injection while the other therapeutic agents of the combination are administered orally. Alternatively, for example, all therapeutic agents are administered orally or all therapeutic agents are administered by intravenous injection.

The components of the combination are administered to a patient simultaneously or sequentially. It will be appreciated that the components are present in the same pharmaceutically acceptable carrier and, therefore, are administered simultaneously. Alternatively, the active ingredients are present in separate pharmaceutical carriers, such as, conventional oral dosage forms, that are administered either simultaneously or sequentially.

### Embodiments of Combination Therapies

Embodiment 1 of this disclosure relates to a method of treating a disease or condition comprising administering to a subject in need thereof a therapeutically effective amount of a compound having the structure of Formula (I0), or a pharmaceutically acceptable salt or solvate thereof, in combination with one or more additional therapeutic agents or therapies, wherein Formula (I0) is:

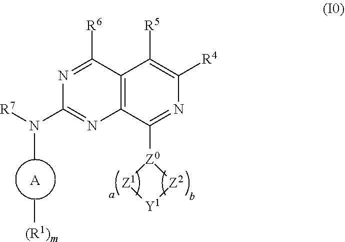

wherein,
   A is a ring selected from optionally substituted carbocycle, optionally substituted 4- to 8-membered heterocycle, optionally substituted tetrahydro-triazolopyrazine, and optionally substituted isoindoline;
   Z⁰ is —C(H)— or nitrogen;
   each of Z¹, Z², and Y¹ are independently selected from —C(R²)₂—, —C(O)—, —NR³—, —N(C(O)R²)—, —NS(O₂)R², —O—, —S—, —S(O)—, and —S(O)₂—;
   each of a and b are independently selected from 1, 2, 3, and 4;
   each R¹ is independently selected from halogen, —CN, —NO₂, optionally substituted alkyl, optionally substituted alkenyl, optionally substituted alkynyl, optionally substituted carbocycle, and optionally substituted heterocycle;
   m is selected from 0 to 5;
   each R² is independently selected from hydrogen, halogen, —CN, —OH, optionally substituted alkoxy, optionally substituted alkyl, optionally substituted cycloalkyl, optionally substituted —O-cycloalkyl, and optionally substituted heterocycloalkyl, or two R² substituents come together to form an optionally substituted heterocycle or an optionally substituted carbocycle, or R² and R³ substituents come together to form an optionally substituted heterocycle;
   each R³ is independently selected from hydrogen, optionally substituted alkyl, optionally substituted C₃₋₄ carbocycle, and optionally substituted 3- to 4-membered heterocycloalkyl;
   R⁴ is selected from hydrogen, halogen, —CN, optionally substituted C₁₋₄ alkyl, optionally substituted C₃₋₄ carbocycle, and optionally substituted 3- to 4-membered heterocycloalkyl;
each of R⁵, R⁶, is independently selected from hydrogen, halogen, —CN, optionally substituted C₁₋₄ alkyl, optionally substituted C₃₋₄ carbocycle, and optionally substituted 3- to 4-membered heterocycloalkyl; and
   R⁷ is selected from hydrogen and optionally substituted C₁₋₄ alkyl, and
   wherein if A is optionally substituted phenyl, m is from 1 to 5 and at least one R¹ is a heterocycloalkyl,
   wherein if A is optionally substituted pyridine, optionally substituted pyridazine, or optionally substituted pyrimidine, R⁴ is selected from hydrogen, halogen, and —CN,
   wherein if A is an optionally substituted piperidine sulfonamide, then either (i) Y¹ is —C(R²)₂— and the two R² substituents come together to form a ring selected from an optionally substituted heterocycle and an optionally substituted carbocycle, or (ii) R⁴ is selected from hydrogen, halogen, methyl, and —CN, and
   wherein if A-R¹ is

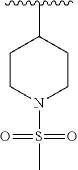 [SMILES: *C1CCN(S(C)(=O)=O)CC1]

and Z⁰ is CH, then R⁴ is methyl or cyclopropyl.

Embodiment 2 of this disclosure relates to the method according to Embodiment 1, wherein the compound, or a pharmaceutically acceptable salt or solvate thereof, has the structure of Formula (I):

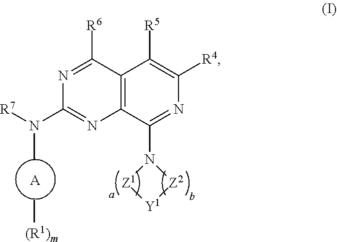

wherein, A is a ring selected from optionally substituted carbocycle, optionally substituted 4- to 6-membered heterocycle, and optionally substituted isoindoline.

Embodiment 3 of this disclosure relates to the method according to Embodiment 1 or 2, wherein the compound, or a pharmaceutically acceptable salt or solvate thereof, has the structure of one or more of Formulae (IA), (IB), (IC), (ID), or (IE):

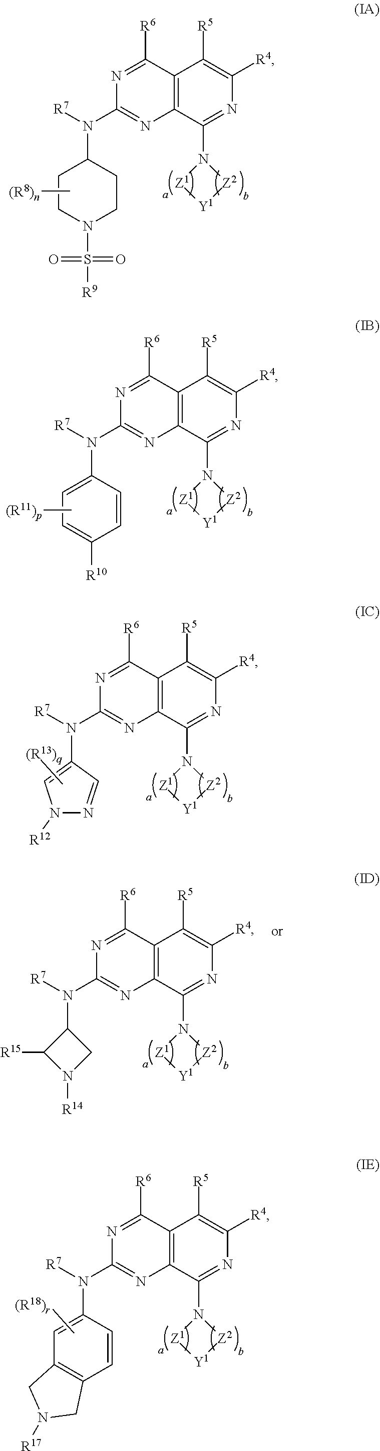

wherein,
   R⁸ is selected from halogen, —CN, and optionally substituted C₁₋₄ alkyl;
   R⁹ is selected from optionally substituted C₃₋₆ carbocycle, optionally substituted C₅₋₆ heteroaryl, and 3- to 6-membered heterocycloalkyl;
   R¹⁰ is optionally substituted alkyl or optionally substituted heterocycloalkyl;
   R¹¹ is selected from halogen, —CN, and optionally substituted C₁₋₄ alkyl;
   R¹² is selected from an optionally substituted heterocycloalkyl, optionally substituted heterocycloalkylalkyl, optionally substituted cycloalkyl, and optionally substituted cycloalkylalkyl; or R¹² and R¹³ come together to form an optionally substituted heterocycle;
   R¹³ is selected from halogen, —CN, and optionally substituted C₁₋₄ alkyl;
   n is selected from 0 to 9;
   R¹⁴ is selected from —SOR¹⁶—, and optionally substituted heterocycloalkyl;
   R¹⁵ is selected from hydrogen, halogen, —CN, and optionally substituted C₁₋₄ alkyl;
   R¹⁶ is selected from optionally substituted C₁₋₄ alkyl, optionally substituted C₃₋₆ carbocycle, and optionally substituted 3- to 6-membered heterocycloalkyl;
   R¹⁷ is selected from —SOR¹⁹—, optionally substituted alkyl, optionally substituted carbocycle, and optionally substituted heterocycloalkyl;
   R¹⁸ is selected from halogen, —CN, and optionally substituted C₁₋₄ alkyl;
   R¹⁹ is selected from optionally substituted C₁₋₄ alkyl, optionally substituted C₃₋₆ carbocycle, and optionally substituted 3- to 6-membered heterocycloalkyl;
   r is selected from 0 to 5.
   p is selected from 0 to 4;
   q is selected from 0 to 2; and
   wherein when the compound is of formula (IA), either (i) Y¹ is —C(R²)₂— and the two R² substituents come together to form a ring selected from an optionally substituted heterocycle and an optionally substituted carbocycle, or (ii) R⁴ is selected from hydrogen, halogen, and —CN.

Embodiment 3(a) relates to the method according to Embodiment 3, wherein the compound is the structure of Formula (IA).

Embodiment 3(b) relates to the method according to Embodiment 3, wherein the compound is the structure of Formula (IB).

Embodiment 3(c) relates to the method according to Embodiment 3, wherein the compound is the structure of Formula (IC).

Embodiment 3(d) relates to the method according to Embodiment 3, wherein the compound is the structure of Formula (IE).

Embodiment 4 of this disclosure relates to the method according to any one of the preceding embodiments, wherein Z¹ and Z² are independently selected from —C(R²)₂—, —NR³—, —O—, and —S—.

Embodiment 5 of this disclosure relates to the method according to Embodiment 4, wherein Z¹ and Z² are independently —C(R²)₂—.

Embodiment 6 of this disclosure relates to the method according to any one the preceding embodiments, wherein each of a and b are independently selected from 1 and 2.

Embodiment 7 of this disclosure relates to the method according to any one of the preceding embodiments, wherein Y¹ is selected from —C(R²)₂—, —NR³—, —NS(O₂)R², —O—, —S(O)₂—, and —S—.

Embodiment 8 of this disclosure relates to the method according to Embodiment 7, wherein Y¹ is selected from —C(R²)₂— and —NR³.

Embodiment 9 of this disclosure relates to any one the preceding embodiments, wherein each R² is independently selected from hydrogen, OH, halogen, —CN, optionally substituted alkyl, optionally substituted —O-alkyl, optionally substituted cycloalkyl, and optionally substituted heterocycloalkyl.

Embodiment 10 of this disclosure relates to the method according to Embodiment 9, wherein each R² is independently selected from hydrogen, halogen, —CN, cyclopropyl, cyclobutyl, optionally substituted cycloalkyl, and optionally substituted heterocycloalkyl.

Embodiment 11 of this disclosure relates to the method according to Embodiment 10, wherein each R² is independently selected from hydrogen, —CN and cyclopropyl.

Embodiment 12 of this disclosure relates to the method according to preceding embodiments, wherein two R² substituents come together to form an optionally substituted heterocycle or an optionally substituted carbocycle.

Embodiment 13 of this disclosure relates to the method according to any one of the preceding Embodiments, wherein each R³ is selected from hydrogen, optionally substituted alkyl, cyclopropyl, cyclobutyl, optionally substituted oxetane, and optionally substituted azetidine.

Embodiment 14 of this disclosure relates to the method according to Embodiment 17, wherein each R³ is selected from methyl, methoxyethylene, CD₃, cyclopropyl and cyclobutyl.

Embodiment 15 of this disclosure relates to the method according to Embodiment 18, wherein R³ is cyclopropyl.

Embodiment 16 of this disclosure relates to the method according to Embodiment 1, wherein the compound, or pharmaceutically acceptable salt thereof, has the structure of one or more of Formulae (IAA), (IBB), (ICC), (IDD), or (IEE):

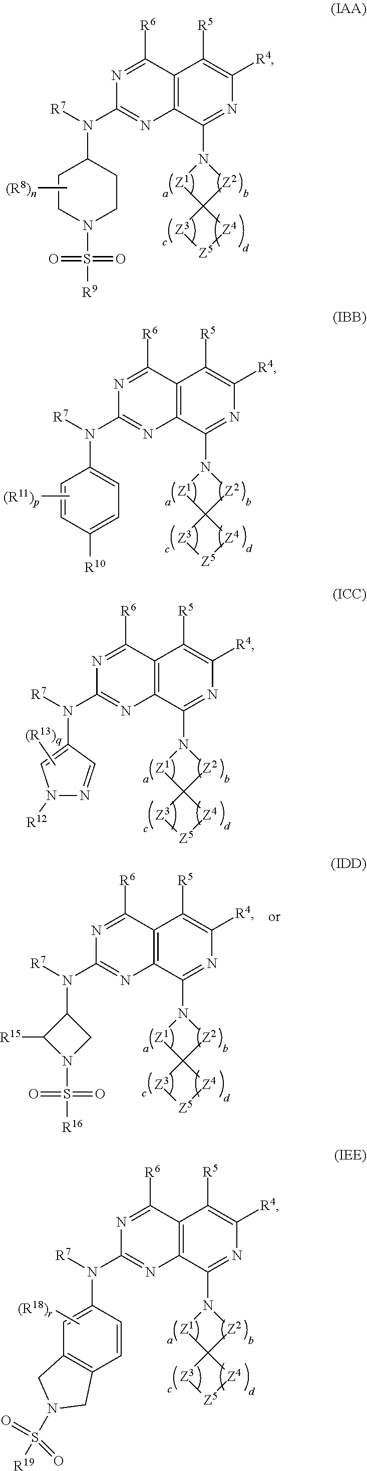

wherein,
   R⁸ is selected from halogen, —CN, and optionally substituted C₁₋₄ alkyl;
   R⁹ is selected from optionally substituted C₃₋₆ carbocycle, optionally substituted C₅₋₆ heteroaryl, and 3- to 6-membered heterocycloalkyl;
   R¹⁰ is optionally substituted heterocycloalkyl;
   R¹¹ is selected from halogen, —CN, and optionally substituted C₁₋₄ alkyl;
   R¹² is optionally substituted heterocycloalkyl; or R¹² and R¹³ come together to form an optionally substituted heterocycle;
   R¹³ is selected from halogen, —CN, and optionally substituted C₁₋₄ alkyl;
   R¹⁵ is selected from hydrogen, halogen, —CN, and optionally substituted C₁₋₄ alkyl;
   R¹⁶ is selected from optionally substituted C₁₋₄ alkyl, optionally substituted C₃₋₆ carbocycle, and optionally substituted 3- to 6-membered heterocycloalkyl;
   R¹⁹ is selected from optionally substituted C₁₋₄ alkyl, optionally substituted C₃₋₆ carbocycle, and optionally substituted 3- to 6-membered heterocycloalkyl;
   n is selected from 0 to 9;
   p is selected from 0 to 4;
   q is selected from 0 to 2;
   each of Z¹, Z², Z³, Z⁴ and Z⁵ are independently selected from —C(R²)₂—, —C(O)—, —NR³—, —N(C(O)R²)—, —NS(O₂)R³, —O—, —S—, —S(O)—, and —S(O)₂—, wherein Z⁵ is additionally selected from a bond; and
   each of a, b, c, and d are independently selected from 1, 2, 3, and 4.

Embodiment 16(a) relates to the method according to Embodiment 16 wherein the compound is the structure of Formula (IAA).

Embodiment 16(b) relates to the method according to Embodiment 16 wherein the compound is the structure of Formula (IBB).

Embodiment 16(c) relates to the method according to Embodiment 16 wherein the compound is the structure of Formula (ICC).

Embodiment 16(d) relates to the method according to Embodiment 16 wherein the compound is the structure of Formula (IEE).

Embodiment 17 of this disclosure relates to the method according to Embodiment 16, wherein each of Z¹, Z², Z³, Z⁴ and Z⁵ are independently selected from —C(R²)₂—, —NR³—, —N(C(O)R²)—, —NS(O₂)R³, —O—, and —S(O)₂—, wherein Z⁵ is additionally selected from a bond.

Embodiment 18 of this disclosure relates to the method according to Embodiment 17, wherein each of Z¹, Z², Z³, Z⁴ and Z⁵ are independently selected from —C(R²)₂—, —NR³—, —O—, and —S(O)₂—, wherein Z⁵ is additionally selected from a bond.

Embodiment 19 of this disclosure relates to the method according to any one of Embodiments 16-18, wherein each R² is independently selected from hydrogen, halogen, —CN, OH, optionally substituted alkyl, optionally substituted cycloalkyl, and optionally substituted heterocycloalkyl, or two R² substituents come together to form an optionally substituted heterocycle or an optionally substituted carbocycle.

Embodiment 20 of this disclosure relates to the method according to Embodiment 19, wherein each R² is selected from hydrogen, fluoro, CN, cyclopropyl, cyclobutyl, —C₁₋₄ alkyl, —C₁₋₄ haloalkyl, —O—C₁₋₄ alkyl, —C₁₋₄ alkylene-O—C₁₋₃ alkyl, —C₁₋₄ alkylene-OH, optionally substituted oxetane, and optionally substituted azetidine.

Embodiment 21 of this disclosure relates to the method according to any one of Embodiments 16-20, wherein R³ is selected from hydrogen, —(CH₂)₂OMe, —S(O)₂CH₃, —C₁₋₄ alkylene-O—C₁₋₃ alkyl, C₁₋₄ alkyl, cyclopropyl, cyclobutyl, optionally substituted oxetane, and optionally substituted azetidine.

Embodiment 22 of this disclosure relates to the method according to Embodiment 21, wherein R³ is selected from hydrogen, —(CH₂)₂OMe, C₁₋₄ alkyl, cyclopropyl, cyclobutyl, optionally substituted oxetane, and optionally substituted azetidine.

Embodiment 23 of this disclosure relates to the method according to Embodiment 22, wherein R³ is selected from hydrogen, methyl, ethyl, propyl, CD₃, and cyclopropyl.

Embodiment 24 of this disclosure relates to the method according to any one of Embodiments 16-23, wherein each a, b, c, and d are each independently selected Embodiments 1 and 2.

Embodiment 25 of this disclosure relates to the method according to any one of Embodiments 1 or 2, wherein A is selected from optionally substituted cyclohexane, optionally substituted pyridine, optionally substituted piperidine, optionally substituted tetrahydropyran, optionally substituted azabicyclo[3.1.0]hexane, optionally substituted azetidine, and optionally substituted tetrahydroisoquinoline.

Embodiment 26 of this disclosure relates to the method according to Embodiment 25, wherein A is optionally substituted piperidine.

Embodiment 27 of this disclosure relates to the method according to Embodiment 26, wherein A is substituted with SO₂R⁹, wherein R⁹ is selected from optionally substituted C₃₋₆ carbocycle, optionally substituted C₅₋₆ heteroaryl, and 3- to 6-membered heterocycloalkyl.

Embodiment 28 of this disclosure relates to the method according to any one of Embodiments 1 or 2, wherein m is selected from 0 to 1.

Embodiment 29 of this disclosure relates to the method according to any one of Embodiments 1 or 2, wherein each R¹ is independently selected from optionally substituted alkyl, optionally substituted carbocycle, and optionally substituted heterocycle.

Embodiment 30 of this disclosure relates to the method according to Embodiment 29, wherein each R¹ is independently selected from optionally substituted alkyl and optionally substituted heterocycle.

Embodiment 31 of this disclosure relates to the method according to Embodiment 29, wherein each R¹ is independently selected from methyl, ethyl, and optionally substituted diazaspiro[3.3]heptane.

Embodiment 32 of this disclosure relates to the method according to any of any one of Embodiments 16-31, wherein Z¹ and Z² are independently selected from —C(R²)₂—, —NR³—, —O—, and —S—.

Embodiment 33 of this disclosure relates to the method according to Embodiment 32, wherein Z¹ and Z² are each —C(R²)₂—.

Embodiment 34 of this disclosure relates to the method according to any one of Embodiments 32 or 33, wherein each of a and b are independently selected from 1 and 2.

Embodiment 35 of this disclosure relates to the method according to Embodiment 34, wherein Y¹ is —C(R²)₂— and two R² substituents come together to form a ring selected from an optionally substituted heterocycle and an optionally substituted carbocycle.

Embodiment 36 of this disclosure relates to the method according to Embodiment Error!Reference source not found., wherein Y¹ is —C(R²)₂— and the two R² substituents come together to form a ring selected from an optionally substituted heterocycle.

Embodiment 37 of this disclosure relates to the method according to any one of Embodiments 1-3, wherein the N containing heterocyclic ring depicted as

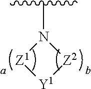

in Formula (I), (IA), (IB), (IC), (ID), and (IE) is selected from optionally substituted azetidine, optionally substituted pyrrolidine, optionally substituted piperidine, optionally substituted piperazine, optionally substituted morpholine, optionally substituted tetrahydrothienopyrroledioxide, and optionally substituted dihydroindole.

Embodiment 38 of this disclosure relates to the method according to Embodiments 1-3, wherein the N containing heterocyclic ring depicted as

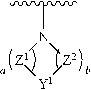

in Formula (I), (IA), (IB), (IC), (ID), and (IE) is selected from

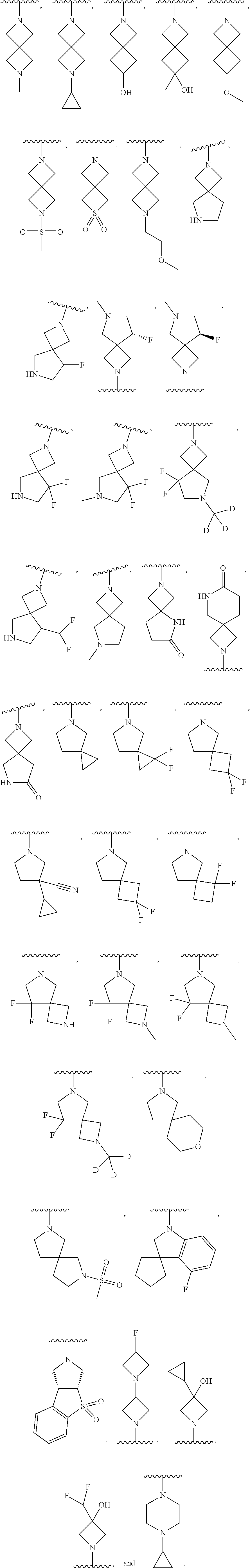

Embodiment 38(a) relates to the method according to Embodiment 38, wherein the N containing heterocyclic ring depicted as

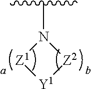

### in Formula (I), (IA), (IB), (IC), (ID), and (IE) is

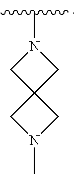 [SMILES: *N1CC2(C1)CN(C)C2]

Embodiment 38(b) relates to the method according to Embodiment 38, wherein the N containing heterocyclic ring depicted as

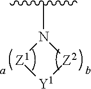

### in Formula (I), (IA), (IB), (IC), (ID), and (IE) is

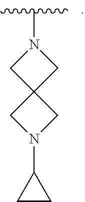 [SMILES: *N1CC2(C1)CN(C1CC1)C2]

Embodiment 38(c) relates to the method according to Embodiment 38, wherein the N containing heterocyclic ring depicted as

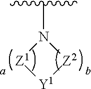

### in Formula (I), (IA), (IB), (IC), (ID), and (IE) is

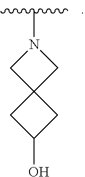 [SMILES: *N1CC2(CC(O)C2)C1]

Embodiment 38(d) relates to the method according to Embodiment 38, wherein the N containing heterocyclic ring depicted as

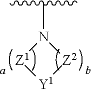

### in Formula (I), (IA), (IB), (IC), (ID), and (IE) is

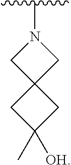 [SMILES: *N1CC2(C1)CC(C)(O)C2]

Embodiment 38(e) relates to the method according to Embodiment 38, wherein the N containing heterocyclic ring depicted as

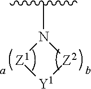

### in Formula (I), (IA), (IB), (IC), (ID), and (IE) is

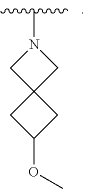 [SMILES: *N1CC2(CC(OC)C2)C1]

Embodiment 38(f) relates to the method according to Embodiment 38, wherein the N containing heterocyclic ring depicted as

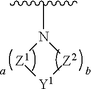

### in Formula (I), (IA), (IB), (IC), (ID), and (IE) is

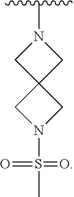 [SMILES: *N1CC2(C1)CN(S(C)(=O)=O)C2]

Embodiment 38(g) relates to the method according to Embodiment 38, wherein the N containing heterocyclic ring depicted as

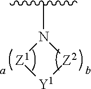

### in Formula (I), (IA), (IB), (IC), (ID), and (IE) is

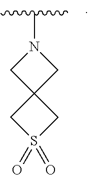 [SMILES: *N1CC2(C1)CS(=O)(=O)C2]

Embodiment 38(h) relates to the method according to Embodiment 38, wherein the N containing heterocyclic ring depicted as

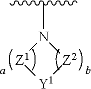

### in Formula (I), (IA), (IB), (IC), (ID), and (IE) is

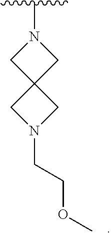 [SMILES: *N1CC2(C1)CN(CCOC)C2]

Embodiment 38(i) relates to the method according to Embodiment 38, wherein the N containing heterocyclic ring depicted as

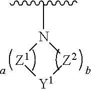

### in Formula (I), (IA), (B), (IC), (ID), and (IE) is

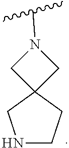 [SMILES: *N1CC2(CCNC2)C1]

Embodiment 38(j) relates to the method according to Embodiment 38, wherein the N containing heterocyclic ring depicted as

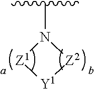

### in Formula (I), (IA), (IB), (IC), (ID), and (IE) is

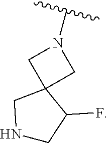 [SMILES: *N1CC2(CNCC2F)C1]

Embodiment 38(k) relates to the method according to Embodiment 38, wherein the N containing heterocyclic ring depicted as

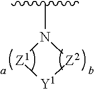

### in Formula (I), (IA), (IB), (IC), (ID), and (IE) is,

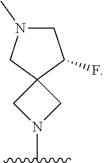 [SMILES: *N1CC2(C1)CN(C)C[C@@H]2F]

Embodiment 38(1) relates to the method according to Embodiment 38, wherein the N containing heterocyclic ring depicted as

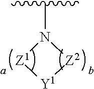

### in Formula (I), (IA), (IB), (IC), (ID), and (IE) is

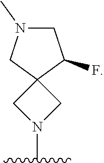 [SMILES: *N1CC2(C1)CN(C)C[C@H]2F]

Embodiment 38(m) relates to the method according to Embodiment 38, wherein the N containing heterocyclic ring depicted as

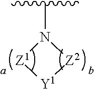

### in Formula (I), (IA), (IB), (IC), (ID), and (IE) is

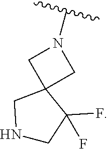 [SMILES: *N1CC2(CNCC2(F)F)C1]

Embodiment 38(n) relates to the method according to Embodiment 38, wherein the N containing heterocyclic ring depicted as

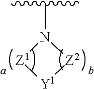

### in Formula (I), (IA), (IB), (IC), (ID), and (IE) is

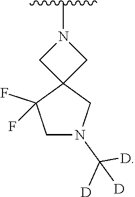

Embodiment 38(o) relates to the method according to Embodiment 38, wherein the N containing heterocyclic ring depicted as

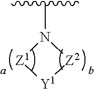

### in Formula (I), (IA), (IB), (IC), (ID), and (IE) is

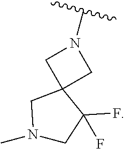 [SMILES: *N1CC2(C1)CN(C)CC2(F)F]

Embodiment 38(p) relates to the method according to Embodiment 38, wherein the N containing heterocyclic ring depicted as

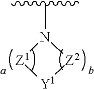

### in Formula (I), (IA), (IB), (IC), (ID), and (IE) is

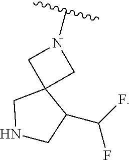 [SMILES: *N1CC2(CNCC2C(F)F)C1]

Embodiment 38(q) relates to the method according to Embodiment 38, wherein the N containing heterocyclic ring depicted as

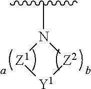

### in Formula (I), (IA), (IB), (IC), (ID), and (IE) is

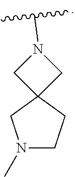 [SMILES: *N1CC2(CCN(C)C2)C1]

Embodiment 38(r) relates to the method according to Embodiment 38, wherein the N containing heterocyclic ring depicted as

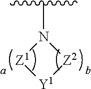

### in Formula (I), (IA), (IB), (IC), (ID), and (IE) is

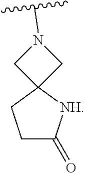 [SMILES: *N1CC2(CCC(=O)N2)C1]

Embodiment 38(s) relates to the method according to Embodiment 38, wherein the N containing heterocyclic ring depicted as

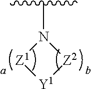

### in Formula (I), (IA), (IB), (IC), (ID), and (IE) is

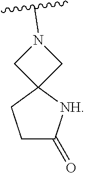 [SMILES: *N1CC2(CCC(=O)N2)C1]

Embodiment 38(t) relates to the method according to Embodiment 38, wherein the N containing heterocyclic ring depicted as

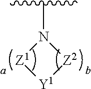

### in Formula (I), (IA), (IB), (IC), (ID), and (IE) is

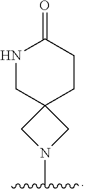 [SMILES: *N1CC2(CCC(=O)NC2)C1]

Embodiment 38(u) relates to the method according to Embodiment 38, wherein the N containing heterocyclic ring depicted as

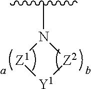

### in Formula (I), (IA), (IB), (IC), (ID), and (IE) is

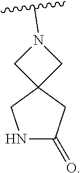 [SMILES: *N1CC2(CNC(=O)C2)C1]

Embodiment 38(v) relates to the method according to Embodiment 38, wherein the N containing heterocyclic ring depicted as

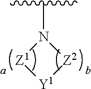

### in Formula (I), (IA), (IB), (IC), (ID), and (IE) is

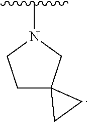 [SMILES: *N1CCC2(CC2)C1]

Embodiment 38(w) relates to the method according to Embodiment 38, wherein the N containing heterocyclic ring depicted as

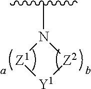

### in Formula (I), (IA), (IB), (IC), (ID), and (IE) is

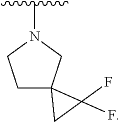 [SMILES: *N1CCC2(C1)CC2(F)F]

Embodiment 38(x) relates to the method according to Embodiment 38, wherein the N containing heterocyclic ring depicted as

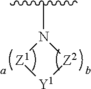

### in Formula (I), (IA), (IB), (IC), (ID), and (IE) is

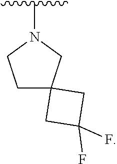 [SMILES: *N1CCC2(C1)CC(F)(F)C2]

Embodiment 38(y) relates to the method according to Embodiment 38, wherein the N containing heterocyclic ring depicted as

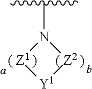

### in Formula (I), (IA), (IB), (IC), (ID), and (IE) is

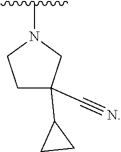 [SMILES: *N1CCC(C#N)(C2CC2)C1]

Embodiment 38(z) relates to the method according to Embodiment 38, wherein the N containing heterocyclic ring depicted as

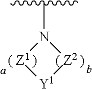

### in Formula (I), (IA), (IB), (IC), (ID), and (IE) is

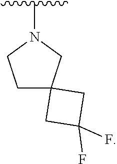 [SMILES: *N1CCC2(C1)CC(F)(F)C2]

Embodiment 38(aa) relates to the method according to Embodiment 38, wherein the N containing heterocyclic ring depicted as

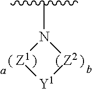

### in Formula (I), (IA), (IB), (IC), (ID), and (IE) is

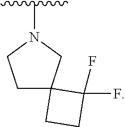 [SMILES: *N1CCC2(CCC2(F)F)C1]

Embodiment 38(ab) relates to the method according to Embodiment 38, wherein the N containing heterocyclic ring depicted as

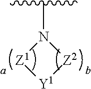

### in Formula (I), (IA), (IB), (IC), (ID), and (IE) is

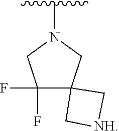 [SMILES: *N1CC(F)(F)C2(CNC2)C1]

Embodiment 38(ac) relates to the method according to Embodiment 38, wherein the N containing heterocyclic ring depicted as

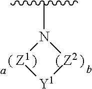

### in Formula (I), (IA), (IB), (IC), (ID), and (IE) is

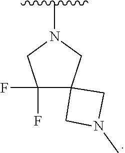 [SMILES: *N1CC(F)(F)C2(C1)CN(C)C2]

Embodiment 38(ad) relates to the method according to Embodiment 38, wherein the N containing heterocyclic ring depicted as

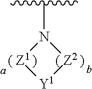

### in Formula (I), (IA), (IB), (IC), (ID), and (IE) is

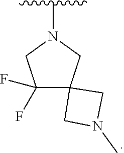 [SMILES: *N1CC(F)(F)C2(C1)CN(C)C2]

Embodiment 38(ae) relates to the method according to Embodiment 38, wherein the N containing heterocyclic ring depicted as

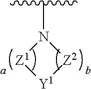

### in Formula (I), (IA), (IB), (IC), (ID), and (IE) is

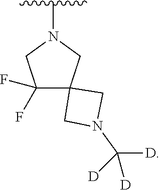

Embodiment 38(af) relates to the method according to Embodiment 38, wherein the N containing heterocyclic ring depicted as

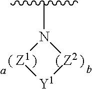

### in Formula (I), (IA), (IB), (IC), (ID), and (IE) is

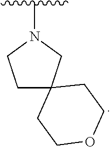 [SMILES: *N1CCC2(CCOCC2)C1]

Embodiment 38(ag) relates to the method according to Embodiment 38, wherein the N containing heterocyclic ring depicted as

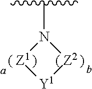

### in Formula (I), (IA), (IB), (IC), (ID), and (IE) is

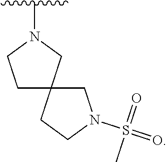 [SMILES: *N1CCC2(CCN(S(C)(=O)=O)C2)C1]

Embodiment 38(ah) relates to the method according to Embodiment 38, wherein the N containing heterocyclic ring depicted as

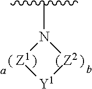

### in Formula (I), (IA), (IB), (IC), (ID), and (IE) is

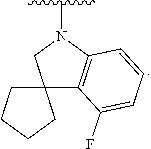 [SMILES: *N1CC2(CCCC2)c2c(F)cccc21]

Embodiment 38(ai) relates to the method according to Embodiment 38, wherein the N containing heterocyclic ring depicted as

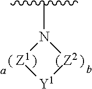

### in Formula (I), (IA), (IB), (IC), (ID), and (IE) is

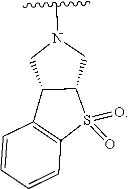 [SMILES: *N1C[C@H]2c3ccccc3S(=O)(=O)[C@H]2C1]

Embodiment 38(aj) relates to the method according to Embodiment 38, wherein the N containing heterocyclic ring depicted as

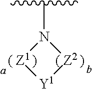

### in Formula (I), (IA), (IB), (IC), (ID), and (IE) is

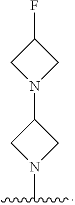 [SMILES: *N1CC(N2CC(F)C2)C1]

Embodiment 38(ak) relates to the method according to Embodiment 38, wherein the N containing heterocyclic ring depicted as

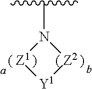

### in Formula (I), (IA), (IB), (IC), (ID), and (IE) is

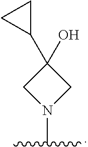 [SMILES: *N1CC(O)(C2CC2)C1]

Embodiment 38(al) relates to the method according to Embodiment 38, wherein the N containing heterocyclic ring depicted as

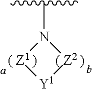

### in Formula (I), (IA), (IB), (IC), (ID), and (IE) is

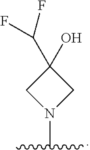 [SMILES: *N1CC(O)(C(F)F)C1]

Embodiment 38(am) relates to the method according to Embodiment 38, wherein the N containing heterocyclic ring depicted as

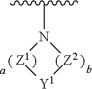

### in Formula (I), (IA), (IB), (IC), (ID), and (IE) is

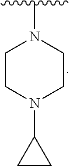 [SMILES: *N1CCN(C2CC2)CC1]

Embodiment 39 of this disclosure relates to the method according to any one of the preceding embodiments, wherein R⁴ is selected from hydrogen, halogen, CN, optionally substituted C₁₋₂ alkyl, optionally substituted C₃₋₄cycloalkyl, and optionally substituted 3- to 4 membered heterocycloalkyl.

Embodiment 40 of this disclosure relates to the compound or pharmaceutically acceptable salt according to Embodiment 39, wherein R⁴ is selected from hydrogen, cyclopropyl, halogen, methyl, and —CHF₂.

Embodiment 41 of this disclosure relates to the method according to any one of the preceding Embodiments, wherein R⁵ is selected from hydrogen, halogen, optionally substituted C₁₋₂ alkyl, optionally substituted C₃₋₄ carbocycle, and optionally substituted 3- to 4 membered heterocycloalkyl.

Embodiment 42 of this disclosure relates to the method according to Embodiment 41, wherein R⁵ is selected from hydrogen, halogen, and optionally substituted C₁₋₂ alkyl.

Embodiment 43 of this disclosure relates to the method according to Embodiment 41, wherein R⁵ is selected from hydrogen and fluoro.

Embodiment 44 of this disclosure relates to the method according to any one of the preceding Embodiments, wherein R⁶ is selected from hydrogen, halogen, optionally substituted C₁₋₂ alkyl, optionally substituted C₃₋₄ cycloalkyl, and optionally substituted 3- to 4 membered heterocycloalkyl.

Embodiment 45 of this disclosure relates to the method according to Embodiment 44, wherein R⁶ is selected from hydrogen, halogen, and optionally substituted C₁₋₂ alkyl.

Embodiment 46 of this disclosure relates to the method according to any one of the preceding Embodiments, wherein R⁶ is hydrogen.

Embodiment 47 of this disclosure relates to the method according to any one of the preceding Embodiments, wherein R⁷ is hydrogen.

Embodiment 48 of this disclosure relates to the method according to any one of the preceding embodiments, wherein R⁹ is selected from cyclopentyl, methylcyclopentyl, cyclobutylmethylene, cyclopentylmethylene, and n-methyl pyrazolyl.

Embodiment 48(a) relates to the method according to Embodiment 48 wherein R⁹ is cyclopentyl.

Embodiment 48(b) relates to the method according to Embodiment 48 wherein R⁹ is methylcyclopentyl.

Embodiment 48(c) relates to the method according to Embodiment 48 wherein R⁹ is cyclobutylmethylene.

Embodiment 48(d) relates to the method according to Embodiment 48 wherein R⁹ is cyclopentylmethylene.

Embodiment 48(e) relates to the method according to Embodiment 48 wherein R⁹ is n-methylpyrazolyl.

Embodiment 49 of this disclosure relates to the method according to Embodiment 1, wherein the compound, or pharmaceutically acceptable salt thereof, is selected from Table 1.

Embodiment 50 of this disclosure relates to the method according to Embodiment 2, wherein the compound, or pharmaceutically acceptable salt thereof, is selected from Table 1.

Embodiment 51 of this disclosure relates to the method according to Embodiment 16, wherein the compound, or pharmaceutically acceptable salt thereof, is selected from Table 1.

Embodiment 52 of this disclosure relates to the method according to Embodiment 38, wherein the compound, or pharmaceutically acceptable salt thereof, is selected from Table 1.

Additional embodiments of the compounds in the methods of this disclosure are also described in this disclosure.

Embodiment 53 of this disclosure relates to a method of treating a disease or condition comprising administering to a subject in need thereof a therapeutically effective amount of a pharmaceutical composition comprising a compound according to any of the preceding embodiments, or pharmaceutically acceptable salt thereof, and a pharmaceutically acceptable excipient.

Additional embodiments of the pharmaceutical formulations in the methods of this disclosure are also described in this disclosure.

Embodiment 54 of this disclosure relates to a method of treating a disease or condition according to any one of the preceding Embodiments, wherein the one or more additional therapeutic agents are selected from the group consisting of a platinum compound, a taxane, apigenin, a Wee-1 inhibitor, a PRMT5 inhibitor, an MDM2 inhibitor, a Src inhibitor, a Raf inhibitor, a MEK inhibitor, an XPO1 inhibitor, a vitamin D analog, a type-I TGF-b receptor inhibitor, a TRK inhibitor, a tankyrase inhibitor, a senolytic agent, a RET inhibitor, a proteosome inhibitor, a menin inhibitor, an LC 3-KAT inhibitor, a KIT inhibitor, a KIFC inhibitor, an IGF-1R inhibitor, an HIF-2 alpha inhibitor, an HER2 antibody drug conjugate, a heat shock protein, an HDAC inhibitor, a GLI1 inhibitor, a FOXM1 inhibitor, an EZH2 inhibitor, an estrogen receptor antagonist, an estrogen receptor alpha antagonist, an elF4A inhibitor, a dihydrofolate reductase inhibitor, a CD73 inhibitor, a BTK inhibitor a BMI inhibitor, a beta-catenin inhibitor, a CBP/p300 dual inhibitor, an ALDH1A3 inhibitor, a dual c-Met/Trk inhibitor, an antrogen receptor inhibitor, a Bcl-2 inhibitor, glyoxalase 1 inhibitor, a KIFC1 inhibitor, a USP10 inhibitor, an antioxidant defense inhibitor, RANKL inhibitor, a FLT3 inhibitor, a notch inhibitor, a BRAF inhibitor, a HER2 inhibitor, an eIF4A inhibitor, SHP2 inhibitor, an ERK1/2 inhibitor, an EGFR inhibitor, an IKK beta inhibitor, a PAK inhibitor, a steroid, a steroidogenesis inhibitor, a KIT D816V inhibitor, BET inhibitor, a PI3K inhibitor, an mTOR inhibitor, an FGFR inhibitor, a pan-ERBB inhibitor, an ALK-inhibitor, an anti-PD-1 monoclonal antibody, an anti-PD-L1 monoclonal antibody, an autophagy inhibitor, a YAP-inhibitor, an androgen receptor inhibitor, a PARP inhibitor, a FOXM1 inhibitor, an aromatase inhibitor, a CDK2 inhibitor, a CDK4 inhibitor, and a CDK4/6 inhibitor.

Embodiment 55 of this disclosure relates to a method of treating a disease or condition according to any one of the preceding Embodiments, wherein the one or more agents are selected from the group consisting of mTOR inhibitors, PI3K inhibitors, PARP inhibitors, Her2 antibody drug conjugates, and Trop2 antibody drug conjugates.

Embodiment 56 of this disclosure relates to a method of treating a disease or condition according to any one of the preceding Embodiments, wherein the method further comprises hormone therapy.

Embodiment 57 of this disclosure relates to a method of treating a disease or condition according to any one of the preceding Embodiments, wherein the one or more additional therapeutic agents are selected from the group consisting of carboplatin, cisplatin oxaliplatin, nedaplatin, phenanthriplatin, lobaplatin, enloplatin, paclitaxel, docetaxel, apigenin, adavosertib, pemrametosta, AZD1775, inecalcitol, SNDX-50469, NVP-CGM097, idasanutlin, nutlin-3, siremadlin, brigimadlin, saracatinib, bosutinib, dasatinib, sorafenib, trametinib, binimetinib, cobimetinib, KPT-330, SB-505124, entrectinib, MSC2504877, alectinib. bortezomib, selumetinib, PD0325901, trimetazidine, midostaurin, avapritinib, nintedanibispinesib, SR31527, ganitumab, NVP-AEW541, PT2399, T-DM1, SHetA2, tucidinostat, suberanilohydroxamic acid, vorinostat, valproate, GANT61, NB55, NB73, NB115AQB, lasofoxifene, CR-1-31-B, celastrol, gambogic acid, pralatrexate, ibrutinib, tirabrutinib, PTC-209, ICG-001, NE02734, N,N-diethylaminobenzaldehyde, altiratinib, seviteronel, RAD140, MLN0128, temsirolimus, sapanisertib, navitoclax, venetoclax, BBGC, ispinesib, spautin-1, auranofin, neratinib, pyrotinib, tucatinib, OPG-Fc, quizartinib, CB-103, encorafenib, vemurafenib, dabrafenib, trastuzumab, CR-1-31-B, TNO155, SCH772984, LY3214996, cetuximab, PF-0647775, erlotinib, Bay 11-7082, PF03758309, progesterone, mitotane, midostaurin, avapritinib, JQ1, ZEN-3694, ARV-825, alpelisib, pictilisib, vistusertib, everolimus, infigratinib, LY2874455, rogaratinib, BLU9931, H3B-6527, FIIN-2, FIIN-3, lenvatinib, ponatinib, regorafenib. Pemigatinib, paxalisib, futibatinib, infigratinib, afatinib, ceritinib, pembrolizumab, nivolumab, cemiplimab, atezolizumab, avelumab, durvalumab, hydroxychloroquine, chloroquine, verteporfin, pyrvinium pamoate, enzalutamide, N-desmethyl enzalutamide, darolutamide, apalutamide, ralaniten, EPI-7170, abiraterone, bafilomycin A1, albendazole, letrozole, fulvestrant, olaparib, talazoparib, NB55, NB73, NB 115, gedatolisib, eribulin, temozolomide, cytarabine, doxorubicin, gemcitabine, pemetrexed, dexamethasone, L-asparaginase, vincristine, 5-FU, sunitinib, and irinotecan.

Embodiment 58 of this disclosure relates to a method of treating a disease or condition according to Embodiment 57, wherein the one or more additional therapeutic agents are selected from the group consisting of vistusertib, MLN0128, alpelisib, buparlisib, paxalisib, gedatolisib, pictilisib, talazoparib, laparib, T-DM1, sacituzumab govtecan, and datopotamab deruxtecan.

Embodiment 59 of this disclosure relates to a method of treating a disease or condition according to Embodiment 57, wherein the one or more additional therapeutic agents are selected from the group consisting of carboplatin, cisplatin, oxaliplatin, nedaplatin, phenanthriplatin, lobaplatin, and enloplatin.

Embodiment 60 of this disclosure relates to a method of treating a disease or condition according to Embodiment 57, wherein the one or more additional therapeutic agents are selected from the group consisting of paclitaxel and docetaxel.

Embodiment 61 of this disclosure relates to a method of treating a disease or condition according to Embodiment 57, wherein the one or more additional therapeutic agents are selected from the group consisting of apigenin.

Embodiment 62 of this disclosure relates to a method of treating a disease or condition according to Embodiment 57, wherein the one or more additional therapeutic agents are selected from the group consisting of adavosertib, pemrametosta, nutlin-3, saracatinib, and dasatinib.

Embodiment 63 of this disclosure relates to a method of treating a disease or condition according to Embodiment 57, wherein the one or more additional therapeutic agents are selected from the group consisting of sorafenib, trametinib, cobimetinib, alpelisib, pictilisib, vistusertib, and irinotecan.

Embodiment 64 of this disclosure relates to a method of treating a disease or condition according to Embodiment 57, wherein the one or more additional therapeutic agents are selected from the group consisting of FIIN-2, FIN-3, lenvatinib, ponatinib, regorafenib, pemigatinib, futibatinib, and infigratinib.

Embodiment 65 of this disclosure relates to a method of treating a disease or condition according to Embodiment 57, wherein the one or more additional therapeutic agents are selected from the group consisting of afatinib and ceritinib.

Embodiment 66 of this disclosure relates to a method of treating a disease or condition according to Embodiment 57, wherein the one or more additional therapeutic agents are selected from the group consisting of pembrolizumab, nivolumab, cemiplimab, atezolizumab, avelumab, and durvalumab.

Embodiment 67 of this disclosure relates to a method of treating a disease or condition according to Embodiment 57, wherein the one or more additional therapeutic agents are selected from the group consisting of hydroxychloroquine, verteporfin, and olaparib.

Embodiment 68 of this disclosure relates to a method of treating a disease or condition according to Embodiment 57, wherein the one or more additional therapeutic agents are selected from the group consisting of enzalutamide, N-desmethyl enzalutamide, darolutamide, apalutamide, and abiraterone.

Embodiment 69 of this disclosure relates to a method of treating a disease or condition according to any one of the preceding Embodiments, wherein therapy is radiotherapy or ultrasound therapy.

Embodiment 69(a) relates to the method according to Embodiment 69, wherein the therapy is radiotherapy.

Embodiment 69(b) relates to the method according to Embodiment 69, wherein the therapy is ultrasound therapy.

Embodiment 70 of this disclosure relates to a method of treating a disease or condition according to any one of the preceding embodiments, wherein the disease or condition is cancer.

Embodiment 71 of this disclosure relates to a method of treating a disease or condition according to Embodiment 70, wherein the cancer is a solid tumor.

Embodiment 72 of this disclosure relates to a method of treating a disease or condition according to Embodiment 70, wherein the cancer is ovarian cancer, pancreatic cancer (such as, for example, pancreatic ductal adenocarcinoma), bladder cancer, brain cancer, sarcoma, melanoma (such as, for example, BRAF-mutant melanoma or NRAS-mutant melanoma), lung cancer (such as, for example, BRAF-mutant non-small cell lung cancer, RAS-mutant non-small cell lung cancer, RET fusion positive non-small lung cancer, squamous cell lung cancer, or KRAS mutant non-small cell lung cancer), mantle cell lymphoma, large B-cell lymphoma (diffuse large B-cell lymphoma), leukemia (such as, for example, acute myeloid leukemia, recurrent leukemia, refractory leukemia, or chronic lymphocytic leukemia), colorectal cancer, adrenocortical carcinoma, breast cancer (such as, for example, triple-negative breast cancer, endocrine-resistant breast cancer, ErbB2+ breast cancer, hormone receptor positive metastatic breast cancer, HER2 positive breast cancer, luminal breast cancer, or estrogen receptor positive breast cancer), medulloblastoma, cholangiocarcinoma, glioma, esophageal squamous cell carcinoma, meningioma, Ewing sarcoma, well-differentiated liposarcoma, dedifferentiated liposarcoma, clear cell renal cell carcinoma, lung squamous cell carcinoma, endometrial cancer, gastric cancer (such as, for example, ErbB2+ gastric cancer), pediatric astrocytoma, rare pediatric undifferentiated sarcoma, mastocytosis (such as, for example, advanced systemic mastocytosis), glioblastoma, glioblastoma multiforme, esophageal carcinoma, thyroid cancer (such as, for example, metastatic medullary thyroid cancer), neuroblastoma, colon cancer (such as, for example, KRAS mutated colon cancer, BRAF mutated colon cancer, colorectal cancer with a PI3K mutation, or colon cancer with an APC mutation), rectal cancer, esophageal carcinoma, lung cancer, liver cancer, kidney cancer, bladder cancer, ovarian cancer, well-differentiated liposarcoma, dedifferentiated liposarcoma, advanced dedifferentiated liposarcoma, leiomyosarcoma, neuroendocrine tumor, peripheral nerve sheath tumors, pediatric cancer (children with recurrent or refractory malignancies), mesothelioma, myeloma (such as, for example, multiple myeloma), chordomas, bladder cancer, nasopharyngeal carcinoma (such as, for example, non-keratyinizaing nasopharyngeal carcinoma), cervical cancer, testicular germ cell tumors, brain metastasis, head and neck squamous cell carcinoma, oral squamous cell carcinoma, osteosarcoma, and prostate cancer (such as, for example, androgen receptor positive prostate cancer, castration resistant prostate cancer or neuroendocrine prostate cancer).

Embodiment 73 of this disclosure relates to a method of treating a disease or condition according to Embodiment 72, wherein the cancer is metastatic triple-negative breast cancer, non-small cell lung cancer, small cell lung cancer, metastatic urothelial carcinoma, HR+ HER2− metastatic breast cancer, colorectal cancer, esophageal carcinoma, endometrial cancer, pancreatic ductal adenocarcinoma, castrate-resistant prostate cancer, epithelial ovarian cancer, gastric adenocarcinoma, glioblastoma multiforme, head and neck squamous cell carcinoma, hepatocellular carcinoma, cervical cancer, or renal cell carcinoma.

Embodiment 74 of this disclosure relates to a method of treating a disease or condition according to Embodiment 72, wherein the cancer is osteosarcoma, glioma, cholangiocarcinoma, glioblastoma, head and neck squamous cell carcinoma, medulloblastoma, advanced dedifferentiated liposarcoma, leiomyosarcoma, chordomas, breast cancer, or pediatric cancer.

Embodiment 75 of this disclosure relates to a method of treating a disease or condition according to Embodiment 72, wherein the cancer is medulloblastoma, head and neck squamous cell cancer, chorodomas, mesothelioma, colorectal cancer, chordomas, medulloblastoma, pancreatic ductal adenocarcinoma, thyroid cancer, or oral squamous cell carcinoma.

Embodiment 76 of this disclosure relates to a method of treating a disease or condition according to Embodiment 72, wherein the cancer is colorectal cancer, prostate cancer, castration-resistant prostate cancer, and neuroendocrine prostate cancer.

Embodiment 77 of this disclosure relates to a method of treating a disease or condition according to any of the preceding embodiments, wherein the compound inhibits CDK 2, CDK 4, CD6, or any combination thereof.

Embodiment 78 of this disclosure relates to a method of treating a disease or condition according to Embodiment 77, wherein the CDK is selected from CDK 2/4, CDK 2/6, CDK 4/6, and CDK 2/4/6.

In other embodiments of the methods of treatment of this disclosure, the additional therapeutic agent is a platinum compound. Non-limiting examples of platinum compounds include carboplatin, cisplatin oxaliplatin, nedaplatin, phenanthriplatin, loboplatin, and enloplatin.

In another embodiment, the additional therapeutic agent is a platinum compound, and the disease or condition that is treated is mesothelioma (such as, for example, malignant pleural mesothelioma), non-small cell lung cancer, triple-negative breast cancer, testicular germ cell tumors, cervical cancer or ovarian cancer.

In other embodiments of the methods of treatment of this disclosure, the additional therapeutic agent is a taxane. Non-limiting examples of taxanes include paclitaxel and docetaxel.

In another embodiment, the additional therapeutic agent is a taxane, and the disease or condition that is treated is pancreatic cancer, squamous cell lung cancer, breast cancer, cervical cancer, prostate cancer (such as, for example, castration-resistant prostate cancer), or ovarian cancer.

In other embodiments of the methods of treatment of this disclosure, the additional therapeutic agent is Apigenin. In another embodiment, the additional therapeutic agent is a taxane, and the disease or condition that is treated is bladder cancer.

In other embodiments of the methods of treatment of this disclosure, the additional therapeutic agent is a Wee-1 inhibitor. Non-limiting examples of Wee-1 inhibitors include adavosertib and AZD1775. In another embodiment the additional therapeutic agent is a Wee-1 inhibitors, and the disease or condition that is treated is sarcoma or breast cancer, including HR+ Breast Cancer

In other embodiments of the methods of treatment of this disclosure, the additional therapeutic agent is a PRMT5 inhibitor. Non-limiting examples of PRMT5 inhibitors include Pemrametosta. In another embodiment the additional therapeutic agent is a PRMT5 inhibitor, and the disease or condition that is treated is melanoma, breast cancer, pancreatic cancer, or esophageal carcinoma.

In other embodiments of the methods of treatment of this disclosure, the additional therapeutic agent is an MDM2 inhibitor, including p53-MDM2 inhibitors. Non-limiting examples of MDM2 inhibitors include nutlin-3, NVP-CGM097, idasanutlin, siremadlin, and brigimadlin. In another embodiment, the additional therapeutic agent is an MDM2 inhibitor, and the disease or condition that is treated is melanoma, ER+ breast cancer, well-differentiated liposarcoma, or dedifferentiated liposarcoma.

In other embodiments of the methods of treatment of this disclosure, the additional therapeutic agent is a Src inhibitor. Non-limiting examples of Src inhibitors include saracatinib and bosutinib. In another embodiment, the additional therapeutic agent is a Src inhibitor, and the disease or condition that is treated is colorectal cancer or breast cancer.

In other embodiments of the methods of treatment of this disclosure, the additional therapeutic agent is a Raf inhibitor. Non-limiting examples of Raf inhibitors include Sorafenib.

In another embodiment, the additional therapeutic agent is a Raf inhibitor, and the disease or condition that is treated is breast cancer, including tripe-negative breast cancer.

In other embodiments of the methods of treatment of this disclosure, the additional therapeutic agent is a MEK inhibitor. Non-limiting examples of MEK inhibitors include selumetinib, PD0325901, binimetinib, trametinib, and cobimetinib. In another embodiment, the additional therapeutic agent is a MEK inhibitor, and the disease or condition that is treated is a neuroendocrine tumor, neuroblastoma, melanoma (such as, for example, NRAS mutant melanoma), head and neck cancer, pancreatic cancer, pancreatic ductal adenocarcinoma, non-small cell lung cancer (such as KRAS-mutant non-small cell lung cancer or RAS-mutant non-small cell lung cancer), thyroid cancer, colon cancer (such as, for example, KRAS mutant colon cancer), or prostate cancer.

In other embodiments of the methods of treatment of this disclosure, the additional therapeutic agent is a PI3K inhibitor. Non-limiting examples of PI3K inhibitors include alpelisib, buparlisib, paxalisib, gedatolisib, and pictilisib. In another embodiment, the additional therapeutic agent is a PI3K inhibitor, and the disease or condition that is treated is medulloblastoma, NUT midline carcinoma, neuroendocrine tumors, chordomas, pancreatic neuroendocrine neoplasms or breast cancer (such as, for example, triple-negative breast cancer, metastatic triple-negative breast cancer, hormone-receptor positive breast cancer, HER2 negative breast cancer, or estrogen receptor positive breast cancer), head and neck squamous cell carcinoma, mesothelioma (such as, for example, malignant pleural mesothelioma), colorectal cancer, chordomas, medulloblastoma, pancreatic ductal adenocarcinoma, thyroid cancer, oral squamous cell carcinoma, or non-keratinizaing nasopharyngeal carcinoma.

In other embodiments of the methods of treatment of this disclosure, the additional therapeutic agent is an mTOR inhibitor optionally in combination with an endocrine therapy. Non-limiting examples of mTOR inhibitors include vistusertib, MLN0128, temsirolimus, sapanisertib, rapamycin, and everolimus. In another embodiment, the additional therapeutic agent is an mTOR inhibitor, and the disease or condition that is treated is osteosarcoma, glioma (such as, for example, diffuse intrinsic pontine glioma), cholangiocarcinoma, glioblastoma, head and neck squamous cell carcinoma, medulloblastoma, advanced dedifferentiated liposarcoma (DDL), leiomyosarcoma (LMS), chordomas, breast cancer (such as, for example, triple-negative cancer, estrogen receptor positive (ER+) breast cancer, or hormone-receptor positive breast cancer), or pediatric cancer (children with recurrent or refractory malignancies).

In other embodiments of the methods of treatment of this disclosure, the additional therapeutic agent is an FGFR inhibitor. Non-limiting examples of FGFR inhibitors include infigratinib, LY2874455, rogaratinib, BLU9931, H3B-6527, FIIN-2, FIIN-3, lenvatinib, ponatinib, regorafenib, dovitinib, lucitanib, cediranib, intedanib, and brivanib. In another embodiment the additional therapeutic agent is an FGFR inhibitor, and the disease or condition that is treated is lung squamous cell carcinoma, advanced differentiated liposarcoma, leiomyosarcoma, or breast cancer, including triple-negative cancer or hormone-receptor positive breast cancer.

In other embodiments of the methods of treatment of this disclosure, and additional agent is a pan-ERBB inhibitor. Non-limiting examples of pan-ERBB inhibitors include afatinib. In another embodiment, the additional therapeutic agent is a pan-ERBB inhibitor, and the disease or condition that is treated is esophageal carcinoma.

In other embodiments of the methods of treatment of this disclosure, the additional therapeutic agent is an ALK-inhibitor. Non-limiting examples of ALK-inhibitor inhibitors include ceritinib. In another embodiment, the additional therapeutic agent is an ALK-inhibitor, and the disease or condition that is treated is non-small cell lung cancer or neuroblastoma.

In other embodiments of the methods of treatment of this disclosure, the additional therapeutic agent is an anti-PD-1 or an anti-PD-L1 monoclonal antibody either of which is optionally in further combination with a CTLA4 antibody. Non-limiting examples of anti-PD-1 monoclonal antibodies include pembrolizumab, nivolumab, cemiplimab. Non-limiting examples of anti-PD-L1 monoclonal antibodies include atezolizumab, avelumab, and durvalumab. In another embodiment, the additional therapeutic agent is an anti-PD-1 or anti-PD-L1 monoclonal antibody, and the disease or condition that is treated is breast cancer (such as, for example, hormone receptor positive breast cancer, hormone receptor positive metastatic breast cancer, or HER2 negative breast cancer), bladder cancer, melanoma, brain metastasis, head and neck squamous cell carcinoma, or colon cancer.

In other embodiments of the methods of treatment of this disclosure, the additional therapeutic agent is an autophagy inhibitor. Non-limiting examples of autophagy inhibitors include hydroxychloroquine. In another embodiment, the additional therapeutic agent is an autophagy inhibitor, and the disease or condition that is treated is acute myleloid leukemia, pancreatic ductal adenocarcinoma or breast cancer, including hormone-receptor positive breast cancer.

In other embodiments of the methods of treatment of this disclosure, the additional therapeutic agent is a YAP-inhibitor. Non-limiting examples of YAP-inhibitors include verteporfin, pyrivinium pamoate, and CA3. In another embodiment, the additional therapeutic agent is a YAP-inhibitor, and the disease or condition that is treated is pancreatic cancer or esophageal carcinoma.

In other embodiments of the methods of treatment of this disclosure, the additional therapeutic agent is an androgen receptor inhibitor. Non-limiting examples of androgen receptor inhibitors include enzalutamide, N-desmethyl enzalutamide, darolutamide, apalutamide, ralaniten, EPI-7170, and abiraterone. In another embodiment, the additional therapeutic agent is an androgen receptor inhibitor and the disease or condition that is treated is prostate cancer, breast cancer (such as, for example, CDK4/6 resistant breast cancer or androgen receptor positive breast cancer), lung cancer (such as, for example, non-small cell lung cancer), liver cancer (such as, for example, hepatocellular carcinoma), kidney cancer, bladder cancer or ovarian cancer.

In other embodiments of the methods of treatment of this disclosure, the additional therapeutic agent is a PARP inhibitor. Non-limiting examples of PARP inhibitors include talazoparib and olaparib. In another embodiment, the additional therapeutic agent is a PARP inhibitor and the disease or condition that is treated is colon cancer (colorectal cancer), prostate cancer, including castration-resistant prostate cancer, and neuroendocrine prostate cancer.

In other embodiments of the methods of treatment of this disclosure, the additional therapeutic agent is a chemotherapeutic agent. Non-limiting examples of chemotherapeutic agents include irinotecan, eribulin, temozolomide, cytarabine, doxorubicin, gemcitabine, pemetrexed, dexamethasone, L-asparaginase, vincristine, and 5-FU. In another embodiment, the additional therapeutic agent is a chemotherapeutic agent and the disease or condition that is treated is medulloblastoma, lung adenocarcinoma, prostate cancer (such as, for example, castration resistant prostate cancer with a PI3K mutation), HPV-negative cervical cancer, colon cancer (colorectal cancer), acute myeloid leukemia, brain cancer, osteosarcoma, pancreatic cancer (such as, for example, pancreatic ductal adenocarcinoma), gastric cancer, head and neck squamous cell carcinoma, breast cancer (such as, for example, ER+ breast cancer, HER2+ breast cancer, HR negative breast cancer, advanced breast cancer or metastatic breast cancer), rectal cancer or colon cancer (such as, for example, colorectal cancer with a PI3K mutation.

In other embodiments of the methods of treatment of this disclosure, the additional therapy is a radiotherapy. In another embodiment, the additional therapy is radiotherapy and the disease or condition that is treated is glioma (such as, for example, diffuse intrinsic pontine gliomas or diffuse midline glioma), esophageal squamous cell carcinoma, meningioma, head and neck squamous cell carcinoma, metastatic breast cancer, nasopharyngeal carcinoma, hepatocellular carcinoma, cholangiocarcinoma, glioblastoma, glioblastoma multiforme, or medulloblastoma.

In other embodiments of the methods of treatment of this disclosure, the additional therapeutic agent is a FOXM1 inhibitor. Non-limiting examples of FOXM1 inhibitors include NB55, NB73 and NB 115. In another embodiment, the additional therapeutic agent is a FOXM1 inhibitor and the disease or condition that is treated is ER+ breast cancer or triple-negative breast cancer.

In other embodiments of the methods of treatment of this disclosure, the additional therapeutic agent is a BET inhibitor optionally in combination with a taxane. Non-limiting examples of BET inhibitors include JQ1, ZEN-3694, and ARV-825. In another embodiment, the additional therapeutic agent is a BET inhibitor and the disease or condition that is treated is triple negative breast cancer, NUT midline carcinoma, large B-cell lymphoma, gastric cancer, estrogen receptor positive breast cancer or medulloblastoma.

In other embodiments of the methods of treatment of this disclosure, the additional therapeutic agent is a KIT D816V inhibitor. Non-limiting examples of KIT D816V inhibitors include midostaurin and avapritinib. In another embodiment, the additional therapeutic agent is a KIT D816V inhibitor and the disease or condition that is treated is mastocytosis such as, for example, advanced systemic mastocytosis.

In other embodiments of the methods of treatment of this disclosure, the additional therapeutic agent is a steroidogenesis inhibitor. Non-limiting examples of steroidogenesis inhibitors include mitotane. In another embodiment, the additional therapeutic agent is a steroidogenesis inhibitor and the disease or condition that is treated is adrenocortical carcinoma.

In other embodiments of the methods of treatment of this disclosure, the additional therapeutic agent is a steroid. Non-limiting examples of steroids include progesterone. In another embodiment, the additional therapeutic agent is a steroid and the disease or condition that is treated is adrenocortical carcinoma.

In other embodiments of the methods of treatment of this disclosure, the additional therapeutic agent is a PAK inhibitor. Non-limiting examples of PAK inhibitors include PF03758309. In another embodiment, the additional therapeutic agent is a PAK inhibitor and the disease or condition that is treated is non-small cell lung cancer.

In other embodiments of the methods of treatment of this disclosure, the additional therapeutic agent is an IKK beta inhibitor. Non-limiting examples of IKK beta inhibitors include Bay 11-7082. In another embodiment, the additional therapeutic agent is an IKK beta inhibitor and the disease or condition that is treated is hepatoblastoma, hepatocellular carcinoma, KRAS mutated lung cancer, or KRAS mutated colon cancer.

In other embodiments of the methods of treatment of this disclosure, the additional therapeutic agent is an EGFR inhibitor. Non-limiting examples of EGFR inhibitors include cetuximab, PF-0647775, afatinib, and erlotinib. In another embodiment, the additional therapeutic agent is an EGFR inhibitor and the disease or condition that is treated is non-small cell lung cancer, triple-negative breast cancer, glioblastoma, nasopharyngeal carcinoma, chordoma, KRAS mutated lung cancer, or KRAS mutated colon cancer.

In other embodiments of the methods of treatment of this disclosure, the additional therapeutic agent is an ERK1/2 inhibitor. Non-limiting examples of ERK1/2 inhibitors include SCH772984 and LY3214996. In another embodiment, the additional therapeutic agent is an ERK1/2 inhibitor and the disease or condition that is treated is a neuroendocrine tumor or a peripheral nerve sheath tumor (such as, for example, NF1-associated plexiform neurofibroma).

In other embodiments of the methods of treatment of this disclosure, the additional therapeutic agent is a SHP2 inhibitor. Non-limiting examples of SHP2 inhibitors include TNO155. In another embodiment, the additional therapeutic agent is a SHP2 inhibitor and the disease or condition that is treated is lung cancer or castration-resistant prostate cancer.

In other embodiments of the methods of treatment of this disclosure, the additional therapeutic agent is an eIF4A inhibitor. Non-limiting examples of eIF4A inhibitors include CR-1-31-B. In another embodiment, the additional therapeutic agent is an eIF4A inhibitor and the disease or condition that is treated is estrogen receptor positive breast cancer or KRAS-mutant non-small cell lung cancer.

In other embodiments of the methods of treatment of this disclosure, the additional therapeutic agent is a BRAF inhibitor optionally in combination with a MEK inhibitor. Non-limiting examples of BRAF inhibitors include encorafenib, vemurafenib, and dabrafenib. In another embodiment, the additional therapeutic agent is a BRAF inhibitor and the disease or condition that is treated is thyroid cancer (such as, for example, papillary thyroid cancer or advanced thyroid cancer), BRAF-mutant melanoma, NRAS mutant melanoma, naive melanoma, leukemia, neuroblastoma, pediatric astrocytoma, BRAF-mutant colon cancer, or BRAF-mutant non-small cell lung cancer.

In other embodiments of the methods of treatment of this disclosure, the additional therapeutic agent is a notch inhibitor. Non-limiting examples of Notch inhibitors include CB-103. In another embodiment, the additional therapeutic agent is a Notch inhibitor and the disease or condition that is treated is estrogen receptor positive breast cancer.

In other embodiments of the methods of treatment of this disclosure, the additional therapeutic agent is a FLT-3 inhibitor. Non-limiting examples of FLT-3 inhibitors include quizartinib. In another embodiment, the additional therapeutic agent is a FLT-3 inhibitor and the disease or condition that is treated is acute myeloid leukemia.

In other embodiments of the methods of treatment of this disclosure, the additional therapeutic agent is a RANKL inhibitor. Non-limiting examples of RANKL inhibitors include OPG-Fc. In another embodiment, the additional therapeutic agent is a RANKL inhibitor and the disease or condition that is treated is luminal breast cancer.

In other embodiments of the methods of treatment of this disclosure, the additional therapeutic agent is an HER2 inhibitor optionally in combination with estrogen therapy, chemotherapy, or hormone receptor therapy. Non-limiting examples of HER2 inhibitors include neratinib, pyrotinib, T-DM1, trastuzumab and tucatinib. In another embodiment, the additional therapeutic agent is an HER2 inhibitor and the disease or condition that is treated is ErbB2+ gastric cancer, hormone receptor positive breast cancer ErbB2+ breast cancer, or HER2+ breast cancer.

In other embodiments of the methods of treatment of this disclosure, the additional therapeutic agent is an antioxidant defense inhibitor. Non-limiting examples of antioxidant defense inhibitors include auranofin. In another embodiment, the additional therapeutic agent is an antioxidant defense inhibitor and the disease or condition that is treated is mesothelioma.

In other embodiments of the methods of treatment of this disclosure, the additional therapeutic agent is a USP10 inhibitor. Non-limiting examples of USP10 inhibitors include spautin-1. In another embodiment, the additional therapeutic agent is a USP10 inhibitor and the disease or condition that is treated is myeloma.

In other embodiments of the methods of treatment of this disclosure, the additional therapeutic agent is a KIFC1 inhibitor. Non-limiting examples of KIFC1 inhibitors include ispinesib and SR31527. In another embodiment, the additional therapeutic agent is a KIFC1 inhibitor and the disease or condition that is treated is bladder cancer.

In other embodiments of the methods of treatment of this disclosure, the additional therapeutic agent is a glyoxalase 1 inhibitor. Non-limiting examples of glyoxalase 1 inhibitors include BBGC. In another embodiment, the additional therapeutic agent is a glyoxalase 1 inhibitor and the disease or condition that is treated is chronic lymphocytic leukemia.

In other embodiments of the methods of treatment of this disclosure, the additional therapeutic agent is a Bcl-2 inhibitor optionally in combination with a hypomethylation agent such as, for example, azacitidine. Non-limiting examples of Bcl-2 inhibitors include venetoclax and navitoclax. In another embodiment, the additional therapeutic agent is a Bcl-2 inhibitor and the disease or condition that is treated is breast cancer (such as, for example, triple-negative breast cancer or ER+ breast cancer), or acute myeloid leukemia.

In other embodiments of the methods of treatment of this disclosure, the additional therapeutic agent is a dual c-Met/Trk inhibitor. Non-limiting examples of dual c-Met/Trk inhibitors include altiratinib. In another embodiment, the additional therapeutic agent is a dual c-Met/Trk inhibitor and the disease or condition that is treated is glioblastoma.

In other embodiments of the methods of treatment of this disclosure, the additional therapeutic agent is an ALDH1A3 inhibitor. Non-limiting examples of ALDH1A3 inhibitors include N,N-diethylaminobenzaldehyde. In another embodiment, the additional therapeutic agent is a dual c-Met/Trk inhibitor and the disease or condition that is treated is glioblastoma.

In other embodiments of the methods of treatment of this disclosure, the additional therapeutic agent is an antrogen receptor inhibitor. Non-limiting examples of antrogen receptor inhibitors include seviteronel and RAD140. In another embodiment, the additional therapeutic agent is an antrogen receptor inhibitor and the disease or condition that is treated is breast cancer (such as, for example, ER+ breast cancer or triple negative breast cancer.

In other embodiments of the methods of treatment of this disclosure, the additional therapeutic agent is an aromatase inhibitor. Non-limiting examples of aromatase inhibitors include letrozole and fulvestrant. In another embodiment, the additional therapeutic agent is an aromatase inhibitor and the disease or condition that is treated is endometrial cancer, cystic brain metastases, or breast cancer (such as, for example, HER2 negative breast cancer).

In other embodiments of the methods of treatment of this disclosure, the additional therapeutic agent is a BET-CBP/p300 dual inhibitor. Non-limiting examples of BET-CBP/p300 dual inhibitors include NE02734. In another embodiment, the additional therapeutic agent is a BET-CBP/p300 dual inhibitor and the disease or condition that is treated is breast cancer or prostate cancer. This combination may involve overcoming resistance of CDK4/6 inhibitors.

In other embodiments of the methods of treatment of this disclosure, the additional therapeutic agent is a beta-catenin inhibitor. Non-limiting examples of beta-catenin inhibitors include ICG-001. In another embodiment, the additional therapeutic agent is a beta-catenin inhibitor and the disease or condition that is treated is endocrine-resistant breast cancer.

In other embodiments of the methods of treatment of this disclosure, the additional therapeutic agent is a BMI inhibitor. Non-limiting examples of BMI inhibitors include PTC-209.

In another embodiment, the additional therapeutic agent is a BMI inhibitor and the disease or condition that is treated is breast cancer, colon cancer or prostate cancer.

In other embodiments of the methods of treatment of this disclosure, the additional therapeutic agent is a BTK inhibitor. Non-limiting examples of BTK inhibitors include ibrutinib and tirabrutinib. In another embodiment, the additional therapeutic agent is a BTK inhibitor and the disease or condition that is treated is mantle cell lymphoma or diffuse large B-cell lymphoma.

In other embodiments of the methods of treatment of this disclosure, the additional therapeutic agent is a CD73 inhibitor. Non-limiting examples of CD73 inhibitors include AB680.

In another embodiment, the additional therapeutic agent is a CD73 inhibitor and the disease or condition that is treated is colorectal cancer.

In other embodiments of the methods of treatment of this disclosure, the additional therapeutic agent is a dihydrofolate reductase inhibitor. Non-limiting examples of dihydrofolate reductase inhibitors include pralatrexate. In another embodiment, the additional therapeutic agent is a dihydrofolate reductase inhibitor and the disease or condition that is treated is bladder cancer.

In other embodiments of the methods of treatment of this disclosure, the additional therapeutic agent is an elF4A inhibitor. Non-limiting examples of elF4A inhibitors include CR-1-31-B. In another embodiment, the additional therapeutic agent is an elF4A inhibitor and the disease or condition that is treated is ER+ breast cancer or KRAS-mutant non-small cell lung cancer.

In other embodiments of the methods of treatment of this disclosure, the additional therapeutic agent is an estrogen receptor antagonist. Non-limiting examples of estrogen receptor antagonists include lasofoxifene. In another embodiment, the additional therapeutic agent is an estrogen receptor antagonist and the disease or condition that is treated is endometrial cancer, such as, for example, ER+ recurrent endometrial cancer.

In other embodiments of the methods of treatment of this disclosure, the additional therapeutic agent is an estrogen receptor alpha antagonist. Non-limiting examples of estrogen receptor alpha antagonists include celastrol and gambogic acid. In another embodiment, the additional therapeutic agent is an estrogen receptor alfa antagonist and the disease or condition that is treated is breast cancer, such as, for example, breast cancer with an estrogen receptor alpha Y537S mutation.

In other embodiments of the methods of treatment of this disclosure, the additional therapeutic agent is an EZH2 inhibitor. Non-limiting examples of EZH2 inhibitors include AQB.

In another embodiment, the additional therapeutic agent is an EZH2 inhibitor and the disease or condition that is treated is glioblastoma.

In other embodiments of the methods of treatment of this disclosure, the additional therapeutic agent is an FOXM1 inhibitor. Non-limiting examples of FOXM1 inhibitors include NB55, NB73 and NB 115. In another embodiment, the additional therapeutic agent is an FOXM1 inhibitor and the disease or condition that is treated is ER+ triple negative breast cancer.

In other embodiments of the methods of treatment of this disclosure, the additional therapeutic agent is a GLI1 inhibitor. Non-limiting examples of GLI1 inhibitors include GANT61. In another embodiment, the additional therapeutic agent is a GLI1 inhibitor and the disease or condition that is treated is acute myeloid leukemia.

In other embodiments of the methods of treatment of this disclosure, the additional therapeutic agent is an HDAC inhibitor. Non-limiting examples of HDAC inhibitors include tucidinostat, suberanilohydroxamic acid, vorinostat, and valproate. In another embodiment, the additional therapeutic agent is an HDAC inhibitor and the disease or condition that is treated is HR+ breast cancer, nasopharyngeal carcinoma, or mantle cell lymphoma.

In other embodiments of the methods of treatment of this disclosure, the additional therapeutic agent is a heat shock protein which is also referred to as a chaperone inhibitor. Non-limiting examples of heat shock proteins include ShetA2. In another embodiment, the additional therapeutic agent is a heat shock protein and the disease or condition that is treated is cervical cancer.

In other embodiments of the methods of treatment of this disclosure, the additional therapeutic agent is an HER2 antibody drug conjugate (ADC). Non-limiting examples of HER2 ADCs include T-DM1. In another embodiment, the additional therapeutic agent is an HER2 ADC and the disease or condition that is treated is HER2+ breast cancer.

In other embodiments of the methods of treatment of this disclosure, the additional therapeutic agent is an HIF-2 alpha inhibitor. Non-limiting examples of HIF-2 alpha inhibitors include PT2399. In another embodiment, the additional therapeutic agent is a HIF-2 alpha inhibitor and the disease or condition that is treated is clear cell renal cell carcinoma.

In other embodiments of the methods of treatment of this disclosure, the additional therapeutic agent is an IGF-1R inhibitor. Non-limiting examples of IGF-1R inhibitors include ganitumab and NVP-AEW541. In another embodiment, the additional therapeutic agent is an IGF-1R inhibitor and the disease or condition that is treated is Ewing sarcoma, well-differentiated liposarcoma, or dedifferentiated liposarcoma.

In other embodiments of the methods of treatment of this disclosure, the additional therapeutic agent is a KIFC inhibitor. Non-limiting examples of KIFC inhibitors include ispinesib and SR31527. In another embodiment, the additional therapeutic agent is a KIFC inhibitor and the disease or condition that is treated is bladder cancer.

In other embodiments of the methods of treatment of this disclosure, the additional therapeutic agent is a KIT inhibitor. Non-limiting examples of KIT inhibitors include midostaurin, avapritinib, and nintedanib. In another embodiment, the additional therapeutic agent is a KIT inhibitor and the disease or condition that is treated is mastocytosis, such as, for example, advanced systemic mastocytosis.

In other embodiments of the methods of treatment of this disclosure, the additional therapeutic agent is a menin inhibitor. Non-limiting examples of menin inhibitors include SNDX-50469. In another embodiment, the additional therapeutic agent is a menin inhibitor and the disease or condition that is treated is acute myeloid leukemia.

In other embodiments of the methods of treatment of this disclosure, the additional therapeutic agent is an LC 3-KAT inhibitor. Non-limiting examples of LC 3-KAT inhibitors include trimetazidine. In another embodiment, the additional therapeutic agent is an LC 3-KAT inhibitor and the disease or condition that is treated is triple negative breast cancer.

In other embodiments of the methods of treatment of this disclosure, the additional therapeutic agent is a proteosome inhibitor. Non-limiting examples of proteosome inhibitors include bortezomib. In another embodiment, the additional therapeutic agent is a proteosome inhibitor and the disease or condition that is treated is multiple myeloma or mantle cell lymphoma.

In other embodiments of the methods of treatment of this disclosure, the additional therapeutic agent is a RET inhibitor. Non-limiting examples of RET inhibitors include alectinib.

In another embodiment, the additional therapeutic agent is a RET inhibitor and the disease or condition that is treated is RET fusion positive non-small cell lung cancer or metastatic medullary thyroid cancer.

In other embodiments of the methods of treatment of this disclosure, the additional therapeutic agent is a synolytic. Non-limiting examples of RET inhibitors include alectinib. In another embodiment, the additional therapeutic agent is a RET inhibitor and the disease or condition that is treated is RET fusion positive non-small cell lung cancer or metastatic medullary thyroid cancer.

In other embodiments of the methods of treatment of this disclosure, the additional therapeutic agent is a senolytic agent. Non-limiting examples of senolytic agents include navitoclax. In another embodiment, the additional therapeutic agent is a synolytic agent and the disease or condition that is treated is triple negative breast cancer or head and neck squamous cell carcinoma.

In other embodiments of the methods of treatment of this disclosure, the additional therapeutic agent is a tankyrase inhibitor. Non-limiting examples of tankyrase inhibitors include MSC2504877. In another embodiment, the additional therapeutic agent is a tankyrase inhibitor and the disease or condition that is treated is colon cancer, such as, for example, APC mutant colon cancer.

In other embodiments of the methods of treatment of this disclosure, the additional therapeutic agent is a TRK inhibitor. Non-limiting examples of TRK inhibitors include entrectinib. In another embodiment, the additional therapeutic agent is a TRK inhibitor and the disease or condition that is treated is glioma, such as, for example, ROS1/NTRK-fusion-positive pediatric high-grade gliomas (pHGG).

In other embodiments of the methods of treatment of this disclosure, the additional therapeutic agent is a type-I TGF-b receptor inhibitor. Non-limiting examples of type-I TGF-b receptor inhibitors include SB-505124. In another embodiment, the additional therapeutic agent is a type-I TGF-b receptor inhibitor and the disease or condition that is treated pancreatic ductal adenocarcinoma.

In other embodiments of the methods of treatment of this disclosure, the additional therapeutic agent is a vitamin D analog. Non-limiting examples of vitamin D analogs include inecalcitol. In another embodiment, the additional therapeutic agent is a vitamin D analog and the disease or condition that is treated is hormone receptor positive breast cancer.

In other embodiments of the methods of treatment of this disclosure, the additional therapeutic agent is an XPO1 inhibitor. Non-limiting examples of XPO1 inhibitors include KPT-330. In another embodiment, the additional therapeutic agent is a XPO1 inhibitor and the disease or condition that is treated is rare pediatric undifferentiated sarcoma.

In other embodiments of the methods of treatment of this disclosure, the additional therapeutic agent is a Trop2 antibody drug conjugate (Trop2 ADC). Non-limiting examples of XPO1 inhibitors include sacituzumab govtecan and datopotamab deruxtecan. In another embodiment, the additional therapeutic agent is a Trop2 antibody drug conjugate and the disease or condition that is treated is metastatic triple-negative breast cancer, non-small cell lung cancer, small cell lung cancer, metastatic urothelial carcinoma, HR+ HER2− metastatic breast cancer, colorectal cancer, esophageal carcinoma, endometrial cancer, pancreatic ductal adenocarcinoma, castrate-resistant prostate cancer, epithelial ovarian cancer, gastric adenocarcinoma, glioblastorna multiforme, squamous cell carcinoma of the head and neck, hepatocellular carcinoma, cervical cancer, or renal cell carcinoma.

In other embodiments of the methods of treatment of this disclosure, the additional therapeutic agent is a Trop2 antibody drug conjugate, a cytokine, an oncolytic virus, a bi-specific immune checkpoint inhibitor, a T-cell engager, a cancer vaccine, a cell therapy, a CD73 inhibitor, HER3 antibody drug conjugates such as patritumab or deruxtecan, or a TLR agonist.

In other embodiments of the methods of treatment of this disclosure, the additional therapy is a cancer gene therapy, for example, gene therapies aimed to normalize tumor suppressors such R^{b} or P53. CDK inhibitors can be combined with gene therapies aimed to normalize tumor suppressors such R^{b}, P53 etc. More specifically, CDK2/4/6 inhibitors cause cell cycle arrest by preventing the phosphorylation of the tumor suppressor protein R^{b}. Loss of R^{b} function is one of the mechanisms of intrinsic and acquired resistance to CDK4/6 inhibitors. R^{b} gene therapies could be used to reintroduce R^{b} into R^{b}-deficient cancers so that they become sensitive to CDK inhibitors. More patients could benefit from this combination strategy as R^{b} loss of function is highly prevalent in human cancers.

Additional embodiments of the compounds in the methods of this disclosure include the following where applicable:

Ring A can be any suitable ring known by one of skill in the art. In some embodiments, A is a ring selected from optionally substituted carbocycle, optionally substituted 4- to 10-membered heterocycle, and optionally substituted isoindoline. In some embodiments, A is a ring selected from optionally substituted carbocycle, optionally substituted 4- to 8-membered heterocycle, and optionally substituted isoindoline. In some embodiments, A is a ring selected from optionally substituted carbocycle, optionally substituted 4- to 6-membered heterocycle, and optionally substituted isoindoline. In some embodiments, A is selected from optionally substituted azetidine, optionally substituted piperidine, optionally substituted azabicyclo[3.1.0]hexane, optionally substituted phenyl, optionally substituted pyridine, optionally substituted pyrazole, optionally substituted isoindoline, and optionally substituted tetrahydroisoquinoline. In some embodiments, A is selected from optionally substituted pyridine, optionally substituted azabicyclo[3.1.0]hexane, optionally substituted azetidine, and optionally substituted tetrahydroisoquinoline. In some embodiments, A is not optionally substituted pyridine.

In some embodiments, A is not optionally substituted pyrimidine.

In some embodiments, A is substituted with methyl, —SO₂Me, methylpiperidine, methylpiperazine, methylazaspiro[3.3]heptane, methyldiazaspiro[3.3]heptane, or a combination thereof. In some embodiments, A is substituted with methyl, —SO₂Me, —SO₂Me, methylpiperidine, methylpiperazine, or a combination thereof. In some embodiments, A is substituted with methyl, —SO₂Me, or a combination thereof. In some embodiments, A is substituted with —SO₂Me.

Z¹ and Z² can be any suitable functional group known by one of skill in the art. In some embodiments, Z¹ and Z² are independently selected from —C(R²)₂—, —C(O)—, —NR³—, —N(C(O)R²)—, —NS(O₂)R², —O—, —S—, —S(O)—, and —S(O)₂—. In some embodiments, Z¹ and Z² are independently selected from —C(R²)₂—, —C(O)—, —NR³—, —NS(O₂)R²—, —O—, and —S—. In some embodiments, Z¹ and Z² are independently selected from —C(R²)₂—, —NR³—, —O—, and —S—. In some embodiments, Z¹ and Z² are independently —C(R²)₂—.

Variables a and b can be any suitable number known by one of skill in the art. In some embodiments, each of a and b are independently selected from 1, 2, 3, and 4. In some embodiments, each of a and b are independently 1, 2, and 3. In some embodiments, each of a and b are independently selected from 1 and 2.

Y¹ can be any suitable functional group known by one of skill in the art. In some embodiments, Y¹ is selected from —C(R²)₂—, —C(O)—, —NR³—, —N(C(O)R²)—, —NS(O₂)R², —O—, —S—, —S(O)—, and —S(O)₂—. In some embodiments, Y¹ is selected from —C(R²)₂—, —C(O)—, —NR³—, —NS(O₂)R²—, —O—, and —S—. In some embodiments, Y¹ is selected from —C(R²)₂—, —NR³—, —O—, and —S—. In some embodiments, Y¹ is selected from —C(R²)₂— and —NR³. In some embodiments, Y¹ is —C(R²)₂— and two R² substituents come together to form a ring selected from an optionally substituted heterocycle and an optionally substituted carbocycle. In some embodiments, Y¹ is —C(R²)₂— and the two R² substituents come together to form a ring selected from an optionally substituted heterocycle.

Variable m can be any suitable number known by one of skill in the art. In some embodiments m is selected from 0 to 5. In some embodiments, m is selected from 0 to 3. In some embodiments, m is selected from 0 to 2. In some embodiments, m is selected from 0 to 1. In some embodiments m is selected from 1 to 5. In some embodiments, m is selected from 1 to 3. In some embodiments, m is selected from 1 to 2. In some embodiments, m is 0. In some embodiments, m is 1. In some embodiments, m is 2. In some embodiments, m is 3.

R¹ can be any suitable functional group known by one of skill in the art. In some embodiments, each R¹ is independently selected from halogen, —CN, —NO₂, optionally substituted alkyl, optionally substituted alkenyl, optionally substituted alkynyl, optionally substituted carbocycle, and optionally substituted heterocycle. In some embodiments, each R¹ is independently selected from optionally substituted alkyl, optionally substituted carbocycle, and optionally substituted heterocycle. In some embodiments, each R¹ is independently selected from optionally substituted alkyl and optionally substituted heterocycle. In some embodiments, each R¹ is independently selected from methyl, optionally substituted piperidine, optionally substituted piperazine, optionally substituted azaspiro[3.3]heptane, and optionally substituted diazaspiro[3.3]heptane. In some embodiments, each R¹ is independently selected from methyl, ethyl, and optionally substituted diazaspiro[3.3]heptane.

R² can be any suitable functional group known by one of skill in the art. In some embodiments, each R² is independently selected from hydrogen, halogen, —CN, —OH, —O—C₁₋₄ alkyl, optionally substituted alkyl, optionally substituted cycloalkyl, and optionally substituted heterocycloalkyl, or two R² substituents come together to form an optionally substituted heterocycle or an optionally substituted carbocycle, or R² and R³ substituents come together to form an optionally substituted heterocycle. In some embodiments, each R² is independently selected from hydrogen, halogen, —CN, optionally substituted cycloalkyl, and optionally substituted heterocycloalkyl. In some embodiments, wherein each R² is independently selected from hydrogen, halogen, —CN, cyclopropyl, cyclobutyl, optionally substituted cycloalkyl, and optionally substituted heterocycloalkyl. In some embodiments, each R² is independently selected from hydrogen, fluoro, chloro, bromo, —CN, cyclopropyl, cyclobutyl, oxetane, and azetidine. In some embodiments, each R² is independently selected from hydrogen, fluoro, —CN, cyclopropyl, cyclobutyl, optionally substituted oxetane, and optionally substituted azetidine. In some embodiments, each R² is selected from hydrogen, fluoro, —CN and cyclopropyl. In some embodiments, each R² is selected from hydrogen, —CN and cyclopropyl.

In some embodiments, or two R² substituents come together to form an optionally substituted heterocycle or an optionally substituted carbocycle. In some embodiments, two R² substituents come together to form an optionally substituted heterocycle. In some embodiments, two R² substituents come together to form an optionally substituted carbocycle. In some embodiments, two R² substituents come together such that this

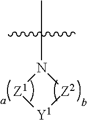

structure is

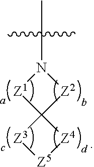

Each of Z¹, Z², Z³, Z⁴ and Z⁵ can be any suitable functional group known by one of skill in the art. In some embodiments, each of Z¹, Z², Z³, Z⁴ and Z⁵ are independently selected from —C(R²)₂—, —C(O)—, —NR³—, —N(C(O)R²)—, —NS(O₂)R², —O—, —S—, —S(O)—, and —S(O)₂—, wherein Z⁵ is additionally selected from a bond. Each of Z¹ and Z² can be any functional group as described previously.

In some embodiments, each of Z¹, Z², Z³, Z⁴ and Z⁵ are independently selected from —C(R²)₂—, —NR³—, —N(C(O)R²)—, —NS(O₂)R³, —O—, and —S(O)₂—, wherein Z⁵ is additionally selected from a bond. In some embodiments, each of Z¹, Z², Z³, Z⁴ and Z⁵ are independently selected from —C(R²)₂—, —NR³—, —N(C(O)R²)—, —NS(O₂)R³, —O—, and —S(O)₂—, wherein Z⁵ is additionally selected from a bond. In some embodiments, each of Z¹, Z², Z³, Z⁴ and Z⁵ are independently selected from —C(R²)₂—, —NR³—, —O—, and —S(O)₂—, wherein Z⁵ is additionally selected from a bond.

Variables a, b, c, and d can be any suitable number known by one of skill in the art. In some embodiments, each of a, b, c, and d are independently selected from 1, 2, 3, and 4. In some embodiments, each of a, b, c, and d are independently 1, 2, and 3. In some embodiments, each of a, b, c, and d are independently selected from 1 and 2.

In some embodiments, each R² is independently selected from hydrogen, halogen, optionally substituted cycloalkyl, and optionally substituted heterocycloalkyl, or two R² substituents come together to form an optionally substituted heterocycle or an optionally substituted carbocycle. In some embodiments, each R² is independently selected from hydrogen, halogen, —CN, optionally substituted cycloalkyl, and optionally substituted heterocycloalkyl. In some embodiments, each R² is independently selected from hydrogen, fluoro, chloro, cyclopropyl, cyclobutyl or two R² substituents come together to form an optionally substituted heterocycle or an optionally substituted carbocycle. In some embodiments, each R² is independently selected from hydrogen, fluoro, or two R² substituents come together to form an optionally substituted heterocycle or an optionally substituted carbocycle.

In some embodiments, each R² is independently selected from hydrogen, halogen, —CN, —OH, and optionally substituted alkyl. In some embodiments, each R² is independently selected from hydrogen, fluoro, and —OH. In some embodiments, each R² is independently selected from hydrogen, halogen, —CN, optionally substituted cycloalkyl, and optionally substituted heterocycloalkyl. In some embodiments, R² is independently selected from hydrogen and fluoro.

R³ can be any suitable functional group known by one of skill in the art. In some embodiments, each R³ is independently selected from hydrogen, optionally substituted alkyl, optionally substituted C₃₋₄ carbocycle, and optionally substituted 3- to 4-membered heterocycloalkyl, or R² and R³ substituents come together to form an optionally substituted heterocycle. In some embodiments, each R³ is independently selected from hydrogen, optionally substituted alkyl, optionally substituted C₃₋₄ carbocycle, and optionally substituted 3- to 4-membered heterocycloalkyl. In some embodiments, each R³ is independently selected from optionally substituted alkyl, optionally substituted C₃₋₄ carbocycle, and optionally substituted 3- to 4-membered heterocycloalkyl. In some embodiments, R³ is selected from optionally substituted alkyl. In some embodiments, each R³ is selected from cyclopropyl, cyclobutyl, optionally substituted oxetane, and optionally substituted azetidine. In some embodiments, each R³ is selected from cyclopropyl and cyclobutyl. In some embodiments, R³ is cyclopropyl.

In some embodiments, R³ is selected from hydrogen, —(CH₂)₂OMe, cyclopropyl, cyclobutyl, optionally substituted oxetane, and optionally substituted azetidine. In some embodiments, R³ is selected from —(CH₂)₂OMe and cyclopropyl. In some embodiments, R³ is selected from hydrogen, methyl, ethyl, propyl, cyclopropyl, cyclobutyl, optionally substituted oxetane, and optionally substituted azetidine. In some embodiments, R³ is selected from hydrogen, methyl, ethyl, and propyl. In some embodiments, R³ is selected from hydrogen, methyl, ethyl, and propyl. In some embodiments, R³ is methyl.

R⁴ can be any suitable functional group known by one of skill in the art. In some embodiments, R⁴ is selected from hydrogen, halogen, —CN, optionally substituted C₁₋₄ alkyl, optionally substituted C₃₋₄ carbocycle, and optionally substituted 3- to 4-membered heterocycloalkyl. In some embodiments, R⁴ is selected from hydrogen, halogen, optionally substituted C₁₋₂ alkyl, optionally substituted C₃₋₄ carbocycle, and optionally substituted 3- to 4 membered heterocycloalkyl. In some embodiments, R⁴ is selected from hydrogen, halogen, —CN, optionally substituted C₁₋₂ alkyl, and optionally substituted C₃₋₄ carbocycle. In some embodiments, R⁴ is selected from hydrogen and optionally substituted C₁ alkyl. In some embodiments, R⁴ is selected from hydrogen, methyl, and —CHF₂. In some embodiments, R⁴ is selected from hydrogen, halogen, and —CN. In some embodiments, R⁴ is selected from hydrogen. In some embodiments, R⁴ is not optionally substituted phenyl. In some embodiments, R⁴ is not optionally substituted alkyl.

R⁵ can be any suitable functional group known by one of skill in the art. In some embodiments, R⁵ is selected from hydrogen, halogen, —CN, optionally substituted C₁₋₄ alkyl, optionally substituted C₃₋₄ carbocycle, and optionally substituted 3- to 4-membered heterocycloalkyl. In some embodiments, R⁵ is selected from hydrogen, halogen, optionally substituted C₁₋₂ alkyl, optionally substituted C₃₋₄ carbocycle, and optionally substituted 3- to 4 membered heterocycloalkyl. In some embodiments, R⁵ is selected from hydrogen, halogen, and optionally substituted C₁₋₂ alkyl. In some embodiments, R⁵ is selected from hydrogen, fluoro, chloro, methyl, ethyl, and propyl. In some embodiments, R⁵ is selected from hydrogen and fluoro.

R⁶ can be any suitable functional group known by one of skill in the art. In some embodiments, R⁶ is selected from hydrogen, halogen, —CN, optionally substituted C₁₋₄ alkyl, optionally substituted C₃₋₄ carbocycle, and optionally substituted 3- to 4-membered heterocycloalkyl. In some embodiments, R⁶ is selected from hydrogen, halogen, optionally substituted C₁₋₂ alkyl, optionally substituted C₃₋₄ carbocycle, and optionally substituted 3- to 4 membered heterocycloalkyl. In some embodiments, R⁶ is selected from hydrogen, halogen, and optionally substituted C₁₋₂ alkyl. In some embodiments, R⁶ is selected from hydrogen, fluoro, chloro, methyl, ethyl, and propyl. In some embodiments, R⁶ is selected from hydrogen and fluoro. In some embodiments, R⁶ is hydrogen.

R⁷ can be any suitable functional group known by one of skill in the art. In some embodiments, R⁷ is selected from hydrogen and optionally substituted C₁₋₄ alkyl. In some embodiments, R⁷ is selected from hydrogen, methyl, ethyl, and propyl. In some embodiments, R⁷ is hydrogen.

R⁸ can be any suitable functional group known by one of skill in the art. In some embodiments, R⁸ is selected from halogen, —CN, and optionally substituted C₁₋₄ alkyl. In some embodiments, R⁸ is selected from halogen and optionally substituted C₁₋₄ alkyl. In some embodiments, R⁸ is selected from optionally substituted C₁₋₄ alkyl. In some embodiments, R⁸ is selected from methyl, ethyl, and propyl.

R⁹ can be any suitable functional group known by one of skill in the art. In some embodiments, R⁹ is selected from optionally substituted C₁₋₄ alkyl, optionally substituted C₃₋₆ carbocycle, 3- to 6-membered heterocycloalkyl, and optionally substituted C₅₋₆ heteroaryl. In some embodiments, R⁹ is selected from optionally substituted C₁₋₄ alkyl, optionally substituted C₃₋₆ carbocycle, and optionally substituted C₅₋₆ heteroaryl. In some embodiments, R⁹ is optionally substituted C₅₋₆ heteroaryl. In some embodiments, R⁹ is optionally substituted C₅ heteroaryl. In some embodiments, R⁹ is optionally substituted C₆ heteroaryl. In some embodiments, R⁹ is optionally substituted pyrazole. In some embodiments, R⁹ is selected from optionally substituted C₁₋₄ alkyl and optionally substituted C₃₋₆ carbocycle. In some embodiments, R⁹ is selected from optionally substituted C₁₋₄ alkyl. In some embodiments, R⁹ is selected from methyl, ethyl, and propyl. In some embodiments, R⁹ is cyclopropyl, cyclobutyl, cyclopentyl, or cyclohexyl. In some embodiments, R⁹ is cyclopropyl, cyclobutyl.

Variable n can be any suitable number known by one of skill in the art. In some embodiments, n is selected from 0 to 9. In some embodiments, n is selected from 0 to 5. In some embodiments, n is selected from 0 to 3. In some embodiments n is selected from 0 to 2. In some embodiments n is 0 or 1. In some embodiments, n is 0.

R¹⁰ can be any suitable functional group known by one of skill in the art. In some embodiments, R¹⁰ is optionally substituted heterocycloalkyl. In some embodiments, R¹⁰ is selected from optionally substituted piperazine, optionally substituted piperidine, and optionally substituted 2,6-diazaspiro[3.3]heptane. In some embodiments, R¹⁰ is selected from optionally substituted piperazine and optionally substituted 2,6-diazaspiro[3.3]heptane. In some embodiments, R¹⁰ is selected from methylpiperazine and methyl-2,6-diazaspiro[3.3]heptane.

R¹¹ can be any suitable functional group known by one of skill in the art. In some embodiments, R¹¹ is selected from halogen, —CN, and optionally substituted C₁₋₄ alkyl. In some embodiments, R¹¹ is selected from halogen and optionally substituted C₁₋₄ alkyl. In some embodiments, R¹¹ is selected from optionally substituted C₁₋₄ alkyl. In some embodiments, R¹¹ is selected from methyl, ethyl, and propyl.

Variable p can be any suitable number known by one of skill in the art. In some embodiments, p is selected from 0 to 4. In some embodiments, p is selected from 0 to 3. In some embodiments, p is selected from 0 to 2. In some embodiments, p is 0 or 1. In some embodiments, p is 0.

R¹² can be any suitable functional group known by one of skill in the art. In some embodiments, R¹² is optionally substituted heterocycloalkyl; or R¹² and R¹³ come together to form an optionally substituted heterocycle. In some embodiments, R¹² is optionally substituted 5- to 6-membered heterocycle, or R¹² and R¹³ come together to form an optionally substituted heterocycle or an optionally substituted carbocycle. In some embodiments, R¹² is optionally substituted 5- to 6-membered heterocycle. In some embodiments, R¹² is selected from optionally substituted piperidine, optionally substituted piperazine, and optionally substituted 2,6-diazaspiro[3.3]heptane. In some embodiments, R¹² is selected from optionally substituted piperidine and optionally substituted 2,6-diazaspiro[3.3]heptane. In some embodiments, R¹² is selected from methylpiperazine and methyl-2,6-diazaspiro[3.3]heptane.

R¹³ can be any suitable functional group known by one of skill in the art. In some embodiments, R¹³ is selected from halogen, —CN, and optionally substituted C₁₋₄ alkyl; or R¹² and R³ come together to form an optionally substituted heterocycle. In some embodiments, R¹³ is selected from halogen and optionally substituted C₁₋₄ alkyl. In some embodiments, R¹³ is selected from methyl, ethyl, and propyl.

In some embodiments, R¹² and R¹³ come together to form an optionally substituted heterocycle or an optionally substituted carbocycle. In some embodiments, R¹² and R¹³ come together to form an optionally substituted heterocycle. In some embodiments, R¹² and R¹³ come together such that

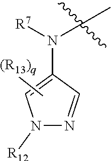

structure is

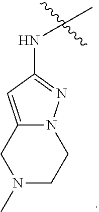 [SMILES: *Nc1cc2n(n1)CCN(C)C2]

Variable q can be any number known by one of skill in the art. In some embodiments, q is selected from 0 to 2. In some embodiments, q is 0 or 1. In some embodiments, q is 0. In some embodiments, q is 1.

R¹⁴ can be any suitable functional group known by one of skill in the art. In some embodiments, R¹⁴ is selected from —SOR¹⁶—, and optionally substituted heterocycloalkyl. In some embodiments, R¹⁴ is —SOR¹⁶—. In some embodiments, R¹⁴ is selected from optionally substituted heterocycloalkyl.

R¹⁵ can be any suitable functional group known by one of skill in the art. In some embodiments, R¹⁵ is selected from hydrogen, halogen, —CN, and optionally substituted C₁₋₄ alkyl. In some embodiments, R¹⁵ is selected from hydrogen, halogen, and optionally substituted C₁₋₄ alkyl. In some embodiments, R¹⁵ is selected from hydrogen and optionally substituted C₁₋₄ alkyl. In some embodiments, R¹⁵ is hydrogen. In some embodiments, R¹⁵ is optionally substituted C₁₋₄ alkyl. In some embodiments, R¹⁵ is methyl, ethyl, and propyl.

R¹⁶ can be any suitable functional group known by one of skill in the art. In some embodiments, R¹⁶ is selected from optionally substituted C₁₋₄ alkyl, optionally substituted C₃₋₆ carbocycle, and optionally substituted 3- to 6-membered heterocycloalkyl. In some embodiments, R¹⁶ is optionally substituted C₃₋₆ carbocycle and optionally substituted 3- to 6-membered heterocycloalkyl. In some embodiments, R¹⁶ is selected from optionally substituted C₁₋₄ alkyl. In some embodiments, R¹⁶ is selected from methyl, ethyl, and propyl.

R¹⁷ can be any suitable functional group known by one of skill in the art. In some embodiments, R¹⁷ is selected from —SOR¹⁹—, optionally substituted alkyl, optionally substituted carbocycle, and optionally substituted heterocycloalkyl. In some embodiments, R¹⁷ is selected from —SOR¹⁹—, optionally substituted alkyl, and optionally substituted 3- to 5 membered heterocycloalkyl. In some embodiments, R¹⁷ is selected from —SOR¹⁹—, methyl, and optionally substituted 4-membered heterocycloalkyl. In some embodiments, R¹⁷ is selected from —SOR¹⁹—, methyl, and 1-(methylsulfonyl)azetidine. In some embodiments, R¹⁷ is —SOR¹⁹—. In some embodiments, R¹⁷ is methyl. In some embodiments, R¹⁷ is 1-(methylsulfonyl)azetidine.

R¹⁸ can be any suitable functional group known by one of skill in the art. In some embodiments, R¹⁸ is selected from halogen, —CN, and optionally substituted C₁₋₄ alkyl. In some embodiments, R¹⁸ is selected from halogen and optionally substituted C₁₋₄ alkyl. In some embodiments, R¹⁸ is selected from optionally substituted C₁₋₄ alkyl. In some embodiments, R¹⁸ is selected from methyl, ethyl, and propyl.

R¹⁹ can be any suitable functional group known by one of skill in the art. In some embodiments, R¹⁹ is selected from optionally substituted C₁₋₄ alkyl, optionally substituted C₃₋₆ carbocycle, and optionally substituted 3- to 6-membered heterocycloalkyl. In some embodiments, R¹⁹ is selected from optionally substituted C₃₋₆ carbocycle and optionally substituted 3- to 6-membered heterocycloalkyl. In some embodiments, R¹⁹ is optionally substituted alkyl. In some embodiments, R¹⁹ is selected from methyl, ethyl, and propyl. In some embodiments, R¹⁹ is methyl.

Variable r can be any suitable number known by one of skill in the art. In some embodiments, r is selected from 0 to 5. In some embodiments, r is selected from 0 to 3. In some embodiments, r is selected from 0 to 2. In some embodiments, r is 0 or 1. In some embodiments, r is 0.

The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, compounds described herein are intended to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S ³⁶S, ³⁵Cl, ³⁷Cl, ⁷⁹Br, ⁸¹Br, and ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)]2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Compounds of this disclosure include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

Included in the present disclosure are salts, particularly pharmaceutically acceptable salts, of the compounds described herein. The compounds of the present disclosure that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

The compounds described herein may in some cases exist as diastereomers, enantiomers, or other stereoisomeric forms. The compounds presented herein include all diastereomeric, enantiomeric, and epimeric forms as well as the appropriate mixtures thereof. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis.

The methods and compositions described herein include the use of amorphous forms as well as crystalline forms (also known as polymorphs). The compounds described herein may be in the form of pharmaceutically acceptable salts. As well, in some embodiments, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

In certain embodiments, compounds or salts of the compounds may be prodrugs, e.g., wherein a hydroxyl in the parent compound is presented as an ester or a carbonate, or carboxylic acid present in the parent compound is presented as an ester. The term “prodrug” is intended to encompass compounds which, under physiologic conditions, are converted into pharmaceutical agents of the present disclosure. One method for making a prodrug is to include one or more selected moieties which are hydrolyzed under physiologic conditions to reveal the desired molecule. In other embodiments, the prodrug is converted by an enzymatic activity of the host animal such as specific target cells in the host animal. For example, esters or carbonates (e.g., esters or carbonates of alcohols or carboxylic acids and esters of phosphonic acids) are preferred prodrugs of the present disclosure.

Prodrug forms of the herein described compounds, wherein the prodrug is metabolized in vivo to produce a compound as set forth herein are included within the scope of the claims. In some cases, some of the herein-described compounds may be a prodrug for another derivative or active compound.

Prodrugs are often useful because, in some situations, they may be easier to administer than the parent drug. They may, for instance, be bioavailable by oral administration whereas the parent is not. Prodrugs may help enhance the cell permeability of a compound relative to the parent drug. The prodrug may also have improved solubility in pharmaceutical compositions over the parent drug. Prodrugs may be designed as reversible drug derivatives, for use as modifiers to enhance drug transport to site-specific tissues or to increase drug residence inside of a cell.

In some embodiments, the design of a prodrug increases the lipophilicity of the pharmaceutical agent. In some embodiments, the design of a prodrug increases the effective water solubility. See, e.g., Fedorak et al., Am. J. Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein for such disclosure). According to another embodiment, the present disclosure provides methods of producing the above-defined compounds. The compounds may be synthesized using conventional techniques. Advantageously, these compounds are conveniently synthesized from readily available starting materials.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

Additional embodiments of the pharmaceutical formulations contemplated in the methods of this disclosure, including Embodiment 53, are described below:

### Pharmaceutical Formulations

Formulation may be modified depending upon the route of administration chosen. Pharmaceutical compositions comprising a compound, salt or conjugate may be manufactured, for example, by lyophilizing the compound, salt or conjugate, mixing, dissolving, emulsifying, encapsulating or entrapping the conjugate. The pharmaceutical compositions may also include the compounds, salts or conjugates in a free-base form or pharmaceutically-acceptable salt form.

Methods for formulation of a compound or pharmaceutically acceptable salt of Formula (I0), (I), (IA), (IAA), (IB), (IBB), (IC), (ICC), (ID), (IDD), (IE), or (IEE) may include formulating any of the compounds, salts or conjugates with one or more inert, pharmaceutically-acceptable excipients or carriers to form a solid, semi-solid, or liquid composition. Solid compositions may include, for example, powders, tablets, dispersible granules and capsules, and in some aspects, the solid compositions further contain nontoxic, auxiliary substances, for example wetting or emulsifying agents, pH buffering agents, and other pharmaceutically-acceptable additives. Alternatively, the compounds, salts or conjugates may be lyophilized or in powder form for re-constitution with a suitable vehicle, e.g., sterile pyrogen-free water, before use.

Pharmaceutical compositions comprising a compound or pharmaceutically acceptable salt of Formula (I0), (I), (IA), (IAA), (IB), (IBB), (IC), (ICC), (ID), (IDD), (IE), or (IEE) may comprise at least one active ingredient (e.g., a compound, salt or conjugate and other agents). The active ingredients may be entrapped in microcapsules prepared, for example, by coacervation techniques or by interfacial polymerization (e.g., hydroxymethylcellulose or gelatin microcapsules and poly-(methylmethacylate) microcapsules, respectively), in colloidal drug-delivery systems (e.g., liposomes, albumin microspheres, microemulsions, nano-particles and nanocapsules) or in macroemulsions.

The compositions and formulations may be sterilized. Sterilization may be accomplished by filtration through sterile filtration.

The compositions comprising a compound or pharmaceutically acceptable salt of Formula (I0), (I), (IA), (IAA), (IB), (IBB), (IC), (ICC), (ID), (IDD), (IE), or (IEE) may be formulated for administration as an injection. Non-limiting examples of formulations for injection may include a sterile suspension, solution or emulsion in oily or aqueous vehicles. Suitable oily vehicles may include, but are not limited to, lipophilic solvents or vehicles such as fatty oils or synthetic fatty acid esters, or liposomes. Aqueous injection suspensions may contain substances which increase the viscosity of the suspension. The suspension may also contain suitable stabilizers. Injections may be formulated for bolus injection or continuous infusion. Alternatively, the compositions may be lyophilized or in powder form for reconstitution with a suitable vehicle, e.g., sterile pyrogen-free water, before use.

For parenteral administration, a compound or pharmaceutically acceptable salt of Formula (I0), (I), (IA), (IAA), (IB), (IBB), (IC), (ICC), (ID), (IDD), (IE), or (IEE) may be formulated in a unit dosage injectable form (e.g., solution, suspension, emulsion) in association with a pharmaceutically acceptable parenteral vehicle. Such vehicles may be inherently non-toxic, and non-therapeutic. Vehicles may be water, saline, Ringer's solution, dextrose solution, and 5% human serum albumin. Non-aqueous vehicles such as fixed oils and ethyl oleate may also be used. Liposomes may be used as carriers. The vehicle may contain minor amounts of additives such as substances that enhance isotonicity and chemical stability (e.g., buffers and preservatives).

In one embodiment of the methods of this disclosure, Formula (I0), (I), (IA), (IAA), (IB), (IBB), (IC), (ICC), (ID), (IDD), (IE), or (IEE) formulated for oral delivery to a subject in need. In one embodiment a composition is formulated so as to deliver one or more pharmaceutically active agents to a subject through a mucosa layer in the mouth or esophagus. In another embodiment the composition is formulated to deliver one or more pharmaceutically active agents to a subject through a mucosa layer in the stomach and/or intestines.

In one embodiment of the methods of this disclosure, Formula (I0), (I), (IA), (IAA), (IB), (IBB), (IC), (ICC), (ID), (IDD), (IE), or (IEE) are provided in modified release dosage forms. Suitable modified release dosage vehicles include, but are not limited to, hydrophilic or hydrophobic matrix devices, water-soluble separating layer coatings, enteric coatings, osmotic devices, multi-particulate devices, and combinations thereof. The compositions may also comprise non-release controlling excipients.

In another embodiment of the methods of this disclosure, compositions of Formula (I0), (I), (IA), (IAA), (IB), (IBB), (IC), (ICC), (ID), (IDD), (IE), or (IEE) are provided in enteric coated dosage forms. These enteric coated dosage forms can also comprise non-release controlling excipients. In one embodiment the compositions are in the form of enteric-coated granules, as controlled-release capsules for oral administration. The compositions can further comprise cellulose, disodium hydrogen phosphate, hydroxypropyl cellulose, pyridazine, lactose, mannitol, or sodium lauryl sulfate. In another embodiment the compositions are in the form of enteric-coated pellets, as controlled-release capsules for oral administration. The compositions can further comprise glycerol monostearate 40-50, hydroxypropyl cellulose, pyridazine, magnesium stearate, methacrylic acid copolymer type C, polysorbate 80, sugar spheres, talc, or triethyl citrate.

In another embodiment of the methods of this disclosure, the compositions of Formula (I0), (I), (IA), (IAA), (IB), (IBB), (IC), (ICC), (ID), (IDD), (IE), or (IEE) are enteric-coated controlled-release tablets for oral administration. The compositions can further comprise carnauba wax, crospovidone, diacetylated monoglycerides, ethylcellulose, hydroxypropyl cellulose, pyridazine phthalate, magnesium stearate, mannitol, sodium hydroxide, sodium stearyl fumarate, talc, titanium dioxide, or yellow ferric oxide.

Sustained-release preparations comprising a compound or pharmaceutically acceptable salt of Formula (I0), (I), (IA), (IAA), (IB), (IBB), (IC), (ICC), (ID), (IDD), (IE), or (TEE) may also be prepared. Examples of sustained-release preparations may include semipermeable matrices of solid hydrophobic polymers that may contain the compound, salt or conjugate, and these matrices may be in the form of shaped articles (e.g., films or microcapsules). Examples of sustained-release matrices may include polyesters, hydrogels (e.g., poly(2-hydroxyethyl-methacrylate), or poly(vinyl alcohol)), polylactides, copolymers of L-glutamic acid and 7 ethyl-L-glutamate, non-degradable ethylene-vinyl acetate, degradable lactic acid-glycolic acid copolymers such as the LUPRON DEPO™ (i.e., injectable microspheres composed of lactic acid-glycolic acid copolymer and leuprolide acetate), and poly-D-(−)-3-hydroxybutyric acid.

Pharmaceutical formulations comprising a compound or pharmaceutically acceptable salt of Formula (I0), (I), (IA), (IAA), (IB), (IBB), (IC), (ICC), (ID), (IDD), (IE), or (IEE) may be prepared for storage by mixing a compound, salt or conjugate with a pharmaceutically acceptable carrier, excipient, and/or a stabilizer. This formulation may be a lyophilized formulation or an aqueous solution. Acceptable carriers, excipients, and/or stabilizers may be nontoxic to recipients at the dosages and concentrations used. Acceptable carriers, excipients, and/or stabilizers may include buffers such as phosphate, citrate, and other organic acids; antioxidants including ascorbic acid and methionine; preservatives, polypeptides; proteins, such as serum albumin or gelatin; hydrophilic polymers; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes; and/or non-ionic surfactants or polyethylene glycol.

In another embodiment of the methods of this disclosure, compositions of Formula (I0), (I), (IA), (IAA), (IB), (IBB), (IC), (ICC), (ID), (IDD), (IE), or (IEE) can further comprise calcium stearate, crospovidone, hydroxypropyl methylcellulose, iron oxide, mannitol, methacrylic acid copolymer, polysorbate 80, povidone, propylene glycol, sodium carbonate, sodium lauryl sulfate, titanium dioxide, and triethyl citrate.

In another embodiment of the methods of this disclosure, compositions of Formula (I0), (I), (IA), (IAA), (IB), (IBB), (IC), (ICC), (ID), (IDD), (IE), or (IEE) are provided in effervescent dosage forms. These effervescent dosage forms can also comprise non-release controlling excipients.

In another embodiment of the methods of this disclosure, compositions of Formula (I0), (I), (IA), (IAA), (IB), (IBB), (IC), (ICC), (ID), (IDD), (IE), or (IEE) can be provided in a dosage form that has at least one component that can facilitate the immediate release of an active agent, and at least one component that can facilitate the controlled release of an active agent. In a further embodiment the dosage form can be capable of giving a discontinuous release of the compound in the form of at least two consecutive pulses separated in time from 0.1 up to 24 hours. The compositions can comprise one or more release controlling and non-release controlling excipients, such as those excipients suitable for a disruptable semi-permeable membrane and as swellable substances.

In another embodiment of the methods of this disclosure, compositions of Formula (I0), (I), (IA), (IAA), (IB), (IBB), (IC), (ICC), (ID), (IDD), (IE), or (IEE) are provided in a dosage form for oral administration to a subject, which comprise one or more pharmaceutically acceptable excipients or carriers, enclosed in an intermediate reactive layer comprising a gastric juice-resistant polymeric layered material partially neutralized with alkali and having cation exchange capacity and a gastric juice-resistant outer layer.

In some embodiments of the methods of this disclosure, the compositions of (I0), (I), (IA), (IAA), (IB), (IBB), (IC), (ICC), (ID), (IDD), (IE), or (IEE) provided herein can be in unit-dosage forms or multiple-dosage forms. Unit-dosage forms, as used herein, refer to physically discrete units suitable for administration to human or non-human animal subjects and packaged individually. Each unit-dose can contain a predetermined quantity of an active ingredient(s) sufficient to produce the desired therapeutic effect, in association with the required pharmaceutical carriers or excipients. Examples of unit-dosage forms include, but are not limited to, ampoules, syringes, and individually packaged tablets and capsules. In some embodiments, unit-dosage forms may be administered in fractions or multiples thereof. A multiple-dosage form is a plurality of identical unit-dosage forms packaged in a single container, which can be administered in segregated unit-dosage form. Examples of multiple-dosage forms include, but are not limited to, vials, bottles of tablets or capsules, or bottles of pints or gallons. In another embodiment the multiple dosage forms comprise different pharmaceutically active agents.

In some embodiments of the methods of this disclosure, the compositions of (I0), (I), (IA), (IAA), (IB), (IBB), (IC), (ICC), (ID), (IDD), (IE), or (IEE) may also be formulated as a modified release dosage form, including immediate-, delayed-, extended-, prolonged-, sustained-, pulsatile-, controlled-, extended, accelerated- and fast-, targeted-, programmed-release, and gastric retention dosage forms. These dosage forms can be prepared according to known methods and techniques (see, Remington: The Science and Practice of Pharmacy, supra; Modified-Release Drug Delivery Technology, Rathbone et al., Eds., Drugs and the Pharmaceutical Science, Marcel Dekker, Inc.: New York, N.Y., 2002; Vol. 126, which are herein incorporated by reference in their entirety).

### EXAMPLES

The invention now being generally described, it will be more readily understood by reference to the following examples which are included merely for purposes of illustration of certain aspects and embodiments of the present invention, and are not intended to limit the invention in any way.

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

### General Synthetic Schemes 1-3

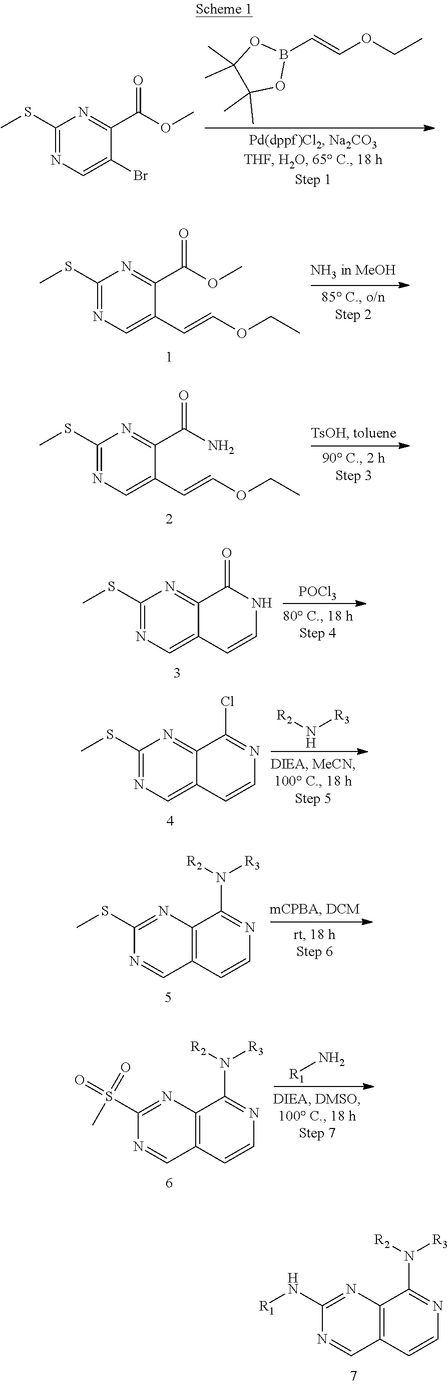

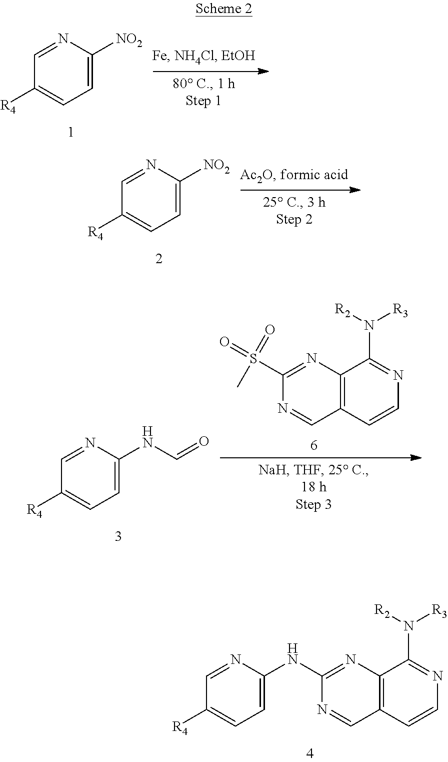

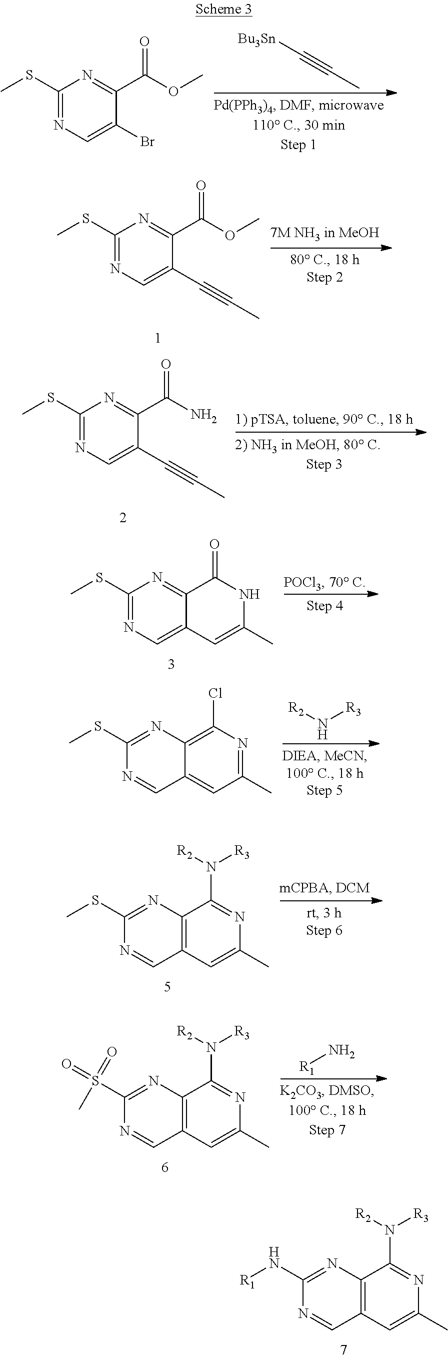

### INTERMEDIATES

### Intermediate 1: 8-chloro-2-(methylthio)pyrido[3,4-d]pyrimidine

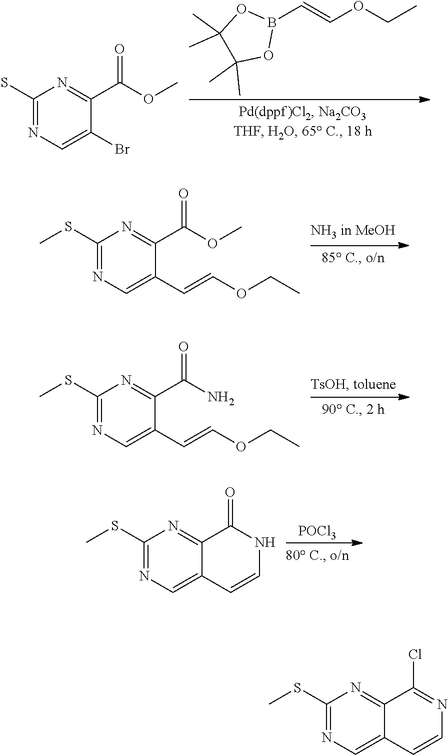

### Step 1: methyl (E)-5-(2-ethoxyvinyl)-2-(methylthio)pyrimidine-4-carboxylate

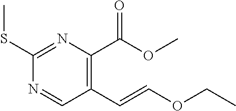 [SMILES: CCO/C=C/c1cnc(SC)nc1C(=O)OC]

To a stirred mixture of methyl 5-bromo-2-(methylsulfanyl)pyrimidine-4-carboxylate (7.8 g, 29.64 mmol) and 2-[(E)-2-ethoxyethenyl]-4,4,5,5-tetramethyl-1,3,2-dioxaborolane (9.45 g, 44.46 mmol) in tetrahydrofuran (60 mL) were added sodium carbonate (4.71 g, 44.46 mmol) in water (20 mL) and Pd(dppf)Cl₂ (1.08 g, 1.48 mmol) under a nitrogen atmosphere. The resulting mixture was heated to 65° C. and stirred for 18 hours. The reaction mixture was allowed to cool to room temperature, diluted with water (200 mL) and extracted with ethyl acetate (3×200 mL). The combined organic layers were washed with brine (200 mL), dried over anhydrous sodium sulfate, filtered, and concentrated under vacuum to afford the crude product. The residue was purified by silica gel column chromatography (ethyl acetate/petroleum ether, 85:15) to afford methyl (E)-5-(2-ethoxyvinyl)-2-(methylthio) pyrimidine-4-carboxylate (5.30 g, 70.3% yield).

LCMS (ESI) m/z=255 [M+H]⁺.

### Step 2: (E)-5-(2-ethoxyvinyl)-2-(methylthio)pyrimidine-4-carboxamide

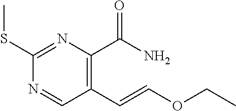 [SMILES: CCO/C=C/c1cnc(SC)nc1C(N)=O]

To a stirred mixture of methyl (E)-5-(2-ethoxyvinyl)-2-(methylthio) pyrimidine-4-carboxylate (4.8 g, 18.87 mmol) in methanol (5 mL) was added a solution of ammonium in methanol (7 M, 50 mL, 350 mmol). The resulting mixture was heated to 85° C. and stirred overnight in a sealed tube. The resulting mixture was allowed to cool to room temperature and concentrated under vacuum to afford the crude title product (4.2 g). The crude product was used for next step directly without further purification.

LCMS (ESI) m/z=240 [M+H]⁺.

### Step 3: 2-(methylthio)pyrido[3,4-d]pyrimidin-8(7H)-one

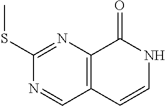

To a stirred mixture of (E)-5-(2-ethoxyvinyl)-2-(methylthio)pyrimidine-4-carboxamide (4.2 g, 17.55 mmol) in toluene (50 mL) was added p-toluene sulfonic acid (0.30 g, 1.75 mmol). The resulting mixture was heated to 90° C. and stirred for 2 hours. The reaction mixture was allowed to cool to room temperature and concentrated under vacuum to afford the crude product. The residue was diluted with water (200 mL) and extracted with ethyl acetate (3×200 mL). The combined organic layers were washed with brine (200 mL), dried over anhydrous sodium sulfate, filtered and concentrated under vacuum to afford the crude title product (4.4 g). The crude product was used for next step directly without further purification.

LCMS (ESI) m/z=194 [M+H]⁺.

### Step 4: 8-chloro-2-(methylthio)pyrido[3,4-d]pyrimidine

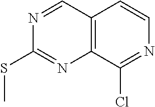

A mixture of 2-(methylthio)pyrido[3,4-d]pyrimidin-8(7H)-one (4.2 g, 21.73 mmol) in phosphorus oxychloride (150 mL) was heated to 80° C. and stirred overnight. The reaction mixture was allowed to cool to room temperature and concentrated under high vacuum. The residue was carefully quenched by addition of saturated aqueous sodium bicarbonate (500 mL) and extracted with ethyl acetate (3×500 mL). The combined organic layers were washed with brine (1000 mL), dried over anhydrous sodium sulfate, filtered, and concentrated under vacuum to afford the crude product. The residue was purified by silica gel column chromatography (petroleum ether/ethyl acetate, 2:1) to afford 8-chloro-2-(methylthio)pyrido[3,4-d]pyrimidine (3.70 g, 92.4% yield).

LCMS (ESI) m/z=212 [M+H]⁺.

### Intermediate 2: 6-methyl-2-(methylthio)pyrido[3,4-d]pyrimidin-8(7H)-one

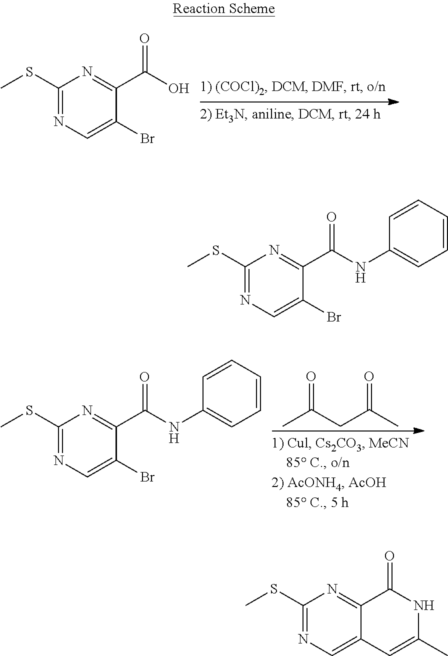

### Detailed Procedure

### Step 1: 5-Bromo-2-(methylthio)-N-phenylpyrimidine-4-carboxamide

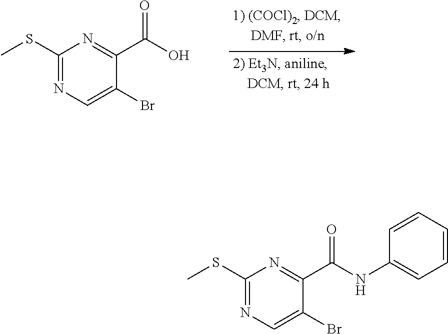 [SMILES: CSc1ncc(Br)c(C(=O)O)n1 ; CSc1ncc(Br)c(C(=O)Nc2ccccc2)n1]

Oxalyl dichloride (13.25 g, 104.4 mmol) was added to a solution of 5-bromo-2-(methylthio)pyrimidine-4-carboxylic acid (20 g, 80.3 mmol) in DCM (800 mL) at 0° C. A drop of DMF was added and the resulting mixture was stirred overnight at room temperature and concentrated under reduced pressure. The residue was dissolved in DCM (800 mL) and aniline (11.96 g, 128.5 mmol) and Et₃N (17.06 g, 168.6 mmol) were added at 0° C. The resulting mixture was stirred at room temperature for 24 h. The reaction was quenched by addition of aqueous HCl (0.5 N, 500 mL) and extracted with DCM (2×500 mL). The combined organic layers were washed with water (2×500 mL) and brine (500 mL), dried over anhydrous sodium sulfate and concentrated under reduced pressure to afford the crude product 5-bromo-2-(methylthio)-N-phenylpyrimidine-4-carboxamide (26 g crude). The crude product was used as such for next step.

LCMS (ESI−MS) m/z=324.0, 326.0 [M+H]⁺.

### Step 2: 6-Methyl-2-(methylthio)pyrido[3,4-d]pyrimidin-8(7H)-one

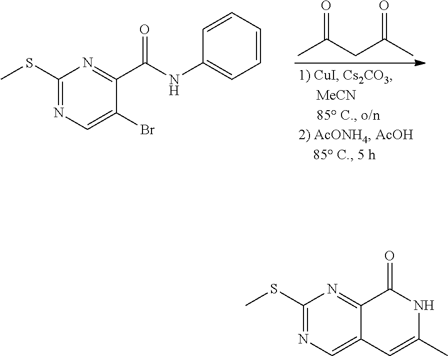

To a solution of 5-bromo-2-(methylthio)-N-phenylpyrimidine-4-carboxamide (26 g, 80.2 mmol) in ACN (270 mL) was added pentane-2,4-dione (16.06 g, 160.4 mmol), CuI (1.53 g, 8.02 mmol) and Cs₂CO₃ (52.26 g, 160.4 mmol). The resulting mixture was heated to 85° C. and stirred overnight. Ammonium acetate (101.8 g, 1.72 mol) and acetic acid (200 mL) were added and the resulting mixture was heated to 85° C. and stirred at for 5 h. After cooling to room temperature, the reaction mixture was concentrated under high vacuum until most of the liquid was evaporated. The residue was then neutralized by the addition of saturated aqueous NaOH (˜500 mL) at 0° C. followed by saturated aqueous NaHCO₃ at 0° C. until the pH value was adjusted to 6-7. The aqueous layer was extracted with DCM (2×1 L). The combined organic layers were washed with brine (2×500 mL), dried over anhydrous sodium sulfate and concentrated under reduced pressure to afford the crude product. The residue was purified by flash column chromatography eluting with 20% to 80% of ethyl acetate in petroleum ether followed by 0% to 10% MeOH in DCM to afford the desired product 6-methyl-2-(methylthio)pyrido[3,4-d]pyrimidin-8(7H)-one (5.8 g, 35% yield for 3 steps).

¹H NMR (400 MHz, DMSO-d₆) δ 11.9 (s, 1H), 9.07 (s, 1H), 6.31 (s, 1H), 2.58 (s, 3H), 2.22 (s, 3H).

LCMS (ESI−MS) m/z=208.0 [M+H]⁺.

### Intermediate 3: 8-chloro-6-methyl-2-(methylthio)pyrido[3,4-d]pyrimidine 8-Chloro-6-methyl-2-(methylthio)pyrido[3,4-d]pyrimidine

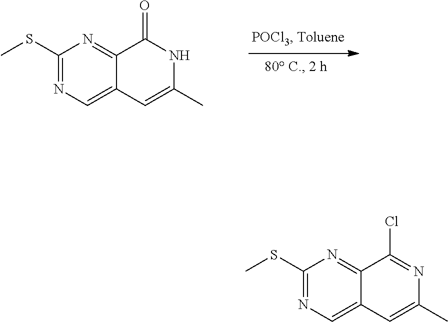

To a solution of 6-methyl-2-(methylthio)pyrido[3,4-d]pyrimidin-8(7H)-one (4 g, 19.30 mmol) in toluene (20 mL) was added POCl₃ (32.00 mL). The mixture was stirred at 80° C. for 2 h. The resulting mixture was concentrated under reduced pressure, quenched with water (200 mL) and extracted with ethyl acetate (3×200 mL). The combined organic layers were dried over anhydrous sodium sulfate, filtered and concentrated under reduced pressure. The crude product was purified by silica gel column chromatography, eluting with ethyl acetate/petroleum ether (0-20%) to afford 8-chloro-6-methyl-2-(methylthio)pyrido[3,4-d]pyrimidine (2.01 g, 46.4% yield).

¹H NMR (400 MHz, DMSO-d₆) δ 9.47 (s, 1H), 7.79 (d, J=0.6 Hz, 1H), 2.66 (s, 3H), 2.60 (s, 3H).

LCMS (ESI−MS) m/z=226.0 [M+H]⁺.

### Intermediate 4: 8-chloro-6-methyl-2-(methylsulfonyl)pyrido[3,4-d]pyrimidine and 8-chloro-6-methyl-2-(methylsulfinyl)pyrido[3,4-d]pyrimidine

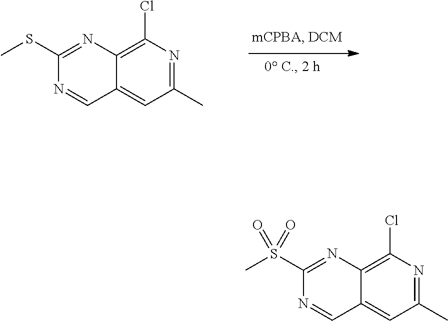

3-Chloroperoxybenzoic acid (6.88 g, 39.87 mmol) was added to 8-chloro-6-methyl-2-(methylthio)pyrido[3,4-d]pyrimidine (3.0 g, 13.29 mmol) in dichloromethane (50 mL) at 0° C. The reaction mixture was stirred for 30 minutes at 0° C., warmed to room temperature and stirred for 2 h. The reaction was quenched with saturated aqueous sodium bicarbonate (200 mL). The resulting mixture was extracted with dichloromethane (3×200 mL). The combined organic layers were dried over anhydrous sodium sulfate. After filtration, the filtrate was concentrated under reduced pressure. The residue was purified by column chromatography (EA in PE 0% to 100%) to afford crude 8-chloro-6-methyl-2-(methylsulfonyl)pyrido[3,4-d]pyrimidine and 8-chloro-6-methyl-2-(methylsulfinyl)pyrido[3,4-d]pyrimidine (1.07 g, 30.4% yield).

¹H NMR (400 MHz, DMSO-d₆) δ 9.99 (s, 1H), 8.09-8.06 (m, 1H), 3.54 (s, 3H), 2.81-2.66 (m, 3H).

LCMS (ESI−MS) m/z=257.9 [M+H]⁺.

### Intermediate 5: 8-bromo-6-methyl-N-(1-(methylsulfonyl)piperidin-4-yl)pyrido[3,4-d]pyrimidin-2-amine

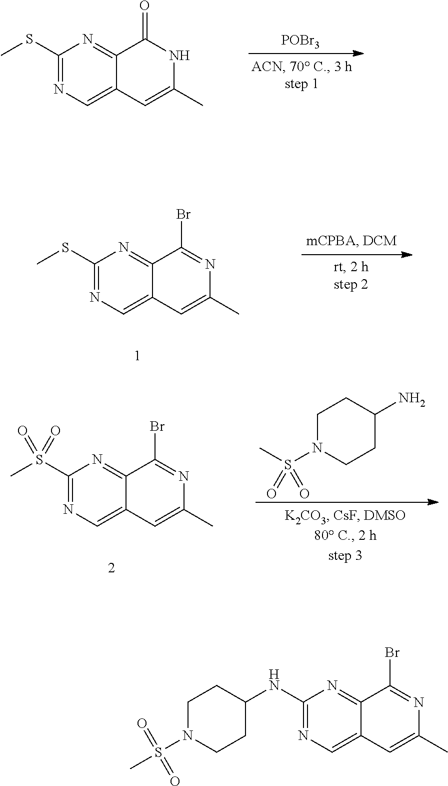

### Step 1: 8-Bromo-6-methyl-2-(methylthio)pyrido[3,4-d]pyrimidine

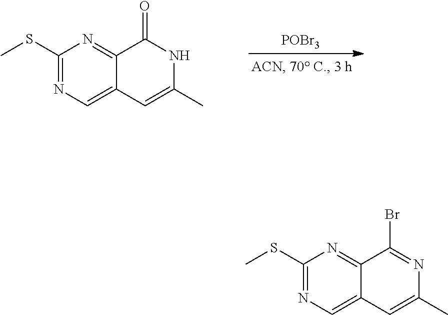

POBr3 (50 mL, 174.4 mmol) was added to 6-methyl-2-(methylthio)pyrido[3,4-d]pyrimidin-8(7H)-one (5 g, 24.12 mmol) in MeCN (50 mL). The mixture was stirred at 70° C. for 3 h and concentrated under reduced pressure. The residue was quenched with aqueous sodium bicarbonate at 0° C. and extracted with EA (3×200 mL). The combined organic layers were dried over anhydrous sodium sulfate. After filtration, the filtrate was concentrated under reduced pressure. The residue was purified by silica gel column chromatography, eluting with EA/PE (0-20%) to afford 8-bromo-6-methyl-2-(methylthio)pyrido[3,4-d]pyrimidine (2.2 g, 33.8% yield).

LCMS (ESI−MS) m/z=270.0, 272.0 [M+H]⁺.

### Step 2: 8-Bromo-6-methyl-2-(methylsulfonyl)pyrido[3,4-d]pyrimidine

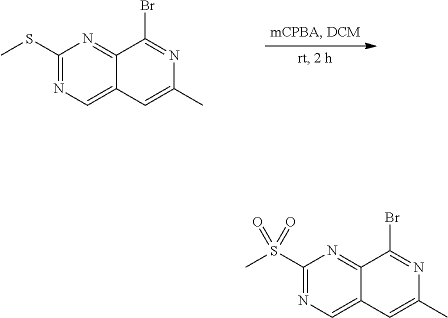

3-Chloroperoxybenzoic acid (2.81 g, 16.28 mmol) was added to 8-bromo-6-methyl-2-(methylthio)pyrido[3,4-d]pyrimidine (2.2 g, 8.14 mmol) in DCM (50 mL) at 0° C. The reaction mixture was stirred for 30 minutes at 0° C., then warmed to room temperature and stirred for 2 h. The reaction was quenched with saturated aqueous sodium bicarbonate (200 mL). The resulting mixture was extracted with DCM (3×200 mL). The combined organic layers were dried over anhydrous sodium sulfate. After filtration, the filtrate was concentrated under reduced pressure. The residue was purified by column chromatography (EA in PE from 0% to 100%) to afford crude 8-bromo-6-methyl-2-(methylsulfonyl)pyrido[3,4-d]pyrimidine (1.3 g, 52.8% yield).

LCMS (ESI−MS) m/z=302.0, 304.0 [M+H]⁺.

### Step 3: 8-Bromo-6-methyl-N-(1-(methylsulfonyl)piperidin-4-yl)pyrido[3,4-d]pyrimidin-2-amine

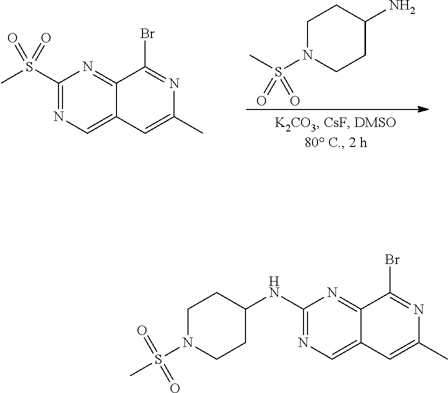

To a solution of 8-bromo-6-methyl-2-(methylsulfonyl)pyrido[3,4-d]pyrimidine (1.3 g, 4.30 mmol) in DMSO (10 mL) was added 1-(methylsulfonyl)piperidin-4-amine (0.77 g, 4.30 mmol), K₂CO₃ (1.19 g, 8.60 mmol) and CsF (1.31 g, 8.60 mmol). The reaction mixture was stirred for 2 h at 80° C. The reaction mixture was diluted with water (50 mL) and extracted with DCM (3×50 mL). The combined organic layers were washed with brine (2×40 mL), dried over anhydrous sodium sulfate and concentrated under reduced pressure. The residue was purified by silica gel column chromatography (EA/PE, 0-20%) to afford 8-bromo-6-methyl-N-(1-(methylsulfonyl)piperidin-4-yl)pyrido[3,4-d]pyrimidin-2-amine (206.1 mg, 10.8% yield).

¹H NMR (400 MHz, DMSO-d₆) δ 9.18 (s, 1H), 8.12-7.96 (m, 1H), 7.57-7.54 (m, 1H), 4.09-3.94 (m, 1H), 3.64-3.51 (m, 2H), 2.90 (s, 6H), 2.57-2.45 (m, 2H), 2.18-2.01 (m, 2H), 1.71-1.56 (m, 2H).

LCMS (ESI−MS) m/z=400.0, 402.0 [M+H]⁺.

### Intermediate 6: 8-chloro-6-methyl-N-(1-(methylsulfonyl)piperidin-4-yl)pyrido[3,4-d]pyrimidin-2-amine

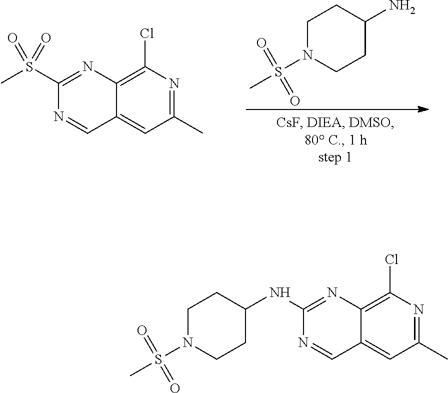

A mixture of 8-chloro-6-methyl-2-(methylsulfonyl)pyrido[3,4-d]pyrimidine (1 g, 3.88 mmol), 1-(methylsulfonyl)piperidin-4-amine (0.8 g, 4.26 mmol), DIEA (1.50 g, 11.64 mmol) and CsF (1.77 g, 11.64 mmol) in DMSO (5 mL) was stirred for 1 h at 80° C. The reaction mixture was diluted with water (100 mL) and extracted with CH₂Cl₂ (3×100 mL). The combined organic layers were dried over anhydrous Na₂SO₄. After filtration, the filtrate was concentrated under reduced pressure. The residue was purified by silica gel column chromatography (PE/EA, 1:1) to afford 8-chloro-6-methyl-N-(1-(methylsulfonyl)piperidin-4-yl)pyrido[3,4-d]pyrimidin-2-amine (505.8 mg, 33.3% yield).

LCMS (ESI−MS) m/z=356.0 [M+H]⁺.

### Intermediate 7: 8-chloro-6-cyclopropyl-2-methanesulfonylpyrido[3,4-d]pyrimidine

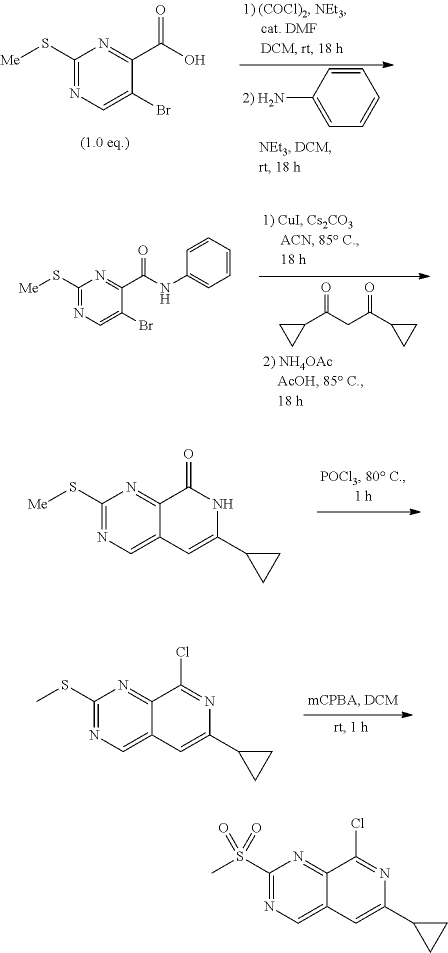

### Step 1: 5-bromo-2-(methylsulfanyl)-N-phenylpyrimidine-4-carboxamide

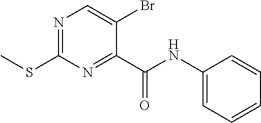 [SMILES: CSc1ncc(Br)c(C(=O)Nc2ccccc2)n1]

A solution of 5-bromo-2-(methylthio)pyrimidine-4-carboxylic acid (15.0 g, 60.222 mmol) in dichloromethane anhydrous (225.0 ml) was cooled down to 0° C. Oxalyl chloride (6.624 ml, 78.288 mmol) and one drop of dimethylformamide anhydrous were added under argon atmosphere and the reaction mixture was stirred at room temperature for 18 hours. Then, the reaction mixture was concentrated under reduced pressure and the residue was redissolved in dichloromethane anhydrous (225.0 ml) and cooled down to 0° C. Aniline (9.329 ml, 102.377 mmol) and triethylamine (18.467 ml, 132.488 mmol) were slowly added and the reaction mixture was stirred at room temperature for additional 18 hours. The reaction mixture was quenched with 0.5 M HCl aq. sol. and layers were separated. Aqueous layer was extracted with dichloromethane (3×200 ml) and the combined organic layers were dried over anhydrous MgSO₄, filtered and concentrated under reduced pressure. Crude material was purified by automated flash column chromatography on silica gel (from hexane to hexane/EtOAc 4:1) to afford the title compound (17.34 g, 89% yield).

¹H NMR (300 MHz, Chloroform-d) δ 9.63 (bs, 1H), 8.86 (s, 1H), 7.77-7.71 (m, 2H), 7.42 (t, J=7.9 Hz, 2H), 7.21 (t, J=7.4 Hz, 1H), 2.66 (s, 3H).

UPLC (ESI) [M+H]⁺32 323.70 and 325.70 (Br isotopic pattern).

### Step 2: 6-cyclopropyl-2-(methylsulfanyl)-7H,8H-pyrido[3,4-d]pyrimidin-8-one

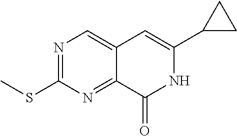 [SMILES: CSc1ncc2cc(C3CC3)[nH]c(=O)c2n1]

A solution of 5-bromo-2-(methylsulfanyl)-N-phenylpyrimidine-4-carboxamide (10.0 g, 30.845 mmol), 1,3-dicyclopropylpropane-1,3-dione (7.633 ml, 61.69 mmol), cesium carbonate (20.1 g, 61.69 mmol) and copper(I) iodide (0.587 g, 3.085 mmol) in acetonitrile anhydrous (88.13 ml) was stirred at 85° C. for 18 hours. Then, acetic acid (88.13 ml) and ammonium acetate (35.665 g, 462.677 mmol) were added and the reaction mixture was stirred at 85° C. for additional 18 hours. The reaction mixture was concentrated under reduced pressure to remove acetonitrile, and acetic acid-containing mixture was diluted with water (100 ml) and extracted with dichloromethane (3×100 ml). The combined organic layers were dried over anhydrous MgSO₄, filtered and concentrated under reduced pressure. Crude material was purified by automated flash column chromatography on silica gel (from hexane/EtOAc 3:2 to EtOAc) to afford the title compound (4.76 g, 66% yield).

¹H NMR (300 MHz, DMSO-d₆) δ 12.01 (bs, 1H), 9.06 (s, 1H), 6.21 (s, 1H), 2.58 (s, 3H), 1.88 (tt, J=8.3, 5.1 Hz, 1H), 1.04-0.92 (m, 2H), 0.89-0.77 (m, 2H).

UPLC (ESI) [M+H]⁺233.90 .

### Step 3: 8-chloro-6-cyclopropyl-2-(methylsulfanyl)pyrido[3,4-d]pyrimidine

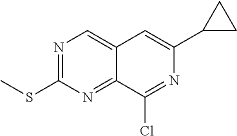 [SMILES: CSc1ncc2cc(C3CC3)nc(Cl)c2n1]

A solution of 6-cyclopropyl-2-(methylsulfanyl)-7H,8H-pyrido[3,4-d]pyrimidin-8-one (4.76 g, 20.404 mmol) in phosphorus oxychloride (32.428 ml, 346.864 mmol) was stirred at 80° C. for 1 hour. The reaction mixture was concentrated under reduced pressure and the residue was redissolved in ethyl acetate (50 ml) and quenched with NaHCO₃ sat. aq. solution. Layers were separated and aqueous layer was extracted with ethyl acetate (3×50 ml). The combined organic layers were dried over anhydrous MgSO₄, filtered and concentrated under reduced pressure. Crude material was purified by automated flash column chromatography on silica gel (from hexane to hexane/EtOAc 7:3) to afford the title compound (4.58 g, 89% yield).

¹H NMR (300 MHz, DMSO-d₆) δ 9.48 (s, 1H), 7.87 (s, 1H), 2.66 (s, 3H), 2.38-2.19 (m, 1H), 1.14-1.02 (m, 2H), 0.97 (ddd, J=7.2, 5.1, 2.9 Hz, 2H).

UPLC (ESI) [M+H]⁺251.85 .

### Step 4: 8-chloro-6-cyclopropyl-2-methanesulfonylpyrido[3,4-d]pyrimidine

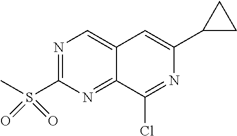 [SMILES: CS(=O)(=O)c1ncc2cc(C3CC3)nc(Cl)c2n1]

To a solution of 8-chloro-6-cyclopropyl-2-(methylsulfanyl)pyrido[3,4-d]pyrimidine (2.5 g, 9.931 mmol) in dichloromethane (125.0 ml), 3-chloroperbenzoic acid (8.569 g, 49.656 mmol) was added portionwise and the reaction mixture was stirred at room temperature for 1 hour. The reaction mixture was quenched by the slowly addition of a 10% Na₂S₂O₃ aq. solution (50 ml) and extracted with dichloromethane (3×70 ml). The combined organic layers were washed with NaHCO₃ sat. aq. solution, dried over anhydrous MgSO₄, filtered and concentrated under reduced pressure. Crude material was purified by automated flash column chromatography on silica gel (from hexane/EtOAc 4:1 to hexane/EtOAc 2:3) to afford the title compound (2.54 g, 89% yield).

¹H NMR (300 MHz, DMSO-d₆) δ 9.94 (s, 1H), 8.12 (s, 1H), 3.52 (s, 3H), 2.42 (dq, J=8.4, 4.7, 4.1 Hz, 1H), 1.17 (dt, J=7.9, 3.0 Hz, 2H), 1.09-1.02 (m, 2H).

UPLC (ESI) [M+H]⁺283.80.

### Intermediate 8: 8-chloro-6-cyclopropyl-N-(1-(methylsulfonyl)piperidin-4-yl)pyrido[3,4-d]pyrimidin-2-amine

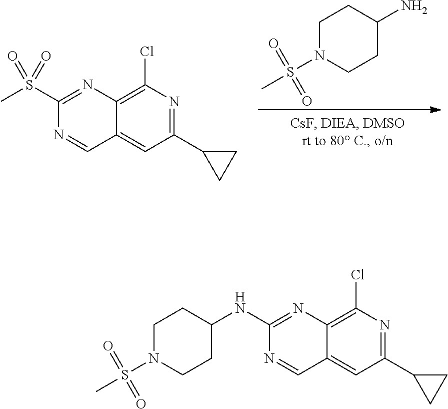 [SMILES: CS(=O)(=O)c1ncc2cc(C3CC3)nc(Cl)c2n1 ; CS(=O)(=O)N1CCC(N)CC1 ; CS(=O)(=O)N1CCC(Nc2ncc3cc(C4CC4)nc(Cl)c3n2)CC1]

A solution of 8-chloro-6-cyclopropyl-2-(methylsulfonyl)pyrido[3,4-d]pyrimidine (100 mg, 352 mol) in dimethylsulfoxide anhydrous (1.8 mL) was charged with 1-(methylsulfonyl)piperidin-4-amine (69.1 mg, 388 mol), cesium fluoride (161 mg, 1.06 mmol), and N-ethyl-N-isopropylpropan-2-amine (137 mg, 184 L, 1.06 mmol). The reaction vial was capped and stirred at 30° C. for 72 hours. After, the crude mixture was diluted with water (50.0 mL) and extracted with dichloromethane (3×20.0 mL). The combined organic fractions washed with brine, dried over anhydrous magnesium sulfate, filtered, and the solvent was removed in vacuo. The crude material was purified by flash chromatography on silica gel (heptane/EtOAc 1:1) to afford the title compound (62.0 mg, 46% yield).

UPLC (ESI) [M+H]⁺382.10 .

### Intermediate 9: 8-chloro-6-(difluoromethyl)-2-(methylsulfanyl)pyrido[3,4-d]pyrimidine

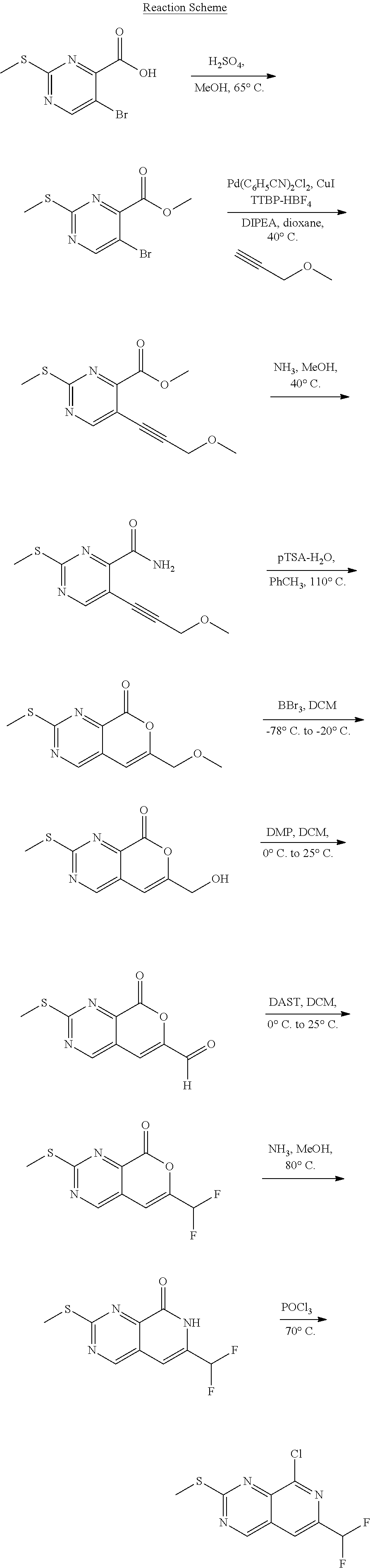

### Detailed Procedure

### Step 1: methyl 5-bromo-2-(methylsulfanyl)pyrimidine-4-carboxylate

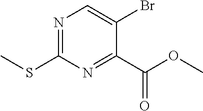

To a solution of 5-bromo-2-(methylthio)pyrimidine-4-carboxylic acid (15.0 g, 60.222 mmol) in methanol (600.0 ml), sulfuric acid (98%, 3.852 ml, 72.266 mmol) was slowly added. The reaction mixture was stirred at 65° C. for 16 hours. Then, the reaction mixture was poured onto ice water and extracted with dichloromethane (300 mL×3). The combined organic layers were washed with NaHCO₃ sat. aq. solution, dried over anhydrous MgSO₄, filtered and concentrated under reduced pressure to provide the title compound (15.02 g, 95% yield). Crude material was used in the next step without further purification.

¹H NMR (300 MHz, Chloroform-d) δ 8.73 (s, 1H), 4.02 (s, 3H), 2.59 (s, 3H).

UPLC (ESI) [M+H]⁺=262.70 and 264.65 (Br isotopic pattern).

### Step 2: methyl 5-(3-methoxyprop-1-yn-1-yl)-2-(methylsulfanyl)pyrimidine-4-carboxylate

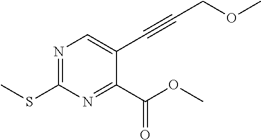

A solution of bis(benzonitrile)palladium chloride (0.729 g, 1.9 mmol), copper(I) iodide (0.362 g, 1.9 mmol) and tri-tert-butylphosphonium tetrafluoroborate (1.103 g, 3.801 mmol) in dioxane anhydrous (50.0 ml) was purged with argon for 10 minutes. Then, N,N-diisopropylethylamine (23.171 ml, 133.024 mmol) was added and the mixture was stirred for 5 min at room temperature. Next, methyl 5-bromo-2-(methylsulfanyl)pyrimidine-4-carboxylate (5.0 g, 19.003 mmol) and 3-methoxyprop-1-yne (4.814 ml, 57.01 mmol) were slowly added and the reaction mixture was stirred at 40° C. overnight. The reaction mixture was cooled down to room temperature, filtered through a pad of celite and washed with ethyl acetate (50 mL) and dichloromethane (50 mL). The filtrate was concentrated under reduced pressure and purified automated flash column chromatography on silica gel (from hexane to hexane/EtOAc 7:3) to provide the title compound (2.47 g, 52% yield).

¹H NMR (300 MHz, Chloroform-d) δ 8.73 (s, 1H), 4.39 (s, 2H), 4.01 (s, 3H), 3.50 (s, 3H), 2.63 (s, 3H).

UPLC (ESI) [M+H]⁺252.80.

### Step 3: 5-(3-methoxyprop-1-yn-1-yl)-2-(methylsulfanyl)pyrimidine-4-carboxamide

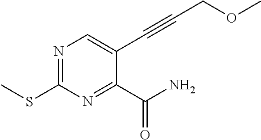

A solution of methyl 5-(3-methoxyprop-1-yn-1-yl)-2-(methylsulfanyl)pyrimidine-4-carboxylate (2.47 g, 9.79 mmol) in ammonia (7.0 N solution in methanol, 34.965 ml, 244.758 mmol) was stirred at 40° C. for 2 hours. The mixture was concentrated under reduced pressure to provide the crude title compound (2.28 g, 98% yield). The crude product was used for the next step without further purification.

¹H NMR (300 MHz, Chloroform-d) δ 8.78 (s, 1H), 7.55 (s, 1H), 5.58 (s, 1H), 4.44 (s, 2H), 3.53 (s, 3H), 2.63 (s, 3H).

UPLC (ESI) [M+H]⁺237.85.

### Step 4: 6-(methoxymethyl)-2-(methylsulfanyl)-8H-pyrano[3,4-d]pyrimidin-8-one

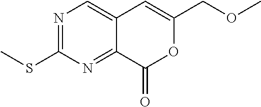

To a solution of 5-(3-methoxyprop-1-yn-1-yl)-2-(methylsulfanyl)pyrimidine-4-carboxamide (1.580 g, 6.660 mmol) in toluene (28.56 ml) was added p-toluenesolfonic acid monohydrate (0.916 g, 4.815 mmol). The reaction mixture was stirred at 110° C. for 48 hours. The reaction mixture was concentrated under reduced pressure and purified by flash column chromatography on silica gel (from hexane/EtOAc 7:3 to hexane/EtOAc 3:7) to provide the title compound (0.685 g, 43% yield).

¹H NMR (300 MHz, Chloroform-d) δ 8.85 (s, 1H), 6.53 (t, J=1.2 Hz, 1H), 4.31 (d, J=1.2 Hz, 2H), 3.53 (s, 3H), 2.70 (s, 3H).

### Step 5: 6-(hydroxymethyl)-2-(methylsulfanyl)-8H-pyrano[3,4-d]pyrimidin-8-one

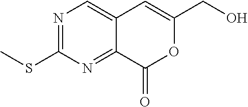

A solution of 6-(methoxymethyl)-2-(methylsulfanyl)-8H-pyrano[3,4-d]pyrimidin-8-one (1.275 g, 5.373 mmol) in dichloromethane anhydrous (44.78 ml) was cooled down to −78° C. Then, boron tribromide (1.0 M solution in dichloromethane, 32.247 ml, 32.247 mmol) was added dropwise via addition funnel and the reaction mixture was stirred at the same temperature for 20 minutes. Next, the reaction mixture was warmed up to −20° C. and stirred for an additional 2 hours and 30 minutes. The reaction mixture was quenched by the dropwise addition of methanol followed by NaHCO₃ sat. aq. solution (until gas release stopped). The mixture was extracted with dichloromethane (50 mL×3). The combined organic layers were dried over anhydrous MgSO₄, filtered, concentrated under reduced pressure and purified by flash column chromatography on silica gel (from hexane/EtOAc 7:3 to hexane/EtOAc 1:9) to provide the title compound (0.787 g, 59% yield).

¹H NMR (300 MHz, DMSO-d₆) δ 9.19 (s, 1H), 6.74 (d, J=1.2 Hz, 1H), 5.74 (t, J=6.0 Hz, 1H), 4.30 (dd, J=6.0, 1.2 Hz, 2H), 2.61 (s, 3H).

### Step 6: 2-(methylsulfanyl)-8-oxo-8H-pyrano[3,4-d]pyrimidine-6-carbaldehyde

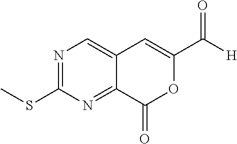

A solution of 6-(hydroxymethyl)-2-(methylsulfanyl)-8H-pyrano[3,4-d]pyrimidin-8-one (0.787 g, 3.173 mmol) in dichloromethane anhydrous (15.74 ml) was cooled down to 0° C. Then, Dess-Martin periodinane (2.692 g, 6.347 mmol) was added portionwise. The reaction mixture was allowed to warm up to room temperature and stirred for 30 minutes. The solid was filtered off and the filtrate was washed with 0.5 M NaOH aq. solution, dried over anhydrous MgSO₄, filtered and concentrated under reduced pressure. The crude material was purified by flash column chromatography on silica gel (from hexane/EtOAc 8:2 to hexane/EtOAc 4:6) to provide the title compound (0.738 g, 100% yield).

¹H NMR (300 MHz, DMSO-d₆) δ 9.58 (s, 1H), 9.35 (s, 1H), 7.78 (s, 1H), 2.65 (s, 3H).

### Step 7: 6-(difluoromethyl)-2-(methylsulfanyl)-8H-pyrano[3,4-d]pyrimidin-8-one

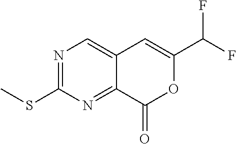 [SMILES: CSc1ncc2cc(C(F)F)oc(=O)c2n1]

A solution of 2-(methylsulfanyl)-8-oxo-8H-pyrano[3,4-d]pyrimidine-6-carbaldehyde (0.738 g, 3.167 mmol) in dichloromethane (22.13 ml) was cooled down to 0° C. Then, DAST (0.418 ml, 3.167 mmol) was added dropwise and the reaction mixture was allowed to warm up to room temperature and stirred for 1 hour. The reaction mixture was quenched by the dropwise addition of a 10% Na₂S₂O₃ aq. solution (10 mL). Layers were separated and aqueous layer was extracted with dichloromethane (15 mL×3). The combined organic layers were dried over anhydrous MgSO₄, filtered and concentrated under reduced pressure to provide the title compound (0.784 g, 92% yield). Crude material was used in the next step without further purification.

¹H NMR (300 MHz, DMSO-d₆) δ 9.24 (s, 1H), 7.25 (d, J=1.6 Hz, 1H), 6.94 (t, J=52.7 Hz, 1H), 2.63 (s, 3H).

UPLC (ESI) [M+H]⁺244.85.

### Step 8: 6-(difluoromethyl)-2-(methylsulfanyl)-7H,8H-pyrido[3,4-d]pyrimidin-8-one

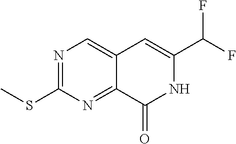 [SMILES: CSc1ncc2cc(C(F)F)[nH]c(=O)c2n1]

A solution of 6-(difluoromethyl)-2-(methylsulfanyl)-8H-pyrano[3,4-d]pyrimidin-8-one (0.734 g, 2.703 mmol) in ammonia (7.0 N solution in methanol, 21.242 ml, 148.691 mmol) was stirred at 80° C. for 16 hours. The volatiles were removed under reduced pressure to provide the crude title compound (0.704 g, 96% yield) as a dark solid. The crude product was used for the next step without further purification.

¹H NMR (300 MHz, DMSO-d₆) δ 9.28 (s, 1H), 6.95 (d, J=1.6 Hz, 1H), 6.88 (t, J=53.8 Hz, 1H), 2.62 (s, 3H).

UPLC (ESI) [M+H]⁺243.75.

### Step 9: 8-chloro-6-(difluoromethyl)-2-(methylsulfanyl)pyrido[3,4-d]pyrimidine

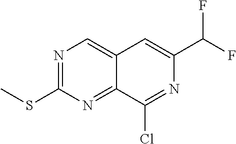 [SMILES: CSc1ncc2cc(C(F)F)nc(Cl)c2n1]

A solution of 6-(difluoromethyl)-2-(methylsulfanyl)-7H,8H-pyrido[3,4-d]pyrimidin-8-one (0.704 g, 2.605 mmol) in phosphorus oxychloride (7.306 ml, 78.148 mmol) was stirred at 70° C. for 3 hours. The reaction mixture was concentrated under reduced pressure and the residue was redissolved in ethyl acetate (15 mL) and washed with sat. aq. NaHCO₃ (15 mL). The aqueous layer was extracted with ethyl acetate (15 mL×3) and the combined organic layers were dried over anhydrous MgSO₄, filtered and concentrated under reduced pressure. Crude material was purified by flash column chromatography on silica gel (from hexane to hexane/EtOAc 7:3) to provide the title compound (0.277 g, 41% yield).

¹H NMR (300 MHz, DMSO-d₆) δ 9.69 (s, 1H), 8.37 (s, 1H), 7.17 (t, J=54.5 Hz, 1H), 2.71 (s, 3H).

UPLC (ESI) [M+H]⁺261.80.

### Intermediate 10: N-(5-(6-ethyl-2,6-diazaspiro[3.3]heptan-2-yl) pyridin-2-yl) formamide

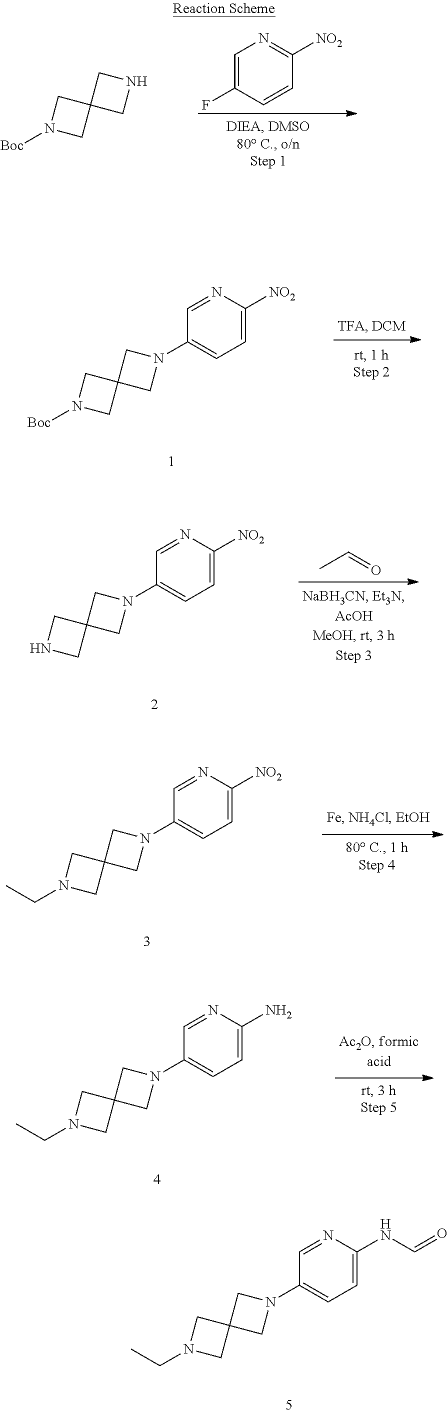

### Detailed Procedure

### Step 1: tert-butyl 6-(6-nitropyridin-3-yl)-2,6-diazaspiro[3.3]heptane-2-carboxylate

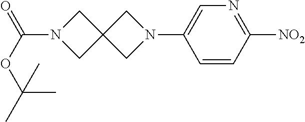 [SMILES: CC(C)(C)OC(=O)N1CC2(C1)CN(c1ccc([N+](=O)[O-])nc1)C2]

To a stirred mixture of tert-butyl 2,6-diazaspiro[3.3]heptane-2-carboxylate (5 g, 25.21 mmol) and 5-fluoro-2-nitropyridine (5.37 g, 37.82 mmol) in dimethyl sulfoxide (30 mL) was N,N-diisopropylethylamine (9.78 g, 75.65 mmol). The resulting mixture was heated to 80° C. and stirred for 3 hours. The reaction mixture was allowed to cool to room temperature, diluted with water (500 mL) and extracted with ethyl acetate (3×500 mL). The combined organic layers were washed with brine (500 mL), dried over anhydrous sodium sulfate, and filtered. The filtrate was concentrated under vacuum to afford the crude product. The residue was purified by trituration with petroleum ether/ethyl acetate (5:1, 100 mL) to afford tert-butyl 6-(6-nitropyridin-3-yl)-2,6-diazaspiro[3.3]heptane-2-carboxylate (6.78 g, 83.7% yield).

LCMS (ESI) m/z=321 [M+H]⁺.

### Step 2: 2-(6-nitropyridin-3-yl)-2,6-diazaspiro[3.3]heptane

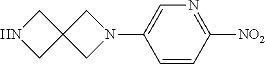 [SMILES: O=[N+]([O-])c1ccc(N2CC3(CNC3)C2)cn1]

To a stirred mixture of tert-butyl 6-(6-nitropyridin-3-yl)-2,6-diazaspiro[3.3]heptane-2-carboxylate (6.78 g, 21.16 mmol) in dichloromethane (80 mL) was added trifluoroacetic acid (16 mL). The resulting mixture was stirred for 1 hour at room temperature and concentrated under vacuum. The residue was diluted with dichloromethane (100 mL) and concentrated under vacuum again to afford crude 2-(6-nitropyridin-3-yl)-2,6-diazaspiro[3.3]heptane trifluoroacetic acid salt (6 g). The crude product was used for next step directly without further purification.

LCMS (ESI) m/z=221 [M+H]⁺.

### Step 3: 2-ethyl-6-(6-nitropyridin-3-yl)-2,6-diazaspiro[3.3]heptane

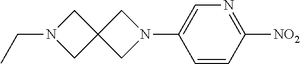 [SMILES: CCN1CC2(C1)CN(c1ccc([N+](=O)[O-])nc1)C2]

A solution of 2-(6-nitropyridin-3-yl)-2,6-diazaspiro[3.3]heptane trifluoroacetic acid salt (6 g, 18.9 mmol) in methanol (100 mL) was treated with triethylamine (5.73 g, 56.7 mmol) for 10 minutes followed by the addition of acetaldehyde (4.16 g, 94.5 mmol), acetic acid (0.23 mL, 4.08 mmol) and sodium cyanoborohydride (2.51 g, 39.8 mmol). The resulting mixture was stirred for 3 hours at room temperature and concentrated under vacuum. The residue was diluted with water (500 mL) and extracted with ethyl acetate (3×500 mL). The combined organic layers were washed with brine (1000 mL), dried over anhydrous sodium sulfate, and filtered. The filtrate was concentrated under reduced pressure to afford the crude product. The residue was purified by trituration with dichloromethane (100 mL) to afford 2-ethyl-6-(6-nitropyridin-3-yl)-2,6-diazaspiro[3.3]heptane (4 g, 58.9% yield) as an orange solid.

LCMS (ESI) m/z=249 [M+h]⁺.

### Step 4: 5-(6-ethyl-2,6-diazaspiro[3.3]heptan-2-yl) pyridin-2-amine

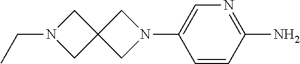 [SMILES: CCN1CC2(C1)CN(c1ccc(N)nc1)C2]

A solution of 2-ethyl-6-(6-nitropyridin-3-yl)-2,6-diazaspiro[3.3]heptane (4 g, 16.11 mmol), ammonium chloride (4.31 g, 80.55 mmol), iron powder (9.00 g, 161.100 mmol) and water (20 mL) in ethanol (60 mL) was stirred for 1 hour at 80° C. The resulting mixture was filtered and the filter cake was washed with ethanol (100 mL). The filtrate was concentrated under vacuum to afford the crude product. The residue was purified by reversed-phase flash chromatography (C18 silica gel, acetonitrile/water (with 10 mmol/L NH₄HCO₃) gradient) to afford 5-(6-ethyl-2,6-diazaspiro[3.3]heptan-2-yl)pyridin-2-amine (2 g, 56.6% yield).

LCMS (ESI) m/z=219 [M+H]⁺.

### Step 5: N-(5-(6-ethyl-2,6-diazaspiro[3.3]heptan-2-yl) pyridin-2-yl) formamide

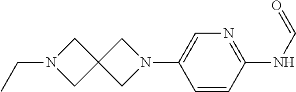 [SMILES: CCN1CC2(C1)CN(c1ccc(NC=O)nc1)C2]

A solution of acetic anhydride (2 mL) in formic acid (4 mL) was stirred for 1 hour at room temperature followed by the addition of 5-(6-ethyl-2,6-diazaspiro[3.3]heptan-2-yl)pyridin-2-amine (400 mg, 1.83 mmol) in portions at room temperature. The resulting mixture was stirred for 3 hours at room temperature. The reaction mixture was neutralized to PH=7 with saturated aqueous sodium bicarbonate (200 mL) and extracted with ethyl acetate (3×100 mL). The combined organic layers were washed with brine, dried over anhydrous sodium sulfate, and filtered. The filtrate was concentrated under vacuum and the residue was purified by preparative reverse phase HPLC (acetonitrile/water (with 10 mM NH₄HCO₃ and 0.1% NH₃·H₂O) gradient) to afford the title compound (70 mg, 15.3% yield).

LCMS (ESI) m/z=247 [M+H]⁺.

### Intermediate 12: 1-(cyclopropylsulfonyl)piperidin-4-amine

### Reaction Scheme

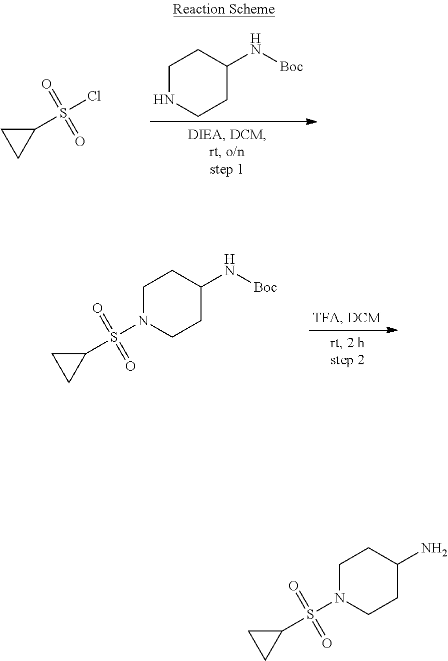 [SMILES: O=S(=O)(Cl)C1CC1 ; CC(C)(C)OC(=O)NC1CCNCC1 ; CC(C)(C)OC(=O)NC1CCN(S(=O)(=O)C2CC2)CC1 ; NC1CCN(S(=O)(=O)C2CC2)CC1]

### Detailed Procedure

### Step 1: Tert-butyl (1-(cyclopropylsulfonyl)piperidin-4-yl)carbamate

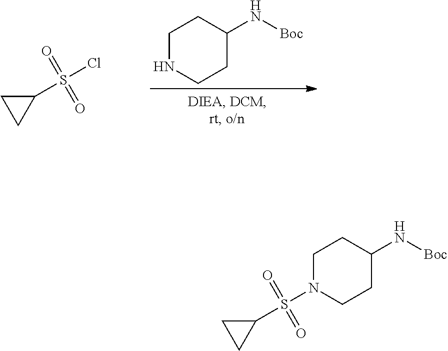 [SMILES: O=S(=O)(Cl)C1CC1 ; CC(C)(C)OC(=O)NC1CCNCC1 ; CC(C)(C)OC(=O)NC1CCN(S(=O)(=O)C2CC2)CC1]

To a solution of cyclopropanesulfonyl chloride (7.02 g, 49.93 mmol) and DIEA (19.36 g, 149.79 mmol) in DCM (100 mL) was added tert-butyl N-(piperidin-4-yl)carbamate (10 g, 49.93 mmol) dropwise at 0° C. The resulting mixture was stirred overnight at room temperature. The resulting mixture was diluted with water (500 mL). The aqueous solution was extracted with CH₂Cl₂ (3×500 mL). The combined organic layers were dried over anhydrous Na₂SO₄. After filtration, the filtrate was concentrated under reduced pressure. The residue was purified by trituration with EA (100 mL) to afford tert-butyl (1-(cyclopropylsulfonyl)piperidin-4-yl)carbamate (10 g, 59.2% o yield).

LCMS (ESI) m/z=249.1 [M+H−56]⁺.

### Step 2: 1-(cyclopropylsulfonyl)piperidin-4-amine

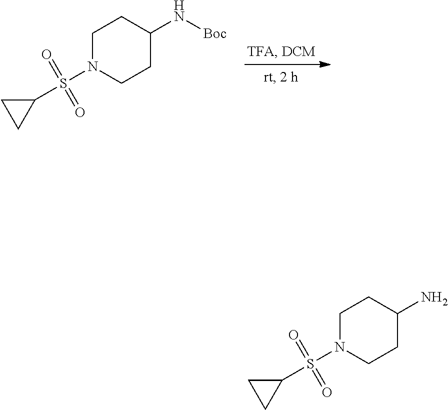 [SMILES: CC(C)(C)OC(=O)NC1CCN(S(=O)(=O)C2CC2)CC1 ; NC1CCN(S(=O)(=O)C2CC2)CC1]

A solution of tert-butyl (1-(cyclopropylsulfonyl)piperidin-4-yl)carbamate (10 g, 32.87 mmol) in TFA (15 mL) and DCM (45 mL) was stirred for 2 h at room temperature. The resulting mixture was concentrated under reduced pressure to afford 1-(cyclopropylsulfonyl)piperidin-4-amine (8 g).

LCMS (ESI−MS) m/z=205.1 [M+H]⁺.

### Intermediate 13: 8-chloro-N-(1-(cyclopropylsulfonyl)piperidin-4-yl)-6-methylpyrido[3,4-d]pyrimidin-2-amine

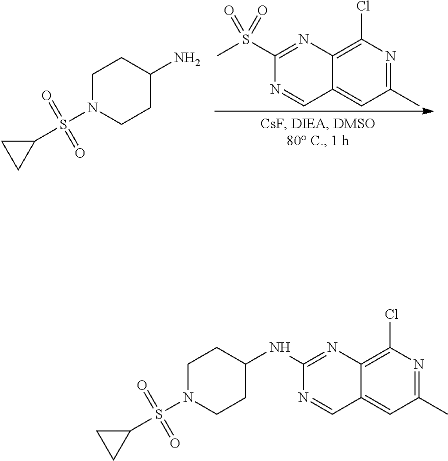

A solution of 8-chloro-6-methyl-2-(methylsulfonyl)pyrido[3,4-d]pyrimidine (1 g, 3.88 mmol), 1-(cyclopropanesulfonyl)piperidin-4-amine (1.59 g, 7.76 mmol), DIEA (1.50 g, 11.64 mmol) and CsF (1.77 g, 11.64 mmol) in DMSO (10 mL) was stirred for 1 h at 80° C. The reaction mixture was diluted with water (200 mL) and extracted with EA (3×200 mL). The combined organic layers were dried over anhydrous Na₂SO₄. After filtration, the filtrate was concentrated under reduced pressure. The residue was purified by silica gel column chromatography (PE/EA, 1:1) to afford 8-chloro-N-(1-(cyclopropylsulfonyl)piperidin-4-yl)-6-methylpyrido[3,4-d]pyrimidin-2-amine (505.8 mg, 33.3% yield).

¹H NMR (400 MHz, DMSO-d6) δ 9.22 (s, 1H), 8.08 (d, J=7.4 Hz, 1H), 7.56 (s, 1H), 4.03 (s, 1H), 3.69-3.57 (m, 2H), 3.09-2.92 (m, 2H), 2.69-2.55 (m, 1H), 2.17-1.89 (m, 2H), 1.71-1.55 (m, 2H), 1.42-1.30 (m, 1H), 1.29-1.13 (m, 2H), 1.05-0.97 (m, 2H), 0.97-0.91 (m, 2H).

LCMS (ESI−MS) m/z=382.2 [M+H]⁺.

### Intermediate 14: 1-((1-methyl-1H-pyrazol-4-yl)sulfonyl)piperidin-4-amine

### Reaction Scheme

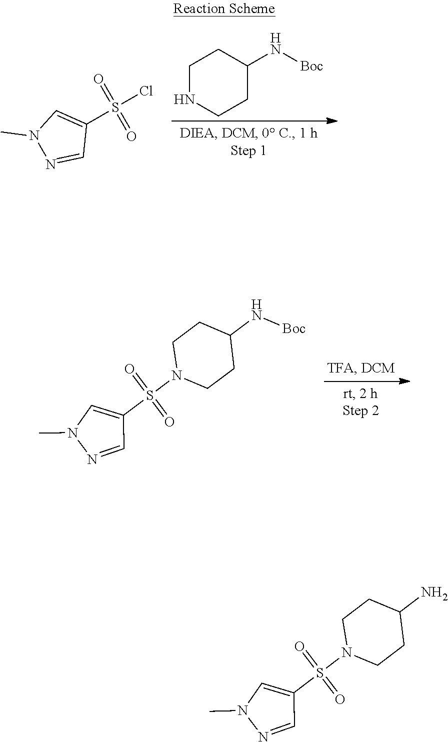

### Step 1: Tert-butyl (1-((1-methyl-1H-pyrazol-4-yl)sulfonyl)piperidin-4-yl)carbamate

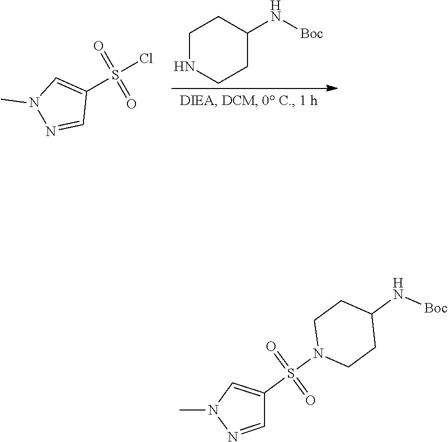

To a stirred mixture of 1-methyl-1H-pyrazole-4-sulfonyl chloride (7 g, 38.75 mmol) and DIEA (15.03 g, 116.27 mmol) in DCM (70 mL) was added tert-butyl piperidin-4-ylcarbamate (7.76 g, 38.75 mmol) dropwise at 0° C. The resulting mixture was stirred for 1 h at 0° C. The resulting mixture was diluted with water (500 mL). The aqueous solution was extracted with CH₂Cl₂ (3×500 mL). The combined organic layers were dried over anhydrous Na₂SO₄. After filtration, the filtrate was concentrated under reduced pressure. The residue was purified by silica gel column chromatography (CH₂Cl₂/MeOH, 99:1) to afford tert-butyl (1-((1-methyl-1H-pyrazol-4-yl)sulfonyl)piperidin-4-yl)carbamate (9 g, 64.1% yield).

LCMS (ESI−MS) m/z=289.2 [M+H−56]⁺.

### Step 2: 1-((1-methyl-1H-pyrazol-4-yl)sulfonyl)piperidin-4-amine

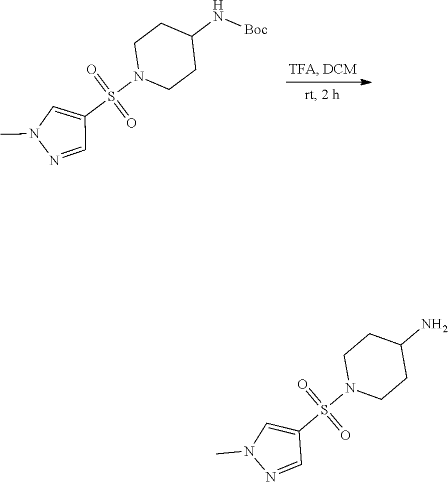 [SMILES: Cn1cc(S(=O)(=O)N2CCC(NC(=O)OC(C)(C)C)CC2)cn1 ; Cn1cc(S(=O)(=O)N2CCC(N)CC2)cn1]

A solution of tert-butyl (1-((1-methyl-1H-pyrazol-4-yl)sulfonyl)piperidin-4-yl)carbamate (6 g, 17.42 mmol) in TFA (12 mL) and DCM (36 mL) was stirred for 2 h at room temperature. The resulting mixture was concentrated under reduced pressure to afford 1-(1-methylpyrazol-4-ylsulfonyl)piperidin-4-amine (6.5 g crude).

LCMS (ESI−MS) m/z=245.2 [M+H]⁺.

### Intermediate 15: 8-chloro-6-methyl-N-(1-((1-methyl-1H-pyrazol-4-yl)sulfonyl)piperidin-4-yl)pyrido[3,4-d]pyrimidin-2-amine

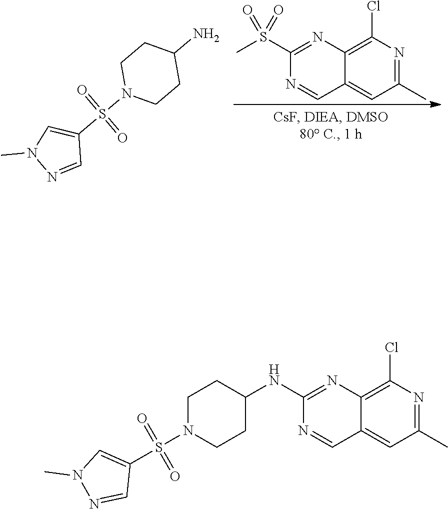

A solution of 1-(1-methylpyrazol-4-ylsulfonyl)piperidin-4-amine (6.07 g, 24.83 mmol), 8-chloro-6-methyl-2-(methylsulfonyl)pyrido[3,4-d]pyrimidine (4 g, 15.52 mmol), DIEA (6.02 g, 46.56 mmol) and CsF (7.07 g, 46.56 mmol) in DMSO (20 mL) was stirred for 1 h at 80° C. The reaction mixture was diluted with water (500 mL) and then extracted with EA (3×500 mL). The combined organic layers were dried over anhydrous Na₂SO₄. After filtration, the filtrate was concentrated under reduced pressure. The crude product was purified by silica gel column chromatography (PE/EA, 1:4) to afford 8-chloro-6-methyl-N-(1-((1-methyl-1H-pyrazol-4-yl)sulfonyl)piperidin-4-yl)pyrido[3,4-d]pyrimidin-2-amine (2.5 g, 37.4% yield).

LCMS (ESI−MS) m/z=422.0 [M+H]⁺.

### Intermediate 16: 8-chloro-N-(6-fluoro-2-(methylsulfonyl)isoindolin-5-yl)-6-methylpyrido[3,4-d]pyrimidin-2-amine

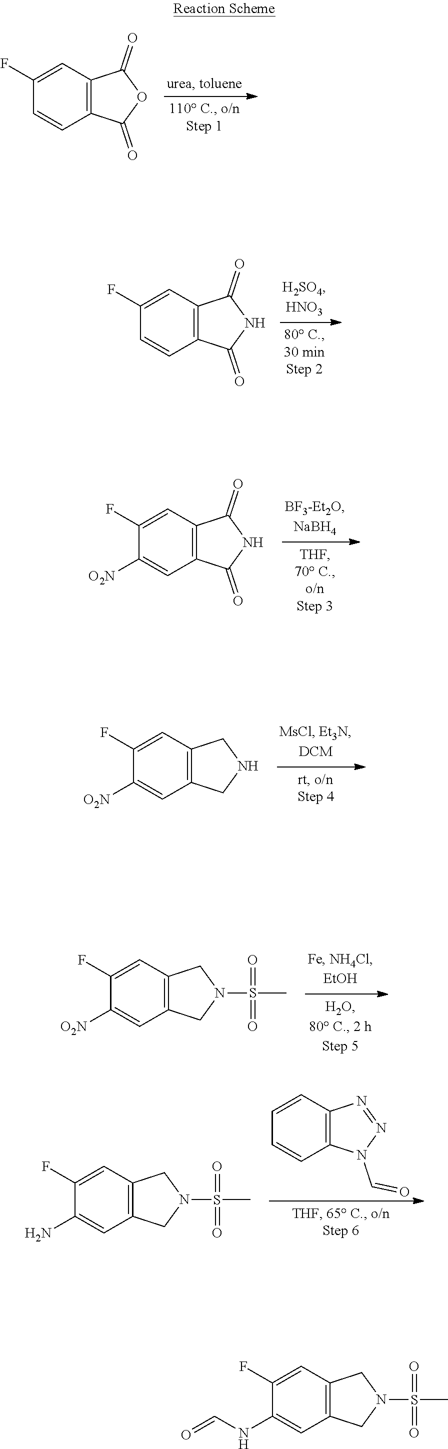

### Detailed Procedure

### Step 1: 5-fluoroisoindoline-1,3-dione

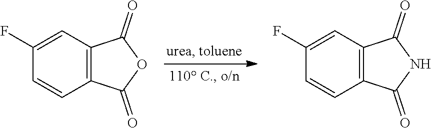 [SMILES: O=C1OC(=O)c2cc(F)ccc21 ; O=C1NC(=O)c2cc(F)ccc21]

To a solution of 5-fluoro-2-benzofuran-1,3-dione (10 g, 60.20 mmol) in toluene (30 mL) was added urea (4.34 g, 72.24 mmol) in portions at 110° C. The resulting mixture was stirred for 18 h at 110° C. The mixture was allowed to cool down to room temperature. The residue was purified by trituration with H₂O (100 mL). This resulted in 5-fluoroisoindoline-1,3-dione (9 g, 38.9% yield).

LCMS (ESI−MS) m/z=166.0 [M+H]⁺.

### Step 2: 5-fluoro-6-nitroisoindoline-1,3-dione

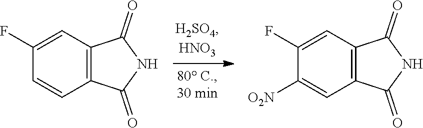 [SMILES: O=C1NC(=O)c2cc(F)ccc21 ; O=C1NC(=O)c2cc([N+](=O)[O-])c(F)cc21]

To a solution of 5-fluoroisoindoline-1,3-dione (9 g, 54.50 mmol) in H₂SO₄ (90 mL) were added HNO₃ (9 mL) in portions at 0° C. The resulting mixture was stirred for additional 30 min at 80° C. The mixture was allowed to cool down to 0° C. The reaction was quenched with cold water at 0° C. The resulting mixture was stirred for an additional 30 min at 0° C. The precipitated solids were collected by filtration and washed with H₂O (3×500 mL) to afford the desired product 5-fluoro-6-nitroisoindoline-1,3-dione (4 g, 34.9% yield).

LCMS (ESI−MS) m/z=211.0 [M+H]⁺.

### Step 3: 5-fluoro-6-nitroisoindoline

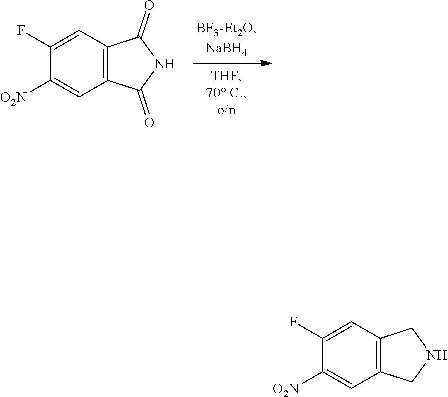 [SMILES: O=C1NC(=O)c2cc([N+](=O)[O-])c(F)cc21 ; O=[N+]([O-])c1cc2c(cc1F)CNC2]

To a solution of 5-fluoro-6-nitroisoindoline-1,3-dione (5 g, 23.79 mmol) in THF (217.5 mL) were added NaBH₄ (0.87 g, 22.99 mmol) and BF3-Et2O (35.56 g, 250.57 mmol) in portions at −10° C. The resulting mixture was stirred for 18 h at 70° C. under a nitrogen atmosphere and concentrated under vacuum to afford crude5-fluoro-6-nitroisoindoline (1.5 g crude).

LCMS (ESI−MS) m/z=183.0 [M+H]⁺.

### Step 4: 5-fluoro-2-(methylsulfonyl)-6-nitroisoindoline

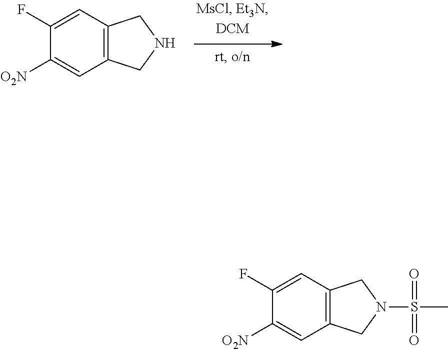 [SMILES: O=[N+]([O-])c1cc2c(cc1F)CNC2 ; CS(=O)(=O)N1Cc2cc(F)c([N+](=O)[O-])cc2C1]

To a solution of 5-fluoro-6-nitroisoindoline (1.5 g, 8.23 mmol) in DCM (10 mL) was added Et₃N (4.50 mL, 32.36 mmol) over 3 min at 0° C. under a nitrogen atmosphere. MsCl (1.71 g, 14.90 mmol) was then added in portions at 0° C. The resulting mixture was stirred overnight at room temperature. The aqueous layer was extracted with EtOAc (2×200 mL). The resulting mixture was washed with saturated aq. sodium chloride solution (2×200 mL). The combined organic layers were dried over anhydrous Na₂SO₄. After filtration, the filtrate was concentrated under vacuum. The residue was purified by silica gel column chromatography, eluting with PE/EA and CH₂Cl₂/MeOH (5:1) to afford 5-fluoro-2-(methylsulfonyl)-6-nitroisoindoline (500 mg, 23.3% yield).

LCMS (ESI−MS) m/z=261.0 [M+H]⁺.

### Step 5: 6-fluoro-2-(methylsulfonyl)isoindolin-5-amine

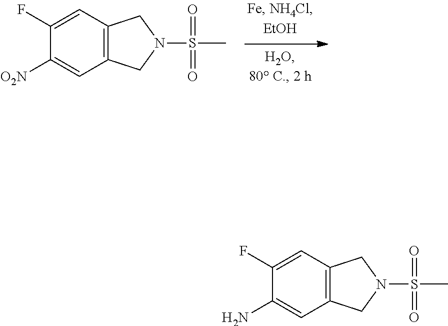 [SMILES: CS(=O)(=O)N1Cc2cc(F)c([N+](=O)[O-])cc2C1 ; CS(=O)(=O)N1Cc2cc(N)c(F)cc2C1]

To a solution of 5-fluoro-2-(methylsulfonyl)-6-nitroisoindoline (500 mg, 1.92 mmol) in EtOH (12 mL) and H₂O (4 mL) were added Fe (1.07 g, 19.21 mmol) and NH₄Cl (411.08 mg, 7.68 mmol) in portions at 80° C. The resulting mixture was stirred for 2 h at 80° C. under a nitrogen atmosphere and concentrated under vacuum. The residue was purified by silica gel column chromatography, eluting with PE/EA (1:1) to afford 6-fluoro-2-(methylsulfonyl)isoindolin-5-amine (280 mg, 63.3% yield).

LCMS (ESI−MS) m/z=231.0 [M+H]⁺.

### Step 6: N-(6-fluoro-2-(methylsulfonyl)isoindolin-5-yl)formamide

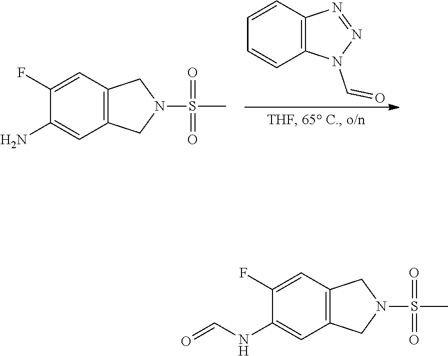 [SMILES: CS(=O)(=O)N1Cc2cc(N)c(F)cc2C1 ; O=Cn1nnc2ccccc21 ; CS(=O)(=O)N1Cc2cc(F)c(NC=O)cc2C1]

To a solution of 6-fluoro-2-(methylsulfonyl)isoindolin-5-amine (110 mg, 0.47 mmol) in THF (2 mL) was added 1,2,3-benzotriazole-1-carbaldehyde (70.29 mg, 0.47 mmol). The resulting mixture was stirred overnight at 65° C. under a nitrogen atmosphere and allowed to cool down to room temperature. The mixture was concentrated under reduced pressure and purified by silica gel column chromatography, eluting with PE/EA (1:1) to afford N-(6-fluoro-2-(methylsulfonyl)isoindolin-5-yl)formamide (60 mg, 48.6% yield).

LCMS (ESI−MS) m/z=259.0 [M+H]⁺.

### Step 7: 8-chloro-N-(6-fluoro-2-(methylsulfonyl)isoindolin-5-yl)-6-methylpyrido[3,4-d]pyrimidin-2-amine

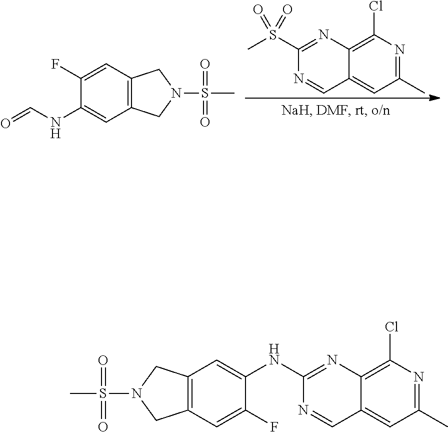

To a solution of N-(6-fluoro-2-(methylsulfonyl)isoindolin-5-yl)formamide (55 mg, 0.21 mmol) in dimethylformamide (2 mL) was added sodium hydride (25.55 mg, 1.06 mmol, 60% in mineral oil). The resulting mixture was stirred for 1 h at 0° C. under a nitrogen atmosphere. 8-chloro-6-methyl-2-(methylsulfonyl)pyrido[3,4-d]pyrimidine (54.88 mg, 0.21 mmol) was added and the resulting mixture was stirred overnight at room temperature. The reaction was quenched by the addition of a saturated aq. NH₄Cl solution (60 mL) at room temperature, diluted with EtOAc (500 mL) and washed with a saturated aq. sodium chloride solution (3×500 mL). The combined organic layers were dried over anhydrous Na₂SO₄. After filtration, the filtrate was concentrated under reduced pressure to give 8-chloro-N-(6-fluoro-2-(methylsulfonyl)isoindolin-5-yl)-6-methylpyrido[3,4-d]pyrimidin-2-amine (60 mg, 69.1% yield).

LCMS (ESI−MS) m/z=408.1 [M+H]⁺.

### Intermediate 17: 1-(2-methyl-2-azaspiro[3.3]heptan-6-yl)-1H-pyrazol-4-amine

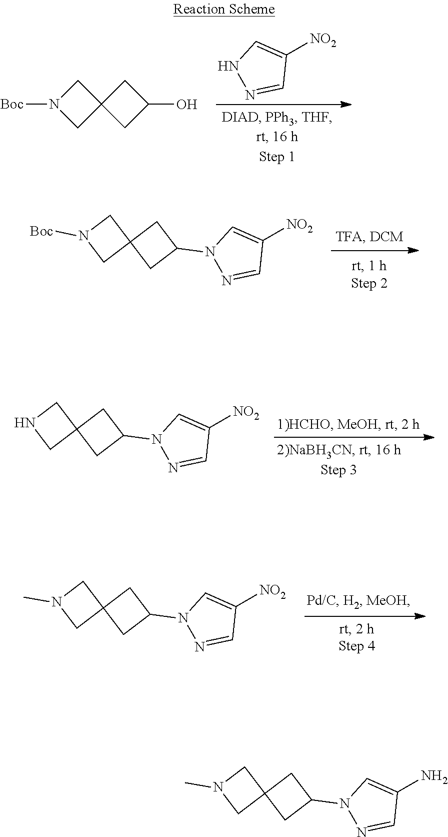 [SMILES: CC(C)(C)OC(=O)N1CC2(CC(O)C2)C1 ; O=[N+]([O-])c1cn[nH]c1 ; CC(C)(C)OC(=O)N1CC2(CC(n3cc([N+](=O)[O-])cn3)C2)C1 ; O=[N+]([O-])c1cnn(C2CC3(CNC3)C2)c1 ; CN1CC2(CC(n3cc([N+](=O)[O-])cn3)C2)C1 ; CN1CC2(CC(n3cc(N)cn3)C2)C1]

### Detailed Procedure

### Step 1: Tert-butyl 6-(4-nitro-1H-pyrazol-1-yl)-2-azaspiro[3.3]heptane-2-carboxylate

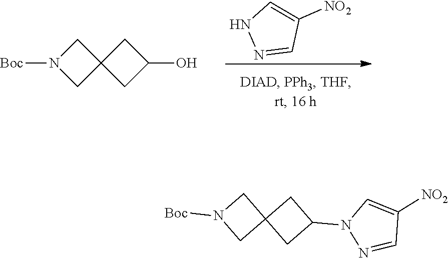

To a solution of tert-butyl 6-hydroxy-2-azaspiro[3.3]heptane-2-carboxylate (10 g, 46.88 mmol) in THF (100 mL) was added 4-nitropyrazole (5.30 g, 46.88 mmol), DIAD (9.48 g, 46.88 mmol) and PPh₃ (12.30 g, 46.88 mmol). The resulting mixture was stirred at room temperature for 16 h. The reaction mixture was concentrated under vacuum and purified by silica gel column eluting with DCM/MeOH=10:1 to afford the title product (8 g, 49.8% yield).

LCMS (ESI−MS) m/z=309.1 [M+H]⁺.

### Step 2: 6-(4-nitro-1H-pyrazol-1-yl)-2-azaspiro[3.3]heptane

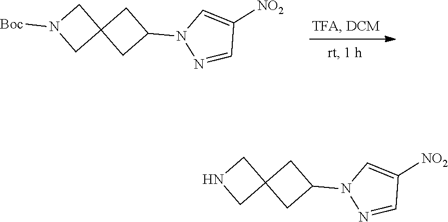

A mixture of tert-butyl 6-(4-nitropyrazol-1-yl)-2-azaspiro[3.3]heptane-2-carboxylate (8 g, 25.94 mmo), TFA (3 mL) and DCM (6 mL) was stirred at room temperature for 1 h. The reaction mixture was concentrated under vacuum to afford 6-(4-nitro-1H-pyrazol-1-yl)-2-azaspiro[3.3]heptane (5 g, 83.3% yield).

LCMS (ESI−MS) m/z=209.1 [M+H]⁺.

### Step 3: 2-methyl-6-(4-nitro-1H-pyrazol-1-yl)-2-azaspiro[3.3]heptane

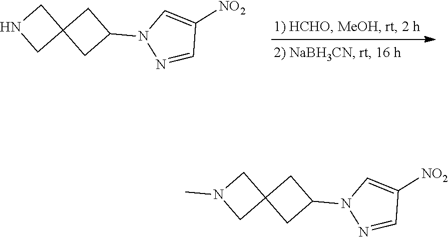

A mixture of 6-(4-nitropyrazol-1-yl)-2-azaspiro[3.3]heptane (4.5 g, 21.61 mmol) and HCHO (1.95 g, 64.83 mmol) in MeOH (50 mL) was stirred at room temperature for 2 h. NaBH₃CN (2.72 g, 43.22 mmol) was added and the resulting mixture was stirred for 16 h at room temperature. The mixture was purified by silica gel column chromatography, eluting with DCM/MeOH=10:1 to afford 2-methyl-6-(4-nitro-1H-pyrazol-1-yl)-2-azaspiro[3.3]heptane (3.5 g, 58.3% yield).

LCMS (ESI−MS) m/z=223.1 [M+H]⁺.

### Step 4: 1-(2-methyl-2-azaspiro[3.3]heptan-6-yl)-1H-pyrazol-4-amine

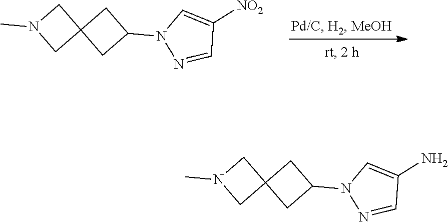

Pd/C (350 mg, 10% on carbon) was added to a solution of 2-methyl-6-(4-nitropyrazol-1-yl)-2-azaspiro[3.3]heptane (3.5 g, 15.74 mmol) in MeOH (50 mL) under a nitrogen atmosphere. The resulting mixture was stirred at room temperature for 2 h under hydrogen atmosphere. The reaction mixture was filtered and the filtrate was concentrated under reduced pressure to afford crude 1-(2-methyl-2-azaspiro[3.3]heptan-6-yl)-1H-pyrazol-4-amine (3 g, 99.1% yield).

LCMS (ESI-MS) m/z=193.1 [M+H]⁺.

### Intermediate 18: 2-(methylsulfonyl) isoindolin-5-amine

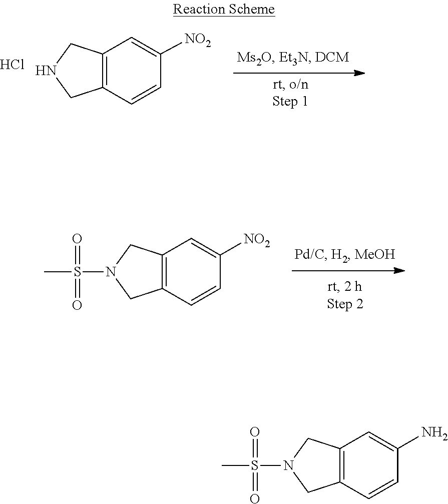 [SMILES: Cl ; O=[N+]([O-])c1ccc2c(c1)CNC2 ; CS(=O)(=O)N1Cc2ccc([N+](=O)[O-])cc2C1 ; CS(=O)(=O)N1Cc2ccc(N)cc2C1]

### Step 1: 2-(methylsulfonyl)-5-nitroisoindoline

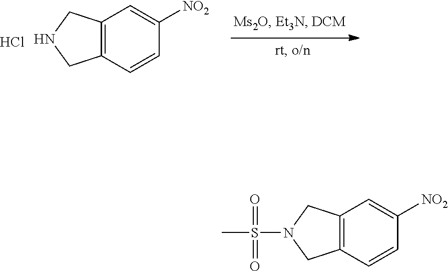 [SMILES: Cl ; O=[N+]([O-])c1ccc2c(c1)CNC2 ; CS(=O)(=O)N1Cc2ccc([N+](=O)[O-])cc2C1]

Methanesulfonyl methanesulfonate (1326.39 mg, 7.61 mmol) was added to a stirred solution of 5-nitro-2,3-dihydro-1H-isoindole (500 mg, 3.04 mmol) and Et₃N (462.31 mg, 4.56 mmol) in DCM (10 mL) at 0° C. The resulting mixture was stirred overnight at room temperature. The reaction was quenched by the addition of water (100 mL) at 0° C. The mixture was extracted with CH₂Cl₂ (3×150 mL). The combined organic layers were dried over anhydrous Na₂SO₄. After filtration, the filtrate was concentrated under reduced pressure to afford crude2-(methylsulfonyl)-5-nitroisoindoline (500 mg, 67.7% yield).

No mass signal.

### Step 2: 2-(methylsulfonyl)isoindolin-5-amine

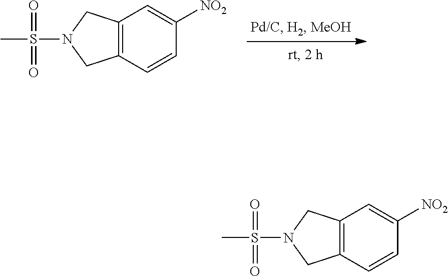 [SMILES: CS(=O)(=O)N1Cc2ccc([N+](=O)[O-])cc2C1 ; CS(=O)(=O)N1Cc2ccc([N+](=O)[O-])cc2C1]

Pd/C (219.6 mg, 2.06 mmol, 10% on carbon) was added to a solution of 2-(methylsulfonyl)-5-nitroisoindoline (500 mg, 2.06 mmol) in MeOH (10 mL) under a nitrogen atmosphere. The resulting mixture was stirred for 2 h at room temperature under a hydrogen atmosphere. The reaction mixture was filtered. The filter cake was washed with MeOH (4×100 mL) and the combined filtrate was concentrated under reduced pressure to afford crude 2-(methylsulfonyl) isoindolin-5-amine (400 mg, 91.3% yield).

LCMS (ESI−MS) m/z=213.1 [M+H]⁺.

### Intermediate 19: 2-(1-(methylsulfonyl)azetidin-3-yl)-5-nitroisoindoline

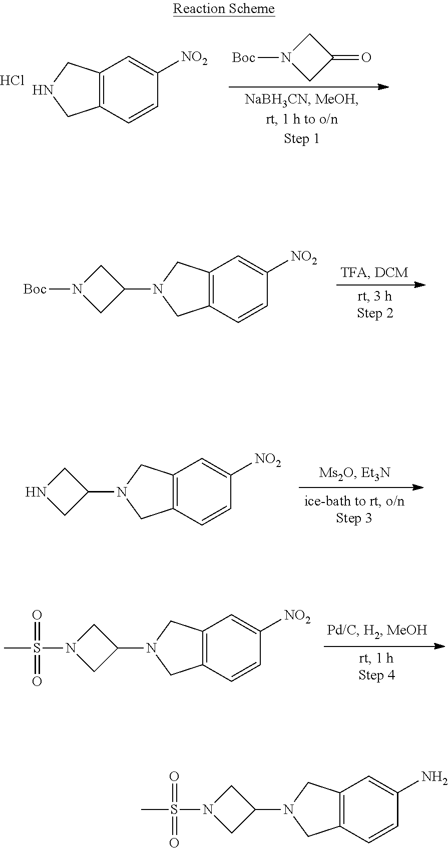

### Detailed Procedure

### Step 1: Tert-butyl 3-(5-nitroisoindolin-2-yl)azetidine-1-carboxylate

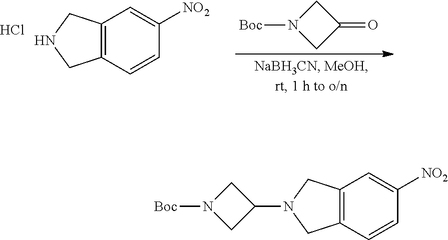 [SMILES: Cl ; O=[N+]([O-])c1ccc2c(c1)CNC2 ; CC(C)(C)OC(=O)N1CC(=O)C1 ; CC(C)(C)OC(=O)N1CC(N2Cc3ccc([N+](=O)[O-])cc3C2)C1]

A mixture of 5-nitro-2,3-dihydro-1H-isoindole hydrochloride (1.8 g, 8.97 mmol) and tert-butyl 3-oxoazetidine-1-carboxylate (1.54 g, 8.97 mmol) in MeOH (40 mL) was stirred for 1 h at room temperature. NaBH₃CN (1.13 g, 17.94 mmol) was added and the resulting mixture was stirred overnight at room temperature. The reaction mixture was concentrated and quenched with water (100 mL). The mixture was extracted with EA (3×100 mL). The combined organic layers were dried over anhydrous Na₂SO₄. After filtration, the filtrate was concentrated under reduced pressure. The crude product was purified by silica gel column chromatography, eluting with EA/PE (30%) to afford tert-butyl 3-(5-nitroisoindolin-2-yl)azetidine-1-carboxylate (2.4 g, 80.8% yield).

LCMS (ESI−MS) m/z=320.2 [M+H]⁺.

### Step 2: 2-(azetidin-3-yl)-5-nitroisoindoline

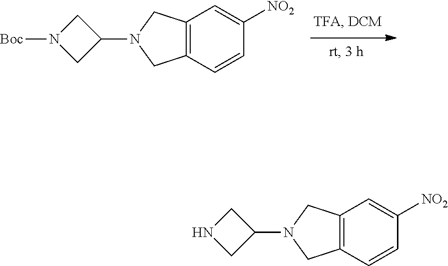 [SMILES: CC(C)(C)OC(=O)N1CC(N2Cc3ccc([N+](=O)[O-])cc3C2)C1 ; O=[N+]([O-])c1ccc2c(c1)CN(C1CNC1)C2]

A mixture of tert-butyl 3-(5-nitroisoindolin-2-yl)azetidine-1-carboxylate (2.4 g, 7.51 mmol) in TFA (4 mL) and DCM (12 mL) was stirred for 3 h at room temperature. The reaction mixture was concentrated purified by silica gel column chromatography, eluting with DCM (0.5% Et₃N)/MeOH (1:1) to afford 2-(azetidin-3-yl)-5-nitroisoindoline (850 mg, 51.6% yield).

LCMS (ESI−MS) m/z=220.1 [M+H]⁺.

### Step 3: 2-(1-(methylsulfonyl)azetidin-3-yl)-5-nitroisoindoline

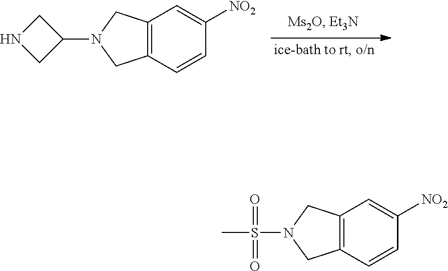 [SMILES: O=[N+]([O-])c1ccc2c(c1)CN(C1CNC1)C2 ; CS(=O)(=O)N1Cc2ccc([N+](=O)[O-])cc2C1]

Methanesulfonic anhydride (699.16 mg, 4.01 mmol) was added to a cooled to 0° C. mixture of 2-(azetidin-3-yl)-5-nitroisoindoline (800 mg, 3.64 mmol) and Et₃N (1107 mg, 10.94 mmol) in DCM (12 mL). The reaction mixture was stirred at room temperature overnight and quenched with water (60 mL). The resulting mixture was extracted with DCM (3×60 mL). The combined organic layers were dried over anhydrous Na₂SO₄. After filtration, the filtrate was concentrated under reduced pressure and purified by silica gel column chromatography, eluting with PE/EA (61%) to afford 2-(1-(methylsulfonyl)azetidin-3-yl)-5-nitroisoindoline (850 mg, 66.6% yield).

LCMS (ESI−MS) m/z=298.1 [M+H]⁺.

### Step 4: 2-(1-(methylsulfonyl)azetidin-3-yl)isoindolin-5-amine

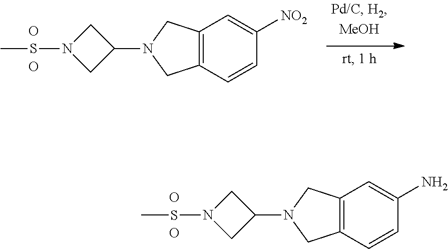

Pd/C (143.17 mg, 1.34 mmol) was added to a mixture of 2-(1-(methylsulfonyl)azetidin-3-yl)-5-nitroisoindoline (800 mg, 2.69 mmol) in MeOH (15 mL) under a nitrogen atmosphere, the reaction mixture was stirred for 1 h at room temperature under a hydrogen atmosphere. The mixture was filtered and the filter cake was washed with MeOH (2×50 mL). The filtrate was concentrated under reduced pressure to afford crude 2-(1-(methylsulfonyl)azetidin-3-yl)isoindolin-5-amine (600 mg, 65.3% yield).

LCMS (ESI−MS) m/z=268.1 [M+H]⁺.

### Intermediate 20: 2-(3-(4-amino-1H-pyrazol-1-yl)azetidin-1-yl)acetonitrile

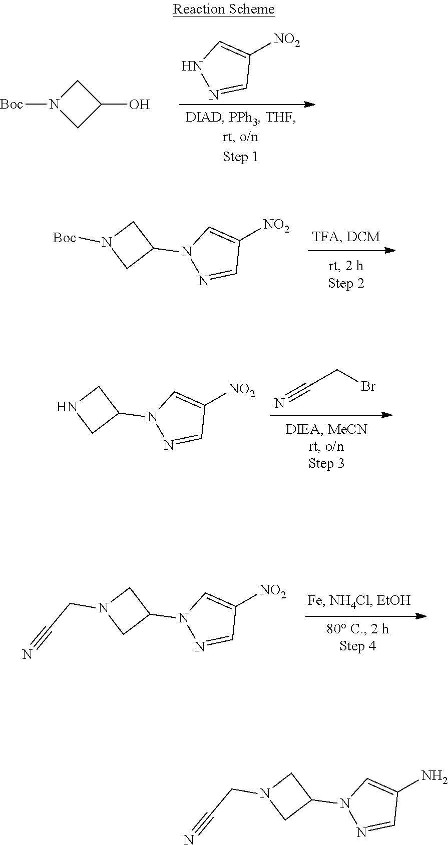

### Step 1: Tert-butyl 3-(4-nitro-1H-pyrazol-1-yl)azetidine-1-carboxylate

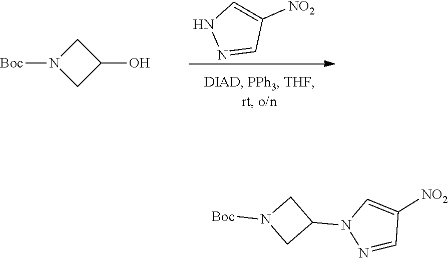

To a solution of tert-butyl 3-hydroxyazetidine-1-carboxylate (3 g, 17.32 mmol) and 4-nitropyrazole (1.96 g, 17.32 mmol) in THF (20 mL) was added PPh₃ (6.81 g, 25.98 mmol) in portions at 0° C. and DIAD (5.25 g, 25.98 mmol) at room temperature. The resulting mixture was stirred overnight at room temperature under a nitrogen atmosphere. The resulting mixture was concentrated under vacuum and purified by silica gel column chromatography (PE/EA, 5:1) to afford tert-butyl 3-(4-nitro-1H-pyrazol-1-yl)azetidine-1-carboxylate (5 g).

LCMS (ESI−MS) m/z=269.1 [M+H]⁺.

### Step 2: 1-(azetidin-3-yl)-4-nitro-1H-pyrazole

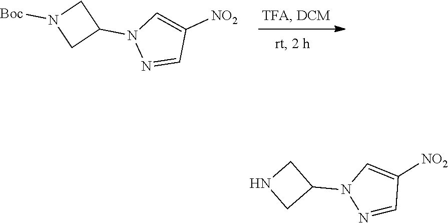

A mixture of tert-butyl 3-(4-nitro-1H-pyrazol-1-yl)azetidine-1-carboxylate (6 g, 22.36 mmol) and TFA (20 mL) in DCM (60 mL) was stirred for 2 h at room temperature. The resulting mixture was concentrated under vacuum to afford the crude 1-(azetidin-3-yl)-4-nitro-1H-pyrazole (4 g).

LCMS (ESI−MS) m/z=169.1 [M+H]⁺.

### Step 3: 2-(3-(4-nitro-1H-pyrazol-1-yl)azetidin-1-yl)acetonitrile

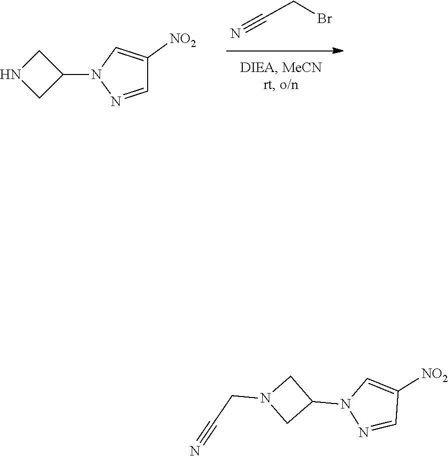

To a solution of 1-(azetidin-3-yl)-4-nitro-1H-pyrazole (1.5 g, 8.92 mmol) in MeCN (50 mL) was added 2-bromoacetonitrile (1.60 g, 13.38 mmol) and N,N-diisopropylethylamine (3.46 g, 26.76 mmol). The resulting mixture was stirred overnight at room temperature. The reaction mixture was concentrated under reduced pressure and purified by silica gel column chromatography (PE/EA, 1:1) to afford the desired product 2-(3-(4-nitro-1H-pyrazol-1-yl)azetidin-1-yl)acetonitrile (300 mg, 16.23%).

LCMS (ESI−MS) m/z=208.1 [M+H]⁺.

### Step 4: 2-(3-(4-amino-1H-pyrazol-1-yl)azetidin-1-yl)acetonitrile

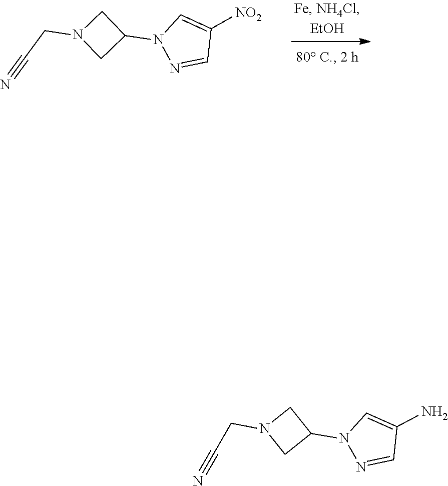 [SMILES: N#CCN1CC(n2cc([N+](=O)[O-])cn2)C1 ; N#CCN1CC(n2cc(N)cn2)C1]

To a solution of 2-(3-(4-nitro-1H-pyrazol-1-yl)azetidin-1-yl)acetonitrile (200 mg, 0.96 mmol) and NH₄Cl (206.53 mg, 3.86 mmol) in water (3 mL) and EtOH (9 mL) was added Fe (539.06 mg, 9.65 mmol) in portions at 80° C. The resulting mixture was stirred for 2 h at 80° C. under a nitrogen atmosphere. The reaction mixture was filtered and the filtrate was concentrated, diluted with water (30 mL) and extracted with EA (3×30 mL). The combined organic layers were dried over anhydrous Na₂SO₄, filtered and concentrated under vacuum to afford crude 2-(3-(4-amino-1H-pyrazol-1-yl)azetidin-1-yl)acetonitrile (120 mg, 70.2% yield).

LCMS (ESI−MS) m/z=178.1 [M+H]⁺.

### Intermediate 21: 1-((1-(dimethylamino)cyclopropyl)methyl)-1H-pyrazol-4-amine

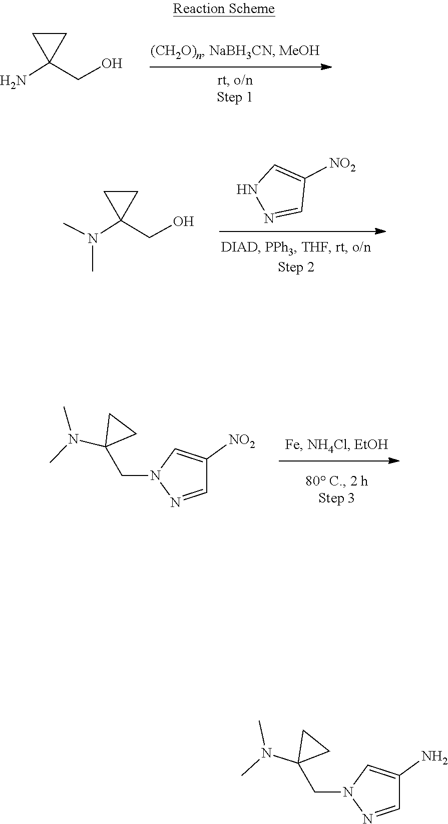

### Detailed Procedure

### Step 1: (1-(dimethylamino)cyclopropyl)methanol

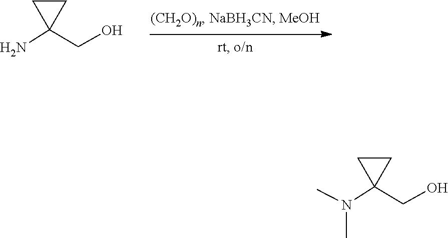

NaBH₃CN (2.89 g, 45.9 mmol) was added to a solution of (1-aminocyclopropyl)methanol (2 g, 22.9 mmol) and paraformaldehyde (6.20 g, 68.8 mmol) in MeOH (20 mL). The resulting mixture was stirred overnight at room temperature. The reaction mixture was concentrated under vacuum and purified by silica gel column chromatography, eluting with MeOH in DCM (0% to 20%). The fractions with the desired mass signal were combined and concentrated under vacuum to afford the desired product (1-(dimethylamino)cyclopropyl)methanol (600 mg, 22.7% yield).

LCMS (ESI−MS) m/z=116.1 [M+H]⁺.

### Step 2: N,N-dimethyl-1-((4-nitro-1H-pyrazol-1-yl)methyl)cyclopropan-1-amine

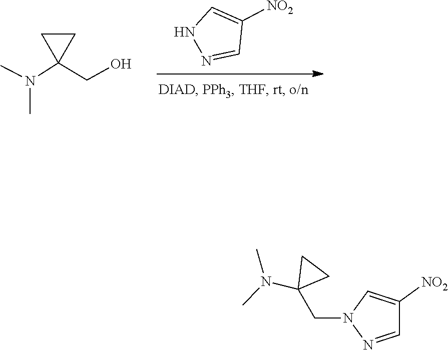

DIAD (1.4 g, 7.16 mmol) was added to a solution of (1-(dimethylamino)cyclopropyl)methanol (550 mg, 4.77 mmol), 4-nitropyrazole (648 mg, 5.73 mmol) and PPh₃ (1.8 g, 7.16 mmol) in THF (6 mL) under a nitrogen atmosphere. The resulting mixture was stirred overnight at room temperature. The reaction mixture was concentrated under vacuum and purified by silica gel column chromatography, eluting with EA in PE (0% to 30%). The fractions with desired mass signal were combined and concentrated under vacuum to afford the desired product N,N-dimethyl-1-((4-nitro-1H-pyrazol-1-yl)methyl)cyclopropan-1-amine (400 mg, 39.8% yield).

LCMS (ESI−MS) m/z=211.1 [M+H]⁺.

### Step 3:1-((1-(dimethylamino)cyclopropyl)methyl)-1H-pyrazol-4-amine

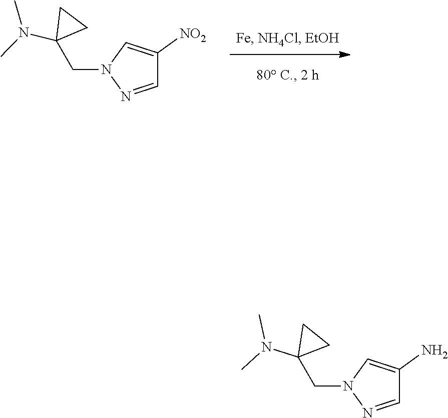

Fe (1.0 g, 19.0 mmol) was added to a solution of N,N-dimethyl-1-((4-nitro-1H-pyrazol-1-yl)methyl)cyclopropan-1-amine (400 mg, 1.90 mmol) and NH₄Cl (407 mg, 7.61 mmol) in a mixture of EtOH (3 mL) and H₂O (1 mL). The resulting mixture was heated to 80° C. and stirred for 2 h. After cooling to room temperature, the resulting mixture was filtered and the filter cake was washed with ethanol (2×10 mL). The filtrate was concentrated under reduced pressure to afford crude 1-((1-(dimethylamino)cyclopropyl)methyl)-1H-pyrazol-4-amine (300 mg, 87.5% yield).

LCMS (ESI−MS) m/z=181.1 [M+H]⁺.

### Intermediate 22: 2-cyclopropylisoindolin-5-amine

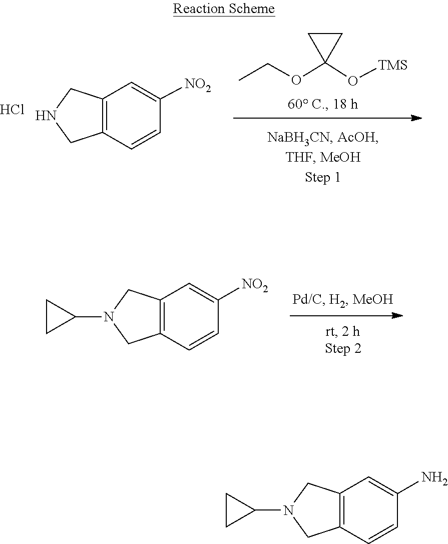

### Detailed Procedure

### Step 1: 2-cyclopropyl-5-nitroisoindoline

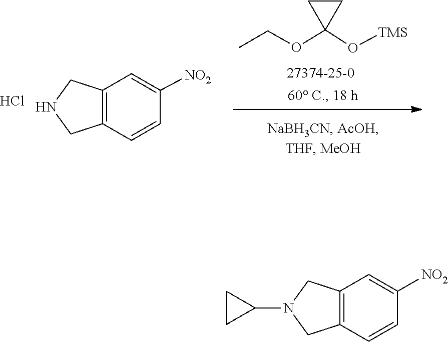

To a solution of 5-nitro-2,3-dihydro-1H-isoindole (2.6 g, 15.8 mmol), AcOH (1.90 g, 31.6 mmol) and (1-ethoxycyclopropoxy)trimethylsilane (11.04 g, 63.3 mmol) in THE (100 mL) and MeOH (10 mL) was added NaBH₃CN (1.49 g, 23.7 mmol) at 20° C. The resulting mixture was stirred for 18 h at 60° C. The reaction mixture was cooled to room temperature and quenched with 1N HCl and extracted with EtOAc. The aqueous layer was basified to PH=10 with solid K₂CO₃ and extracted with DCM. The combined organic phase was washed with water and brine, dried over Na₂SO₄ and concentrated to afford 2-cyclopropyl-5-nitroisoindoline (2 g, 49.47%).

LCMS (ESI−MS) m/z=205.1 [M+H]⁺.

### Step 2: 2-cyclopropylisoindolin-5-amine

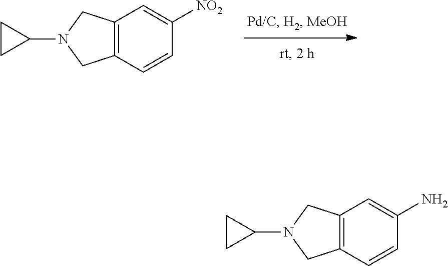 [SMILES: O=[N+]([O-])c1ccc2c(c1)CN(C1CC1)C2 ; Nc1ccc2c(c1)CN(C1CC1)C2]

Pd/C (14.59 mg, 0.14 mmol) was added to the solution of 2-cyclopropyl-5-nitroisoindoline (350 mg, 1.7 mmol) in MeOH (5 mL) under a nitrogen atmosphere. The resulting mixture was stirred for 2 h at room temperature under a hydrogen atmosphere. The reaction mixture was filtered and the filter cake was washed with MeOH (4×10 mL). The combined filtrate was concentrated under reduced pressure to afford crude product 2-cyclopropylisoindolin-5-amine (270 mg, 88.6% yield).

LCMS (ESI−MS) m/z=175.1 [M+H]⁺.

### Intermediate 23: 1-((cyclopentylmethyl)sulfonyl)piperidin-4-amine

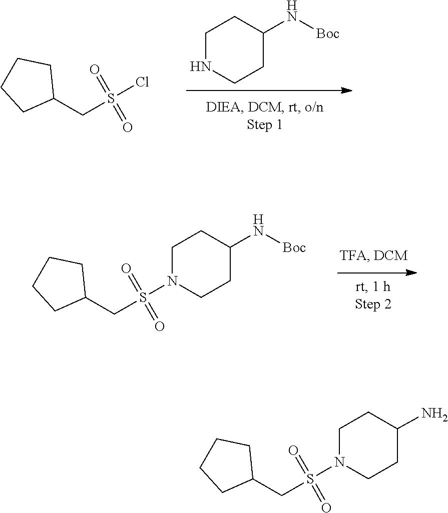 [SMILES: O=S(=O)(Cl)CC1CCCC1 ; CC(C)(C)OC(=O)NC1CCNCC1 ; CC(C)(C)OC(=O)NC1CCN(S(=O)(=O)CC2CCCC2)CC1 ; NC1CCN(S(=O)(=O)CC2CCCC2)CC1]

### Step 1: Tert-butyl (1-((cyclopentylmethyl)sulfonyl)piperidin-4-yl)carbamate

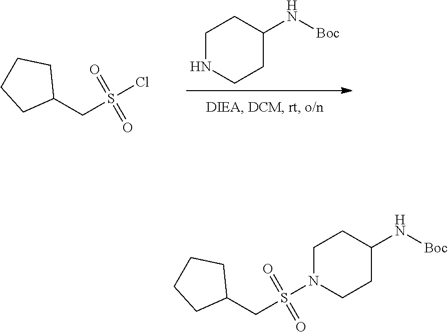 [SMILES: O=S(=O)(Cl)CC1CCCC1 ; CC(C)(C)OC(=O)NC1CCNCC1 ; CC(C)(C)OC(=O)NC1CCN(S(=O)(=O)CC2CCCC2)CC1]

To a solution of cyclopentylmethanesulfonyl chloride (470 mg, 2.57 mmol) and DIEA (831.41 mg, 6.43 mmol) in DCM (10 mL) was added tert-butyl N-(piperidin-4-yl)carbamate (429.45 mg, 2.14 mmol) dropwise at 0° C. The resulting mixture was stirred overnight at room temperature. The resulting mixture was diluted with water (200 mL) and extracted with CH₂Cl₂ (3×250 mL). The combined organic layers were dried over anhydrous Na₂SO₄. After filtration, the filtrate was concentrated under reduced pressure to afford tert-butyl (1-((cyclopentylmethyl)sulfonyl)piperidin-4-yl)carbamate (800 mg, 96.9% yield).

LCMS (ESI−MS) m/z=347.2 [M+H]⁺.

### Step 2: 1-((cyclopentylmethyl)sulfonyl)piperidin-4-amine

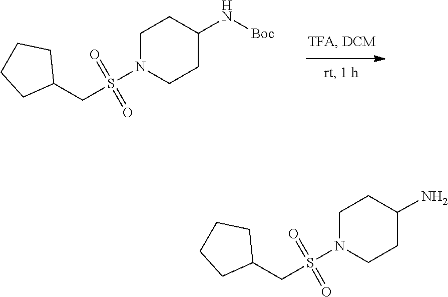 [SMILES: CC(C)(C)OC(=O)NC1CCN(S(=O)(=O)CC2CCCC2)CC1 ; NC1CCN(S(=O)(=O)CC2CCCC2)CC1]

A solution of tert-butyl (1-((cyclopentylmethyl)sulfonyl)piperidin-4-yl)carbamate (400 mg, 1.15 mmol) and TFA (3 mL) in DCM (10 mL) was stirred for 1 h at room temperature. The resulting mixture was concentrated under reduced pressure to afford 1-((cyclopentylmethyl)sulfonyl)piperidin-4-amine (350 mg crude).

LCMS (ESI−MS) m/z=247.1 [M+H]⁺.

### 8-chloro-N-(1-((cyclopentylmethyl)sulfonyl)piperidin-4-yl)-6-methylpyrido[3,4-d]pyrimidin-2-amine

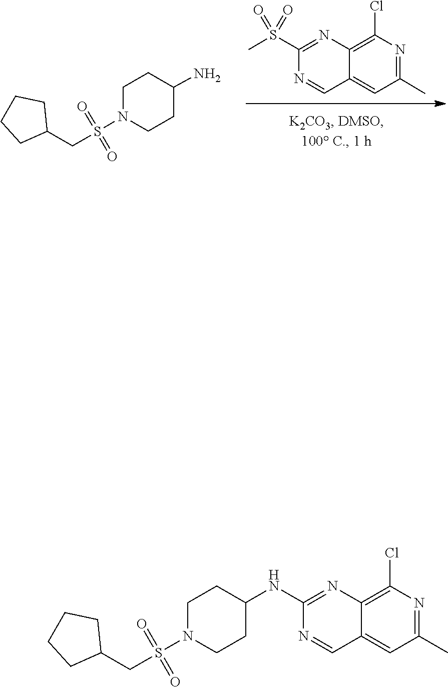

A mixture of 1-cyclopentylmethanesulfonylpiperidin-4-amine (350 mg, 1.42 mmol), 8-chloro-2-methanesulfonyl-6-methylpyrido[3,4-d]pyrimidine (366.1 mg, 1.42 mmol) and K₂CO₃ (588.98 mg, 4.26 mmol) in DMSO (3 mL) was stirred for 1 h at 100° C. The reaction mixture was diluted with water (150 mL) and extracted with DCM (3×150 mL). The combined organic layers were dried over anhydrous Na₂SO₄, filtered and concentrated under vacuum to afford crude 8-chloro-N-(1-((cyclopentylmethyl)sulfonyl) piperidin-4-yl)-6-methylpyrido[3,4-d]pyrimidin-2-amine (300 mg).

LCMS (ESI−MS) m/z=424.1 [M+H]⁺.

### Intermediate 24: 1-cyclopropylcyclobutane-1,3-diol

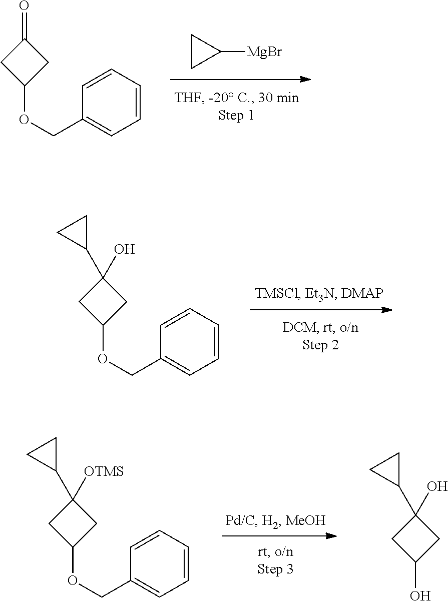

### Step 1: 3-(benzyloxy)-1-cyclopropylcyclobutan-1-ol

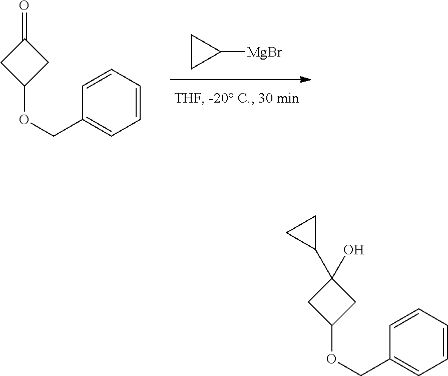

To a stirred solution of 3-(benzyloxy)-1-cyclopropylcyclobutan-1-ol (3 g, 17.0 mmol) in THF (30 mL) was added 1M cyclopropylmagnesium bromide in THF (20.43 mL, 20.4 mmol) dropwise at −20° C. under a nitrogen atmosphere. The mixture was stirred at −20° C. for 30 minutes. The reaction was quenched by the addition of sat. aq. NH₄Cl (30 mL) at 0° C. The resulting mixture was extracted with EA (3×30 mL), dried over anhydrous Na₂SO₄. After filtration, the filtrate was concentrated under reduced pressure. The residue was purified by silica gel column chromatography, eluting with PE/EA (3:1) to afford 3-(benzyloxy)-1-cyclopropylcyclobutan-1-ol (1.6 g, 43.0% yield).

LCMS (ESI−MS) m/z=219.1 [M+H]⁺.

### Step 2: (3-(benzyloxy)-1-cyclopropylcyclobutoxy)trimethylsilane

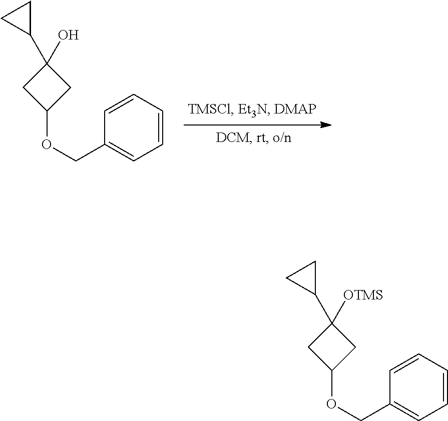

Et₃N (1.95 g, 19.2 mmol) was added to a mixture of 3-(benzyloxy)-1-cyclopropylcyclobutan-1-ol (1.4 g, 6.4 mmol), TMSCl (1.05 g, 9.6 mmol) and DMAP (0.08 g, 0.64 mmol) in DCM (20 mL). The mixture was stirred at room temperature overnight. The reaction was quenched by the addition of sat. aq. NH₄Cl (30 mL) at room temperature. The resulting mixture was extracted with DCM (3×30 mL) and dried over anhydrous Na₂SO₄. After filtration, the filtrate was concentrated under reduced pressure. The residue was purified by silica gel column chromatography, eluting with PE/EA (8:1) to afford (3-(benzyloxy)-1-cyclopropylcy-clobutoxy)trimethylsilane (700 mg, 37.5% yield).

LCMS (ESI−MS) m/z=291.2 [M+H]⁺.

### Step 3: 1-cyclopropylcyclobutane-1,3-diol

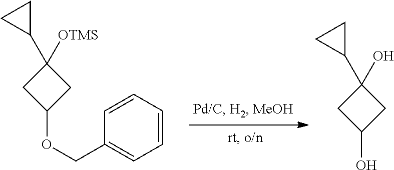

Pd/C (50 mg, 0.47 mmol) was added to (3-(benzyloxy)-1-cyclopropylcyclobutoxy)trimethylsilane (200 mg, 0.69 mmol) in MeOH (5 mL) under a nitrogen atmosphere. The mixture was stirred at room temperature overnight under a hydrogen atmosphere. The resulting mixture was filtered and the filter cake was washed with MeOH (3×30 mL). The filtrate was concentrated under reduced pressure. The residue was purified by Prep-TLC (PE/EA 1:2) to afford 1-cyclopropylcyclobutane-1,3-diol (50 mg, 56.6% yield).

LCMS (ESI−MS) m/z=129.1 [M+H]⁺.

### Intermediate 25: 1-(difluoromethyl)-3-hydroxycyclobutyl benzoate

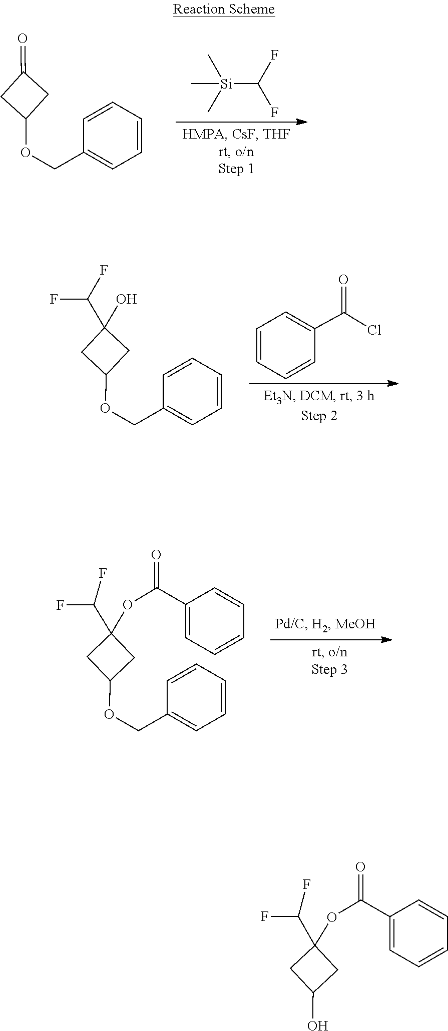

### Step 1: 3-(Benzyloxy)-1-(difluoromethyl)cyclobutan-1-ol

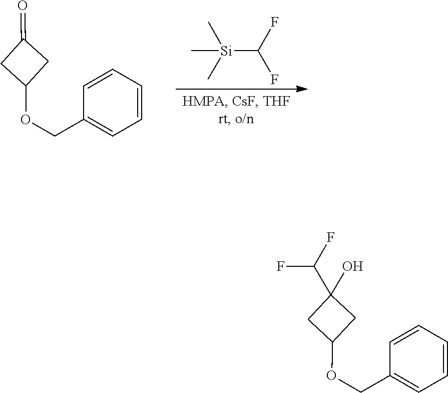

To a solution of 3-(benzyloxy)cyclobutan-1-one (10 g, 56.74 mmol) in THE (100 mL) was added (difluoromethyl)trimethylsilane (8.46 g, 68.09 mmol), HMPA (101.70 g, 567.49 mmol) and CsF (8.62 g, 56.74 mmol) at room temperature under a nitrogen atmosphere. The resulting mixture was stirred overnight at room temperature. The reaction mixture was diluted with water (500 mL) and extracted with EA (2×500 mL). The combined organic layers were dried over anhydrous Na₂SO₄. After filtration, the filtrate was concentrated under reduced pressure. The residue was purified by silica gel column chromatography (PE/EA, 5:1) to afford 3-(benzyloxy)-1-(difluoromethyl)cyclobutan-1-ol (3.5 g, 27.0% yield) as colorless oil.

LCMS (ESI−MS) m/z=229.1 [M+H]⁺.

### Step 2: 3-(benzyloxy)-1-(difluoromethyl)cyclobutyl benzoate

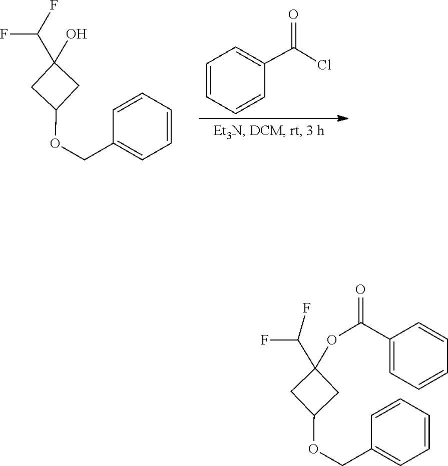

A solution of 3-(benzyloxy)-1-(difluoromethyl)cyclobutan-1-ol (3.4 g, 14.89 mmol) in DCM (30 mL) was treated with Et₃N (4.52 g, 44.69 mmol) for 3 min at 0° C. Benzoyl chloride (2.30 g, 16.38 mmol) was then added in portions at 0° C. The resulting mixture was stirred 3 h at room temperature. The reaction mixture was diluted with water (200 mL) and extracted with DCM (3×200 mL). The combined organic layers were washed with saturated sodium chloride solution (2×200 mL) and dried over anhydrous Na₂SO₄. After filtration, the filtrate was concentrated under reduced pressure. The residue was purified by silica gel column chromatography (PE/EA, 1:1) to afford 3-(benzyloxy)-1-(difluoromethyl)cyclobutyl benzoate (1 g, 20.2% yield) as colorless oil.

LCMS (ESI−MS) m/z=333.1 [M+H]⁺.

### Step 3: 1-(difluoromethyl)-3-hydroxycyclobutyl benzoate

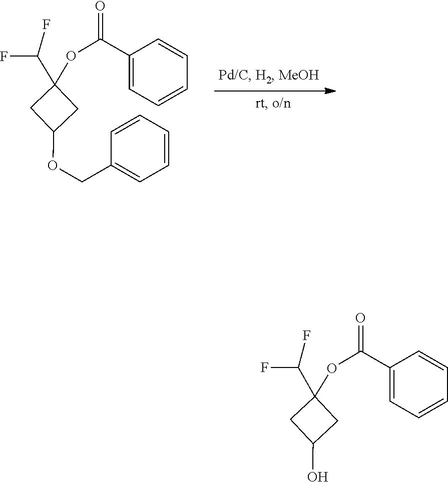 [SMILES: O=C(OC1(C(F)F)CC(OCc2ccccc2)C1)c1ccccc1 ; O=C(OC1(C(F)F)CC(O)C1)c1ccccc1]

Pd/C (200 mg, 10% on carbon) was added to a solution of 3-(benzyloxy)-1-(difluoromethyl)cyclobutyl benzoate (800 mg, 2.40 mmol) in MeOH (10 mL) under a nitrogen atmosphere, the resulting mixture was stirred overnight at room temperature under a hydrogen atmosphere. The reaction mixture was filtered and the filtrate was concentrated under reduced pressure to afford crude product 1-(difluoromethyl)-3-hydroxycyclobutyl benzoate (583.1 mg, 90.0% yield) as a colorless oil.

¹H NMR (400 MHz, Chloroform-d) δ 8.12-8.03 (m, 2H), 7.63-7.59 (m, 1H), 7.49-7.45 (m, 2H), 6.46-6.17 (m, 1H), 4.28-4.21 (m, 1H), 3.26-3.14 (m, 3H), 2.48-2.43 (m, 2H).

LCMS (ESI−MS) m/z=243.0 [M+H]⁺.

### Intermediate 26: Tert-butyl 2-hydroxy-5-oxa-8-azaspiro[3.5]nonane-8-carboxylate

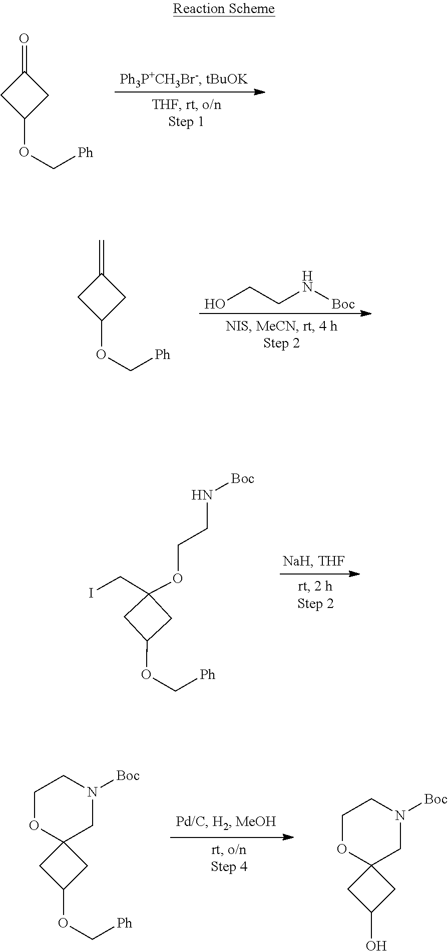 [SMILES: O=C1CC(OCc2ccccc2)C1 ; C=C1CC(OCc2ccccc2)C1 ; CC(C)(C)OC(=O)NCCO ; CC(C)(C)OC(=O)NCCOC1(CI)CC(OCc2ccccc2)C1 ; CC(C)(C)OC(=O)N1CCOC2(CC(OCc3ccccc3)C2)C1 ; CC(C)(C)OC(=O)N1CCOC2(CC(O)C2)C1]

### Detailed Procedure

### Step 1: ((3-methylenecyclobutoxy)methyl)benzene

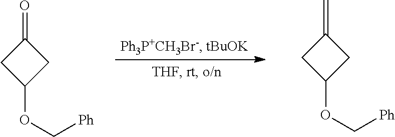

To a stirred mixture of methyltriphenylphosphanium bromide (3.24 g, 90.79 mmol) in THF (100 mL) was added t-BuOK (1.14 g, 102.14 mmol) at room temperature. The reaction mixture was stirred for 3 h at room temperature. 3-(Benzyloxy)cyclobutan-1-one (10 g, 56.74 mmol) was added and the reaction was stirred overnight at room temperature. The reaction mixture was diluted with water (300 mL) and extracted with ether (3×300 mL). The combined organic layers were dried and concentrated. The residue was purified by silica gel column chromatography (PE/EA, 96:4) to afford ((3-methylenecyclobutoxy)methyl)benzene (1.8 g, 18.2% yield) as a colorless oil.

LCMS (ESI−MS) m/z=175.1 [M+H]⁺.

### Step 2: Tert-butyl (2-(3-(benzyloxy)-1-(iodomethyl)cyclobutoxy)ethyl)carbamate

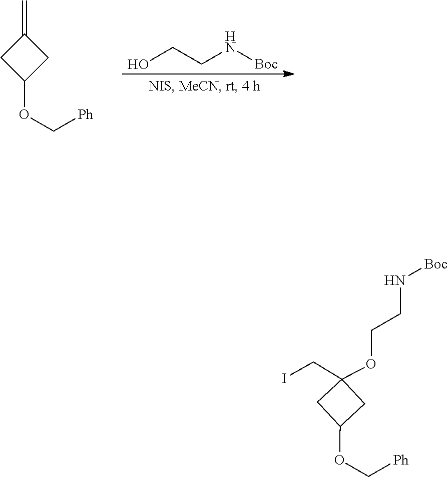 [SMILES: C=C1CC(OCc2ccccc2)C1 ; CC(C)(C)OC(=O)NCCO ; CC(C)(C)OC(=O)NCCOC1(CI)CC(OCc2ccccc2)C1]

To a solution of tert-butyl N-(2-hydroxyethyl)carbamate (7.77 g, 48.20 mmol) and ((3-methylenecyclobutoxy)methyl)benzene (7 g, 40.17 mmol) in MeCN (70 mL) was added 1-iodo-5-pyrrolidinedione (1.08 g, 48.20 mmol), the mixture was stirred at room temperature for 4 h. The reaction mixture was diluted with water (100 mL) and extracted with ether (3×100 mL). The combined organic layers were dried and concentrated. The residue was purified by silica gel column chromatography (PE/EA, 85:15) to afford tert-butyl (2-(3-(benzyloxy)-1-(iodomethyl)cyclobutoxy)ethyl)carbamate (2 g, 41.9% yield) as a colorless oil.

LCMS (ESI−MS) m/z=462.1 [M+H]⁺.

### Step 3: Tert-butyl 2-(benzyloxy)-5-oxa-8-azaspiro[3.5]nonane-8-carboxylate

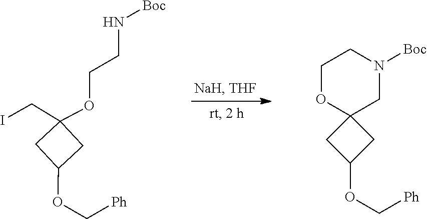 [SMILES: CC(C)(C)OC(=O)NCCOC1(CI)CC(OCc2ccccc2)C1 ; CC(C)(C)OC(=O)N1CCOC2(CC(OCc3ccccc3)C2)C1]

To a cooled to 0° C. solution of tert-butyl (2-(3-(benzyloxy)-1-(iodomethyl)cyclobutoxy)ethyl)carbamate (7.2 g, 15.60 mmol) in anhydrous THE (70 mL) was added NaH (0.75 g, 31.21 mmol, 60% in mineral oil). The mixture was stirred at room temperature for 2 h. The reaction was quenched with aq. NH₄Cl (100 mL) and extracted with EA (3×200 mL). The combined organic layers were dried and concentrated under reduced pressure. The residue was purified by silica gel chromatography (EA/PE, 20:80) to afford tert-butyl 2-(benzyloxy)-5-oxa-8-azaspiro[3.5]nonane-8-carboxylate (3 g, 57.7% yield) as a colorless oil.

LCMS (ESI−MS) m/z=334.1 [M+H]⁺.

### Step 4: Tert-butyl 2-hydroxy-5-oxa-8-azaspiro[3.5]nonane-8-carboxylate

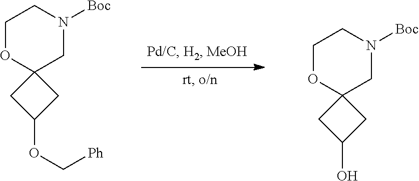 [SMILES: CC(C)(C)OC(=O)N1CCOC2(CC(OCc3ccccc3)C2)C1 ; CC(C)(C)OC(=O)N1CCOC2(CC(O)C2)C1]

A solution of Pd/C (2.87 g, 26.99 mmol, 10% on carbon) and tert-butyl 2-(benzyloxy)-5-oxa-8-azaspiro[3.5]nonane-8-carboxylate (3 g, 8.99 mmol) in MeOH (30 mL) was stirred at room temperature overnight under a hydrogen atmosphere. The reaction was filtered, and the filtrate was concentrated under reduced pressure. The crude product was purified by silica gel column chromatography (PE/EA, 70:30) to afford tert-butyl 2-hydroxy-5-oxa-8-azaspiro[3.5]nonane-8-carboxylate (2 g, ⁹1.4% yield) as a colorless oil.

¹H NMR (400 MHz, DMSO-d₆) δ 5.08 (dd, J=41.6, 5.7 Hz, 1H), 4.26-3.70 (m, 1H), 3.45 (q, J=4.7 Hz, 2H), 3.33 (d, J=23.4 Hz, 1H), 3.28-3.19 (m, 2H), 3.19-3.11 (m, 1H), 2.33-2.26 (m, 1H), 2.25-2.17 (m, 1H), 1.80-1.66 (m, 2H), 1.40 (s, 9H).

### Intermediate 27: Tert-butyl 8,8-difluoro-2-hydroxy-6-azaspiro[3.4]octane-6-carboxylate

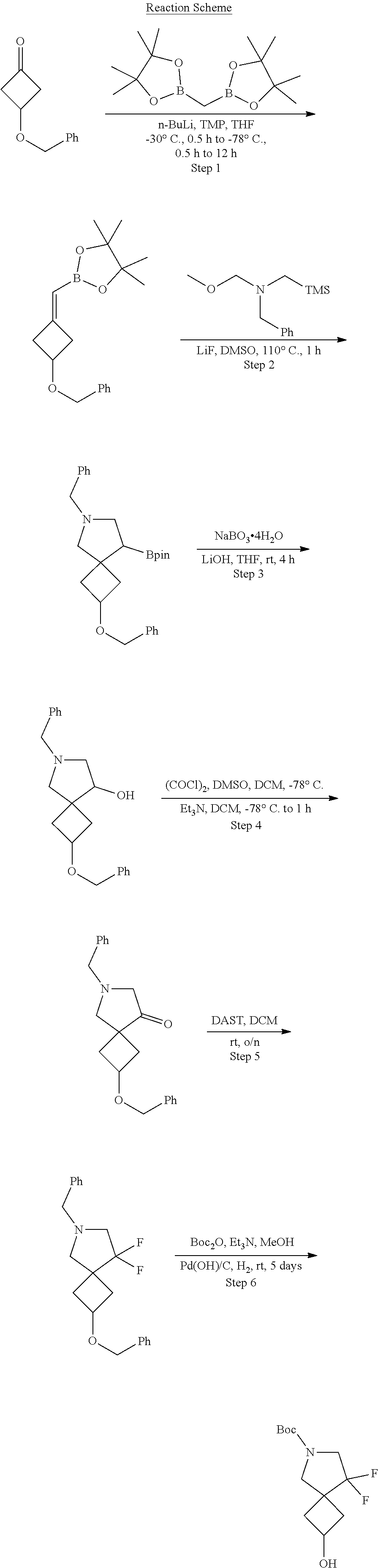

### Detailed Procedure

### Step 1: 2-((3-(benzyloxy)cyclobutylidene)methyl)-4,4,5,5-tetramethyl-1,3,2-dioxaborolane

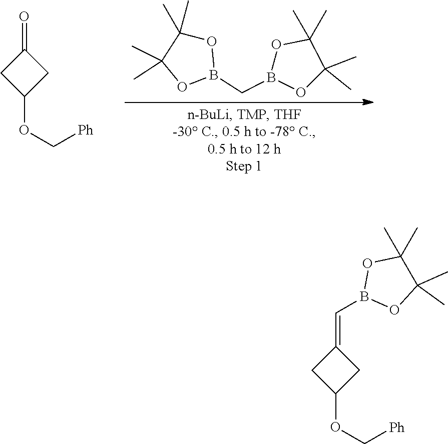

To a cooled to −30° C. solution of 2,2,6,6-tetramethylpiperidine (9.62 g, 68.09 mmol) in dry THF (100 mL) was added n-BuLi (2.5 M, 27.2 mL) dropwise under an N₂ atmosphere. The mixture was stirred at −30° C. for 0.5 h. The reaction was then cooled to −78° C. and a solution of bis(4,4,5,5-tetramethyl-1,3,2-dioxaborolan-2-yl)methane (15.21 g, 56.74 mmol) in 50 mL of dry THF was added dropwise. The reaction mixture was stirred at −78° C. for 0.5 h and a solution of 3-(benzyloxy)cyclobutan-1-one (10 g, 56.74 mmol) in 50 mL of dry THF was added dropwise. The reaction mixture was then warmed to 20° C. and stirred for an additional 12 h. The reaction mixture was slowly poured into 20 mL of saturated aqueous NH₄Cl at 0° C. and after stirring for 1 h, the solution was diluted with H₂O (200 mL) and extracted with EtOAc (400 mL×3). The organic phase was dried over Na₂SO₄, filtered and concentrated under reduced pressure to afford 2-((3-(benzyloxy)cyclobutylidene)methyl)-4,4,5,5-tetramethyl-1,3,2-dioxaborolane (13.7 g crude) which was used without further purification.

LCMS (ESI−MS) m/z=301.1 [M+H]⁺.

### Step 2: 6-benzyl-2-(benzyloxy)-8-(4,4,5,5-tetramethyl-1,3,2-dioxaborolan-2-yl)-6-azaspiro[3.4]octane

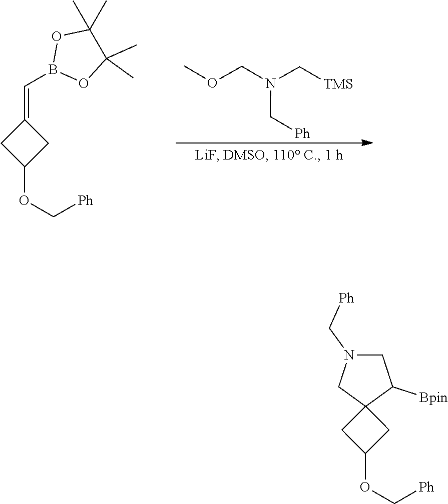

A solution of 2-((3-(benzyloxy)cyclobutylidene)methyl)-4,4,5,5-tetramethyl-1,3,2-dioxaborolane (13.7 g crude), N-benzyl-1-methoxy-N-((trimethylsilyl)methyl)methanamine (13.00 g, 54.76 mmol) and LiF (3.55 g, 136.90 mmol) in DMSO (200 mL) was stirred at 110° C. for 1 h. The reaction mixture was diluted with H₂O (200 mL) and extracted with EA (1000 mL). The combined organic layers were washed with brine, dried over anhydrous Na₂SO₄, filtered and concentrated under reduced pressure to afford crude 6-benzyl-2-(benzyloxy)-8-(4,4,5,5-tetramethyl-1,3,2-dioxaborolan-2-yl)-6-azaspiro[3.4]octane (20 g) as a colorless oil which was used for the next step without further purification.

LCMS (ESI−MS) m/z=434.2 [M+H]⁺.

### Step 3: 6-benzyl-2-(benzyloxy)-6-azaspiro[3.4]octan-8-ol

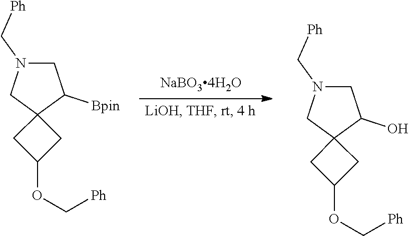

A solution of 6-benzyl-2-(benzyloxy)-8-(4,4,5,5-tetramethyl-1,3,2-dioxaborolan-2-yl)-6-azaspiro[3.4]octane (20 g crude), sodium perborate (4.53 g, 55.37 mmol) and LiOH (3.32 g, 138.44 mmol) in THE (50 mL) and H₂O (200 mL) was stirred at room temperature for 4 h. The reaction mixture was diluted with H₂O (200 mL) and extracted with EA (3×500 mL). The combined organic layers were washed with brine, dried over anhydrous Na₂SO₄, filtered and concentrated under reduced pressure. The crude product was purified by silica gel column chromatography (MeOH/DCM, 3:97) to obtain 6-benzyl-2-(benzyloxy)-6-azaspiro[3.4]octan-8-ol (10 g, 67.0%) as colorless oil.

LCMS (ESI−MS) m/z=324.1 [M+H]⁺.

### Step 4: 6-benzyl-2-(benzyloxy)-6-azaspiro[3.4]octan-8-one

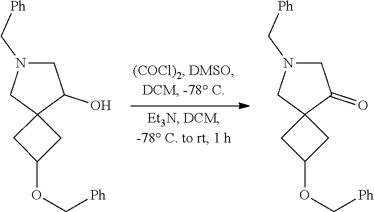 [SMILES: OC1CN(Cc2ccccc2)CC12CC(OCc1ccccc1)C2 ; O=C1CN(Cc2ccccc2)CC12CC(OCc1ccccc1)C2]

To a cooled to −78° C. solution of oxalyl chloride (7.85 g, 61.8 mmol) in DCM (100 mL) was added dropwise a solution of DMSO (4.83 g, 61.8 mmol) in DCM (20 mL) under a nitrogen atmosphere. The mixture was stirred at −78° C. for 20 min. A solution of 6-benzyl-2-(benzyloxy)-6-azaspiro[3.4]octan-8-ol (10 g, 30.9 mmol) in DCM (20 mL) was then added dropwise and the mixture stirred for 20 min. Et₃N (12.5 g, 123 mmol) was added dropwise and the mixture stirred for 20 min. The reaction mixture was diluted with water (200 mL) and extracted with DCM (3×200 mL). The combined organic layers were washed with brine, dried over anhydrous Na₂SO₄, filtered and concentrated under reduced pressure. The residue was purified by silica gel column chromatography (EA in PE, 0 to 10%). The fractions with the desired mass signal were combined and concentrated under reduced pressure to afford 6-benzyl-2-(benzyloxy)-6-azaspiro[3.4]octan-8-one (5.8 g, 58.4% yield).

LCMS (ESI−MS) m/z=322.2 [M+H]⁺.

### Step 5: 6-benzyl-2-(benzyloxy)-8,8-difluoro-6-azaspiro[3.4]octane

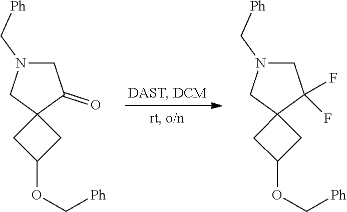 [SMILES: O=C1CN(Cc2ccccc2)CC12CC(OCc1ccccc1)C2 ; FC1(F)CN(Cc2ccccc2)CC12CC(OCc1ccccc1)C2]

DAST (8.73 g, 54.1 mmol) was added to a solution of 6-benzyl-2-(benzyloxy)-6-azaspiro[3.4]octan-8-one (5.8 g, 18.0 mmol) in DCM (60 mL) at 0° C. The resulting mixture was stirred overnight at room temperature. The reaction mixture was concentrated under reduced pressure. The residue was purified by silica gel column chromatography (EA in PE, 0% to 10%). The fractions with the desired mass signal were combined and concentrated under reduced pressure to afford 6-benzyl-2-(benzyloxy)-8,8-difluoro-6-azaspiro[3.4]octane (1.2 g, 19.4% yield).

LCMS (ESI−MS) m/z=344.2 [M+H]⁺.

### Step 6: Tert-butyl 8,8-difluoro-2-hydroxy-6-azaspiro[3.4]octane-6-carboxylate

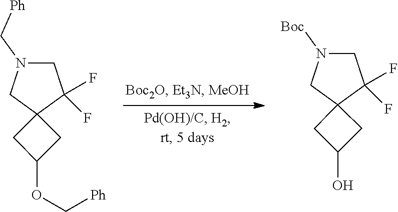 [SMILES: FC1(F)CN(Cc2ccccc2)CC12CC(OCc1ccccc1)C2 ; CC(C)(C)OC(=O)N1CC(F)(F)C2(CC(O)C2)C1]

Pd(OH)₂/C (0.49 g, 3.49 mmol) was added to a solution of 6-benzyl-2-(benzyloxy)-8,8-difluoro-6-azaspiro[3.4]octane (1.2 g, 3.49 mmol), Boc₂O (0.92 g, 4.19 mmol) and Et₃N (1.06 g, 10.48 mmol) in MeOH (120 mL). The resulting mixture was stirred 5 days at room temperature under a H₂ atmosphere. The reaction mixture was filtered and the filtrate was concentrated under reduced pressure. The residue was purified by silica gel column chromatography (MeOH in DCM, 0% to 5%). The fractions with the desired mass signal were combined, concentrated under reduced pressure and lyophilized to afford the tert-butyl 8,8-difluoro-2-hydroxy-6-azaspiro[3.4]octane-6-carboxylate (500 mg, 54.4% yield).

¹H NMR (400 MHz, DMSO-d₆) δ 5.3-5.06 (m, 1H), 4.20-4.00 (m, 1H), 3.69-3.50 (m, 3H), 3.47-3.39 (m, 2H), 2.14-2.10 (m, 1H), 2.04-1.96 (m, 1H), 1.93-1.85 (m, 1H), 1.40 (s, 9H).

### Intermediate 28: 6-benzyl 2-(tert-butyl) 8-(difluoromethyl)-2,6-diazaspiro[3.4]octane-2,6-dicarboxylate

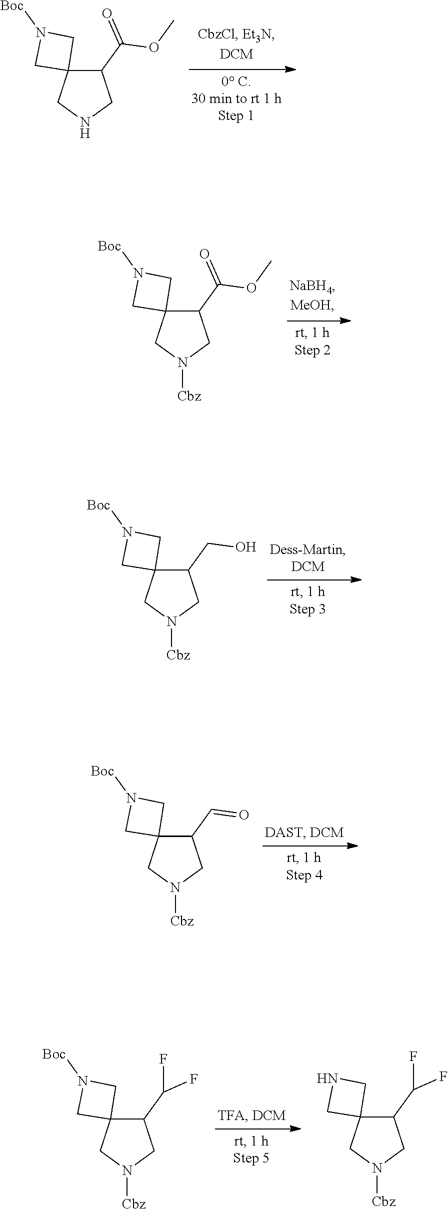 [SMILES: COC(=O)C1CNCC12CN(C(=O)OC(C)(C)C)C2 ; COC(=O)C1CN(C(=O)OCc2ccccc2)CC12CN(C(=O)OC(C)(C)C)C2 ; CC(C)(C)OC(=O)N1CC2(CN(C(=O)OCc3ccccc3)CC2CO)C1 ; CC(C)(C)OC(=O)N1CC2(CN(C(=O)OCc3ccccc3)CC2C=O)C1 ; CC(C)(C)OC(=O)N1CC2(CN(C(=O)OCc3ccccc3)CC2C(F)F)C1 ; O=C(OCc1ccccc1)N1CC(C(F)F)C2(CNC2)C1]

### Step 1: 6-benzyl 2-(tert-butyl) 8-methyl 2,6-diazaspiro[3.4]octane-2,6,8-tricarboxylate

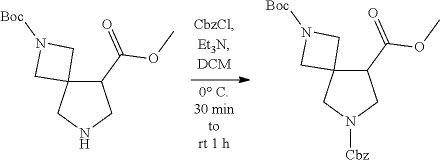 [SMILES: COC(=O)C1CNCC12CN(C(=O)OC(C)(C)C)C2 ; COC(=O)C1CN(C(=O)OCc2ccccc2)CC12CN(C(=O)OC(C)(C)C)C2]

CbzCl (1.14 g, 6.65 mmol) and Et₃N (0.90 g, 8.87 mmol) were added to a cooled to 0° C. solution of 2-(tert-butyl) 8-methyl 2,6-diazaspiro[3.4]octane-2,8-dicarboxylate (1.2 g, 4.43 mmol) in DCM (20 mL). The reaction mixture was stirred for 30 minutes, then warmed to room temperature and stirred for 1 h. The reaction was quenched with water (200 mL). The resulting mixture was extracted with CH₂Cl₂ (3×200 mL). The combined organic layers were dried over anhydrous Na₂SO₄. After filtration, the filtrate was concentrated under reduced pressure. The residue was purified by silica gel column chromatography (PE/EA, 1:1) to afford 6-benzyl 2-(tert-butyl) 8-methyl 2,6-diazaspiro[3.4]octane-2,6,8-tricarboxylate (1.5 g, 83.6% yield).

LCMS (ESI-MS) m/z=305.1 [M+H−100]⁺.

### Step 2: 6-benzyl 2-(tert-butyl) 8-(hydroxymethyl)-2,6-diazaspiro[3.4]octane-2,6-dicarboxylate

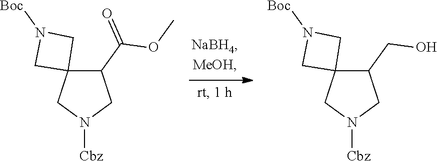 [SMILES: COC(=O)C1CN(C(=O)OCc2ccccc2)CC12CN(C(=O)OC(C)(C)C)C2 ; CC(C)(C)OC(=O)N1CC2(CN(C(=O)OCc3ccccc3)CC2CO)C1]

NaBH₄ (74.83 mg, 1.97 mmol) was added to a solution of 6-benzyl 2-(tert-butyl) 8-methyl 2,6-diazaspiro[3.4]octane-2,6,8-tricarboxylate (200 mg, 0.49 mmol) in MeOH (3 mL). The reaction mixture was stirred for 1 h at room temperature and quenched with water (20 mL). The resulting mixture was extracted with EA (3×20 mL). The combined organic layers were dried over anhydrous Na₂SO₄. After filtration, the filtrate was concentrated under reduced pressure and used as such for the next step.

LCMS (ESI-MS) m/z=277.1 [M+H−100]⁺.

### Step 3: 6-benzyl 2-(tert-butyl) 8-formyl-2,6-diazaspiro[3.4]octane-2,6-dicarboxylate

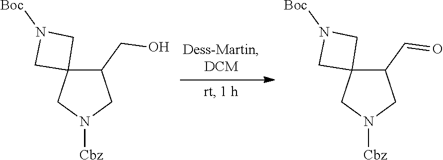 [SMILES: CC(C)(C)OC(=O)N1CC2(CN(C(=O)OCc3ccccc3)CC2CO)C1 ; CC(C)(C)OC(=O)N1CC2(CN(C(=O)OCc3ccccc3)CC2C=O)C1]

Dess-Martin (581.37 mg, 1.37 mmol) was added to a mixture of 6-benzyl 2-(tert-butyl) 8-(hydroxymethyl)-2,6-diazaspiro[3.4]octane-2,6-dicarboxylate (430 mg, 1.14 mmol) in DCM (8 mL). The resulting mixture was stirred for 1 h at room temperature and filtered. The filter cake was washed with CH₂Cl₂ (30 mL) and the filtrate was concentrated under reduced pressure. The residue was purified by Prep-TLC (PE/EA, 1:3) to afford 6-benzyl 2-(tert-butyl) 8-formyl-2,6-diazaspiro[3.4]octane-2,6-dicarboxylate (240 mg, 56.1% yield).

LCMS (ESI-MS) m/z=275.1 [M+H−100]⁺.

### Step 4: 6-benzyl 2-(tert-butyl) 8-(difluoromethyl)-2,6-diazaspiro[3.4]octane-2,6-dicarboxylate

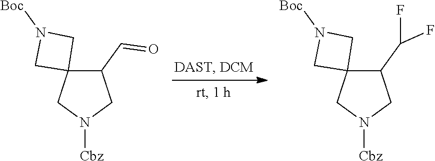 [SMILES: CC(C)(C)OC(=O)N1CC2(CN(C(=O)OCc3ccccc3)CC2C=O)C1 ; CC(C)(C)OC(=O)N1CC2(CN(C(=O)OCc3ccccc3)CC2C(F)F)C1]

DAST (206.63 mg, 1.28 mmol) was added to a mixture of 6-benzyl 2-(tert-butyl) 8-formyl-2,6-diazaspiro[3.4]octane-2,6-dicarboxylate (240 mg, 0.64 mmol) in DCM (5 mL). The mixture was stirred for 1 h at room temperature and quenched with water (20 mL). The resulting mixture was extracted with CH₂Cl₂ (2×20 mL). The combined organic layers were dried over anhydrous Na₂SO₄. After filtration, the filtrate was concentrated under reduced pressure. The residue was purified by Prep-TLC (PE/EA, 1:3) to afford 6-benzyl 2-(tert-butyl) 8-(difluoromethyl)-2,6-diazaspiro[3.4]octane-2,6-dicarboxylate (160 mg, 63.0% yield).

LCMS (ESI-MS) m/z=297.1 [M+H−100]⁺.

### Step 5: Benzyl 8-(difluoromethyl)-2,6-diazaspiro[3.4]octane-6-carboxylate

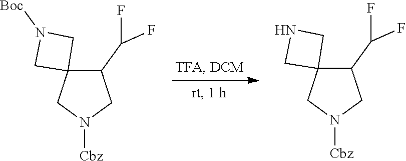 [SMILES: CC(C)(C)OC(=O)N1CC2(CN(C(=O)OCc3ccccc3)CC2C(F)F)C1 ; O=C(OCc1ccccc1)N1CC(C(F)F)C2(CNC2)C1]

A solution of 6-benzyl 2-(tert-butyl) 8-(difluoromethyl)-2,6-diazaspiro[3.4]octane-2,6-dicarboxylate (130 mg, 0.32 mmol) in TFA (0.3 mL) and DCM (0.9 mL) was stirred for 1 h at room temperature. The reaction mixture was concentrated under reduced pressure and used as such for the next step.

LCMS (ESI-MS) m/z=297.1 [M+H]⁺.

### Example Compounds

### Example 2: 8-(2,2-difluoro-6-azaspiro[3.4]octan-6-yl)-N-(1-(methylsulfonyl)piperidin-4-yl)pyrido[3,4-d]pyrimidin-2-amine

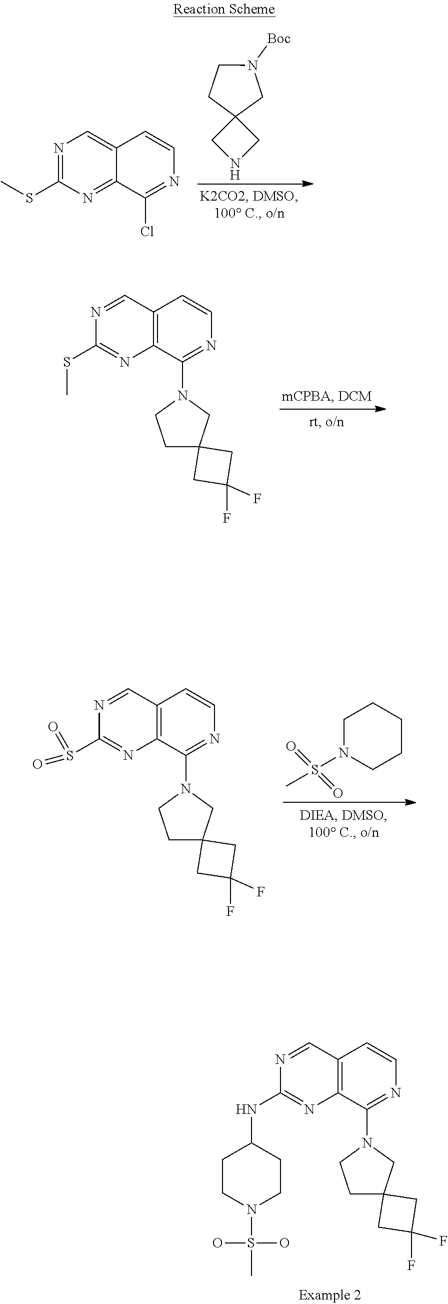

### Detailed Procedure

### Step 1: 8-(2,2-difluoro-6-azaspiro[3.4]octan-6-yl)-2-(methylthio)pyrido[3,4-d]pyrimidine

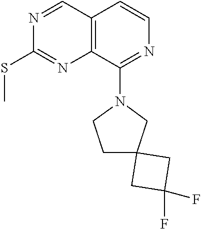 [SMILES: CSc1ncc2ccnc(N3CCC4(C3)CC(F)(F)C4)c2n1]

To a stirred mixture of 8-chloro-2-(methylthio)pyrido[3,4-d]pyrimidine (900 mg, 4.25 mmol) and 2,2-difluoro-6-azaspiro[3.4]octane hydrochloride (780.4 mg, 4.25 mmol) in acetonitrile (10 mL) was added N,N-diisopropylethylamine (1.65 g, 12.75 mmol). The resulting mixture was heated to 100° C. and stirred overnight. The reaction mixture was allowed to cool to room temperature and concentrated under vacuum. The residue was diluted with water (100 mL) and extracted with ethyl acetate (3×100 mL). The combined organic layers were washed with brine, dried over anhydrous sodium sulfate and filtered. The filtrate was concentrated under vacuum to afford the crude product 8-(2,2-difluoro-6-azaspiro[3.4]octan-6-yl)-2-(methylthio)pyrido[3,4-d]pyrimidine (880 mg). The crude product was used for next step directly without further purification.

LCMS (ESI) m/z=323 [M+H]⁺.

### Step 2: 8-(2,2-difluoro-6-azaspiro[3.4]octan-6-yl)-2-(methylsulfonyl)pyrido[3,4-d]pyrimidine

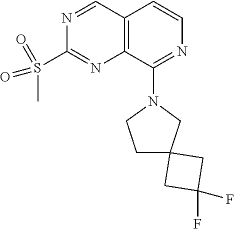 [SMILES: CS(=O)(=O)c1ncc2ccnc(N3CCC4(C3)CC(F)(F)C4)c2n1]

To a stirred solution of 8-(2,2-difluoro-6-azaspiro[3.4]octan-6-yl)-2-(methylthio)pyrido[3,4-d]pyrimidine (780 mg, 2.42 mmol) in dichloromethane (20 mL) was added 3-chloroperoxybenzoic acid (1.04 g, 6.05 mmol). The resulting mixture was stirred overnight at room temperature and concentrated under vacuum to afford the crude 8-(2,2-difluoro-6-azaspiro[3.4]octan-6-yl)-2-(methylsulfonyl)pyrido[3,4-d]pyrimidine (910 mg), the crude product was used for next step directly without further purification. LCMS (ESI) m/z=355 [M+H]⁺.

### Step 3: 8-(2,2-difluoro-6-azaspiro[3.4]octan-6-yl)-N-(1-(methylsulfonyl)piperidin-4-yl)pyrido[3,4-d]pyrimidin-2-amine

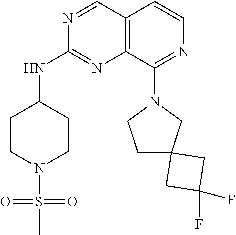 [SMILES: CS(=O)(=O)N1CCC(Nc2ncc3ccnc(N4CCC5(C4)CC(F)(F)C5)c3n2)CC1]

To a stirred mixture of 8-(2,2-difluoro-6-azaspiro[3.4]octan-6-yl)-2-(methylsulfonyl)pyrido[3,4-d]pyrimidine (150 mg, 0.42 mmol) and 1-(methylsulfonyl)piperidin-4-amine (75.45 mg, 0.42 mmol) in dimethyl sulfoxide (2 mL) were added N,N-diisopropylethylamine (164.12 mg, 1.26 mmol). The resulting mixture was heated to 100° C. and stirred overnight. The reaction mixture was allowed to cool to room temperature, diluted with water (20 mL) and extracted with ethyl acetate (3×20 mL). The combined organic layers were washed with brine (50 mL), dried with anhydrous sodium sulfate, filtered and the filtrate was concentrated under vacuum to afford the crude product. The crude product was purified by preparative reverse phase HPLC (acetonitrile/water (with 10 mM NH₄HCO₃ and 0.1% NH₃·H₂O) gradient) to afford the title compound (48.9 mg, 23.8% yield).

¹H NMR (400 MHz, DMSO-d₆) δ 8.99 (s, 1H), 7.73 (d, J=5.6 Hz, 1H), 7.52 (s, 1H), 6.81 (d, J=5.2 Hz, 1H), 4.10 (S, 2H), 3.89 (S, 3H), 3.59 (d, J=12.4 Hz, 2H), 2.93-2.86 (m, 5H), 2.75-2.56 (m, 4H), 2.13-1.96 (m, 4H), 1.69-1.56 (m, 2H).

LCMS (ESI-MS) m/z=453 [M+H]⁺.

### Example 6: 8-(2,2-difluoro-6-azaspiro[3.4]octan-6-yl)-N-(5-(6-ethyl-2,6-diazaspiro[3.3]heptan-2-yl)pyridin-2-yl)pyrido[3,4-d]pyrimidin-2-amine

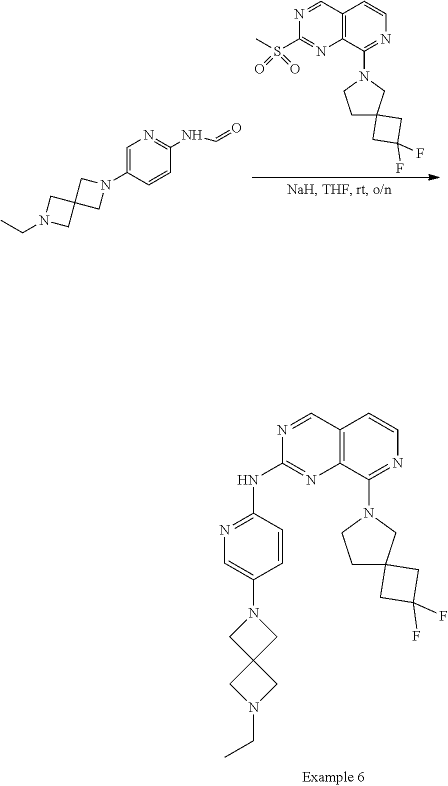 [SMILES: CCN1CC2(C1)CN(c1ccc(NC=O)nc1)C2 ; CS(=O)(=O)c1ncc2ccnc(N3CCC4(C3)CC(F)(F)C4)c2n1 ; CCN1CC2(C1)CN(c1ccc(Nc3ncc4ccnc(N5CCC6(C5)CC(F)(F)C6)c4n3)nc1)C2]

To a stirred mixture of N-(5-(6-ethyl-2,6-diazaspiro[3.3]heptan-2-yl)pyridin-2-yl)formamide (50 mg, 0.20 mmol) in tetrahydrofuran (2 mL) was added sodium hydride (60% in mineral oil, 24 mg, 0.60 mmol) at 0° C. The resulting mixture was stirred for 15 minutes at 0° C. Then 8-(2,2-difluoro-6-azaspiro[3.4]octan-6-yl)-2-(methylsulfonyl)pyrido[3,4-d]pyrimidine (78 mg, 0.22 mmol) was added to the mixture and the resulting mixture was warmed to room temperature and stirred overnight. The reaction mixture was quenched by addition of water (50 mL) and extracted with dichloromethane (3×50 mL). The combined organic layers were washed with brine (100 mL), dried over anhydrous sodium sulfate, and filtered. The filtrate was concentrated under vacuum and the residue was purified by preparative reverse phase HPLC (acetonitrile/water (with 10 mM NH₄HCO₃ and 0.1% NH₃·H₂O) gradient) to afford the title compound (12.7 mg, 10.0% yield).

¹H NMR (400 MHz, DMSO-d₆) δ (ppm) 9.13 (s, 1H), 7.94-7.62 (m, 3H), 7.14-6.97 (m, 2H), 4.39 (s, 4H), 4.12-4.18 (m, 6H), 3.98-3.92 (m, 2H), 3.27-3.25 (m, 2H), 2.67-2.61 (m, 4H), 2.16-2.13 (m, 2H), 1.42-1.34 (m, 1H), 1.31-1.23 (m, 3H).

LCMS (ESI) m/z=493 [M+H]⁺.

### Example 6: 8-(2,2-difluoro-6-azaspiro[3.4]octan-6-yl)-N-(5-(6-ethyl-2,6-diazaspiro[3.3]heptan-2-yl)pyridin-2-yl)pyrido[3,4-d]pyrimidin-2-amine

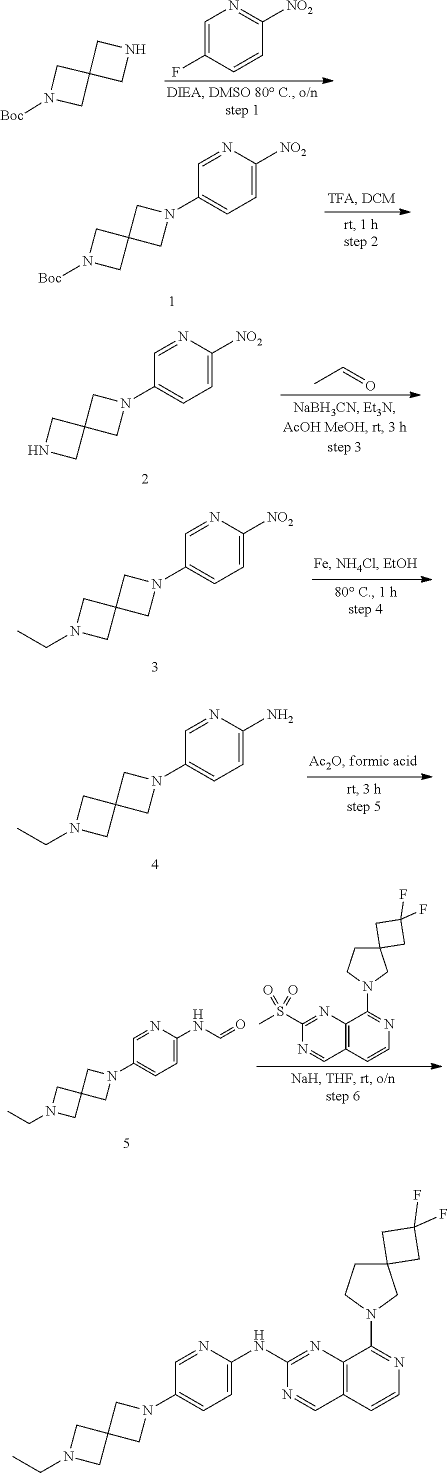

### Step 1: tert-butyl 6-(6-nitropyridin-3-yl)-2,6-diazaspiro[3.3]heptane-2-carboxylate

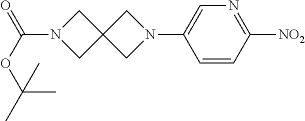 [SMILES: CC(C)(C)OC(=O)N1CC2(C1)CN(c1ccc([N+](=O)[O-])nc1)C2]

To a stirred mixture of tert-butyl 2,6-diazaspiro[3.3]heptane-2-carboxylate (5 g, 25.21 mmol) and 5-fluoro-2-nitropyridine (5.37 g, 37.82 mmol) in dimethyl sulfoxide (30 mL) was N,N-diisopropylethylamine (9.78 g, 75.65 mmol). The resulting mixture was heated to 80° C. and stirred for 3 hours. The reaction mixture was allowed to cool to room temperature, diluted with water (500 mL) and extracted with ethyl acetate (3×500 mL). The combined organic layers were washed with brine (500 mL), dried over anhydrous sodium sulfate, and filtered. The filtrate was concentrated under vacuum to afford the crude product. The residue was purified by trituration with petroleum ether/ethyl acetate (5:1, 100 mL) to afford tert-butyl 6-(6-nitropyridin-3-yl)-2,6-diazaspiro[3.3]heptane-2-carboxylate (6.78 g, 83.7% yield) as a yellow solid.

LCMS (ESI) m/z=321 [M+H]⁺.

### Step 2: 2-(6-nitropyridin-3-yl)-2,6-diazaspiro[3.3]heptane

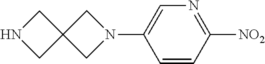 [SMILES: O=[N+]([O-])c1ccc(N2CC3(CNC3)C2)cn1]

To a stirred mixture of tert-butyl 6-(6-nitropyridin-3-yl)-2,6-diazaspiro[3.3]heptane-2-carboxylate (6.78 g, 21.16 mmol) in dichloromethane (80 mL) was added trifluoroacetic acid (16 mL). The resulting mixture was stirred for 1 hour at room temperature and concentrated under vacuum. The residue was diluted with dichloromethane (100 mL) and concentrated under vacuum again to afford crude 2-(6-nitropyridin-3-yl)-2,6-diazaspiro[3.3]heptane trifluoroacetic acid salt (6 g) as a brown yellow solid. The crude product was used for next step directly without further purification.

LCMS (ESI) m/z=221 [M+H]⁺.

### Step 3: 2-ethyl-6-(6-nitropyridin-3-yl)-2,6-diazaspiro[3.3]heptane

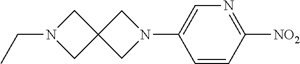 [SMILES: CCN1CC2(C1)CN(c1ccc([N+](=O)[O-])nc1)C2]

A solution of 2-(6-nitropyridin-3-yl)-2,6-diazaspiro[3.3]heptane trifluoroacetic acid salt (6 g, 18.9 mmol) in methanol (100 mL) was treated with triethylamine (5.73 g, 56.7 mmol) for 10 minutes followed by the addition of acetaldehyde (4.16 g, 94.5 mmol), acetic acid (0.23 mL, 4.08 mmol) and sodium cyanoborohydride (2.51 g, 39.8 mmol). The resulting mixture was stirred for 3 hours at room temperature and concentrated under vacuum. The residue was diluted with water (500 mL) and extracted with ethyl acetate (3×500 mL). The combined organic layers were washed with brine (1000 mL), dried over anhydrous sodium sulfate, and filtered. The filtrate was concentrated under reduced pressure to afford the crude product. The residue was purified by trituration with dichloromethane (100 mL) to afford 2-ethyl-6-(6-nitropyridin-3-yl)-2,6-diazaspiro[3.3]heptane (4 g, 58.9% yield) as an orange solid. LCMS (ESI) m/z=249 [M+H]⁺.

### Step 4: 5-(6-ethyl-2,6-diazaspiro[3.3]heptan-2-yl) pyridin-2-amine

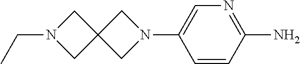 [SMILES: CCN1CC2(C1)CN(c1ccc(N)nc1)C2]

A solution of 2-ethyl-6-(6-nitropyridin-3-yl)-2,6-diazaspiro[3.3]heptane (4 g, 16.11 mmol), ammonium chloride (4.31 g, 80.55 mmol), iron powder (9.00 g, 161.100 mmol) and water (20 mL) in ethanol (60 mL) was stirred for 1 hour at 80° C. The resulting mixture was filtered and the filter cake was washed with ethanol (100 mL). The filtrate was concentrated under vacuum to afford the crude product. The residue was purified by reversed-phase flash chromatography (C18 silica gel, acetonitrile/water (with 10 mmol/L NH₄HCO₃) gradient) to afford 5-(6-ethyl-2,6-diazaspiro[3.3]heptan-2-yl)pyridin-2-amine (2 g, 56.6% yield) as a black solid.

LCMS (ESI) m/z=219 [M+H]⁺.

### Step 5: N-(5-(6-ethyl-2,6-diazaspiro[3.3]heptan-2-yl) pyridin-2-yl) formamide

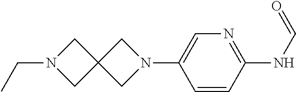 [SMILES: CCN1CC2(C1)CN(c1ccc(NC=O)nc1)C2]

A solution of acetic anhydride (2 mL) in formic acid (4 mL) was stirred for 1 hour at room temperature followed by the addition of 5-(6-ethyl-2,6-diazaspiro[3.3]heptan-2-yl)pyridin-2-amine (400 mg, 1.83 mmol) in portions at room temperature. The resulting mixture was stirred for 3 hours at room temperature. The reaction mixture was neutralized to PH=7 with saturated aqueous sodium bicarbonate (200 mL) and extracted with ethyl acetate (3×100 mL). The combined organic layers were washed with brine, dried over anhydrous sodium sulfate, and filtered. The filtrate was concentrated under vacuum and the residue was purified by preparative reverse phase HPLC (acetonitrile/water (with 10 mM NH₄HCO₃ and 0.1% NH₃·H₂O) gradient) to afford the title compound (70 mg, 15.3% yield) as an off-white solid. LCMS (ESI) m/z=247 [M+H]⁺.

### Step 6: 8-(2,2-difluoro-6-azaspiro[3.4]octan-6-yl)-N-(5-(6-ethyl-2,6-diazaspiro[3.3]heptan-2-yl)pyridin-2-yl) pyrido[3,4-d]pyrimidin-2-amine

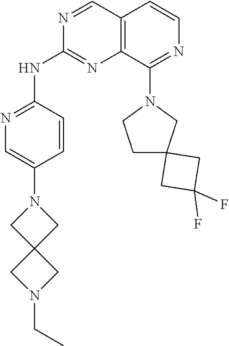 [SMILES: CCN1CC2(C1)CN(c1ccc(Nc3ncc4ccnc(N5CCC6(C5)CC(F)(F)C6)c4n3)nc1)C2]

To a stirred mixture of N-(5-(6-ethyl-2,6-diazaspiro[3.3]heptan-2-yl)pyridin-2-yl)formamide (50 mg, 0.20 mmol) in tetrahydrofuran (2 mL) was added sodium hydride (60% in mineral oil, 24 mg, 0.60 mmol) at 0° C. The resulting mixture was stirred for 15 minutes at 0° C. Then 8-(2,2-difluoro-6-azaspiro[3.4]octan-6-yl)-2-(methylsulfonyl)pyrido[3,4-d]pyrimidine (78 mg, 0.22 mmol) was added to the mixture and the resulting mixture was warmed to room temperature and stirred overnight. The reaction mixture was quenched by addition of water (50 mL) and extracted with dichloromethane (3×50 mL). The combined organic layers were washed with brine (100 mL), dried over anhydrous sodium sulfate, and filtered. The filtrate was concentrated under vacuum and the residue was purified by preparative reverse phase HPLC (acetonitrile/water (with 10 mM NH₄HCO₃ and 0.1% NH₃·H₂O) gradient) to afford the title compound (12.7 mg, 10.0% yield) as an orange solid.

LCMS (ESI) m/z=493 [M+H]⁺. ¹H NMR (400 MHz, DMSO-d₆) δ (ppm) 9.13 (s, 1H), 7.94-7.62 (m, 3H), 7.14-6.97 (m, 2H), 4.39 (s, 4H), 4.12-4.18 (m, 6H), 3.98-3.92 (m, 2H), 3.27-3.25 (m, 2H), 2.67-2.61 (m, 4H), 2.16-2.13 (m, 2H), 1.42-1.34 (m, 1H), 1.31-1.23 (m, 3H).

### Example 11: 8-(8,8-difluoro-2-(methyl-d3)-2,6-diazaspiro[3.4]octan-6-yl)-6-methyl-N-(1-((1-methyl-1H-pyrazol-4-yl)sulfonyl)piperidin-4-yl)pyrido[3,4-d]pyrimidin-2-amine

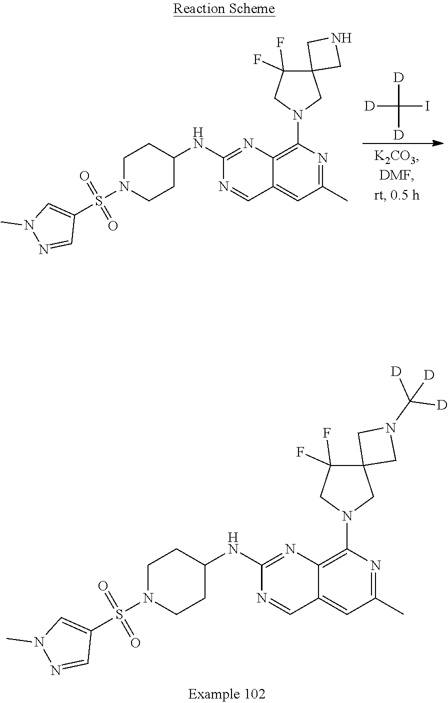

Iodomethane-d₃ (9.51 mg, 0.06 mmol) was added to a mixture of 8-(8,8-difluoro-2,6-diazaspiro[3.4]octan-6-yl)-6-methyl-N-(1-((1-methyl-1H-pyrazol-4-yl)sulfonyl)piperidin-4-yl)pyrido[3,4-d]pyrimidin-2-amine (70 mg, 0.13 mmol) and K₂CO₃ (36.3 mg, 0.26 mmol) in DMF (1 mL). The reaction mixture was stirred for 30 minutes at room temperature, quenched by addition of water (5 mL) and extracted with DCM (3×5 mL). The combined organic layers were washed with brine (10 mL), dried over anhydrous Na₂SO₄, filtered and the filtrate was concentrated under reduced pressure to afford the crude product. The residue was purified by Prep-TLC (MeOH/DCM=1:10) to afford the desired product 8-(8,8-difluoro-2-(methyl-d3)-2,6-diazaspiro[3.4]octan-6-yl)-6-methyl-N-(1-((1-methyl-1H-pyrazol-4-yl)sulfonyl)piperidin-4-yl)pyrido[3,4-d]pyrimidin-2-amine (23.6 mg, 32.0% yield).

¹H NMR (400 MHz, DMSO-d₆) δ 8.96 (s, 1H), 8.34 (s, 1H), 7.77 (s, 1H), 7.53 (s, 1H), 6.76 (s, 1H), 4.33-4.24 (m, 2H), 4.23-4.15 (m, 1H), 3.92 (s, 3H), 3.70 (s, 1H), 3.60-3.50 (m, 2H), 3.31-3.25 (m, 2H), 3.18-2.99 (m, 2H), 2.49-2.40 (m, 3H), 2.33 (s, 3H), 2.08-1.97 (m, 2H), 1.70-1.54 (m, 2H).

LCMS (ESI−MS) m/z=551.3 [M+H]⁺.

### Example 12: 8-(8,8-difluoro-2-(methyl-d3)-2,6-diazaspiro[3.4]octan-6-yl)-6-methyl-N-(1-(methylsulfonyl)piperidin-4-yl)pyrido[3,4-d]pyrimidin-2-amine

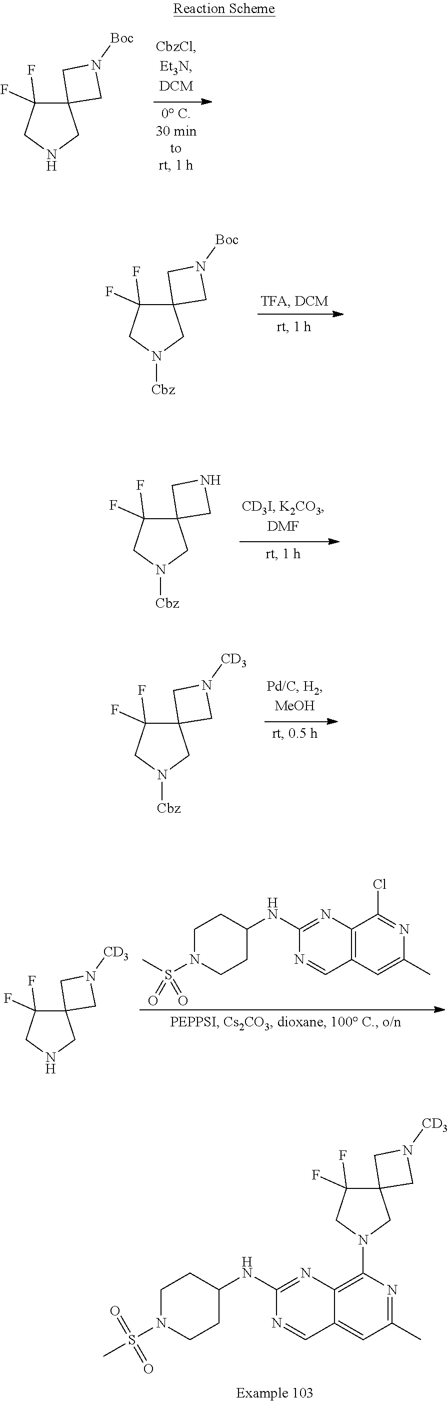

### Detailed Procedure

### Step 1: 6-benzyl 2-(tert-butyl) 8,8-difluoro-2,6-diazaspiro[3.4]octane-2,6-dicarboxylate

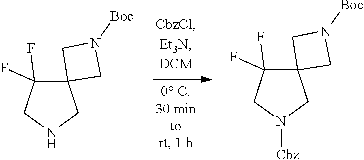 [SMILES: CC(C)(C)OC(=O)N1CC2(CNCC2(F)F)C1 ; CC(C)(C)OC(=O)N1CC2(CN(C(=O)OCc3ccccc3)CC2(F)F)C1]

Benzyl chloroformate (412 mg, 2.42 mmol) was added to a cooled to 0° C. solution of tert-butyl 8,8-difluoro-2,6-diazaspiro[3.4]octane-2-carboxylate (500 mg, 2.01 mmol) and Et₃N (408 mg, 4.03 mmol) in DCM (6 mL). The resulting mixture was stirred for 30 minutes at 0° C. and then warmed to room temperature and stirred for another hour. The resulting mixture was quenched by addition of water (15 mL) and extracted with DCM (3×15 mL). The combined organic layers were washed with brine (50 mL), dried over anhydrous Na₂SO₄, filtered and the filtrate was concentrated under vacuum. The residue was purified by Prep-TLC (PE/EA, 2:1) to afford the desired product 6-benzyl 2-(tert-butyl) 8,8-difluoro-2,6-diazaspiro[3.4]octane-2,6-dicarboxylate (700 mg, 90.9% yield).

LCMS (ESI−MS) m/z=383.2 [M+H]⁺.

### Step 2: benzyl 8,8-difluoro-2,6-diazaspiro[3.4]octane-6-carboxylate

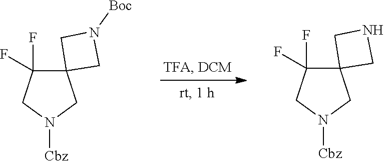 [SMILES: CC(C)(C)OC(=O)N1CC2(CN(C(=O)OCc3ccccc3)CC2(F)F)C1 ; O=C(OCc1ccccc1)N1CC(F)(F)C2(CNC2)C1]

TFA (2 mL) was added to a stirred mixture of 6-benzyl 2-(tert-butyl) 8,8-difluoro-2,6-diazaspiro[3.4]octane-2,6-dicarboxylate (500 mg, 1.31 mmol) in DCM (6 mL). The resulting mixture was stirred for 1 hour at room temperature and concentrated under high vacuum to afford the crude product (350 mg). The crude product was used for the next step without further purification.

LCMS (ESI−MS) m/z=283.2 [M+H]⁺.

### Step 3: Benzyl 8,8-difluoro-2-(methyl-d3)-2,6-diazaspiro[3.4]octane-6-carboxylate

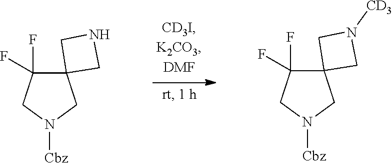 [SMILES: O=C(OCc1ccccc1)N1CC(F)(F)C2(CNC2)C1 ; [2H]C([2H])([2H])N1CC2(CN(C(=O)OCc3ccccc3)CC2(F)F)C1]

Iodomethane-d₃ (64.2 mg, 0.44 mmol) was slowly added to a mixture of benzyl 8,8-difluoro-2,6-diazaspiro[3.4]octane-6-carboxylate (250 mg, 0.89 mmol) and K₂CO₃ (245 mg, 1.77 mmol) in DMF (3 mL). The resulting mixture was stirred for 1 hour at room temperature, quenched with water (8 mL) and extracted with DCM (3×8 mL). The combined organic layers were washed with brine (15 mL), dried over anhydrous Na₂SO₄, filtered and the filtrate was concentrated under vacuum. The residue was purified by Prep-TLC (PE/EA, 1:1) to afford the desired product benzyl 8,8-difluoro-2-(methyl-d₃)-2,6-diazaspiro[3.4]octane-6-carboxylate (90 mg, 33.9% yield).

LCMS (ESI−MS) m/z=303.3 [M+H]⁺.

### Step 4: 8,8-difluoro-2-(methyl-d3)-2,6-diazaspiro[3.4]octane

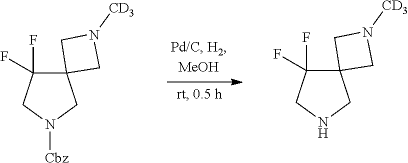 [SMILES: [2H]C([2H])([2H])N1CC2(CN(C(=O)OCc3ccccc3)CC2(F)F)C1 ; [2H]C([2H])([2H])N1CC2(CNCC2(F)F)C1]

Pd/C (10% on carbon, 10 mg) was added to the mixture of benzyl 8,8-difluoro-2-(methyl-d₃)-2,6-diazaspiro[3.4]octane-6-carboxylate (70 mg, 0.23 mmol) in MeOH (4 mL) under a nitrogen atmosphere. The resulting mixture was stirred for 0.5 hour at room temperature under a hydrogen atmosphere. The reaction mixture was filtered, the filter cake was washed with MeOH (20 mL). The filtrate was concentrated under reduced pressure to afford the crude product 8,8-difluoro-2-(methyl-d₃)-2,6-diazaspiro[3.4]octane (60 mg) as a colorless oil. The crude product was used for the next step without further purification.

LCMS (ESI−MS) m/z=166.2 [M+H]⁺.

### Step 5: 8-(8,8-difluoro-2-(methyl-d3)-2,6-diazaspiro[3.4]octan-6-yl)-6-methyl-N-(1-(methylsulfonyl)piperidin-4-yl)pyrido[3,4-d]pyrimidin-2-amine

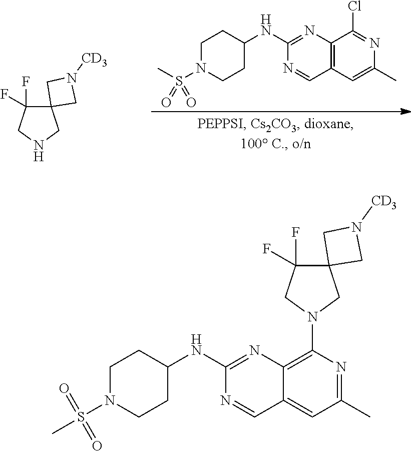

Pd-PEPPSI-iHeptCl 3-chloropyridine (13.7 mg, 0.014 mmol) was added to a mixture of 8-chloro-6-methyl-N-(1-(methylsulfonyl)piperidin-4-yl)pyrido[3,4-d]pyrimidin-2-amine (50 mg, 0.141 mmol), 8,8-difluoro-2-(methyl-d₃)-2,6-diazaspiro[3.4]octane (23.2 mg, 0.141 mmol) and Cs₂CO₃ (91.6 mg, 0.28 mmol) in 1,4-dioxane (1 mL) under a nitrogen atmosphere. The resulting mixture was heated to 100° C. and stirred overnight under a nitrogen atmosphere. After cooling to room temperature, the resulting mixture was filtered and the filter cake was washed with DCM (10 mL). The filtrate was concentrated under reduced pressure. The residue was purified by Prep-TLC (DCM/MeOH, 10:1). The product was further purified by preparative RP-HPLC to afford the desired product 8-(8,8-difluoro-2-(methyl-d3)-2,6-diazaspiro[3.4]octan-6-yl)-6-methyl-N-(1-(methylsulfonyl) piperidin-4-yl)pyrido[3,4-d]pyrimidin-2-amine (2.7 mg, 3.79% yield).

¹H NMR (400 MHz, DMSO-d₆) δ 8.98 (s, 1H), 7.54 (s, 1H), 6.78 (s, 1H), 4.40-4.22 (m, 4H), 3.90-3.79 (m, 1H), 3.61 (d, J=11.6 Hz, 3H), 3.26-3.15 (m, 3H), 2.97-2.84 (m, 5H), 2.34 (s, 3H), 2.13-1.98 (m, 2H), 1.69-1.55 (m, 2H).

LCMS (ESI−MS) m/z=485.3 [M+H]⁺.

### Example 36: N-(6-fluoro-2-(methylsulfonyl)isoindolin-5-yl)-6-methyl-8-(2,6-diazaspiro[3.4]octan-2-yl)pyrido[3,4-d]pyrimidin-2-amine

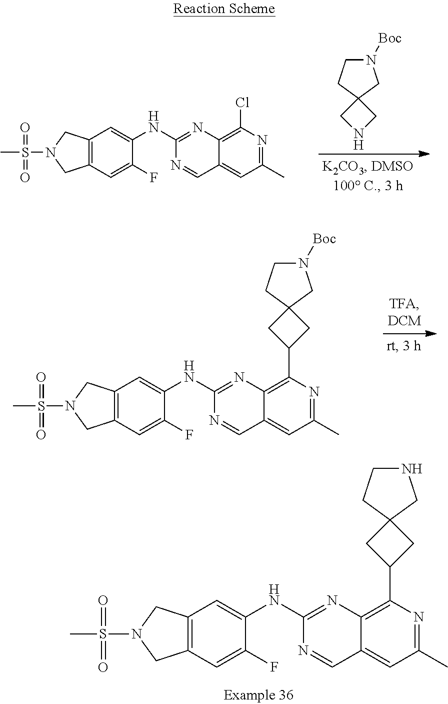

### Step 1: Tert-butyl 2-(2-((6-fluoro-2-(methylsulfonyl)isoindolin-5-yl)amino)-6-methylpyrido[3,4-d]pyrimidin-8-yl)-2,6-diazaspiro[3.4]octane-6-carboxylate

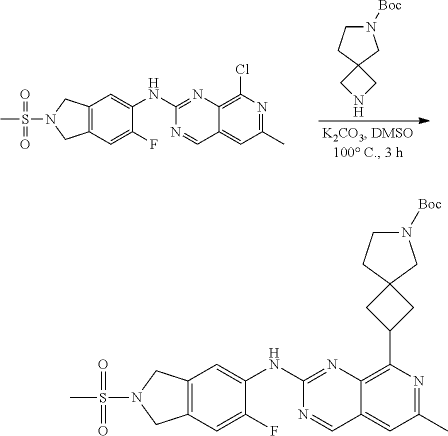

To a solution of 8-chloro-N-(6-fluoro-2-(methylsulfonyl)isoindolin-5-yl)-6-methylpyrido[3,4-d]pyrimidin-2-amine (65 mg, 0.16 mmol) in dimethyl sulfoxide (5 mL) was added tert-butyl 2,6-diazaspiro[3.4]octane-6-carboxylate (67.67 mg, 0.31 mmol) and K₂CO₃ (66.56 mg, 0.47 mmol). The resulting mixture was stirred for 3 h at 100° C. under a nitrogen atmosphere. The mixture was allowed to cool down to room temperature, diluted with EA (200 mL) and washed with a saturated aq. sodium chloride solution (3×200 mL). The combined organic layers were dried over anhydrous Na₂SO₄. After filtration, the filtrate was concentrated under reduced pressure to give tert-butyl 2-(2-((6-fluoro-2-(methylsulfonyl)isoindolin-5-yl)amino)-6-methylpyrido[3,4-d]pyrimidin-8-yl)-2,6-diazaspiro[3.4]octane-6-carboxylate (60 mg, 64.5% yield).

LCMS (ESI−MS) m/z=584.2 [M+H]⁺.

### Step 2: N-(6-fluoro-2-(methylsulfonyl)isoindolin-5-yl)-6-methyl-8-(2,6-diazaspiro[3.4]octan-2-yl)pyrido[3,4-d]pyrimidin-2-amine

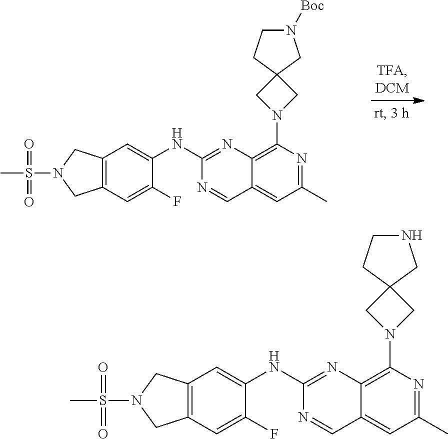

To a solution of tert-butyl 2-(2-((6-fluoro-2-(methylsulfonyl)isoindolin-5-yl)amino)-6-methylpyrido[3,4-d]pyrimidin-8-yl)-2,6-diazaspiro[3.4]octane-6-carboxylate (55 mg, 0.09 mmol) in DCM (2 mL) was added TFA (0.2 mL). The resulting mixture was stirred for 3 h at room temperature under a nitrogen atmosphere. The crude product (55 mg) was purified by preparative RP-HPLC to afford N-(6-fluoro-2-(methylsulfonyl)isoindolin-5-yl)-6-methyl-8-(2,6-diazaspiro[3.4]octan-2-yl)pyrido[3,4-d]pyrimidin-2-amine (17.8 mg, 37.70% yield).

¹H NMR (400 MHz, DMSO-d₆) δ 9.38 (s, 1H), 9.18-9.09 (m, 1H), 7.82-7.55 (m, 1H), 7.32-7.21 (m, 1H), 6.81-6.75 (m, 1H), 4.70-4.60 (m, 5H), 4.15 (s, 4H), 3.10-2.98 (m, 5H), 2.95-2.88 (m, 2H), 2.35 (s, 3H), 2.01-1.95 (m, 2H).

LCMS (ESI−MS) m/z=484.2 [M+H]⁺.

### Example 22: N-(1-(cyclopropylsulfonyl)piperidin-4-yl)-6-methyl-8-(2,6-diazaspiro[3.4]octan-2-yl)pyrido[3,4-d]pyrimidin-2-amine

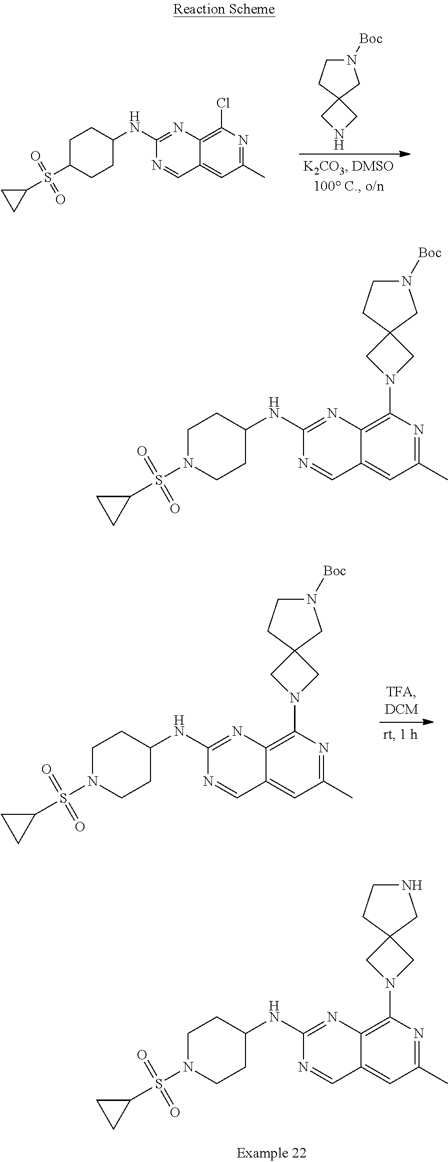 [SMILES: Cc1cc2cnc(NC3CCC(S(=O)(=O)C4CC4)CC3)nc2c(Cl)n1 ; CC(C)(C)OC(=O)N1CCC2(CNC2)C1 ; Cc1cc2cnc(NC3CCN(S(=O)(=O)C4CC4)CC3)nc2c(N2CC3(CCN(C(=O)OC(C)(C)C)C3)C2)n1 ; Cc1cc2cnc(NC3CCN(S(=O)(=O)C4CC4)CC3)nc2c(N2CC3(CCN(C(=O)OC(C)(C)C)C3)C2)n1 ; Cc1cc2cnc(NC3CCN(S(=O)(=O)C4CC4)CC3)nc2c(N2CC3(CCNC3)C2)n1]

### Step 1: Tert-butyl 2-(2-((1-(cyclopropylsulfonyl)piperidin-4-yl)amino)-6-methylpyrido[3,4-d]pyrimidin-8-yl)-2,6-diazaspiro[3.4]octane-6-carboxylate

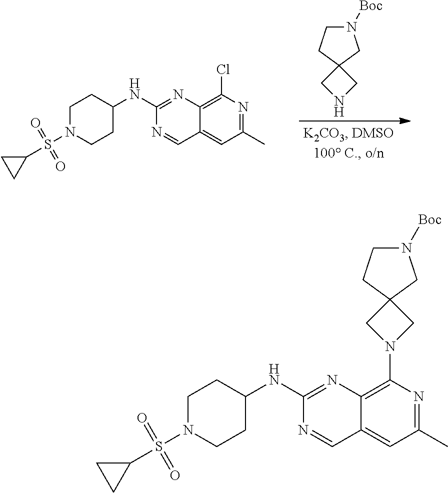 [SMILES: Cc1cc2cnc(NC3CCN(S(=O)(=O)C4CC4)CC3)nc2c(Cl)n1 ; CC(C)(C)OC(=O)N1CCC2(CNC2)C1 ; Cc1cc2cnc(NC3CCN(S(=O)(=O)C4CC4)CC3)nc2c(N2CC3(CCN(C(=O)OC(C)(C)C)C3)C2)n1]

A solution of 8-chloro-N-(1-(cyclopropylsulfonyl)piperidin-4-yl)-6-methylpyrido[3,4-d]pyrimidin-2-amine (100 mg, 0.26 mmol), tert-butyl 2,6-diazaspiro[3.4]octane-6-carboxylate (55.59 mg, 0.26 mmol) and K₂CO₃ (108.57 mg, 0.78 mmol) in DMSO (1 mL) was stirred overnight at 100. The resulting mixture was diluted with water (20 mL) and extracted with CH₂Cl₂ (3×20 mL). The combined organic layers were dried over anhydrous Na₂SO₄. After filtration, the filtrate was concentrated under reduced pressure. The residue was purified by Prep-TLC (DCM/MeOH, 10:1) to afford tert-butyl 2-(2-((1-(cyclopropylsulfonyl) piperidin-4-yl)amino)-6-methylpyrido[3,4-d]pyrimidin-8-yl)-2,6-diazaspiro[3.4]octane-6-carboxylate (130 mg, 80.11% yield).

LCMS (ESI−MS) m/z=558.3 [M+H]⁺.

### Step 2: N-(1-(cyclopropylsulfonyl)piperidin-4-yl)-6-methyl-8-(2,6-diazaspiro[3.4]octan-2-yl)pyrido[3,4-d]pyrimidin-2-amine

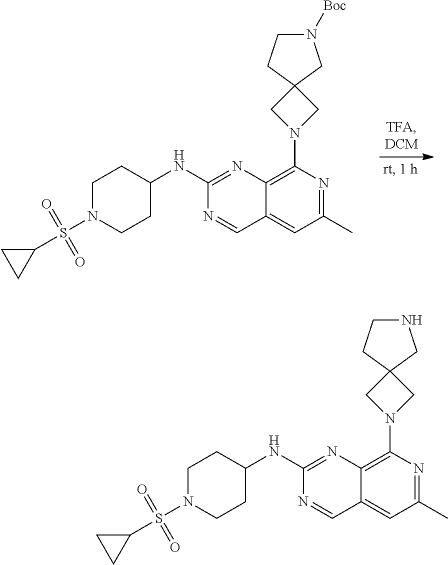 [SMILES: Cc1cc2cnc(NC3CCN(S(=O)(=O)C4CC4)CC3)nc2c(N2CC3(CCN(C(=O)OC(C)(C)C)C3)C2)n1 ; Cc1cc2cnc(NC3CCN(S(=O)(=O)C4CC4)CC3)nc2c(N2CC3(CCNC3)C2)n1]

A solution of tert-butyl 2-(2-((1-(cyclopropylsulfonyl) piperidin-4-yl)amino)-6-methylpyrido[3,4-d]pyrimidin-8-yl)-2,6-diazaspiro[3.4]octane-6-carboxylate (120 mg, 0.21 mmol) in TFA (1 mL) and DCM (3 mL) was stirred for 1 h at room temperature. The reaction mixture was concentrated and purified by Prep-TLC (EA). The product was further purified by preparative RP-HPLC to afford N-(1-(cyclopropylsulfonyl)piperidin-4-yl)-6-methyl-8-(2,6-diazaspiro[3.4]octan-2-yl)pyrido[3,4-d]pyrimidin-2-amine (50.1 mg, 49.56% yield).

¹H NMR (400 MHz, DMSO-d6) δ 8.93 (d, J=2.7 Hz, 1H), 7.45 (s, 1H), 6.67 (d, J=6.0 Hz, 1H), 4.25 (s, 4H), 3.81 (s, 1H), 3.63 (d, J=12.1 Hz, 2H), 3.47 (s, 1H), 3.07-2.78 (m, 5H), 2.68-2.54 (m, 2H), 2.30 (s, 3H), 2.17-2.07 (m, 1H), 2.06-1.92 (m, 3H), 1.66-1.48 (m, 2H), 1.07-0.86 (m, 4H).

LCMS (ESI−MS) m/z=458.3 [M+H]⁺.

### Example 43: 1-(cyclopropanesulfonyl)-N-(8-{2,6-diazaspiro[3.4]octan-2-yl}-6-(difluoromethyl)pyrido[3,4-d]pyrimidin-2-yl)piperidin-4-amine

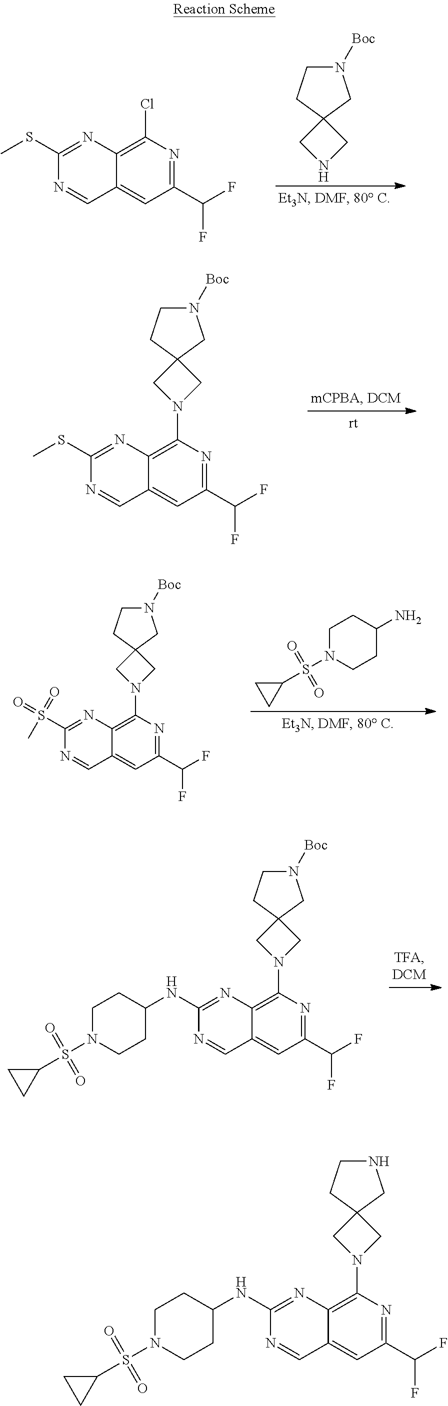

### Step 1: Tert-butyl 2-[6-(difluoromethyl)-2-(methylsulfanyl)pyrido[3,4-d]pyrimidin-8-yl]-2,6-diazaspiro[3.4]octane-6-carboxylate

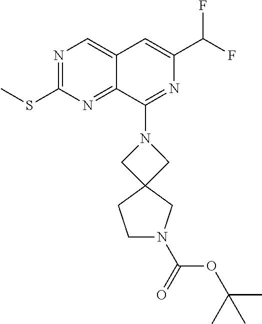 [SMILES: CSc1ncc2cc(C(F)F)nc(N3CC4(CCN(C(=O)OC(C)(C)C)C4)C3)c2n1]

A solution of tert-butyl 2,6-diazaspiro[3.4]octane-6-carboxylate (0.225 g, 1.059 mmol) and triethylamine anhydrous (0.738 ml, 5.293 mmol) in dimethylformamide (5.54 ml) was stirred at room temperature for 5 minutes. Then, 8-chloro-6-(difluoromethyl)-2-(methylsulfanyl)pyrido[3,4-d]pyrimidine (0.277 g, 1.059 mmol) was added and the reaction mixture was stirred at 80° C. for 18 h. The reaction mixture was diluted with water (10 mL) and dichloromethane (10 mL) and the layers were separated. The aqueous layer was extracted with dichloromethane (10 mL×3) and the combined organic layers were washed with K₂CO₃ sat. aq. solution, dried over anhydrous MgSO₄, filtered and concentrated under reduced pressure. The crude material was purified by flash column chromatography on silica gel (from hexane to hexane/EtOAc 6:4) to provide the title compound (0.389 g, 84% yield).

¹H NMR (300 MHz, Chloroform-d) δ 9.07 (s, 1H), 7.14 (s, 1H), 6.53 (t, J=55.8 Hz, 1H), 3.68-3.56 (m, 2H), 3.56-3.35 (m, 2H), 2.66 (s, 3H), 2.19 (t, J=7.1 Hz, 2H), 1.58 (s, 4H), 1.50 (s, 9H).

UPLC (ESI) [M+H]⁺438.45.

### Step 2: Tert-butyl 2-[6-(difluoromethyl)-2-methanesulfonylpyrido[3,4-d]pyrimidin-8-yl]-2,6-diazaspiro[3.4]octane-6-carboxylate

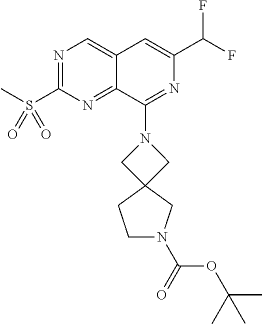 [SMILES: CC(C)(C)OC(=O)N1CCC2(C1)CN(c1nc(C(F)F)cc3cnc(S(C)(=O)=O)nc13)C2]

To a solution of tert-butyl 2-[6-(difluoromethyl)-2-(methylsulfanyl)pyrido[3,4-d]pyrimidin-8-yl]-2,6-diazaspiro[3.4]octane-6-carboxylate (0.389 g, 0.890 mmol) in dichloromethane (11.68 ml), was added portionwise 3-chloroperbenzoic acid (0.768 g, 4.450 mmol). The reaction mixture was stirred at room temperature for 90 minutes, quenched by the dropwise addition of a 10% Na₂S₂O₃ aq. solution (10 mL) and extracted with dichloromethane (10 mL×3). The combined organic layers were washed with NaHCO₃ sat. aq. solution, dried over anhydrous MgSO₄, filtered and concentrated under reduced pressure. The crude material was purified by flash column chromatography on silica gel (from hexane/EtOAc 9:1 to hexane/EtOAc 4:6) to provide the title compound (0.326 g, 74%).

¹H NMR (300 MHz, Chloroform-d) δ 9.42 (s, 1H), 7.27 (s, 1H), 6.55 (t, J=55.4 Hz, 1H), 3.63 (s, 2H), 3.57-3.43 (m, 2H), 3.42 (s, 3H), 2.31-2.15 (m, 2H), 1.68-1.57 (m, 3H), 1.57-1.40 (m, 10H).

UPLC (ESI) [M+H]⁺470.00.

### Step 3: Tert-butyl 2-(2-{1[1-(cyclopropanesulfonyl)piperidin-4-yl]amino}-6-(difluoromethyl)pyrido[3,4-d]pyrimidin-8-yl)-2,6-diazaspiro[3.4]octane-6-carboxylate

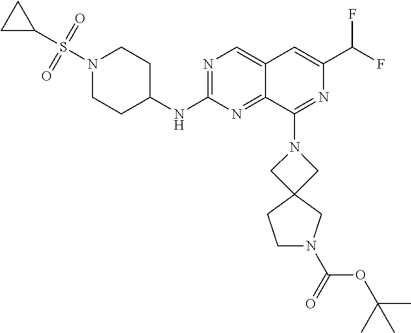 [SMILES: CC(C)(C)OC(=O)N1CCC2(C1)CN(c1nc(C(F)F)cc3cnc(NC4CCN(S(=O)(=O)C5CC5)CC4)nc13)C2]

A solution of 1-(cyclopropanesulfonyl)piperidin-4-amine hydrochloride (0.051 g, 0.202 mmol) and triethylamine anhydrous (0.141 ml, 1.012 mmol) in dimethylformamide (2.0 ml) was stirred at room temperature for 5 minutes. Then, tert-butyl 2-[6-(difluoromethyl)-2-methanesulfonylpyrido[3,4-d]pyrimidin-8-yl]-2,6-diazaspiro[3.4]octane-6-carboxylate (0.1 g, 0.202 mmol) was added and the reaction mixture was stirred at 80° C. for 24 h. The reaction mixture was concentrated under reduced pressure and purified by flash column chromatography on silica gel (from hexane to hexane/EtOAc 4:6) to provide the title compound (0.079 g, 53% yield).

¹H NMR (300 MHz, Chloroform-d) δ 8.92 (s, 1H), 7.09 (s, 1H), 6.53 (t, J=56.1 Hz, 1H), 5.33 (s, 1H), 4.01 (s, 1H), 3.81 (d, J=12.5 Hz, 2H), 3.61 (d, J=9.7 Hz, 2H), 3.47 (s, 2H), 3.09 (t, J=11.3 Hz, 2H), 2.33 (tt, J=8.3, 4.9 Hz, 1H), 2.20 (q, J=9.4, 7.1 Hz, 4H), 1.86-1.54 (m, 6H), 1.50 (s, 9H), 1.26-1.15 (m, 2H), 1.05 (d, J=6.8 Hz, 2H).

UPLC (ESI) [M+H]⁺594.50.

### Step 4: 1-(cyclopropanesulfonyl)-N-(8-{2,6-diazaspiro[3.4]octan-2-yl}-6-(difluoromethyl)pyrido[3,4-d]pyrimidin-2-yl)piperidin-4-amine

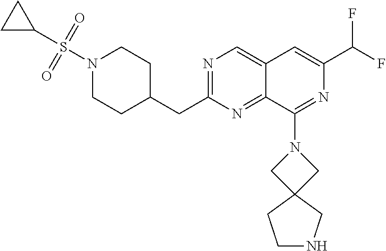 [SMILES: O=S(=O)(C1CC1)N1CCC(Cc2ncc3cc(C(F)F)nc(N4CC5(CCNC5)C4)c3n2)CC1]

A solution of tert-butyl 2-(2-{[1-(cyclopropanesulfonyl) piperidin-4-yl]amino}-6-(difluoromethyl)pyrido[3,4-d]pyrimidin-8-yl)-2,6-diazaspiro[3.4]octane-6-carboxylate (0.079 g, 0.107 mmol) in dichloromethane anhydrous (3.97 ml) was cooled down to 0° C. Then, trifluoroacetic acid (0.79 ml) was added dropwise and the reaction mixture was stirred at 0° C. for 2 h. Volatiles were removed under reduced pressure and the crude material was purified by preparative HPLC (0.1% formic acid) to provide the title compound (0.043 g, 80% yield).

¹H NMR (400 MHz, DMSO-d₆) δ 9.12 (s, 1H), 8.80 (s, 1H), 7.88 (s, 1H), 7.19 (s, 1H), 6.75 (t, J=55.5 Hz, 1H), 4.37 (s, 4H), 3.87 (s, 1H), 3.63 (dd, J=12.5, 4.5 Hz, 2H), 3.36 (s, 2H), 3.22-3.14 (m, 2H), 3.11-3.01 (m, 2H), 2.60 (tt, J=7.8, 4.9 Hz, 1H), 2.21 (t, J=7.2 Hz, 2H), 2.02 (d, J=12.8 Hz, 2H), 1.63 (q, J=11.3 Hz, 2H), 1.01 (dt, J=7.9, 2.8 Hz, 2H), 0.98-0.92 (m, 2H).

UPLC (ESI) [M+H]⁺494.3.

### Example 48: N-(1-((cyclopentylmethyl)sulfonyl)piperidin-4-yl)-6-methyl-8-(2,6-diazaspiro[3.4]octan-2-yl)pyrido[3,4-d]pyrimidin-2-amine

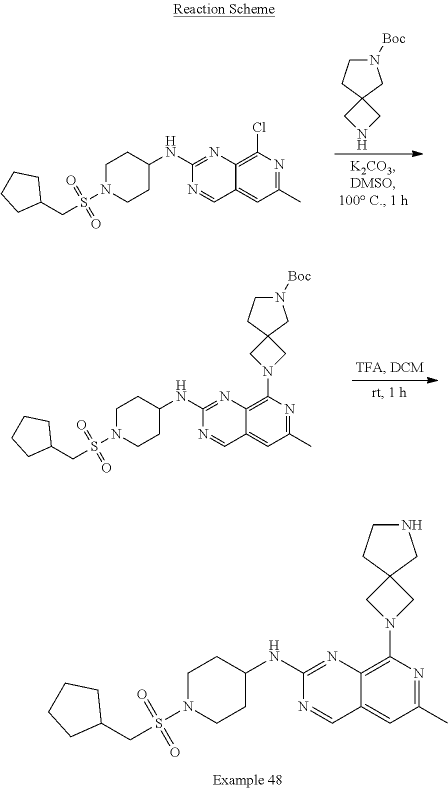 [SMILES: Cc1cc2cnc(NC3CCN(S(=O)(=O)CC4CCCC4)CC3)nc2c(Cl)n1 ; CC(C)(C)OC(=O)N1CCC2(CNC2)C1 ; Cc1cc2cnc(NC3CCN(S(=O)(=O)CC4CCCC4)CC3)nc2c(N2CC3(CCN(C(=O)OC(C)(C)C)C3)C2)n1 ; Cc1cc2cnc(NC3CCN(S(=O)(=O)CC4CCCC4)CC3)nc2c(N2CC3(CCNC3)C2)n1]

### Step 1: Tert-butyl 2-(2-((1-((cyclopentylmethyl)sulfonyl)piperidin-4-yl)amino)-6-methylpyrido[3,4-d]pyrimidin-8-yl)-2,6-diazaspiro[3.4]octane-6-carboxylate

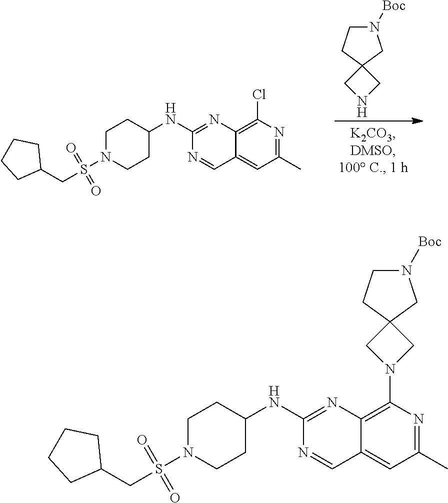 [SMILES: Cc1cc2cnc(NC3CCN(S(=O)(=O)CC4CCCC4)CC3)nc2c(Cl)n1 ; CC(C)(C)OC(=O)N1CCC2(CNC2)C1 ; Cc1cc2cnc(NC3CCN(S(=O)(=O)CC4CCCC4)CC3)nc2c(N2CC3(CCN(C(=O)OC(C)(C)C)C3)C2)n1]

A mixture of 8-chloro-N-(1-((cyclopentylmethyl)sulfonyl)piperidin-4-yl)-6-methylpyrido[3,4-d]pyrimidin-2-amine (300 mg, 0.70 mmol), K₂CO₃ (293.39 mg, 2.12 mmol) and tert-butyl 2,6-diazaspiro[3.4]octane-6-carboxylate (150.22 mg, 0.70 mmol) in DMSO (5 mL) was stirred for 1 h at 100° C. The reaction mixture was diluted with water (250 mL) and extracted with DCM (3×250 mL). The combined organic layers were dried over anhydrous Na₂SO₄, filtered and concentrated under vacuum. The crude product was purified by Prep-TLC (EA) to afford tert-butyl 2-(2-((1-((cyclopentylmethyl)sulfonyl) piperidin-4-yl)amino)-6-methylpyrido[3,4-d]pyrimidin-8-yl)-2,6-diazaspiro[3.4]octane-6-carboxylate (150 mg, 31.81% yield).

UPLC (ESI−MS) m/z=600.3 [M+H]⁺.

### Step 2: N-(1-((cyclopentylmethyl)sulfonyl)piperidin-4-yl)-6-methyl-8-(2,6-diazaspiro[3.4]octan-2-yl) pyrido[3,4-d]pyrimidin-2-amine

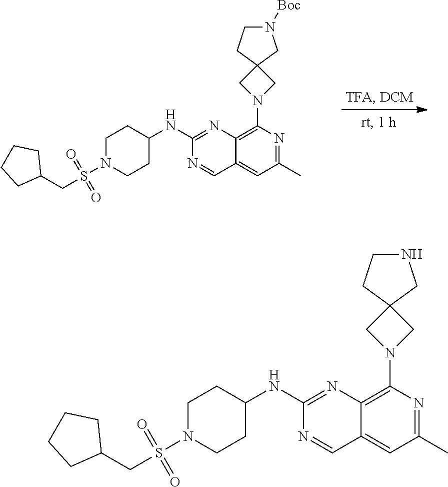 [SMILES: Cc1cc2cnc(NC3CCN(S(=O)(=O)CC4CCCC4)CC3)nc2c(N2CC3(CCN(C(=O)OC(C)(C)C)C3)C2)n1 ; Cc1cc2cnc(NC3CCN(S(=O)(=O)CC4CCCC4)CC3)nc2c(N2CC3(CCNC3)C2)n1]

A mixture of tert-butyl 2-(2-((1-((cyclopentylmethyl)sulfonyl)piperidin-4-yl)amino)-6-methylpyrido[3,4-d]pyrimidin-8-yl)-2,6-diazaspiro[3.4]octane-6-carboxylate (130 mg, 0.21 mmol) and TFA (1 mL) in DCM (3 mL) was stirred for 1 h at room temperature. The residue was purified by Prep-TLC (EA) followed by preparative RP-HPLC to afford N-(1-((cyclopentylmethyl)sulfonyl)piperidin-4-yl)-6-methyl-8-(2,6-diazaspiro[3.4]octan-2-yl)pyrido[3,4-d]pyrimidin-2-amine (39.5 mg, 34.4% yield).

¹H NMR (400 MHz, DMSO-d6) δ 8.92 (s, 1H), 7.40 (s, 1H), 6.66 (d, J=7.9 Hz, 1H), 4.24 (s, 4H), 3.82 (s, 1H), 3.63-3.55 (m, 2H), 3.06 (d, J=6.9 Hz, 2H), 2.94 (d, J=14.2 Hz, 4H), 2.84-2.77 (m, 2H), 2.30 (s, 3H), 2.26-2.15 (m, 1H), 2.03-1.91 (m, 4H), 1.90-1.81 (m, 2H), 1.67-1.48 (m, 6H), 1.35-1.21 (m, 3H).

UPLC (ESI−MS) m/z=500.3 [M+H]⁺.

### Compound 85: 1-cyclopropyl-3-(6-methyl-2-((1-(methylsulfonyl)piperidin-4-yl)amino)pyrido[3,4-d]pyrimidin-8-yl)cyclobutan-1-ol

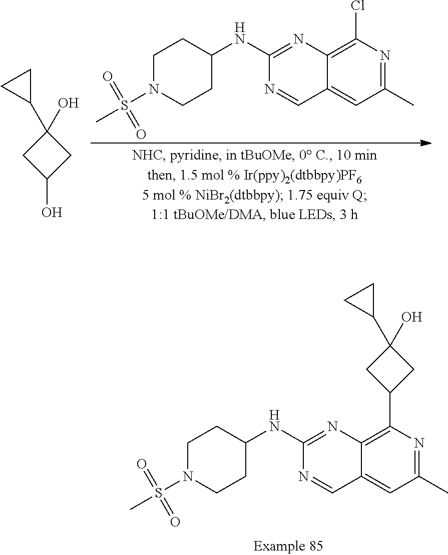

An over-dried 20 mL vial was charged with 1-cyclopropylcyclobutane-1,3-diol (43 mg, 0.49 mmol) and NHC (177 mg, 0.45 mmol). After the vial was vacuumed and refilled with nitrogen atmosphere, METB (4 mL) was added and the reaction stirred at room temperature for 5 minutes. A mixture of pyridine (81 mg, 1.03 mmol) in METB (1 mL) was added and the mixture stirred at room temperature for 10 minutes. Another over-dried 40 mL vial was charged with 8-chloro-6-methyl-N-(1-(methylsulfonyl)piperidin-4-yl)pyrido[3,4-d]pyrimidin-2-amine (100 mg, 0.28 mmol), NHC (177.72 mg, 0.45 mmol), Ir(ppy)₂(dtbbpy)PF₆ (3.85 mg, 0.004 mmol), NiBr₂(dtbbpy) (6.84 mg, 0.014 mmol) and 1-azabicyclo[2.2.2]octane (54.68 mg, 0.49 mmol) in DMA (5 mL) under a nitrogen atmosphere. The mixture was stirred at 800 rpm for 3 h under a 450 nm blue LEDs. The resulting mixture was concentrated under vacuum. The residue was purified by preparative RP-HPLC to afford 1-cyclopropyl-3-(6-methyl-2-((1-(methylsulfonyl)piperidin-4-yl)amino)pyrido[3,4-d]pyrimidin-8-yl)cyclobutan-1-ol (2.1 mg, 1.54%).

¹H NMR (400 MHz, Chloroform-d) δ 8.97-8.95 (m, 2H), 7.29-7.26 (m, 1H), 4.63-4.60 (m, 1H), 4.10-3.83 (m, 3H), 2.99-2.93 (m, 4H), 2.89-2.87 (m, 4H), 2.72-2.70 (m, 3H), 2.54-2.20 (m, 4H), 1.84-1.78 (m, 3H), 0.47-0.41 (m, 4H).

UPLC (ESI−MS) m/z=432.2 [M+H]⁺.

### Example 57: 6-cyclopropyl-8-(8,8-difluoro-2,6-diazaspiro[3.4]octan-6-yl)-N-(1-(methylsulfonyl)piperidin-4-yl)pyrido[3,4-d]pyrimidin-2-amine

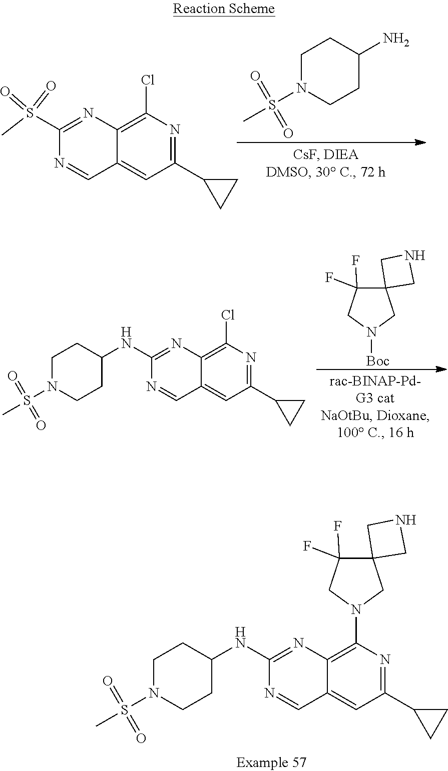 [SMILES: CS(=O)(=O)c1ncc2cc(C3CC3)nc(Cl)c2n1 ; CS(=O)(=O)N1CCC(N)CC1 ; CS(=O)(=O)N1CCC(Nc2ncc3cc(C4CC4)nc(Cl)c3n2)CC1 ; CC(C)(C)OC(=O)N1CC(F)(F)C2(CNC2)C1 ; CS(=O)(=O)N1CCC(Nc2ncc3cc(C4CC4)nc(N4CC(F)(F)C5(CNC5)C4)c3n2)CC1]

### Detailed Procedure

### Step 1: 8-chloro-6-cyclopropyl-N-(1-(methylsulfonyl)piperidin-4-yl)pyrido[3,4-d]pyrimidin-2-amine

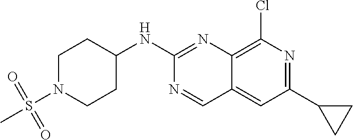 [SMILES: CS(=O)(=O)N1CCC(Nc2ncc3cc(C4CC4)nc(Cl)c3n2)CC1]

A solution of 8-chloro-6-cyclopropyl-2-(methylsulfonyl)pyrido[3,4-d]pyrimidine (100 mg, 352 mol) in anhydrous dimethylsulfoxide (1.8 mL) was charged with 1-(methylsulfonyl)piperidin-4-amine (69.1 mg, 388 mol), cesium fluoride (161 mg, 1.06 mmol), and N-ethyl-N-isopropylpropan-2-amine (137 mg, 184 L, 1.06 mmol). The reaction vial was capped and stirred at 30° C. for 72 h. The crude mixture was diluted with water (50.0 mL) and extracted with dichloromethane (3×20.0 mL). The combined organic layers were washed with brine, dried over anhydrous magnesium sulfate, filtered, and concentrated under reduced pressure. The residue was purified by flash chromatography on silica gel (heptane/EtOAc, 1:1) to afford the title compound (62.0 mg, 46% yield).

LCMS (ES) [M+H]⁺=382.10

### Step 2: 6-cyclopropyl-8-(8,8-difluoro-2,6-diazaspiro[3.4]octan-6-yl)-N-(1-(methylsulfonyl)piperidin-4-yl)pyrido[3,4-d]pyrimidin-2-amine

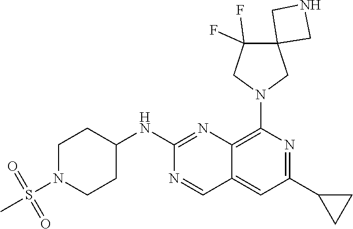 [SMILES: CS(=O)(=O)N1CCC(Nc2ncc3cc(C4CC4)nc(N4CC(F)(F)C5(CNC5)C4)c3n2)CC1]

To a solution of 8-chloro-6-cyclopropyl-N-(1-(methylsulfonyl)piperidin-4-yl)pyrido[3,4-d]pyrimidin-2-amine (22.0 mg, 58.0 mol) in anhydrous dioxane (290 L) was added tert-butyl-8,8-difluoro-2,6-diazaspiro[3.4]octane-2-carboxylate (17.0 mg, 69.0 mol), sodium tert-butoxide (17.0 mg, 170 mol), and [2′-(amino-κN)[1,1′-biphenyl]-2-yl-κC][[2′-(diphenylphosphino)[1,1′-binaphthalen]-2-yl]diphenylphosphine-κP](methanesulfonato-κO)-palladium (5.7 mg, 5.8 mol). The reaction vial was capped and stirred at 100° C. for 16 h. The mixture was cooled to rt, diluted with ethyl acetate (30.0 mL) and water (20.0 mL), extracted (3×20.0 mL ethyl acetate), washed with brine, dried over anhydrous MgSO₄, filtered, and concentrated to dryness.

To the crude material was added trifluoroacetic acid (800 L) at 0° C. After 10 minutes, the reaction was allowed to warm to rt and continuously stirred until completion of reaction as determined by LC-MS. Afterwards, the reaction was concentrated to dryness. The resulting solid was dissolved in dichloromethane (10 mL), neutralized with NaHCO₃ sat. aq. solution, and extracted with dichloromethane (3×10.0 mL). The combined organic phases were concentrated to dryness and purified by preparative RP-HPLC to provide the title compound (14.0 mg, 48% yield).

¹H NMR (DMSO-d₆, 499 MHz) δ 8.98 (s, 1H), 7.4-7.6 (m, 1H), 6.8-6.9 (m, 1H), 4.9-5.0 (m, 1H), 4.2-4.4 (m, 4H), 3.80 (d, 2H, J=8.5 Hz), 3.60 (d, 2H, J=12.3 Hz), 3.46 (d, 1H, J=8.5 Hz), 2.8-3.0 (m, 5H), 2.04 (d, 2H, J=12.3 Hz), 1.9-2.0 (m, 2H), 1.5-1.7 (m, 3H), 0.92 (m, 2H), 0.7-0.8 (m, 2H).

LCMS (ES) [M+H]⁺=494.30

### Example 59: N-(1-(cyclopropylsulfonyl)piperidin-4-yl)-8-(8,8-difluoro-2,6-diazaspiro[3.4]octan-6-yl)-6-methylpyrido[3,4-d]pyrimidin-2-amine

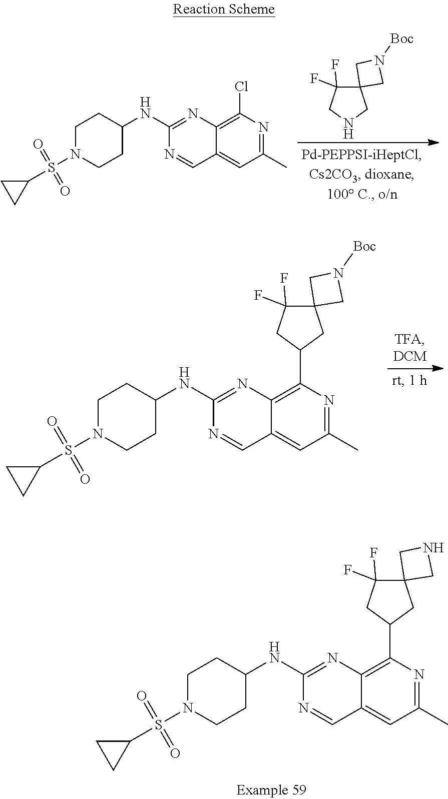 [SMILES: Cc1cc2cnc(NC3CCN(S(=O)(=O)C4CC4)CC3)nc2c(Cl)n1 ; CC(C)(C)OC(=O)N1CC2(CNCC2(F)F)C1 ; Cc1cc2cnc(NC3CCN(S(=O)(=O)C4CC4)CC3)nc2c(C2CC(F)(F)C3(C2)CN(C(=O)OC(C)(C)C)C3)n1 ; Cc1cc2cnc(NC3CCN(S(=O)(=O)C4CC4)CC3)nc2c(C2CC(F)(F)C3(CNC3)C2)n1]

### Detailed Procedure

### Step 1: Tert-butyl 6-(2-((1-(cyclopropylsulfonyl)piperidin-4-yl)amino)-6-methylpyrido[3,4-d]pyrimidin-8-yl)-8,8-difluoro-2,6-diazaspiro[3.4]octane-2-carboxylate

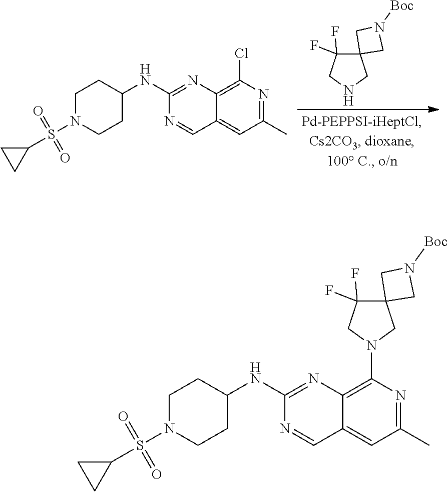 [SMILES: Cc1cc2cnc(NC3CCN(S(=O)(=O)C4CC4)CC3)nc2c(Cl)n1 ; CC(C)(C)OC(=O)N1CC2(CNCC2(F)F)C1 ; Cc1cc2cnc(NC3CCN(S(=O)(=O)C4CC4)CC3)nc2c(N2CC(F)(F)C3(CN(C(=O)OC(C)(C)C)C3)C2)n1]

Pd-PEPPSI-iHeptCl3-chloropyridine (255 mg, 0.26 mmol) was added to a mixture of 8-chloro-N-(1-(cyclopropylsulfonyl)piperidin-4-yl)-6-methylpyrido[3,4-d]pyrimidin-2-amine (2 g, 5.23 mmol), tert-butyl 8,8-difluoro-2,6-diazaspiro[3.4]octane-2-carboxylate (1.30 g, 5.23 mmol) and Cs₂CO₃ (3.41 g, 10.47 mmol) in dioxane (30 mL) under a nitrogen atmosphere. The resulting mixture was stirred overnight at 100° C. The resulting mixture was filtered and the filter cake was washed with DCM (50 mL). The filtrate was concentrated under reduced pressure. The residue was purified by silica gel column chromatography (CH₂Cl₂/MeOH, 3:97) to afford tert-butyl 6-(2-((1-(cyclopropylsulfonyl)piperidin-4-yl)amino)-6-methylpyrido[3,4-d]pyrimidin-8-yl)-8,8-difluoro-2,6-diazaspiro[3.4]octane-2-carboxylate (2.8 g, 70.60% yield).

LCMS (ES−MS) m/z=594.3 [M+H]⁺.

### Step 2: N-(1-(cyclopropylsulfonyl)piperidin-4-yl)-8-(8,8-difluoro-2,6-diazaspiro[3.4]octan-6-yl)-6-methylpyrido[3,4-d]pyrimidin-2-amine

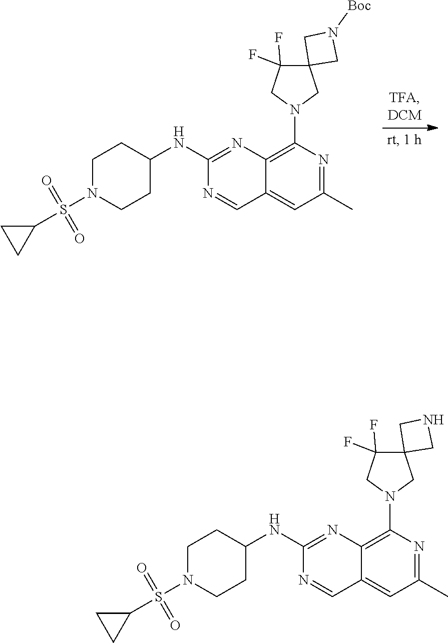 [SMILES: Cc1cc2cnc(NC3CCN(S(=O)(=O)C4CC4)CC3)nc2c(N2CC(F)(F)C3(CN(C(=O)OC(C)(C)C)C3)C2)n1 ; Cc1cc2cnc(NC3CCN(S(=O)(=O)C4CC4)CC3)nc2c(N2CC(F)(F)C3(CNC3)C2)n1]

A solution of tert-butyl 6-(2-((1-(cyclopropylsulfonyl)piperidin-4-yl)amino)-6-methylpyrido[3,4-d]pyrimidin-8-yl)-8,8-difluoro-2,6-diazaspiro[3.4]octane-2-carboxylate (2.6 g, 4.37 mmol) in TFA (5 mL) and DCM (15 mL) was stirred for 1 h at room temperature. The reaction mixture was concentrated and the residue was purified by silica gel column chromatography (CH₂Cl₂/MeOH, 90:10) to afford N-(1-(cyclopropylsulfonyl)piperidin-4-yl)-8-(8,8-difluoro-2,6-diazaspiro[3.4]octan-6-yl)-6-methylpyrido[3,4-d]pyrimidin-2-amine (2.0686 g, 91.97% yield).

¹H NMR (400 MHz, DMSO-d6) δ 9.20 (s, 1H), 9.00 (s, 1H), 7.60 (s, 1H), 6.82 (s, 1H), 4.58-4.41 (m, 2H), 4.38-4.32 (m, 2H), 4.31-4.21 (m, 2H), 4.19-4.09 (m, 2H), 3.87 (s, 1H), 3.72-3.57 (m, 2H), 3.09-2.97 (m, 2H), 2.63-2.54 (m, 1H), 2.36 (s, 3H), 2.10-1.97 (m, 2H), 1.69-1.52 (m, 2H), 1.07-0.91 (m, 4H).

LCMS (ES−MS) m/z=492.2 [M+H]⁺.

### Compound 86: 8-(8,8-difluoro-2,6-diazaspiro[3.4]octan-6-yl)-6-methyl-N-(1-((1-methyl-1H-pyrazol-4-yl)sulfonyl)piperidin-4-yl)pyrido[3,4-d]pyrimidin-2-amine

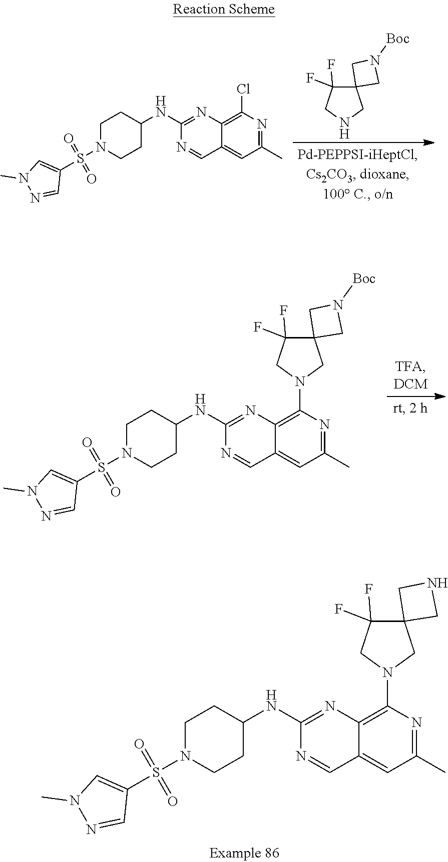

### Detailed Procedure

### Step 1: Tert-butyl 8,8-difluoro-6-(6-methyl-2-((1-((1-methyl-1H-pyrazol-4-yl)sulfonyl)piperidin-4-yl)amino)pyrido[3,4-d]pyrimidin-8-yl)-2,6-diazaspiro[3.4]octane-2-carboxylate

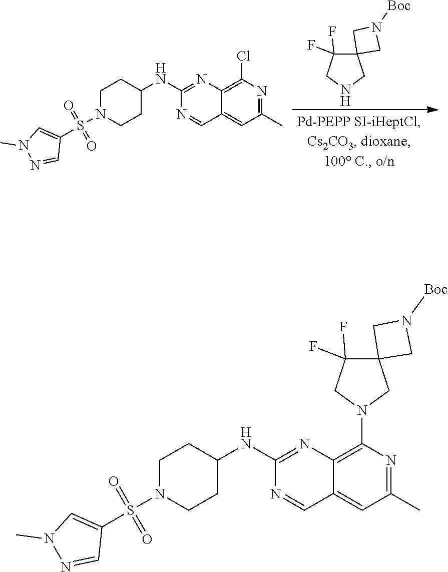

A solution of 8-chloro-6-methyl-N-(1-((1-methyl-1H-pyrazol-4-yl)sulfonyl)piperidin-4-yl)pyrido[3,4-d]pyrimidin-2-amine (500 mg, 1.18 mmol), tert-butyl 8,8-difluoro-2,6-diazaspiro[3.4]octane-2-carboxylate (293.83 mg, 1.18 mmol), Cs₂CO₃ (1158.40 mg, 3.55 mmol) and Pd-PEPPSI-iHeptCl 3-chloropyridine (115.41 mg, 0.11 mmol) in dioxane (10 mL) was stirred overnight at 100° C. under a nitrogen atmosphere. The reaction mixture was diluted with water (100 mL) and extracted with EA (3×100 mL). The combined organic layers were dried over anhydrous Na₂SO₄. After filtration, the filtrate was concentrated under reduced pressure. The residue was purified by silica gel column chromatography (PE/EA, 1:1) to afford tert-butyl 8,8-difluoro-6-(6-methyl-2-((1-((1-methyl-1H-pyrazol-4-yl)sulfonyl)piperidin-4-yl)amino)pyrido[3,4-d]pyrimidin-8-yl)-2,6-diazaspiro[3.4]octane-2-carboxylate (600 mg, 75.9% yield).

LCMS (ES−MS) m/z=643.3 [M+H]⁺. 4-yl)sulfonyl)piperidin-4-yl)pyrido[3,4-d]pyrimidin-2-amine

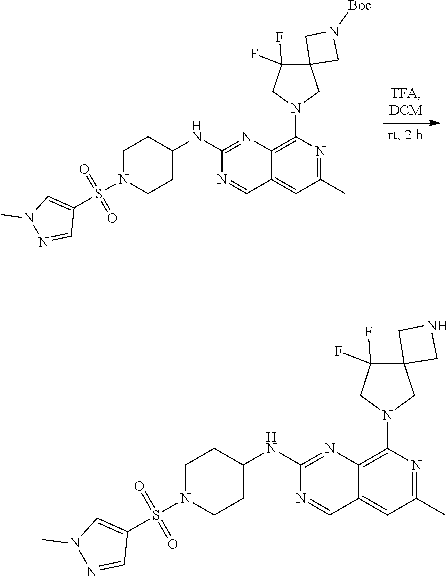

A solution of tert-butyl 8,8-difluoro-6-(6-methyl-2-((1-((1-methyl-1H-pyrazol-4-yl)sulfonyl)piperidin-4-yl)amino)pyrido[3,4-d]pyrimidin-8-yl)-2,6-diazaspiro[3.4]octane-2-carboxylate (3 g, 4.73 mmol) in TFA (5 mL) and DCM (15 mL) was stirred for 2 h at room temperature. The resulting mixture was concentrated under reduced pressure. The residue was purified by silica gel column chromatography (CH₂Cl₂/MeOH, 10:1) to afford the TFA salt of 8-(8,8-difluoro-2,6-diazaspiro[3.4]octan-6-yl)-6-methyl-N-(1-((1-methyl-1H-pyrazol-4-yl)sulfonyl)piperidin-4-yl)pyrido[3,4-d]pyrimidin-2-amine (2.3177 g, 91.7% yield).

¹H NMR (400 MHz, DMSO-d6) δ 9.09 (s, 2H), 8.98 (s, 1H), 8.34 (s, 1H), 7.78 (s, 1H), 7.56 (s, 1H), 6.81 (s, 1H), 4.42 (s, 2H), 4.29 (s, 2H), 4.22 (d, J=11.6 Hz, 2H), 4.09 (d, J=11.5 Hz, 2H), 3.92 (s, 3H), 3.72 (s, 1H), 3.49 (d, J=11.3 Hz, 2H), 2.53 (s, 2H), 2.35 (s, 3H), 2.02 (d, J=12.2 Hz, 2H), 1.69-1.57 (m, 2H).

LCMS (ES−MS) m/z=534.2 [M+H]⁺.

### Example 94: 8-(8,8-difluoro-2-methyl-2,6-diazaspiro[3.4]octan-6-yl)-6-methyl-N-(1-((1-methyl-1H-pyrazol-4-yl)sulfonyl)piperidin-4-yl)pyrido[3,4-d]pyrimidin-2-amine

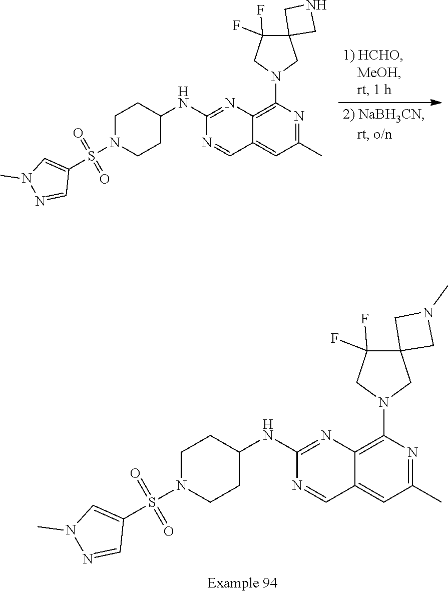

A mixture of 8-(8,8-difluoro-2,6-diazaspiro[3.4]octan-6-yl)-6-methyl-N-(1-((1-methyl-1H-pyrazol-4-yl)sulfonyl)piperidin-4-yl)pyrido[3,4-d]pyrimidin-2-amine (250 mg, 0.46 mmol) and formaldehyde (28.14 mg, 0.93 mmol) in MeOH (10 mL) was stirred for 1 h at room temperature. NaBH₃CN (117.77 mg, 1.87 mmol) was added and the resulting mixture was stirred overnight at room temperature. The mixture was concentrated under reduced pressure and the residue purified by silica gel column chromatography (CH₂Cl₂/MeOH, 10:1) to afford 8-(8,8-difluoro-2-methyl-2,6-diazaspiro[3.4]octan-6-yl)-6-methyl-N-(1-((1-methyl-1H-pyrazol-4-yl)sulfonyl)piperidin-4-yl)pyrido[3,4-d]pyrimidin-2-amine (96.2 mg, 37.1% yield).

¹H NMR (400 MHz, DMSO-d₆) δ 8.96 (s, 1H), 8.34 (s, 1H), 7.77 (d, J=0.6 Hz, 1H), 7.53 (s, 1H), 6.76 (d, J=0.9 Hz, 1H), 4.28 (s, 2H), 4.23 (s, 1H), 3.92 (s, 3H), 3.70 (s, 1H), 3.55 (d, J=11.6 Hz, 2H), 3.39 (d, J=7.8 Hz, 2H), 3.18 (s, 2H), 2.45 (d, J=11.4 Hz, 1H), 2.33 (s, 3H), 2.26 (s, 3H), 2.08 (s, 2H), 2.06-1.99 (m, 2H), 1.69-1.59 (m, 2H).

LCMS (ES−MS) m/z=548.4 [M+H]⁺.

### Example 66: (1-methyl-3-(6-methyl-2-((1-(methylsulfonyl)piperidin-4-yl)amino)pyrido[3,4-d]pyrimidin-8-yl)cyclobutyl)methanol

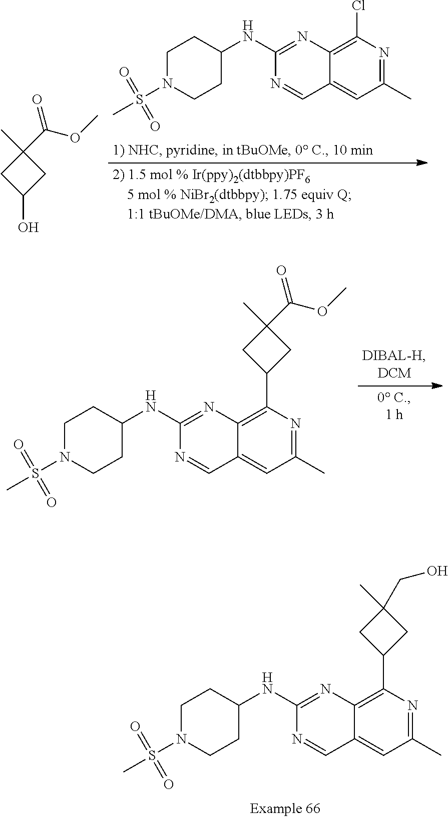

### Step 1: Methyl 1-methyl-3-(6-methyl-2-((1-(methylsulfonyl)piperidin-4-yl)amino)pyrido[3,4-d]pyrimidin-8-yl)cyclobutane-1-carboxylate

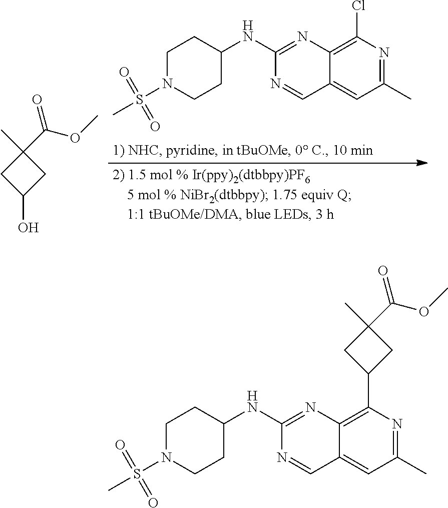

An oven-dried 20 mL vial was charged with methyl 3-hydroxy-1-methylcyclobutane-1-carboxylate (100 mg, 0.69 mmol) and 5,7-di-tert-butyl-3-phenylbenzo[d]oxazol-3-ium tetrafluoroborate (250 mg, 0.63 mmol). Under nitrogen, tert-butyl methyl ether (4 mL) was added and the reaction stirred at room temperature for 5 minutes. A mixture of pyridine (50.16 mg, 0.63 mmol) in tert-butyl methyl ether (1 mL) was added and the mixture stirred at room temperature for 10 minutes (mixture A). Another oven-dried 40 mL vial was charged with 8-chloro-6-methyl-N-(1-(methylsulfonyl)piperidin-4-yl)pyrido[3,4-d]pyrimidin-2-amine (141.04 mg, 0.39 mmol), NiBr₂(dtbbpy) (14.66 mg, 0.03 mmol), Ir(ppy)₂(dtbbpy)PF₆ (5.43 mg, 0.006 mmol), phatalamide (9.65 mg, 0.02 mmol) and 1-azabicyclo[2.2.2]octane (77.1 mg, 0.69 mmol). DMA (5 mL) was added under nitrogen (mixture B). The mixture A was added to the mixture B under a nitrogen atmosphere and the resulting mixture was stirred and irradiated with a 450 nm LED lamp under a fan for 3 h. The residue was dissolved in water (20 mL) and extracted with EA (3×20 mL). The combined organic layers were dried over anhydrous Na₂SO₄. After filtration, the filtrate was concentrated under reduced pressure. The residue was purified by Prep-TLC (PE/EA, 1:2) to afford crude methyl 1-methyl-3-(6-methyl-2-((1-(methylsulfonyl)piperidin-4-yl)amino)pyrido[3,4-d]pyrimidin-8-yl)cyclobutane-1-carboxylate (90 mg).

LCMS (ESI-MS) m/z=448.2 [M+H]⁺.

### Step 2: (1-methyl-3-(6-methyl-2-((1-(methylsulfonyl)piperidin-4-yl)amino)pyrido[3,4-d]pyrimidin-8-yl)cyclobutyl)methanol

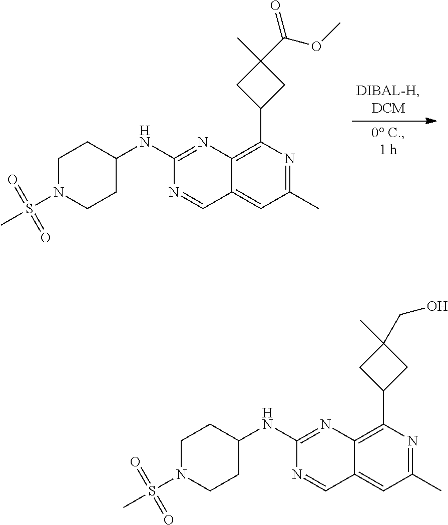

DIBAL-H (47.67 mg, 0.336 mmol) was added to a solution of methyl 1-methyl-3-(6-methyl-2-((1-(methylsulfonyl)piperidin-4-yl)amino)pyrido[3,4-d]pyrimidin-8-yl)cyclobutane-1-carboxylate (50 mg, 0.112 mmol) in DCM (1 mL) at 0° C. The resulting mixture was stirred for 1 h at 0° C. and quenched with water (20 mL) at 0° C. The resulting mixture was extracted with DCM (3×20 mL) and dried over anhydrous Na₂SO₄. After filtration, the filtrate was concentrated under reduced pressure. The residue was purified by Prep-TLC (EA=100%) to afford (1-methyl-3-(6-methyl-2-((1-(methylsulfonyl)piperidin-4-yl)amino)pyrido[3,4-d]pyrimidin-8-yl)cyclobutyl)methanol (1.1 mg, 2.33% yield).

¹H NMR (400 MHz, CDCl₃) δ 8.95 (s, 1H), 7.22-7.11 (m, 1H), 5.31 (s, 1H), 4.65-4.51 (m, 1H), 4.41-4.26 (m, 1H), 4.18-3.99 (m, 1H), 3.87-3.68 (m, 3H), 3.52 (s, 1H), 3.11-2.93 (m, 2H), 2.93-2.78 (m, 4H), 2.76-2.68 (m, 2H), 2.44-2.16 (m, 5H), 1.87-1.60 (m, 5H), 1.19 (s, 1H).

LCMS (ES−MS) m/z=420.2 [M+H]⁺.

### Example 67: 6-cyclopropyl-N-(1-(methylsulfonyl)piperidin-4-yl)-8-(5-oxa-8-azaspiro[3.5]nonan-2-yl)pyrido[3,4-d]pyrimidin-2-amine

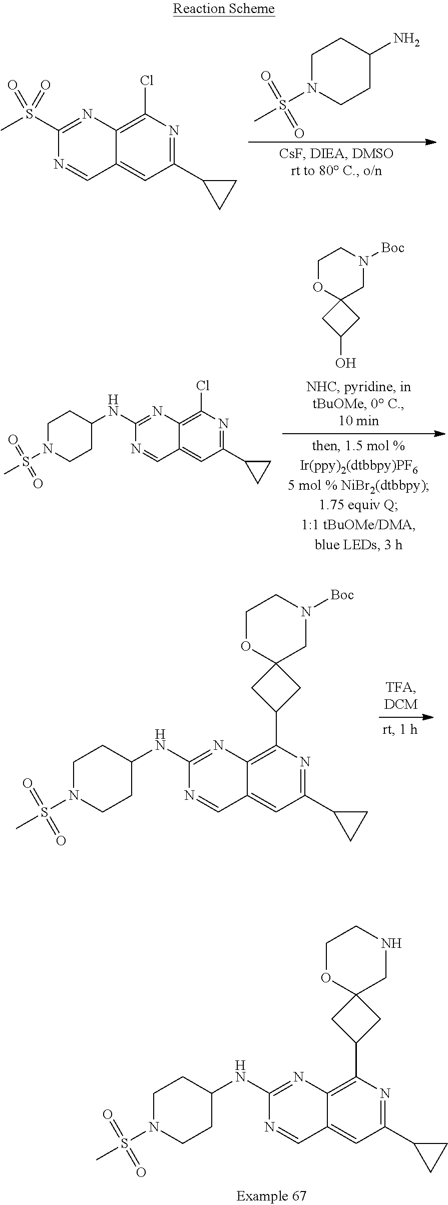 [SMILES: CS(=O)(=O)c1ncc2cc(C3CC3)nc(Cl)c2n1 ; CS(=O)(=O)N1CCC(N)CC1 ; CC(C)(C)OC(=O)N1CCOC2(CC(O)C2)C1 ; CS(=O)(=O)N1CCC(Nc2ncc3cc(C4CC4)nc(Cl)c3n2)CC1 ; CC(C)(C)OC(=O)N1CCOC2(CC(c3nc(C4CC4)cc4cnc(NC5CCN(S(C)(=O)=O)CC5)nc34)C2)C1 ; CS(=O)(=O)N1CCC(Nc2ncc3cc(C4CC4)nc(C4CC5(CNCCO5)C4)c3n2)CC1]

### Detailed Procedure

### Step 1: 8-chloro-6-cyclopropyl-N-(1-(methylsulfonyl)piperidin-4-yl)pyrido[3,4-d]pyrimidin-2-amine

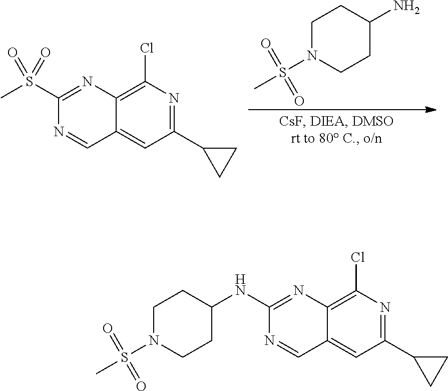 [SMILES: CS(=O)(=O)c1ncc2cc(C3CC3)nc(Cl)c2n1 ; CS(=O)(=O)N1CCC(N)CC1 ; CS(=O)(=O)N1CCC(Nc2ncc3cc(C4CC4)nc(Cl)c3n2)CC1]

DIEA (137 mg, 1.05 mmol) was added to a mixture of 8-chloro-6-cyclopropyl-2-(methylsulfonyl)pyrido[3,4-d]pyrimidine (100 mg, 0.35 mmol), 1-(methylsulfonyl)piperidin-4-amine (62.8 mg, 0.35 mmol) and CsF (161 mg, 1.05 mmol) in DMSO (2 mL) at room temperature. The resulting mixture was stirred overnight at 80° C. The residue was dissolved in water (20 mL) and extracted with EA (3×20 mL). The combined organic layers were washed with brine (3×20 mL), dried over anhydrous Na₂SO₄. After filtration, the filtrate was concentrated under reduced pressure. The residue was purified by Prep-TLC (PE/EA, 1:2) to afford crude 8-chloro-6-cyclopropyl-N-(1-(methylsulfonyl)piperidin-4-yl)pyrido[3,4-d]pyrimidin-2-amine (80 mg).

LCMS (ES−MS) m/z=382.2 [M+H]⁺.

### Step 2: Tert-butyl 2-(6-cyclopropyl-2-((1-(methylsulfonyl)piperidin-4-yl)amino)pyrido[3,4-d]pyrimidin-8-yl)-5-oxa-8-azaspiro[3.5]nonane-8-carboxylate

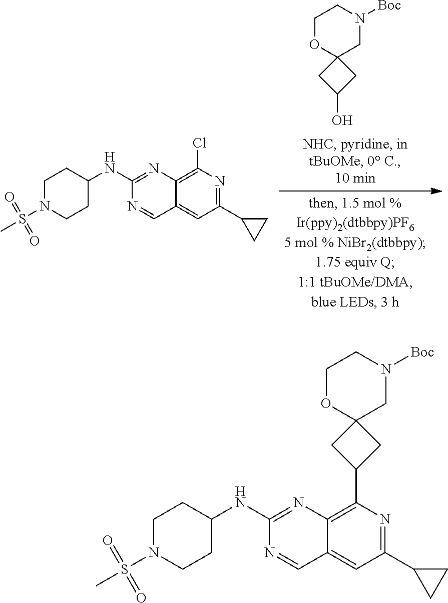 [SMILES: CC(C)(C)OC(=O)N1CCOC2(CC(O)C2)C1 ; CS(=O)(=O)N1CCC(Nc2ncc3cc(C4CC4)nc(Cl)c3n2)CC1 ; CC(C)(C)OC(=O)N1CCOC2(CC(c3nc(C4CC4)cc4cnc(NC5CCN(S(C)(=O)=O)CC5)nc34)C2)C1]

An oven-dried 20 mL vial was charged with tert-butyl 2-hydroxy-5-oxa-8-azaspiro[3.5]nonane-8-carboxylate (55.7 mg, 0.229 mmol) and 5,7-di-tert-butyl-3-phenylbenzo[d]oxazol-3-ium tetrafluoroborate (82.8 mg, 0.21 mmol). Under nitrogen, tert-butyl methyl ether (4 mL) was added and the reaction stirred at room temperature for 5 minutes. A mixture of pyridine (16.57 mg, 0.21 mmol) in tert-butyl methyl ether (1 mL) was added and the mixture stirred at room temperature for 10 minutes (mixture A). Another oven-dried 40 mL vial was charged with 8-chloro-6-cyclopropyl-N-(1-(methylsulfonyl)piperidin-4-yl)pyrido[3,4-d]pyrimidin-2-amine (50 mg, 0.13 mmol), NiBr₂(dtbbpy) (4.88 mg, 0.01 mmol), Ir(ppy)₂(dtbbpy)PF₆ (1.79 mg, 0.002 mmol), 1-azabicyclo[2.2.2]octane (25.89 mg, 0.23 mmol) and quinuclidone (25.48 mg, 0.229 mmol). DMA (5 mL) was added under nitrogen (mixture B). The mixture A was added to the mixture B under a nitrogen atmosphere and the resulting mixture was stirred and irradiated with a 450 nm LED lamp under a fan for 3 h. The residue was dissolved in water (20 mL) and extracted with EA (3×20 mL). The combined organic layers were dried over anhydrous Na2SO4. After filtration, the filtrate was concentrated under reduced pressure. The residue was purified by Prep-TLC (PE/EA, 1:2) to afford crude tert-butyl 2-(6-cyclopropyl-2-((1-(methylsulfonyl)piperidin-4-yl)amino)pyrido[3,4-d]pyrimidin-8-yl)-5-oxa-8-azaspiro[3.5]nonane-8-carboxylate (50 mg).

LCMS (ES−MS) m/z=573.2 [M+H]⁺.

### Step 3: 6-cyclopropyl-N-(1-(methylsulfonyl)piperidin-4-yl)-8-(5-oxa-8-azaspiro[3.5]nonan-2-yl)pyrido[3,4-d]pyrimidin-2-amine

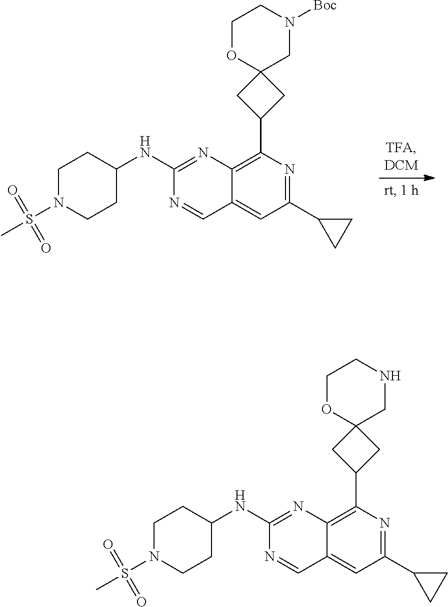 [SMILES: CC(C)(C)OC(=O)N1CCOC2(CC(c3nc(C4CC4)cc4cnc(NC5CCN(S(C)(=O)=O)CC5)nc34)C2)C1 ; CS(=O)(=O)N1CCC(Nc2ncc3cc(C4CC4)nc(C4CC5(CNCCO5)C4)c3n2)CC1]

TFA (0.1 mL) was added to a solution of tert-butyl 2-(6-cyclopropyl-2-((1-(methylsulfonyl)piperidin-4-yl)amino)pyrido[3,4-d]pyrimidin-8-yl)-5-oxa-8-azaspiro[3.5]nonane-8-carboxylate (50 mg, 0.087 mmol) in DCM (2 mL). The resulting mixture was stirred for 1 h at room temperature. The reaction mixture was concentrated under reduced pressure and the residue was purified by preparative RP-HPLC to afford 6-cyclopropyl-N-(1-(methylsulfonyl)piperidin-4-yl)-8-(5-oxa-8-azaspiro[3.5]nonan-2-yl)pyrido[3,4-d]pyrimidin-2-amine (1.0 mg, 2.30% yield).

¹H NMR (400 MHz, CDCl₃) δ 8.93 (s, 1H), 7.16 (s, 1H), 5.30-5.20 (m, 1H), 4.12-3.91 (m, 2H), 3.87-3.67 (m, 4H), 3.19-2.98 (m, 4H), 2.96-2.80 (m, 5H), 2.61-2.49 (m, 4H), 2.34-2.20 (m, 3H), 2.17-2.00 (m, 1H), 1.83-1.65 (m, 2H), 1.15-1.05 (m, 2H), 1.02-0.93 (m, 2H).

LCMS (ES−MS) m/z=473.2 [M+H]⁺.

### Example 90: 1-(difluoromethyl)-3-(6-methyl-2-((1-(methylsulfonyl)piperidin-4-yl)amino)pyrido[3,4-d]pyrimidin-8-yl)cyclobutan-1-ol

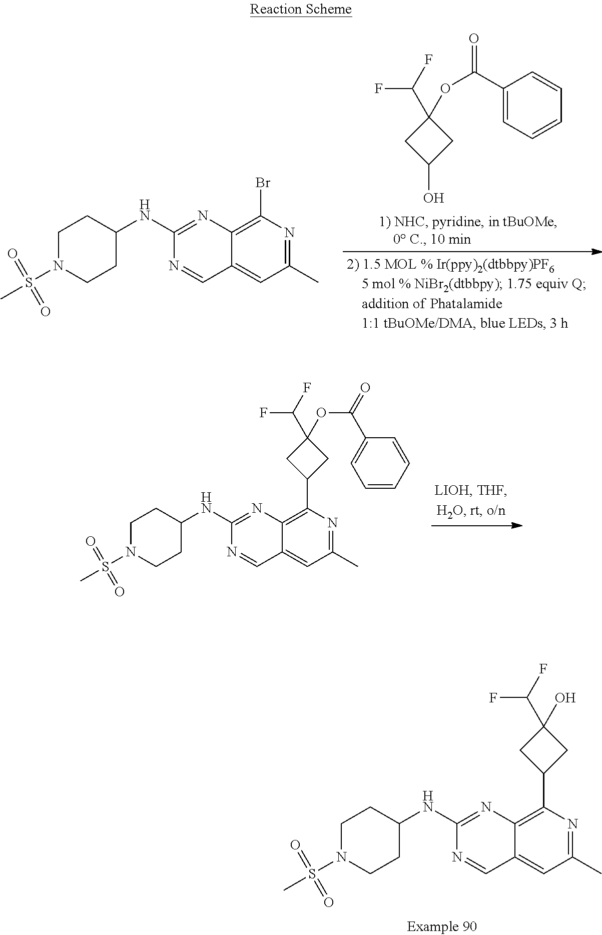

### Detailed Procedure

### Step 1: 1-(difluoromethyl)-3-(6-methyl-2-((1-(methylsulfonyl)piperidin-4-yl)amino)pyrido[3,4-d]pyrimidin-8-yl)cyclobutyl benzoate

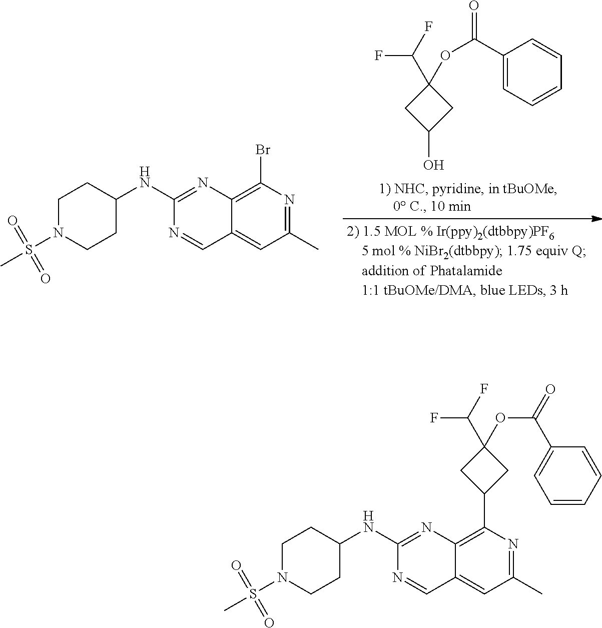

An oven-dried 20 mL vial was charged with 1-(difluoromethyl)-3-hydroxycyclobutyl benzoate (106 mg, 0.44 mmol) and 5,7-di-tert-butyl-3-phenylbenzo[d]oxazol-3-ium tetrafluoroborate (158 mg, 0.40 mmol). Under nitrogen, tert-butyl methyl ether (4 mL) was added and the reaction stirred at room temperature for 5 minutes. A mixture of pyridine (31.6 mg, 0.40 mmol) in tert-butyl methyl ether (1 mL) and the mixture stirred at room temperature for 10 minutes (mixture A). Another oven-dried 40 mL vial was charged with 8-bromo-6-methyl-N-(1-(methylsulfonyl)piperidin-4-yl)pyrido[3,4-d]pyrimidin-2-amine (100 mg, 0.25 mmol), Ir(ppy)₂(dtbbpy)PF₆ (3.43 mg, 0.004 mmol), NiBr₂(dtbbpy) (9.78 mg, 0.02 mmol), phthalimide (8.27 mg, 0.056 mmol) and 1-azabicyclo[2.2.2]octane (48.6 mg, 0.44 mmol). DMA (5 mL) was added under nitrogen (mixture B). The mixture A was added to the mixture B under a nitrogen atmosphere and the resulting mixture was stirred and irradiated with a 450 nm LED lamp under a fan for 3 h. The residue was dissolved in water (20 mL) and extracted with EA (3×20 mL). The combined organic layers were dried over anhydrous Na2SO4. After filtration, the filtrate was concentrated under reduced pressure. The residue was purified by Prep-TLC (PE/EA, 1:2) to afford crude 1-(difluoromethyl)-3-(6-methyl-2-((1-(methylsulfonyl)piperidin-4-yl)amino)pyrido[3,4-d]pyrimidin-8-yl)cyclobutyl benzoate (50 mg, 36.68% yield).

LCMS (ES−MS) m/z=546.2 [M+H]⁺.

### Step 2: 1-(difluoromethyl)-3-(6-methyl-2-((1-(methylsulfonyl)piperidin-4-yl)amino)pyrido[3,4-d]pyrimidin-8-yl)cyclobutan-1-ol

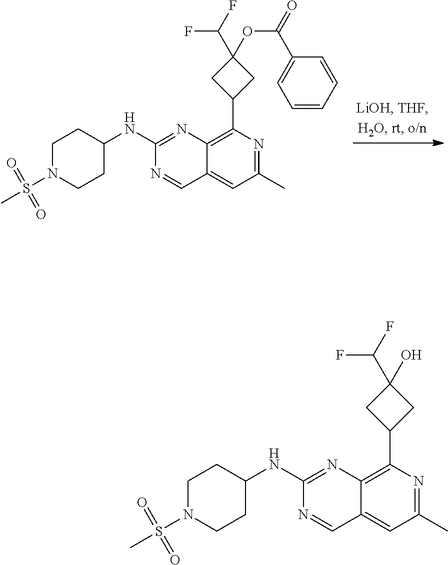

LiOH (10.9 mg, 0.46 mmol) was added to a solution of 1-(difluoromethyl)-3-(6-methyl-2-((1-(methylsulfonyl)piperidin-4-yl)amino)pyrido[3,4-d]pyrimidin-8-yl)cyclobutyl benzoate (50 mg, 0.09 mmol,) in THF (3 mL) and H₂O (1 mL). The resulting mixture was stirred overnight at room temperature. The reaction mixture was diluted with H₂O (10 mL) and extracted with EA (3×20 mL). The combined organic layers were washed with brine (3×20 mL), dried over anhydrous Na₂SO₄ and concentrated under reduced pressure. The residue purified by preparative RP-HPLC to afford 1-(difluoromethyl)-3-(6-methyl-2-((1-(methylsulfonyl)piperidin-4-yl)amino)pyrido[3,4-d]pyrimidin-8-yl)cyclobutan-1-ol (5.8 mg, 13.36%).

¹H NMR (400 MHz, DMSO-d₆) δ 9.16 (s, 1H), 7.70 (s, 1H), 7.37 (s, 1H), 6.06-5.85 (m, 2H), 4.08-3.86 (m, 2H), 3.60-3.50 (m, 2H), 2.96-2.83 (m, 5H), 2.72-2.62 (m, 2H), 2.59-2.52 (m, 4H), 2.14-2.01 (m, 3H), 1.67-1.55 (m, 2H).

LCMS (ES−MS) m/z=442.1 [M+H]⁺.

### Example 96: 8-(8,8-difluoro-6-azaspiro[3.4]octan-2-yl)-6-methyl-N-(1-(methylsulfonyl)piperidin-4-yl)pyrido[3,4-d]pyrimidin-2-amine

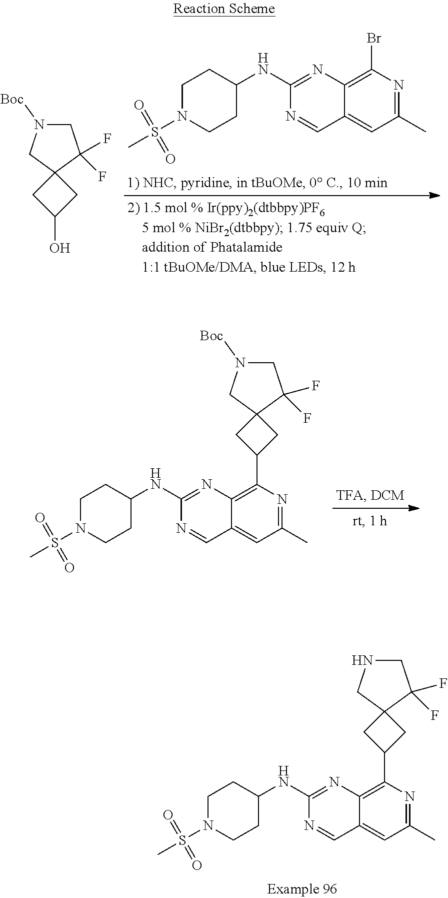

### Step 1: Tert-butyl 8,8-difluoro-2-(6-methyl-2-((1-(methylsulfonyl)piperidin-4-yl)amino)pyrido[3,4-d]pyrimidin-8-yl)-6-azaspiro[3.4]octane-6-carboxylate

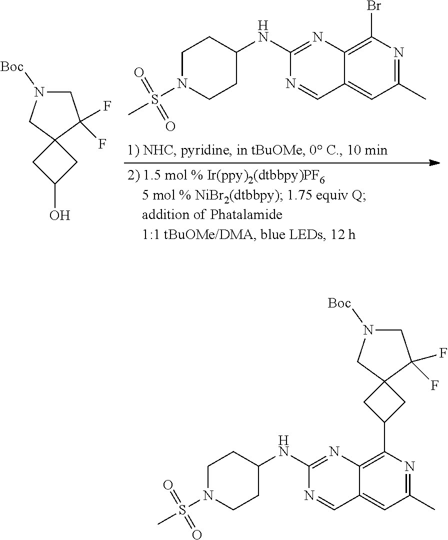

An oven-dried 20 mL vial was charged with tert-butyl 8,8-difluoro-2-hydroxy-6-azaspiro[3.4]octane-6-carboxylate (207 mg, 0.78 mmol) and 5,7-di-tert-butyl-3-phenylbenzo[d]oxazol-3-ium tetrafluoroborate (284 mg, 0.72 mmol). Under nitrogen, tert-butyl methyl ether (4 mL) was added and the reaction stirred at room temperature for 5 minutes. A mixture of pyridine (56.9 mg, 0.72 mmol) in tert-butyl methyl ether (1 mL) was added and the mixture was stirred at room temperature for 10 minutes (mixture A). Another oven-dried 40 mL vial was charged with 8-bromo-6-methyl-N-(1-(methylsulfonyl)piperidin-4-yl)pyrido[3,4-d]pyrimidin-2-amine (180 mg, 0.45 mmol), Ir(ppy)₂(dtbbpy)PF₆ (6.16 mg, 0.007 mmol), NiBr₂(dtbbpy) (16.42 mg, 0.034 mmol), phthalimide (26.46 mg, 0.18 mmol) and 1-azabicyclo[2.2.2]octane (87.49 mg, 0.78 mmol). DMA (5 mL) was added under nitrogen (mixture B). The mixture A was added to the mixture B under a nitrogen atmosphere and the resulting mixture was stirred and irradiated with a 450 nm LED lamp under a fan for 3 h. The residue was dissolved in water (20 mL) and extracted with EA (3×20 mL). The combined organic layers were dried over anhydrous Na2SO4. After filtration, the filtrate was concentrated under reduced pressure. The residue was purified by Prep-TLC (PE/EA, 1:2) to afford tert-butyl 8,8-difluoro-2-(6-methyl-2-((1-(methylsulfonyl)piperidin-4-yl)amino)pyrido[3,4-d]pyrimidin-8-yl)-6-azaspiro[3.4]octane-6-carboxylate (20 mg, 7.85% yield).

LCMS (ES−MS) m/z=567.2 [M+H]⁺.

### Step 2: 8-(8,8-difluoro-6-azaspiro[3.4]octan-2-yl)-6-methyl-N-(1-(methylsulfonyl)piperidin-4-yl)pyrido[3,4-d]pyrimidin-2-amine

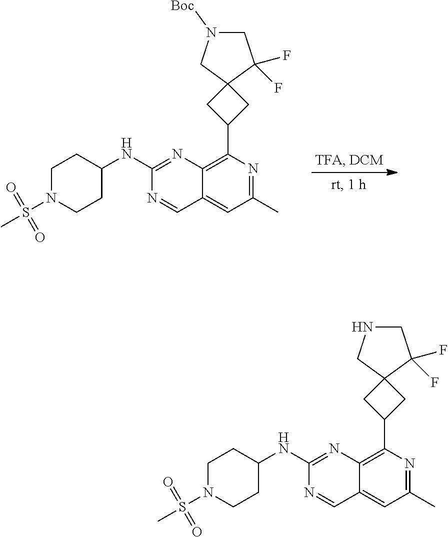 [SMILES: Cc1cc2cnc(NC3CCN(S(C)(=O)=O)CC3)nc2c(C2CC3(C2)CN(C(=O)OC(C)(C)C)CC3(F)F)n1 ; Cc1cc2cnc(NC3CCN(S(C)(=O)=O)CC3)nc2c(C2CC3(CNCC3(F)F)C2)n1]

TFA (0.23 mL) was added to a solution of tert-butyl 8,8-difluoro-2-(6-methyl-2-((1-(methylsulfonyl)piperidin-4-yl)amino)pyrido[3,4-d]pyrimidin-8-yl)-6-azaspiro[3.4]octane-6-carboxylate (20 mg, 0.035 mmol) in DCM (3 mL). The resulting mixture was stirred for 1 h at room temperature and concentrated under reduced pressure. The residue was purified by Prep-TLC (DCM/MeOH, 10:1) to afford 8-(8,8-difluoro-6-azaspiro[3.4]octan-2-yl)-6-methyl-N-(1-(methylsulfonyl)piperidin-4-yl)pyrido[3,4-d]pyrimidin-2-amine (1.0 mg, 5.65% yield).

¹H NMR (400 MHz, CD₃OD) δ 8.04 (s, 1H), 7.32 (s, 1H), 4.10-3.99 (m, 1H), 3.80-3.70 (m, 2H), 3.24-3.13 (m, 2H), 3.09-2.96 (m, 2H), 2.91-2.88 (m, 4H), 2.84-2.77 (m, 2H), 2.61 (s, 3H), 2.52-2.44 (m, 1H), 2.36-2.15 (m, 4H), 1.84-1.66 (m, 2H), 0.94-0.83 (m, 2H).

LCMS (ESI-MS) m/z=467.3 [M+H]⁺.

### Example 97: 8-(8-(difluoromethyl)-2,6-diazaspiro[3.4]octan-2-yl)-6-methyl-N-(1-(methylsulfonyl)piperidin-4-yl)pyrido[3,4-d]pyrimidin-2-amine

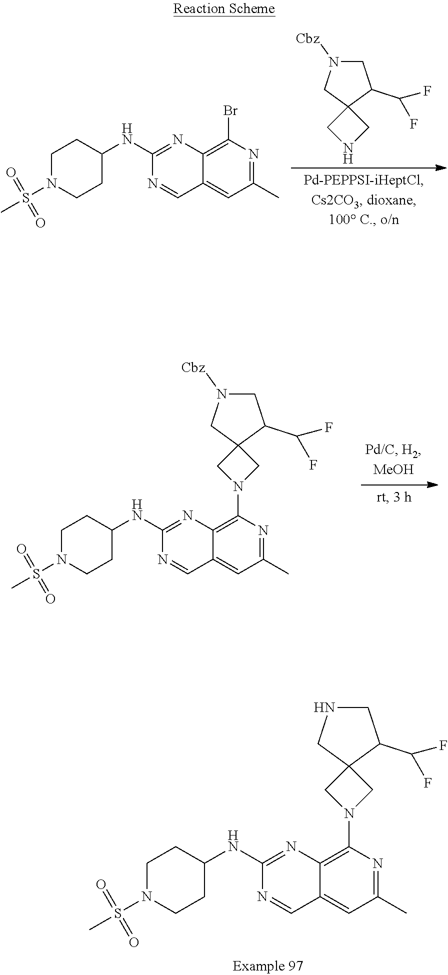

### Step 1: Benzyl 8-(difluoromethyl)-2-(6-methyl-2-((1-(methylsulfonyl)piperidin-4-yl)amino)pyrido[3,4-d]pyrimidin-8-yl)-2,6-diazaspiro[3.4]octane-6-carboxylate

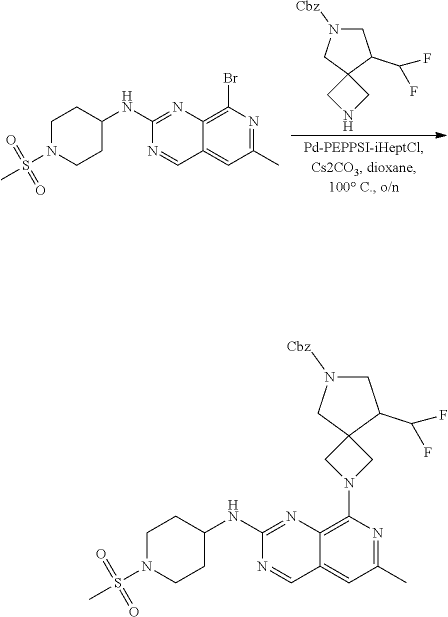

Pd-PEPPSI-iHeptCl3-chloropyridine (31.55 mg, 0.03 mmol) was added to a mixture of benzyl 8-(difluoromethyl)-2,6-diazaspiro[3.4]octane-6-carboxylate (96 mg, 0.32 mmol), 8-bromo-6-methyl-N-(1-(methylsulfonyl)piperidin-4-yl)pyrido[3,4-d]pyrimidin-2-amine (129.69 mg, 0.32 mmol) and Cs₂CO₃ (211.12 mg, 0.64 mmol) in dioxane (3 mL) under a nitrogen atmosphere. The reaction mixture was stirred overnight at 100° C. under a nitrogen atmosphere. The resulting mixture was filtered and the filter cake was washed with DCM (20 mL). The filtrate was concentrated under reduced pressure. The residue was purified by Prep-TLC (CH₂Cl₂/MeOH, 10:1) to afford benzyl 8-(difluoromethyl)-2-(6-methyl-2-((1-(methylsulfonyl)piperidin-4-yl)amino)pyrido[3,4-d]pyrimidin-8-yl)-2,6-diazaspiro[3.4]octane-6-carboxylate (150 mg, 58.43% yield).

LCMS (ES−MS) m/z=616.3 [M+H]⁺.

### Step 2: 8-(8-(difluoromethyl)-2,6-diazaspiro[3.4]octan-2-yl)-6-methyl-N-(1-(methylsulfonyl)piperidin-4-yl)pyrido[3,4-d]pyrimidin-2-amine

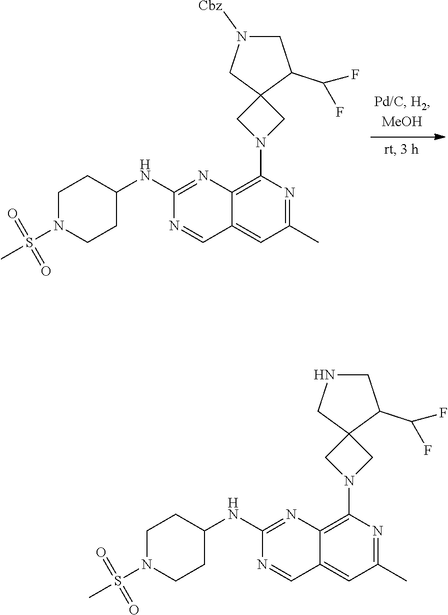 [SMILES: Cc1cc2cnc(NC3CCN(S(C)(=O)=O)CC3)nc2c(N2CC3(CN(C(=O)OCc4ccccc4)CC3C(F)F)C2)n1 ; Cc1cc2cnc(NC3CCN(S(C)(=O)=O)CC3)nc2c(N2CC3(CNCC3C(F)F)C2)n1]

Pd/C (116.67 mg, 0.11 mmol, 10% on carbon) was added to a solution of benzyl 8-(difluoromethyl)-2-(6-methyl-2-((1-(methylsulfonyl)piperidin-4-yl)amino)pyrido[3,4-d]pyrimidin-8-yl)-2,6-diazaspiro[3.4]octane-6-carboxylate (135 mg, 0.21 mmol) in MeOH (5 mL) under a nitrogen atmosphere. The reaction mixture was stirred for 3 h at room temperature under a hydrogen atmosphere. The resulting mixture was filtered and the filter cake was washed with MeOH (20 mL). The filtrate was concentrated under reduced pressure. The residue was purified by Prep-TLC (CH₂Cl₂/MeOH, 10:1). The product was further purified by preparative RP-HPLC to afford 8-(8-(difluoromethyl)-2,6-diazaspiro[3.4]octan-2-yl)-6-methyl-N-(1-(methylsulfonyl)piperidin-4-yl)pyrido[3,4-d]pyrimidin-2-amine (14.6 mg, 13.25% yield).

¹H NMR (400 MHz, Chloroform-d) δ 9.04-8.68 (m, 1H), 7.45-7.11 (m, 1H), 6.64 (d, J=4.1 Hz, 1H), 6.03 (t, J=55.8 Hz, 1H), 5.32-5.06 (m, 1H), 4.92-4.68 (m, 1H), 4.34 (s, 3H), 4.10-3.91 (m, 1H), 3.89-3.73 (m, 2H), 3.51-3.07 (m, 4H), 3.05-2.92 (m, 2H), 2.91-2.78 (m, 3H), 2.73-2.56 (m, 1H), 2.56-2.35 (m, 3H), 2.22 (s, 2H), 1.83-1.65 (m, 2H).

LCMS (ES−MS) m/z=482.4 [M+H]⁺.

### Examples 100 and 101: (R)-8-(8-fluoro-6-methyl-2,6-diazaspiro[3.4]octan-2-yl)-6-methyl-N-(1-(methylsulfonyl)piperidin-4-yl)pyrido[3,4-d]pyrimidin-2-amine and (S)-8-(8-fluoro-6-methyl-2,6-diazaspiro[3.4]octan-2-yl)-6-methyl-N-(1-(methylsulfonyl)piperidin-4-yl)pyrido[3,4-d]pyrimidin-2-amine

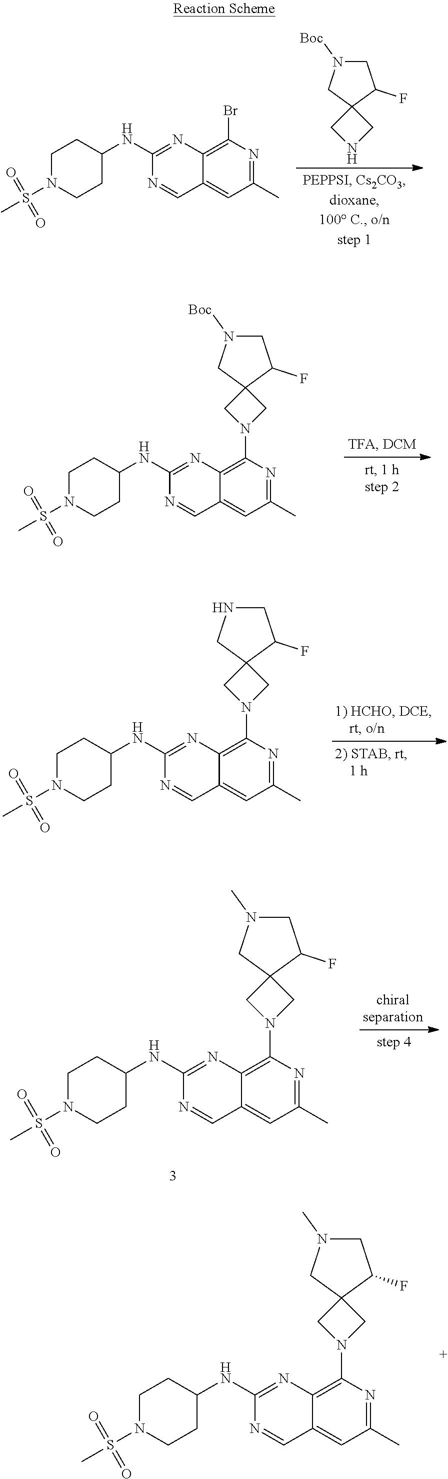

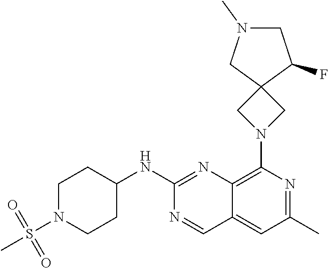 [SMILES: Cc1cc2cnc(NC3CCN(S(C)(=O)=O)CC3)nc2c(N2CC3(CN(C)C[C@H]3F)C2)n1]

### Detailed Procedure

### Step 1: Tert-butyl8-fluoro-2-(6-methyl-2-((1-(methylsulfonyl)piperidin-4-yl)amino)pyrido[3,4-d]pyrimidin-8-yl)-2,6-diazaspiro[3.4]octane-6-carboxylate

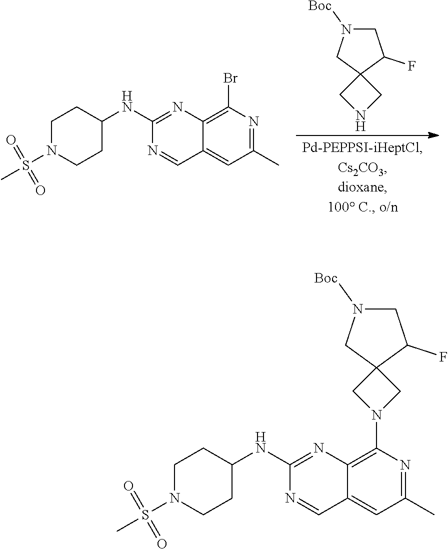

To a solution of 8-bromo-6-methyl-N-(1-(methylsulfonyl)piperidin-4-yl)pyrido[3,4-d]pyrimidin-2-amine (180 mg, 0.45 mmol) in dioxane (1 mL, 11.80 mmol) was added tert-butyl 8-fluoro-2,6-diazaspiro[3.4]octane-6-carboxylate (103.55 mg, 0.45 mmol), Pd-PEPPSI-iHeptCl 3-chloropyridine (43.79 mg, 0.04 mmol) and Cs₂CO₃ (293.02 mg, 0.90 mmol) under a nitrogen atmosphere. The resulting mixture was stirred overnight at 100° C. under a nitrogen atmosphere. The mixture was filtered and the filter cake was washed with DCM (20 mL). The filtrate was concentrated under reduced pressure. The residue was purified by silica gel column chromatography (PE/EA, 1:9) to afford tert-butyl8-fluoro-2-(6-methyl-2-((1-(methylsulfonyl)piperidin-4-yl)amino)pyrido[3,4-d]pyrimidin-8-yl)-2,6-diazaspiro[3.4]octane-6-carboxylate (180 mg, 58.26% yield).

LCMS (ES−MS) m/z=550.2 [M+H]⁺.

### Step 2: 8-(8-fluoro-2,6-diazaspiro[3.4]octan-2-yl)-6-methyl-N-(1-(methylsulfonyl)piperidin-4-yl)pyrido[3,4-d]pyrimidin-2-amine

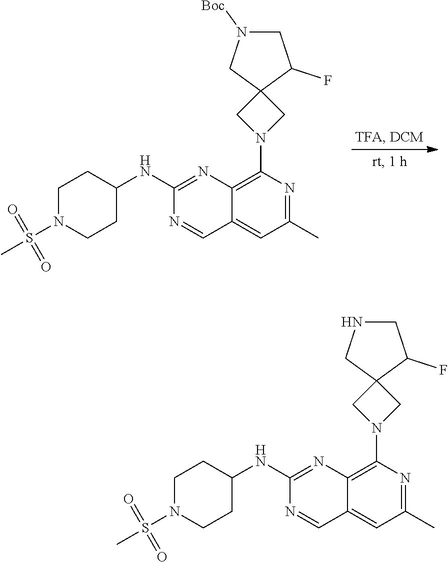 [SMILES: Cc1cc2cnc(NC3CCN(S(C)(=O)=O)CC3)nc2c(N2CC3(CN(C(=O)OC(C)(C)C)CC3F)C2)n1 ; Cc1cc2cnc(NC3CCN(S(C)(=O)=O)CC3)nc2c(N2CC3(CNCC3F)C2)n1]

A solution of tert-butyl8-fluoro-2-(6-methyl-2-((1-(methylsulfonyl)piperidin-4-yl)amino)pyrido[3,4-d]pyrimidin-8-yl)-2,6-diazaspiro[3.4]octane-6-carboxylate (180 mg, 0.327 mmol) in TFA (0.6 mL) and DCM (2 mL) was stirred at room temperature for 1 h. The reaction mixture was concentrated under reduced pressure to afford crude 8-(8-fluoro-2,6-diazaspiro[3.4]octan-2-yl)-6-methyl-N-(1-(methylsulfonyl)piperidin-4-yl)pyrido[3,4-d]pyrimidin-2-amine (145 mg, 78.80% yield).

LCMS (ES−MS) m/z=450.2 [M+H]⁺.

### Step 3: 8-(8-fluoro-6-methyl-2,6-diazaspiro[3.4]octan-2-yl)-6-methyl-N-(1-(methylsulfonyl)piperidin-4-yl)pyrido[3,4-d]pyrimidin-2-amine

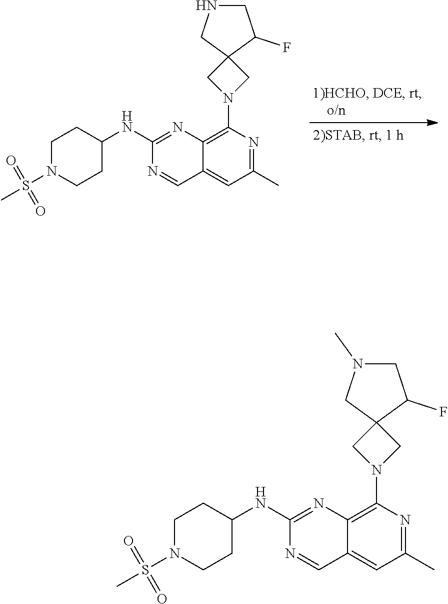 [SMILES: Cc1cc2cnc(NC3CCN(S(C)(=O)=O)CC3)nc2c(N2CC3(CNCC3F)C2)n1 ; Cc1cc2cnc(NC3CCN(S(C)(=O)=O)CC3)nc2c(N2CC3(CN(C)CC3F)C2)n1]

A mixture of 8-(8-fluoro-2,6-diazaspiro[3.4]octan-2-yl)-6-methyl-N-(1-(methylsulfonyl)piperidin-4-yl) pyrido[3,4-d]pyrimidin-2-amine (135 mg, 0.300 mmol) and HCHO (10.82 mg, 0.36 mmol) in DCE (1.5 mL) was stirred overnight at room temperature. STAB (95.47 mg, 0.45 mmol) was added and the resulting mixture was stirred for 1 h at room temperature. The mixture was purified by silica gel column chromatography (DCM/MeOH, 10:1) to afford racemic 8-(8-fluoro-6-methyl-2,6-diazaspiro[3.4]octan-2-yl)-6-methyl-N-(1-(methylsulfonyl)piperidin-4-yl)pyrido[3,4-d]pyrimidin-2-amine 10.2 mg, 7.09%).

LCMS (ES−MS) m/z=464.2 [M+H]⁺.

### Step 4: (R)-8-(8-fluoro-6-methyl-2,6-diazaspiro[3.4]octan-2-yl)-6-methyl-N-(1-(methylsulfonyl)piperidin-4-yl)pyrido[3,4-d]pyrimidin-2-amine and (S)-8-(8-fluoro-6-methyl-2,6-diazaspiro[3.4]octan-2-yl)-6-methyl-N-(1-(methylsulfonyl)piperidin-4-yl)pyrido[3,4-d]pyrimidin-2-amine

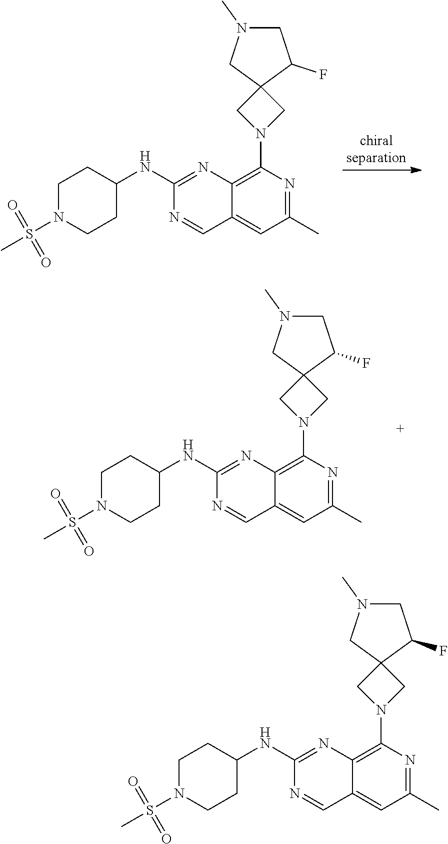 [SMILES: Cc1cc2cnc(NC3CCN(S(C)(=O)=O)CC3)nc2c(N2CC3(CN(C)CC3F)C2)n1 ; Cc1cc2cnc(NC3CCN(S(C)(=O)=O)CC3)nc2c(N2CC3(CN(C)C[C@@H]3F)C2)n1 ; Cc1cc2cnc(NC3CCN(S(C)(=O)=O)CC3)nc2c(N2CC3(CN(C)C[C@H]3F)C2)n1]

Racemic 8-(8-fluoro-6-methyl-2,6-diazaspiro[3.4]octan-2-yl)-6-methyl-N-(1-(methylsulfonyl) piperidin-4-yl)pyrido[3,4-d]pyrimidin-2-amine was separated by Prep-Chiral-HPLC using the following conditions:
Column: CHIRAL Cellulose-SB, 4.6*100 mm, 3 μm.
Mobile Phase: Hex (0.1% DEA)/(EtOH/DCM, 1:1), 75:25.

The desired fractions were combined and lyophilized to afford the title products as single enantiomers.

### First Eluting Peak:

17.4 mg, 99.8% ee, 7.09% yield.

¹H NMR (400 MHz, DMSO-d₆) δ 8.94 (s, 1H), 7.45-7.41 (m, 1H), 6.70 (s, 1H), 5.30-5.15 (m, 1H), 4.54-4.17 (m, 3H), 3.82 (s, 1H), 3.57-3.54 (m, 2H), 2.96-2.88 (m, 5H), 2.84-2.82 (m, 2H), 2.67-2.50 (m, 3H), 2.49-2.28 (m, 6H), 2.12-1.95 (m, 2H), 1.71-1.52 (m, 2H).

LCMS (ES−MS) m/z=464.2 [M+H]⁺.

### Second Eluting Peak:

14.4 mg, 99.6% ee, 9.85% yield

¹H NMR (400 MHz, DMSO-d₆) δ 8.94 (s, 1H), 7.49-7.43 (m, 1H), 6.70 (s, 1H), 5.30-5.15 (m, 1H), 4.53-4.13 (m, 3H), 3.82 (s, 1H), 3.58-3.56 (m, 2H), 3.01-2.82 (m, 7H), 2.67-2.63 (m, 3H), 2.50-2.28 (m, 6H), 2.02-1.99 (m, 2H), 1.71-1.59 (m, 2H).

LCMS (ES−MS) m/z=464.2 [M+H]⁺.

In some embodiments, compounds of the disclosure are below in Table 1.

TABLE 1

| Com- | | | Mass |
|---|---|---|---|
| pound | | | Spec. |
| No. | Structure | Name | M + H/1 |
| 1 | 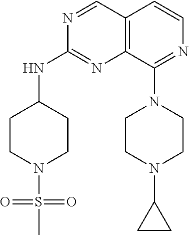 [SMILES: CS(=O)(=O)N1CCC(Nc2ncc3ccnc(N4CCN(C5CC5)CC4)c3n2)CC1] | 8-(4- cyclopropylpiperazin- 1-yl)-N-(1- (methylsulfonyl) piperidin-4- yl)pyrido[3,4- d]pyrimidin-2-amine | 432.2 |
| 2 | 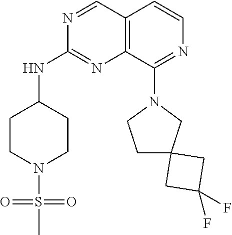 [SMILES: CS(=O)(=O)N1CCC(Nc2ncc3ccnc(N4CCC5(C4)CC(F)(F)C5)c3n2)CC1] | 8-(2,2-difluoro-6- azaspiro[3.4]octan- 6-yl)-N-(1- (methylsulfonyl) piperidin-4- yl)pyrido[3,4- d]pyrimidin-2-amine | 453 |
| 3 | 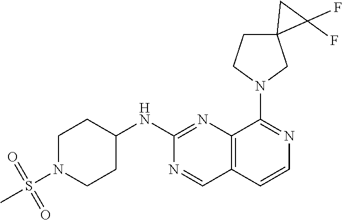 [SMILES: CS(=O)(=O)N1CCC(Nc2ncc3ccnc(N4CCC5(C4)CC5(F)F)c3n2)CC1] | 8-(1,1-difluoro-5- azaspiro[2.4]heptan- 5-yl)-N-(1- (methylsulfonyl) piperidin-4- yl)pyrido[3,4- d]pyrimidin-2-amine | 439.2 |
| 4 | 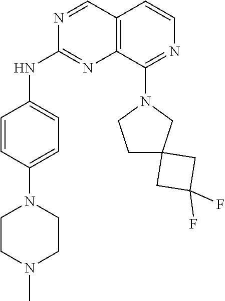 [SMILES: CN1CCN(c2ccc(Nc3ncc4ccnc(N5CCC6(C5)CC(F)(F)C6)c4n3)cc2)CC1] | 8-(2,2-difluoro-6- azaspiro[3.4]octan- 6-yl)-N-(4-(4- methylpiperazin-1- yl)phenyl)pyrido [3,4-d]pyrimidin- 2-amine | 466.2 |
| 5 | 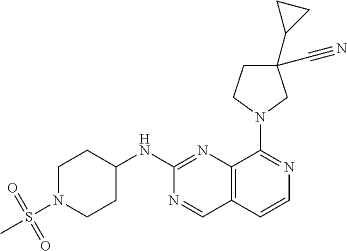 [SMILES: CS(=O)(=O)N1CCC(Nc2ncc3ccnc(N4CCC(C#N)(C5CC5)C4)c3n2)CC1] | 3-cyclopropyl-1-(2- ((1- (methylsulfonyl) piperidin-4- yl)amino)pyrido[3,4 -d]pyrimidin-8- yl)pyrrolidine-3- carbonitrile | 442.2 |
| 6 | 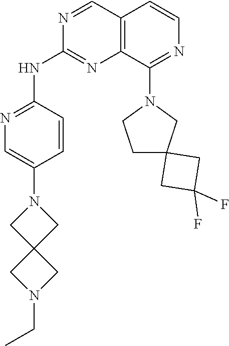 [SMILES: CCN1CC2(C1)CN(c1ccc(Nc3ncc4ccnc(N5CCC6(C5)CC(F)(F)C6)c4n3)nc1)C2] | 8-(2,2-difluoro-6- azaspiro[3.4]octan- 6-yl)-N-(5-(6-ethyl- 2,6- diazaspiro[3.3] heptan-2-yl) pyridin-2- yl)pyrido[3,4- d]pyrimidin- 2-amine | 493 |
| 7 | 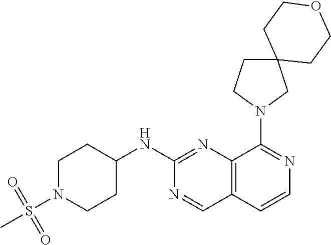 [SMILES: CS(=O)(=O)N1CCC(Nc2ncc3ccnc(N4CCC5(CCOCC5)C4)c3n2)CC1] | N-(1- (methylsulfonyl) piperidin-4-yl)- 8-(8-oxa-2- azaspiro[4.5]decan- 2-yl)pyrido[3,4- d]pyrimidin- 2-amine | 447.2 |
| 8 | 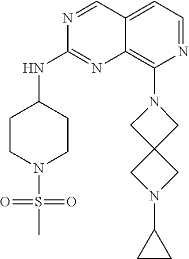 [SMILES: CS(=O)(=O)N1CCC(Nc2ncc3ccnc(N4CC5(C4)CN(C4CC4)C5)c3n2)CC1] | 8-(6-cyclopropyl- 2,6- diazaspiro[3.3] heptan-2-yl)-N-(1- (methylsulfonyl) piperidin-4- yl)pyrido[3,4- d]pyrimidin- 2-amine | 444.2 |
| 9 | 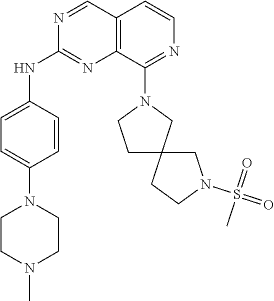 [SMILES: CN1CCN(c2ccc(Nc3ncc4ccnc(N5CCC6(CCN(S(C)(=O)=O)C6)C5)c4n3)cc2)CC1] | N-(4-(4- methylpiperazin-1- yl)phenyl)-8-(7- (methylsulfonyl)- 2,7- diazaspiro[4.4] nonan-2-yl) pyrido[3,4- d]pyrimidin- 2-amine | 523.3 |
| 10 | 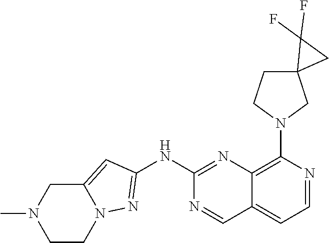 [SMILES: CN1CCn2nc(Nc3ncc4ccnc(N5CCC6(C5)CC6(F)F)c4n3)cc2C1] | 8-(1,1-difluoro-5- azaspiro[2.4]heptan- 5-yl)-N-(5-methyl- 4,5,6,7- tetrahydropyrazolo [1,5-a]pyrazin-2- yl)pyrido[3,4- d]pyrimidin- 2-amine | 413.1 |
| 11 | 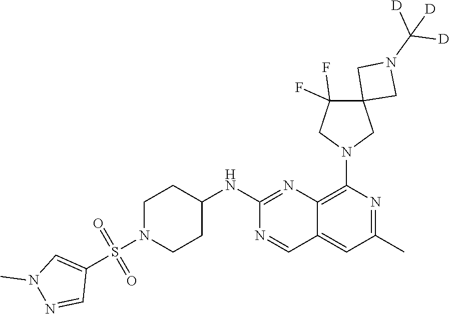 [SMILES: [2H]C([2H])([2H])N1CC2(CN(c3nc(C)cc4cnc(NC5CCN(S(=O)(=O)c6cnn(C)c6)CC5)nc34)CC2(F)F)C1] | 8-(8,8-difluoro-2- (methyl-d3)-2,6- diazaspiro[3.4]octan -6-yl)-6-methyl-N- (1-((1-methyl-1H- pyrazol-4- yl)sulfonyl) piperidin-4- yl)pyrido[3,4- d]pyrimidin- 2-amine | 551.3 |
| 12 | 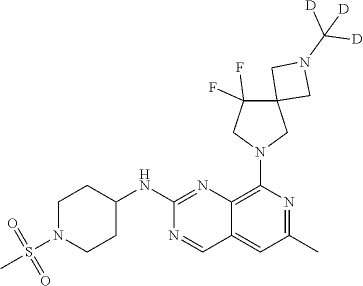 [SMILES: [2H]C([2H])([2H])N1CC2(CN(c3nc(C)cc4cnc(NC5CCN(S(C)(=O)=O)CC5)nc34)CC2(F)F)C1] | 8-(8,8-difluoro-2- (methyl-d3)-2,6- diazaspiro[3.4] octan-6-yl)-6- methyl-N-(1- (methylsulfonyl) piperidin-4- yl)pyrido [3,4-d]pyrimidin- 2-amine | 485.3 |
| 13 | 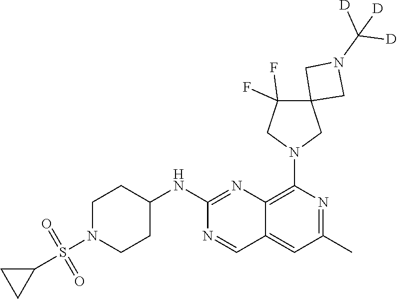 [SMILES: [2H]C([2H])([2H])N1CC2(CN(c3nc(C)cc4cnc(NC5CCN(S(=O)(=O)C6CC6)CC5)nc34)CC2(F)F)C1] | N-(1- (cyclopropylsulfonyl) piperidin-4-yl)-8- (8,8-difluoro-2- (methyl-d3)-2,6- diazaspiro[3.4] octan-6-yl)-6- methylpyrido[3,4- d]pyrimidin- 2-amine | 511.3 |
| 14 | 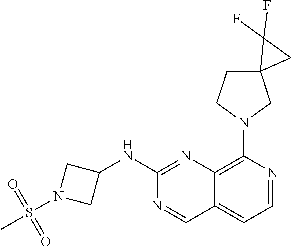 [SMILES: CS(=O)(=O)N1CC(Nc2ncc3ccnc(N4CCC5(C4)CC5(F)F)c3n2)C1] | 8-(1,1-difluoro-5- azaspiro[2.4]heptan- 5-yl)-N-(1- (methylsulfonyl) azetidin-3-yl) pyrido[3,4- d]pyrimidin- 2-amine | 411.1 |
| 15 | 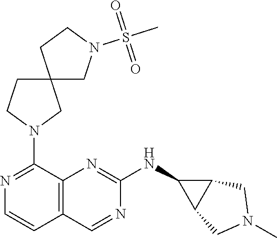 [SMILES: CN1C[C@@H]2[C@H](C1)[C@H]2Nc1ncc2ccnc(N3CCC4(CCN(S(C)(=O)=O)C4)C3)c2n1] | N-((1R,5S,6s)-3- methyl-3- azabicyclo[3.1.0] hexan-6-yl)-8-(7- (methylsulfonyl)- 2,7- diazaspiro[4.4] nonan-2-yl) pyrido[3,4- d]pyrimidin- 2-amine | 444.1 |
| 16 | 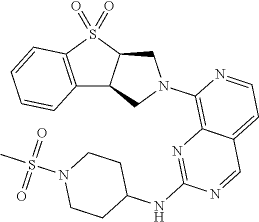 [SMILES: CS(=O)(=O)N1CCC(Nc2ncc3ccnc(N4C[C@H]5c6ccccc6S(=O)(=O)[C@H]5C4)c3n2)CC1] | (3aR,8bR)-2-(2-((1- (methylsulfonyl) piperidin-4- yl)amino)pyrido[3,4 -d]pyrimidin-8-yl)- 2,3,3a,8b- tetrahydro-1H- benzo[4,5]thieno [2,3-c]pyrrole 4,4-dioxide | 515 |
| 17 | 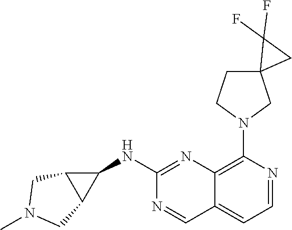 [SMILES: CN1C[C@@H]2[C@H](C1)[C@H]2Nc1ncc2ccnc(N3CCC4(C3)CC4(F)F)c2n1] | 8-(1,1-difluoro-5- azaspiro[2.4]heptan- 5-yl)-N-((1R,5S,6s)- 3-methyl-3- azabicyclo[3.1.0] hexan-6-yl) pyrido[3,4- d]pyrimidin- 2-amine | 373.1 |
| 18 | 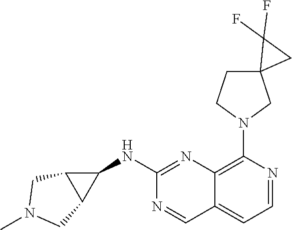 [SMILES: CN1C[C@@H]2[C@H](C1)[C@H]2Nc1ncc2ccnc(N3CCC4(C3)CC4(F)F)c2n1] | N-(5-(6-ethyl-2,6- diazaspiro[3.3] heptan-2-yl) pyridin-2-yl)- 8-(8-oxa-2- azaspiro[4.5]decan- 2-yl)pyrido[3,4- d]pyrimidin- 2-amine | 487.2 |
| 19 | 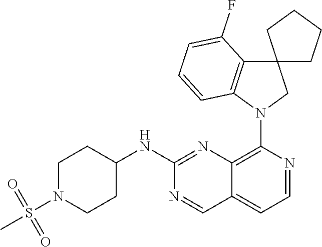 [SMILES: CS(=O)(=O)N1CCC(Nc2ncc3ccnc(N4CC5(CCCC5)c5c(F)cccc54)c3n2)CC1] | 8-(4′- fluorospiro [cyclopentane- 1,3′-indolin]-1′- yl)-N-(1- (methylsulfonyl) piperidin-4- yl)pyrido[3,4- d]pyrimidin- 2-amine | 497.1 |
| 20 | 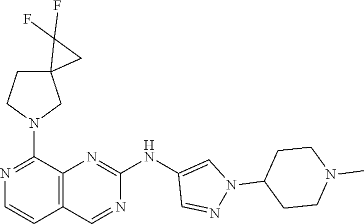 [SMILES: CN1CCC(n2cc(Nc3ncc4ccnc(N5CCC6(C5)CC6(F)F)c4n3)cn2)CC1] | 8-(1,1-difluoro-5- azaspiro[2.4]heptan- 5-yl)-N-(1-(1- methylpiperidin-4- yl)-1H-pyrazol-4- yl)pyrido[3,4- d]pyrimidin- 2-amine | 441.2 |
| 21 | 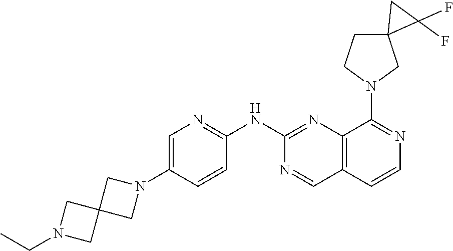 [SMILES: CCN1CC2(C1)CN(c1ccc(Nc3ncc4ccnc(N5CCC6(C5)CC6(F)F)c4n3)nc1)C2] | 8-(1,1-difluoro-5- azaspiro[2.4]heptan- 5-yl)-N-(5-(6-ethyl- 2,6- diazaspiro[3.3] heptan-2-yl) pyridin-2- yl)pyrido[3,4- d]pyrimidin- 2-amine | 479.2 |
| 22 | 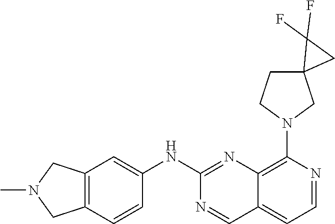 [SMILES: CN1Cc2ccc(Nc3ncc4ccnc(N5CCC6(C5)CC6(F)F)c4n3)cc2C1] | 8-(1,1-difluoro-5- azaspiro[2.4]heptan- 5-yl)-N-(2- methylisoindolin-5- yl)pyrido[3,4- d]pyrimidin- 2-amine | 409.2 |
| 23 | 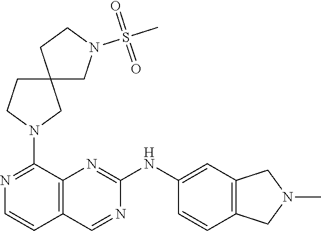 [SMILES: CN1Cc2ccc(Nc3ncc4ccnc(N5CCC6(CCN(S(C)(=O)=O)C6)C5)c4n3)cc2C1] | N-(2- methylisoindolin-5- yl)-8-(7- (methylsulfonyl)- 2,7- diazaspiro[4.4] nonan-2-yl) pyrido[3,4- d]pyrimidin- 2-amine | 480.2 |
| 24 | 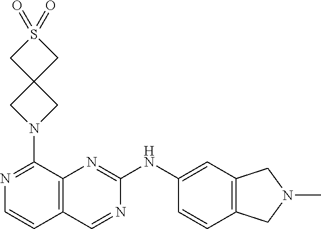 [SMILES: CN1Cc2ccc(Nc3ncc4ccnc(N5CC6(C5)CS(=O)(=O)C6)c4n3)cc2C1] | 6-(2-((2- methylisoindolin-5- yl)amino)pyrido [3,4-d]pyrimidin- 8-yl)-2-thia-6- azaspiro[3.3] heptane2,2-dioxide | 423.1 |
| 25 | 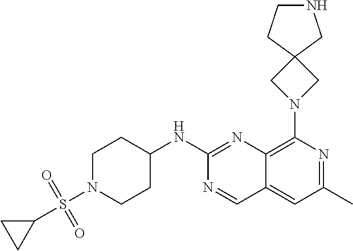 [SMILES: Cc1cc2cnc(NC3CCN(S(=O)(=O)C4CC4)CC3)nc2c(N2CC3(CCNC3)C2)n1] | N-(1- (cyclopropylsulfonyl) piperidin-4-yl)-6- methyl-8-(2,6- diazaspiro[3.4] octan-2-yl) pyrido[3,4- d]pyrimidin- 2-amine | 458.3 |
| 26 | 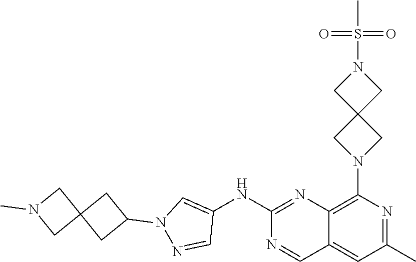 | 6-methyl-N-(1-(2- methyl-2- azaspiro[3.3]heptan- 6-yl)-1H-pyrazol-4- yl)-8-(6- (methylsulfonyl)- 2,6- diazaspiro[3.3] heptan-2-yl) pyrido[3,4- d]pyrimidin- 2-amine | 510.1 |
| 27 | 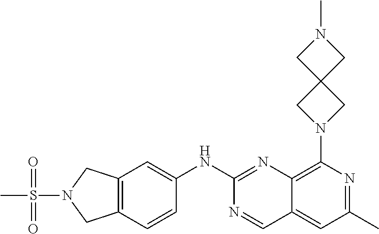 | 6-methyl-8-(6- methyl-2,6- diazaspiro[3.3] heptan-2-yl)-N-(2- (methylsulfonyl) isoindolin-5- yl)pyrido[3,4- d]pyrimidin- 2-amine | 466.2 |
| 28 | 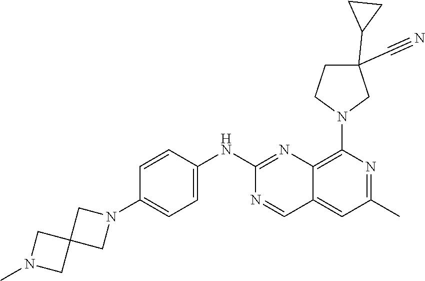 [SMILES: Cc1cc2cnc(Nc3ccc(N4CC5(CN(C)C5)C4)cc3)nc2c(N2CCC(C#N)(C3CC3)C2)n1] | 3-cyclopropyl-1-(6- methyl-2-((4-(6- methyl-2,6- diazaspiro[3.3] heptan-2- yl)phenyl)amino) pyrido[3,4-d] pyrimidin- 8-yl)pyrrolidine-3- carbonitrile | 481.3 |
| 29 | 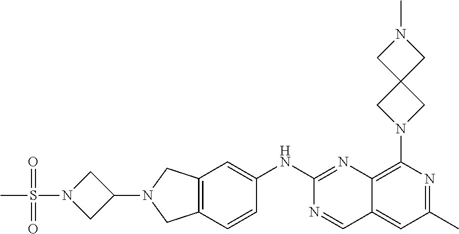 | 6-methyl-8-(6- methyl-2,6- diazaspiro[3.3] heptan-2-yl)- N-(2-(1- (methylsulfonyl) azetidin-3-yl) isoindolin- 5-yl)pyrido[3,4- d]pyrimidin- 2-amine | 521.3 |
| 30 | 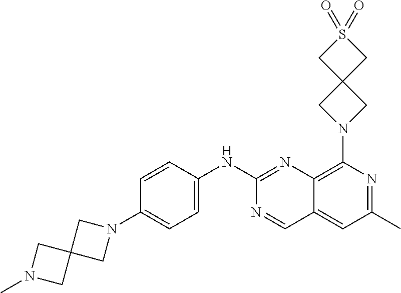 | 6-(6-methyl-2-((4- (6-methyl-2,6- diazaspiro[3.3] heptan-2- yl)phenyl)amino) pyrido[3,4-d] pyrimidin- 8-yl)-2-thia-6- azaspiro[3.3]heptane 2,2-dioxide | 492.1 |
| 31 | 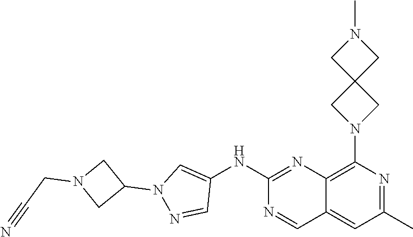 [SMILES: Cc1cc2cnc(Nc3cnn(C4CN(CC#N)C4)c3)nc2c(N2CC3(CN(C)C3)C2)n1] | 2-(3-(4-((6-methyl- 8-(6-methyl-2,6- diazaspiro[3.3] heptan-2-yl) pyrido[3,4- d]pyrimidin-2- yl)amino)-1H- pyrazol-1- yl)azetidin-1- yl)acetonitrile | 431.2 |
| 32 | 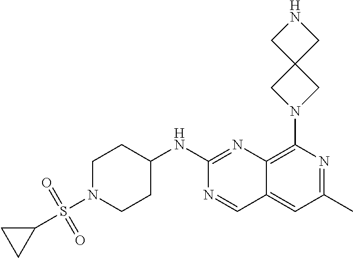 [SMILES: Cc1cc2cnc(NC3CCN(S(=O)(=O)C4CC4)CC3)nc2c(N2CC3(CNC3)C2)n1] | N-(1- (cyclopropylsulfonyl) piperidin-4-yl)-6- methyl-8-(2,6- diazaspiro[3.3] heptan-2-yl) pyrido[3,4- d]pyrimidin- 2-amine | 444.2 |
| 33 | 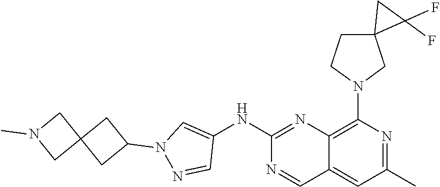 [SMILES: Cc1cc2cnc(Nc3cnn(C4CC5(C4)CN(C)C5)c3)nc2c(N2CCC3(C2)CC3(F)F)n1] | 8-(1,1-difluoro-5- azaspiro[2.4]heptan- 5-yl)-6-methyl-N- (1-(2-methyl-2- azaspiro[3.3]heptan- 6-yl)-1H-pyrazol-4- yl)pyrido[3,4- d]pyrimidin-2-amine | 467.2 |
| 34 | 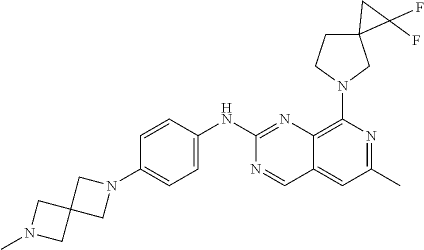 [SMILES: Cc1cc2cnc(Nc3ccc(N4CC5(CN(C)C5)C4)cc3)nc2c(N2CCC3(C2)CC3(F)F)n1] | 8-(1,1-difluoro-5- azaspiro[2.4]heptan- 5-yl)-6-methyl-N- (4-(6-methyl-2,6- diazaspiro[3.3] heptan-2- yl)phenyl)pyrido [3,4-d]pyrimidin-2- amine | 478.2 |
| 35 | 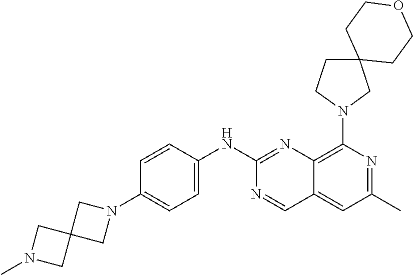 [SMILES: Cc1cc2cnc(Nc3ccc(N4CC5(CN(C)C5)C4)cc3)nc2c(N2CCC3(CCOCC3)C2)n1] | 6-methyl-N-(4-(6- methyl-2,6- diazaspiro[3.3] heptan-2-yl) phenyl)-8-(8- oxa-2- azaspiro[4.5]decan- 2-yl)pyrido[3,4- d]pyrimidin-2-amine | 486.2 |
| 36 | 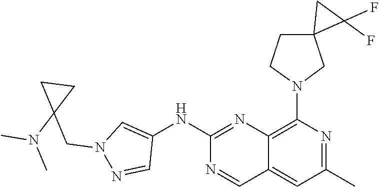 [SMILES: Cc1cc2cnc(Nc3cnn(CC4(N(C)C)CC4)c3)nc2c(N2CCC3(C2)CC3(F)F)n1] | 8-(1,1-difluoro-5- azaspiro[2.4]heptan- 5-yl)-N-(1-((1- (dimethylamino) cyclopropyl)methyl)- 1H-pyrazol-4-yl)-6- methylpyrido[3,4- d]pyrimidin-2-amine | 455.1 |
| 37 | 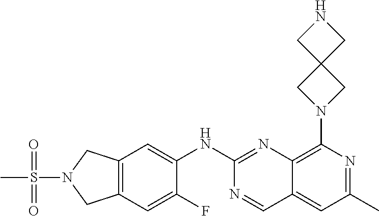 | N-(6-fluoro-2- (methylsulfonyl) isoindolin-5-yl)-6- methyl-8-(2,6- diazaspiro[3.3] heptan-2-yl) pyrido[3,4- d]pyrimidin-2-amine | 470.2 |
| 38 | 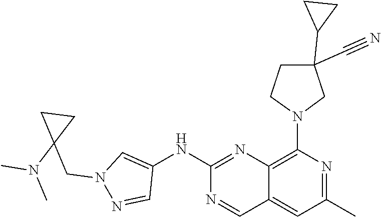 [SMILES: Cc1cc2cnc(Nc3cnn(CC4(N(C)C)CC4)c3)nc2c(N2CCC(C#N)(C3CC3)C2)n1] | 3-cyclopropyl-1-(2- ((1-((1- (dimethylamino) cyclopropyl) methyl)- 1H-pyrazol-4- yl)amino)-6- methylpyrido[3,4- d]pyrimidin-8- yl)pyrrolidine-3- carbonitrile | 458.2 |
| 39 | 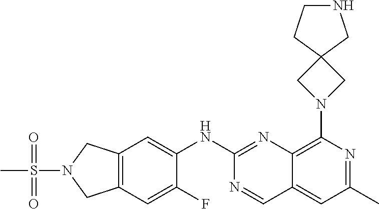 | N-(6-fluoro-2- (methylsulfonyl) isoindolin-5-yl)-6- methyl-8-(2,6- diazaspiro [3.4]octan-2- yl)pyrido[3,4- d]pyrimidin- 2-amine | 484.2 |
| 40 | 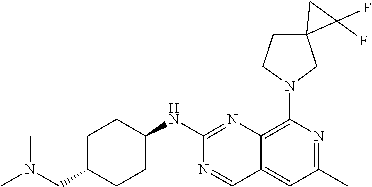 [SMILES: Cc1cc2cnc(N[C@H]3CC[C@H](CN(C)C)CC3)nc2c(N2CCC3(C2)CC3(F)F)n1] | 8-(1,1-difluoro-5- azaspiro[2.4]heptan- 5-yl)-N-((1r,4r)-4- ((dimethylamino) methyl) cyclohexyl)-6- methylpyrido[3,4- d]pyrimidin- 2-amine | 431.2 |
| 41 | 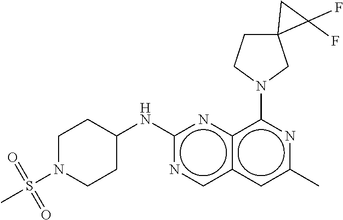 | 8-(1,1-difluoro-5- azaspiro[2.4]heptan- 5-yl)-6-methyl-N-(1- (methylsulfonyl) piperidin-4- yl)pyrido[3,4- d]pyrimidin- 2-amine | 453.3 |
| 42 | 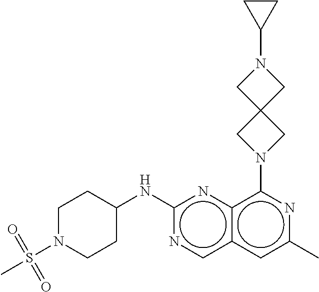 | 8-(6-cyclopropyl- 2,6- diazaspiro[3.3] heptan-2-yl)-6- methyl-N-(1- (methylsulfonyl) piperidin-4- yl)pyrido[3,4- d]pyrimidin- 2-amine | 458.3 |
| 43 | 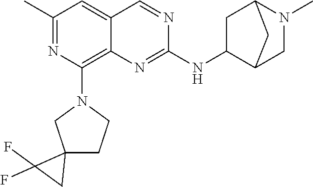 [SMILES: Cc1cc2cnc(NC3CC4CC3CN4C)nc2c(N2CCC3(C2)CC3(F)F)n1] | 8-(1,1-difluoro-5- azaspiro[2.4]heptan- 5-yl)-6-methyl-N- (2-methyl-2- azabicyclo[2.2.1] heptan-5-yl) pyrido[3,4- d]pyrimidin- 2-amine | 401.2 |
| 44 | 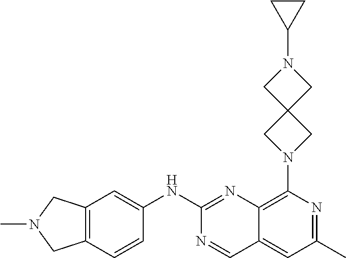 | 8-(6-cyclopropyl- 2,6- diazaspiro[3.3] heptan-2-yl)-6- methyl-N- (2-methylisoindolin- 5-yl)pyrido[3,4- d]pyrimidin-2-amine | 428.3 |
| 45 | 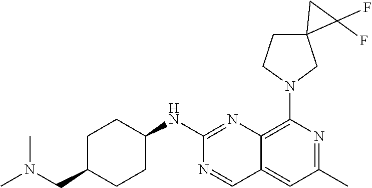 [SMILES: Cc1cc2cnc(N[C@H]3CC[C@@H](CN(C)C)CC3)nc2c(N2CCC3(C2)CC3(F)F)n1] | 8-(1,1-difluoro-5- azaspiro[2.4]heptan- 5-yl)-N-((1s,4s)-4- ((dimethylamino) methyl)cyclohexyl)- 6-methylpyrido[3,4- d]pyrimidin-2-amine | 431.2 |
| 46 | 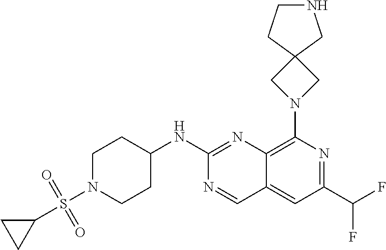 [SMILES: O=S(=O)(C1CC1)N1CCC(Nc2ncc3cc(C(F)F)nc(N4CC5(CCNC5)C4)c3n2)CC1] | 1- (cyclopropanesulfonyl)- N-(8-{2,6- diazaspiro[3.4] octan-2-yl}-6- (difluoromethyl) pyrido[3,4-d]pyrimidin- 2-yl)piperidin-4- amine | 494.3 |
| 47 | 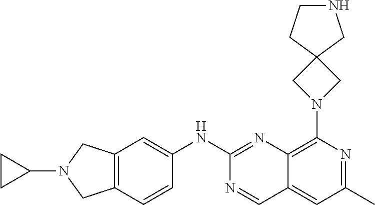 [SMILES: Cc1cc2cnc(Nc3ccc4c(c3)CN(C3CC3)C4)nc2c(N2CC3(CCNC3)C2)n1] | N-(2- cyclopropylisoindolin- 5-yl)-6-methyl-8- (2,6- diazaspiro[3.4]octan- 2-yl)pyrido[3,4- dlpyrimidin-2-amine | 428.3 |
| 48 | 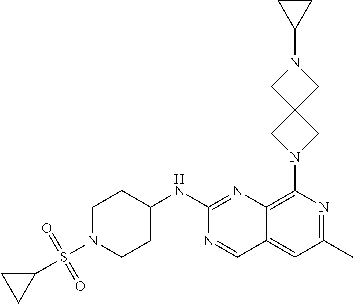 [SMILES: Cc1cc2cnc(NC3CCN(S(=O)(=O)C4CC4)CC3)nc2c(N2CC3(C2)CN(C2CC2)C3)n1] | 8-(6-cyclopropyl- 2,6- diazaspiro[3.3] heptan-2-yl)-N-(1- (cyclopropylsulfonyl) piperidin-4-yl)-6- methylpyrido[3,4- d]pyrimidin-2-amine | 484.2 |
| 49 | 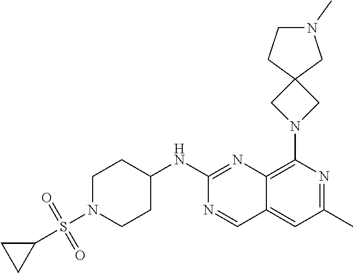 [SMILES: Cc1cc2cnc(NC3CCN(S(=O)(=O)C4CC4)CC3)nc2c(N2CC3(CCN(C)C3)C2)n1] | N-(1- (cyclopropylsulfonyl) piperidin-4-yl)-6- methyl-8-(6-methyl- 2,6- diazaspiro[3.4] octan-2-yl)pyrido[3,4- d]pyrimidin-2-amine | 472.2 |
| 50 | 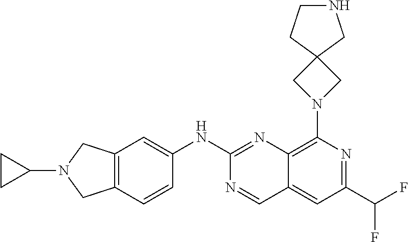 [SMILES: FC(F)c1cc2cnc(Nc3ccc4c(c3)CN(C3CC3)C4)nc2c(N2CC3(CCNC3)C2)n1] | N-(2- cyclopropylisoindolin- 5-yl)-6- (difluoromethyl)-8- (2,6- diazaspiro[3.4]octan- 2-yl)pyrido[3,4- d]pyrimidin-2-amine | 464.2 |
| 51 | 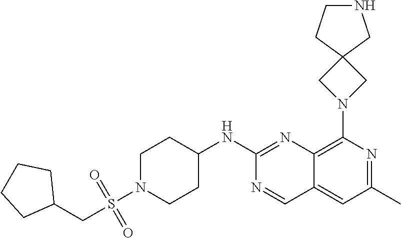 [SMILES: Cc1cc2cnc(NC3CCN(S(=O)(=O)CC4CCCC4)CC3)nc2c(N2CC3(CCNC3)C2)n1] | N-(1- ((cyclopentylmethyl) sulfonyl)piperidin- 4-yl)-6-methyl-8- (2,6- diazaspiro[3.4]octan- 2-yl)pyrido[3,4- d]pyrimidin-2-amine | 500.3 |
| 52 | 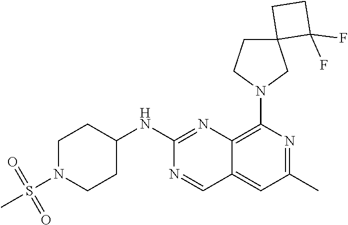 [SMILES: Cc1cc2cnc(NC3CCN(S(C)(=O)=O)CC3)nc2c(N2CCC3(CCC3(F)F)C2)n1] | 8-(1,1-difluoro-6- azaspiro[3.4]octan- 6-yl)-6-methyl-N- (1-(methylsulfonyl) piperidin-4- yl)pyrido[3,4- d]pyrimidin-2-amine | 467.3 |
| 53 | 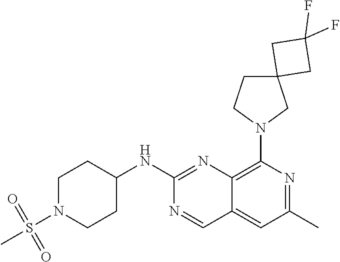 [SMILES: Cc1cc2cnc(NC3CCN(S(C)(=O)=O)CC3)nc2c(N2CCC3(C2)CC(F)(F)C3)n1] | 8-(2,2-difluoro-6- azaspiro[3.4]octan- 6-yl)-6-methyl-N- (1-(methylsulfonyl) piperidin-4- yl)pyrido[3,4- d]pyrimidin-2-amine | 467.3 |
| 54 | 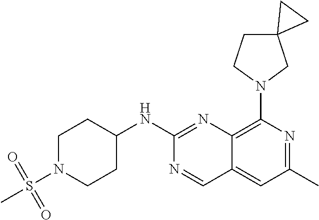 | 6-methyl-N-(1- (methylsulfonyl) piperidin-4-yl)-8-(5- azaspiro[2.4]heptan- 5-yl)pyrido[3,4- d]pyrimidin-2-amine | 417.3 |
| 55 | 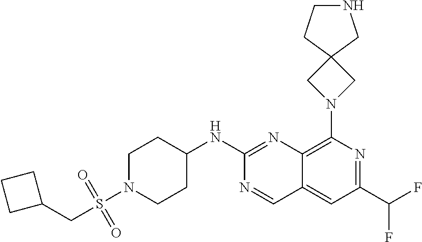 [SMILES: O=S(=O)(CC1CCC1)N1CCC(Nc2ncc3cc(C(F)F)nc(N4CC5(CCNC5)C4)c3n2)CC1] | N-(1- ((cyclobutylmethyl) sulfonyl)piperidin-4- yl)-6- (difluoromethyl)-8- (2,6- diazaspiro[3.4]octan- 2-yl)pyrido[3,4- d\|pyrimidin-2-amine | 522.3 |
| 56 | 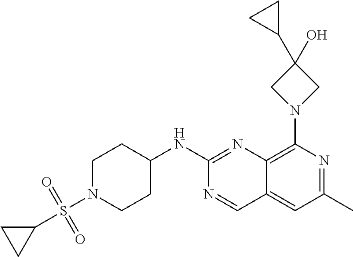 [SMILES: Cc1cc2cnc(NC3CCN(S(=O)(=O)C4CC4)CC3)nc2c(N2CC(O)(C3CC3)C2)n1] | 3-cyclopropyl-1-(2- ((1- (cyclopropylsulfonyl) piperidin-4- yl)amino)-6- methylpyrido[3,4- d]pyrimidin-8- yl)azetidin-3-ol | 459.2 |
| 57 | 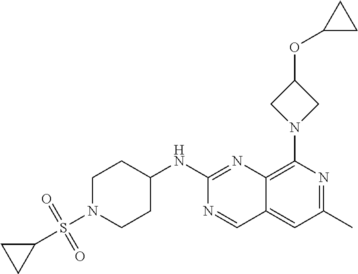 [SMILES: Cc1cc2cnc(NC3CCN(S(=O)(=O)C4CC4)CC3)nc2c(N2CC(OC3CC3)C2)n1] | 8-(3- cyclopropoxyazetidin- 1-yl)-N-(1- (cyclopropylsulfonyl) piperidin-4-yl)-6- methylpyrido[3,4- d]pyrimidin-2-amine | 459.3 |
| 58 | 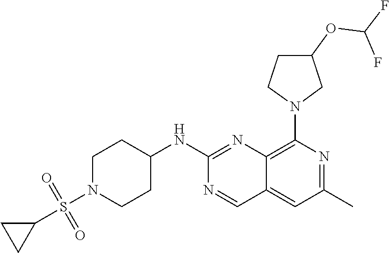 [SMILES: Cc1cc2cnc(NC3CCN(S(=O)(=O)C4CC4)CC3)nc2c(N2CCC(OC(F)F)C2)n1] | N-(1- (cyclopropylsulfonyl) piperidin-4-yl)-8-(3- (difluoromethoxy) pyrrolidin-1-yl)-6- methylpyrido[3,4- d]pyrimidin-2-amine | 483.3 |
| 59 | 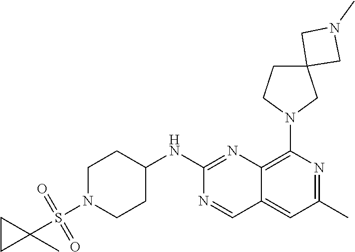 [SMILES: Cc1cc2cnc(NC3CCN(S(=O)(=O)C4(C)CC4)CC3)nc2c(N2CCC3(CN(C)C3)C2)n1] | 6-methyl-8-(2- methyl-2,6- diazaspiro[3.4]octan- 6-yl)-N-(1-((1- methylcyclopropyl) sulfonyl)piperidin-4- yl)pyrido[3,4- d]pyrimidin-2-amine | 486.3 |
| 60 | 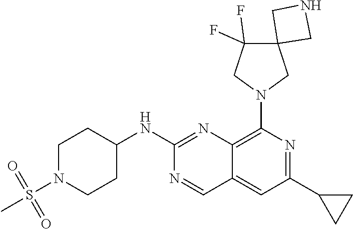 [SMILES: CS(=O)(=O)N1CCC(Nc2ncc3cc(C4CC4)nc(N4CC(F)(F)C5(CNC5)C4)c3n2)CC1] | 6-cyclopropyl-8- (8,8-difluoro-2,6- diazaspiro[3.4]octan- 6-yl)-N-(1- (methylsulfonyl) piperidin-4- yl)pyrido[3,4- d]pyrimidin-2-amine | 494.3 |
| 61 | 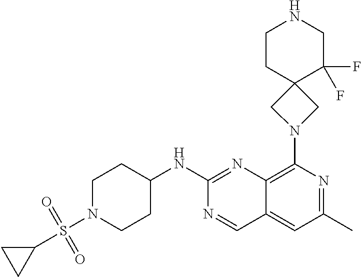 [SMILES: Cc1cc2cnc(NC3CCN(S(=O)(=O)C4CC4)CC3)nc2c(N2CC3(CCNCC3(F)F)C2)n1] | N-(1- (cyclopropylsulfonyl) piperidin-4-yl)-8- (5,5-difluoro-2,7- diazaspiro[3.5] nonan-2-yl)-6- methylpyrido[3,4- d]pyrimidin-2-amine | 508.3 |
| 62 | 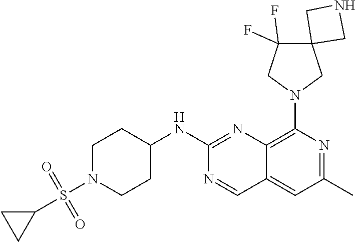 [SMILES: Cc1cc2cnc(NC3CCN(S(=O)(=O)C4CC4)CC3)nc2c(N2CC(F)(F)C3(CNC3)C2)n1] | N-(1- (cyclopropylsulfonyl) piperidin-4-yl)-8- (8,8-difluoro-2,6- diazaspiro[3.4]octan- 6-yl)-6- methylpyrido[3,4- d]pyrimidin-2-amine | 494.2 |
| 63 | 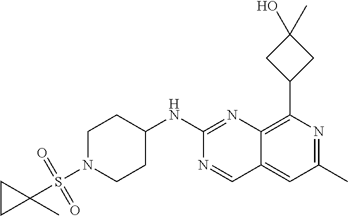 [SMILES: Cc1cc2cnc(NC3CCN(S(=O)(=O)C4(C)CC4)CC3)nc2c(C2CC(C)(O)C2)n1] | 1-methyl-3-(6- methyl-2-((1-((1- methylcyclopropyl)s ulfonyl)piperidin-4- yl)amino)pyrido[3,4 -d]pyrimidin-8- yl)cyclobutan-1-ol | 446.2 |
| 64 | 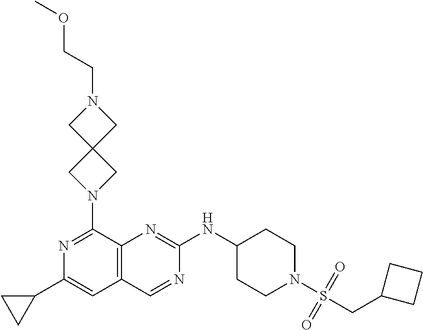 [SMILES: COCCN1CC2(C1)CN(c1nc(C3CC3)cc3cnc(NC4CCN(S(=O)(=O)CC5CCC5)CC4)nc13)C2] | N-(1- ((cyclobutylmethyl) sulfonyl)piperidin-4- yl)-6-cyclopropyl-8- (6-(2- methoxyethyl)-2,6- diazaspiro[3.3] heptan-2-yl)pyrido [3,4-d]pyrimidin- 2-amine | 556.3 |
| 65 | 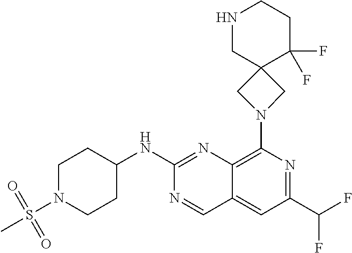 [SMILES: CS(=O)(=O)N1CCC(Nc2ncc3cc(C(F)F)nc(N4CC5(CNCCC5(F)F)C4)c3n2)CC1] | 8-(9,9-difluoro-2,6- diazaspiro[3.5] nonan-2-yl)-6- (difluoromethyl)-N- (1-(methylsulfonyl) piperidin-4- yl)pyrido[3,4- d\|pyrimidin-2-amine | 518.2 |
| 66 | 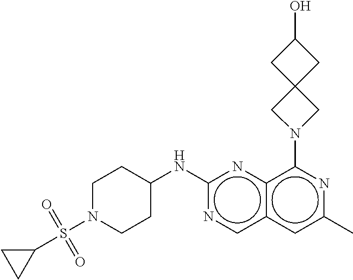 | 2-(2-((1- (cyclopropylsulfonyl) piperidin-4- yl)amino)-6- methylpyrido[3,4- d]pyrimidin-8-yl)-2- azaspiro[3.3]heptan- 6-ol | 459.2 |
| 67 | 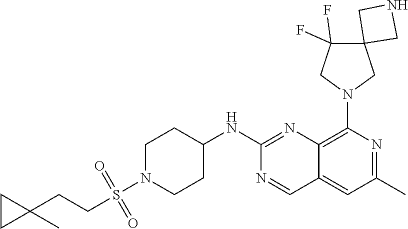 [SMILES: Cc1cc2cnc(NC3CCN(S(=O)(=O)CCC4(C)CC4)CC3)nc2c(N2CC(F)(F)C3(CNC3)C2)n1] | 8-(8,8-difluoro-2,6- diazaspiro[3.4]octan- 6-yl)-6-methyl-N- (1-((2-(1- methylcyclopropyl) ethyl)sulfonyl) piperidin-4-yl) pyrido[3,4- d]pyrimidin-2-amine | 536.3 |
| 68 | 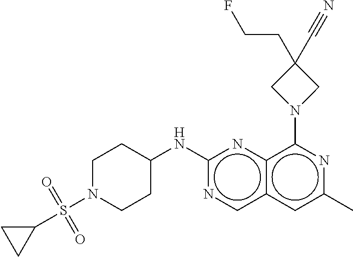 | 1-(2-((1- (cyclopropylsulfonyl) piperidin-4- yl)amino)-6- methylpyrido[3,4- d]pyrimidin-8-yl)-3- (2-fluoroethyl) azetidine-3- carbonitrile | 474.3 |
| 69 | 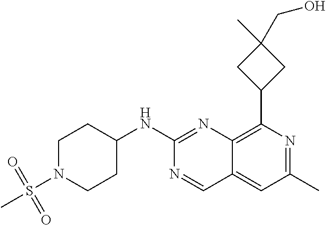 | (1-methyl-3-(6- methyl-2-((1- (methylsulfonyl) piperidin-4- yl)amino)pyrido[3,4- d]pyrimidin-8- yl)cyclobutyl) methanol | 421.2 |
| 70 | 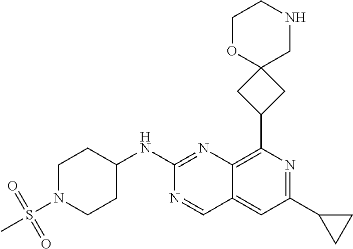 [SMILES: CS(=O)(=O)N1CCC(Nc2ncc3cc(C4CC4)nc(C4CC5(CNCCO5)C4)c3n2)CC1] | 6-cyclopropyl-N-(1- (methylsulfonyl) piperidin-4-yl)-8-(5- oxa-8- azaspiro[3.5]nonan- 2-yl)pyrido[3,4- d]pyrimidin-2-amine | 473.2 |
| 71 | 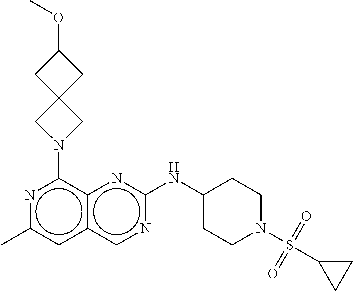 | N-(1- (cyclopropylsulfonyl) piperidin-4-yl)-8- (6-methoxy-2- azaspiro[3.3]heptan- 2-yl)-6- methylpyrido[3,4- d]pyrimidin-2-amine | 473.3 |
| 72 | 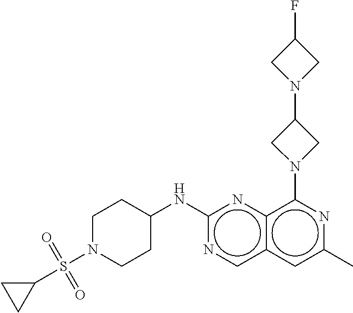 | N-(1- (cyclopropylsulfonyl) piperidin-4-yl)-8- (3-fluoro-[1,3′- biazetidin]-1′-yl)-6- methylpyrido[3,4- d]pyrimidin-2-amine | 476.2 |
| 73 | 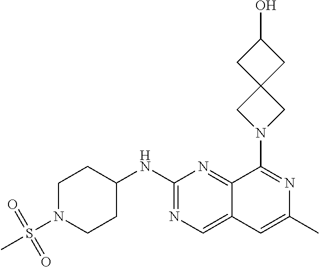 | 2-(6-methyl-2-((1- (methylsulfonyl) piperidin-4- yl)amino)pyrido[3,4- d]pyrimidin-8-yl)-2- azaspiro[3.3]heptan- 6-ol | 433.2 |
| 74 | 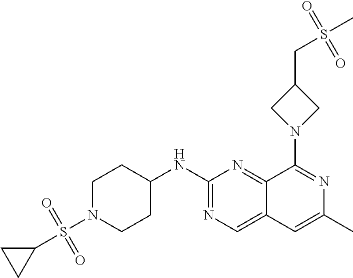 [SMILES: Cc1cc2cnc(NC3CCN(S(=O)(=O)C4CC4)CC3)nc2c(N2CC(CS(C)(=O)=O)C2)n1] | N-(1- (cyclopropylsulfonyl) piperidin-4-yl)-6- methyl-8-(3- ((methylsulfonyl) methyl)azetidin-1- yl)pyrido[3,4- d]pyrimidin-2-amine | 495.2 |
| 75 | 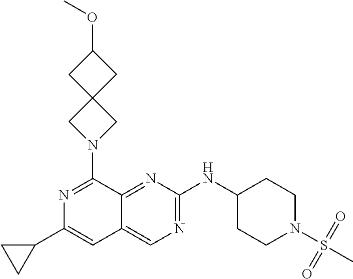 [SMILES: COC1CC2(C1)CN(c1nc(C3CC3)cc3cnc(NC4CCN(S(C)(=O)=O)CC4)nc13)C2] | 6-cyclopropyl-8-(6- methoxy-2- azaspiro[3.3]heptan- 2-yl)-N-(1- (methylsulfonyl) piperidin-4- yl)pyrido[3,4- d]pyrimidin-2-amine | 473.3 |
| 76 | 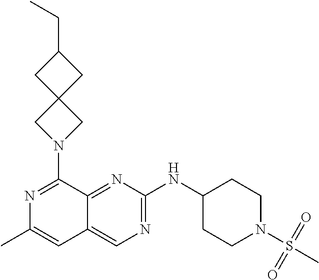 | 8-(6-methoxy-2- azaspiro[3.3]heptan- 2-yl)-6-methyl-N-(1- (methylsulfonyl) piperidin-4- yl)pyrido[3,4- d]pyrimidin-2-amine | 447.2 |
| 77 | 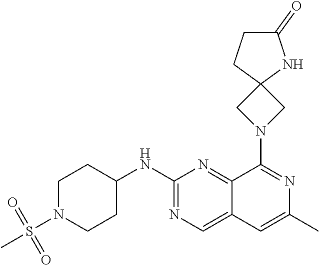 | 2-(6-methyl-2-((1- (methylsulfonyl) piperidin-4- yl)amino)pyrido[3,4- d]pyrimidin-8-yl)- 2,5-diazaspiro[3.4] octan-6-one | 446.2 |
| 78 | 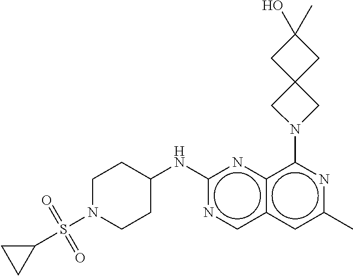 | 2-(2-((1- (cyclopropylsulfonyl) piperidin-4- yl)amino)-6- methylpyrido[3,4- d]pyrimidin-8-yl)-6- methyl-2- azaspiro[3.3]heptan- 6-ol | 473.2 |
| 79 | 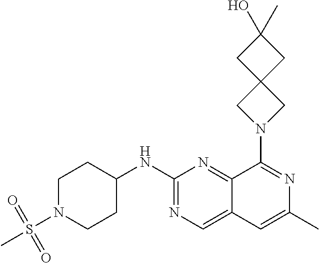 | 6-methyl-2-(6- methyl-2-((1- (methylsulfonyl) piperidin-4- yl)amino)pyrido[3,4- d]pyrimidin-8-yl)- 2- azaspiro[3.3]heptan- 6-ol | 447.2 |
| 80 | 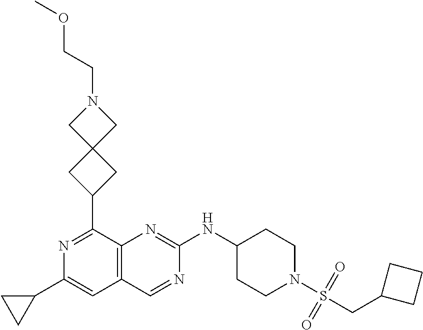 [SMILES: COCCN1CC2(CC(c3nc(C4CC4)cc4cnc(NC5CCN(S(=O)(=O)CC6CCC6)CC5)nc34)C2)C1] | N-(1- ((cyclobutylmethyl) sulfonyl)piperidin-4- yl)-6-cyclopropyl-8- (2-(2- methoxyethyl)-2- azaspiro[3.3]heptan- 6-yl)pyrido[3,4- d]pyrimidin-2-amine | 555.4 |
| 81 | 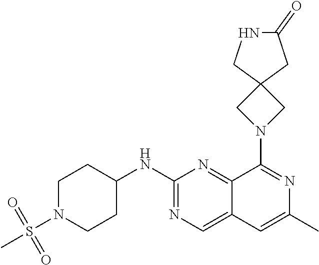 | 2-(6-methyl-2-((1- (methylsulfonyl) piperidin-4- yl)amino)pyrido[3,4- d]pyrimidin-8-yl)- 2,6-diazaspiro[3.4] octan-7-one | 446.2 |
| 82 | 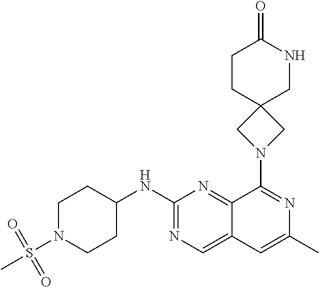 | 2-(6-methyl-2-((1- (methylsulfonyl) piperidin-4- yl)amino)pyrido[3,4- d]pyrimidin-8-yl)- 2,6-diazaspiro[3.5] nonan-7-one | 460.2 |
| 83 | 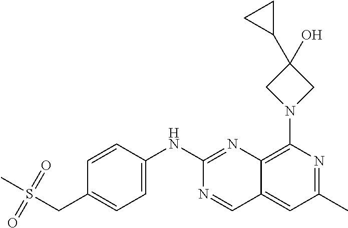 | 3-cyclopropyl-1-(6- methyl-2-((4- ((methylsulfonyl) methyl)phenyl)amino) pyrido[3,4- d]pyrimidin-8- yl)azetidin-3-ol | 440.1 |
| 84 | 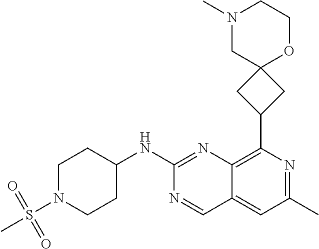 | 6-methyl-8-(8- methyl-5-oxa-8- azaspiro[3.5]nonan- 2-yl)-N-(1- (methylsulfonyl) piperidin-4- yl)pyrido[3,4- d]pyrimidin-2-amine | 461.3 |
| 85 | 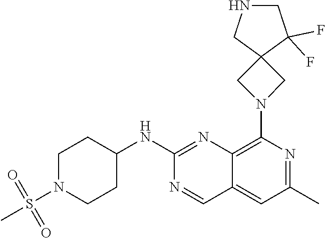 [SMILES: Cc1cc2cnc(NC3CCN(S(C)(=O)=O)CC3)nc2c(N2CC3(CNCC3(F)F)C2)n1] | 8-(8,8-difluoro-2,6- diazaspiro[3.4]octan- 2-yl)-6-methyl-N- (1-(methylsulfonyl) piperidin-4- yl)pyrido[3,4- d]pyrimidin-2-amine | 468.3 |
| 86 | 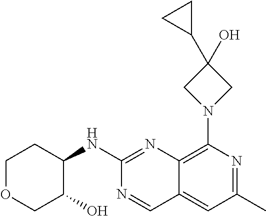 [SMILES: Cc1cc2cnc(N[C@@H]3CCOC[C@H]3O)nc2c(N2CC(O)(C3CC3)C2)n1] | 3-cyclopropyl-1-(2- (((3S,4R)-3- hydroxytetrahydro- 2H-pyran-4- yl)amino)-6- methylpyrido[3,4- d]pyrimidin-8- yl)azetidin-3-ol | 372.2 |
| 87 | 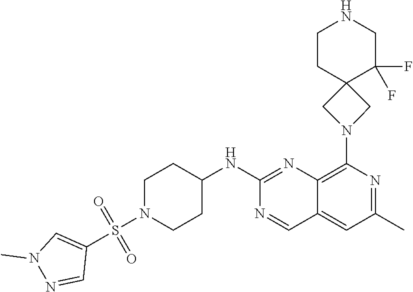 [SMILES: Cc1cc2cnc(NC3CCN(S(=O)(=O)c4cnn(C)c4)CC3)nc2c(N2CC3(CCNCC3(F)F)C2)n1] | 8-(5,5-difluoro-2,7- diazaspiro[3.5]nonan- 2-yl)-6-methyl-N- (1-((1-methyl-1H- pyrazol-4- yl)sulfonyl)piperidin- 4-yl)pyrido[3,4- d]pyrimidin-2-amine | 548.3 |
| 88 | 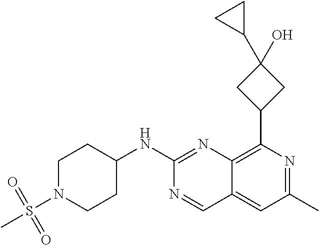 | 1-cyclopropyl-3-(6- methyl-2-((1- (methylsulfonyl) piperidin-4- yl)amino)pyrido[3,4- d]pyrimidin-8- yl)cyclobutan-1-ol | 432.2 |
| 89 | 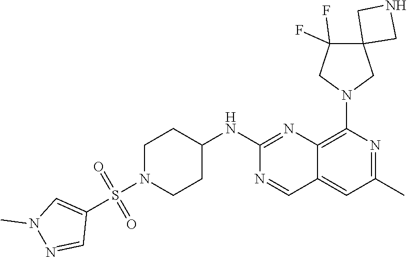 | 8-(8,8-difluoro-2,6- diazaspiro[3.4]octan- 6-yl)-6-methyl-N- (1-((1-methyl-1H- pyrazol-4- yl)sulfonyl)piperidin- 4-yl)pyrido[3,4- d]pyrimidin-2-amine | 534.2 |
| 90 | 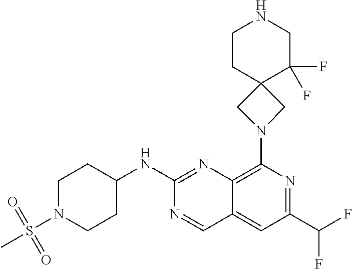 [SMILES: CS(=O)(=O)N1CCC(Nc2ncc3cc(C(F)F)nc(N4CC5(CCNCC5(F)F)C4)c3n2)CC1] | 8-(5,5-difluoro-2,7- diazaspiro[3.5] nonan-2-yl)-6- (difluoromethyl)-N- (1-(methylsulfonyl) piperidin-4- yl)pyrido[3,4- d]pyrimidin-2-amine | 518.2 |
| 91 | 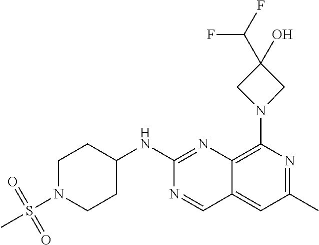 | 3-(difluoromethyl)- 1-(6-methyl-2-((1- (methylsulfonyl) piperidin-4- yl)amino)pyrido[3,4 -d]pyrimidin-8- yl)azetidin-3-ol | 443.1 |
| 92 | 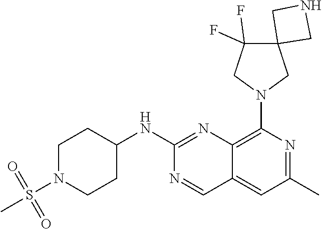 | 8-(8,8-difluoro-2,6- diazaspiro[3.4]octan- 6-yl)-6-methyl-N- (1-(methylsulfonyl) piperidin-4- yl)pyrido[3,4- d]pyrimidin-2-amine | 468.1 |
| 93 | 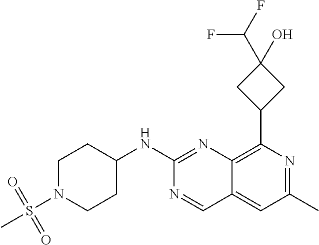 | 1-(difluoromethyl)- 3-(6-methyl-2-((1- (methylsulfonyl) piperidin-4-yl)amino) pyrido[3,4- d]pyrimidin-8- yl)cyclobutan-1-ol | 442.1 |
| 94 | 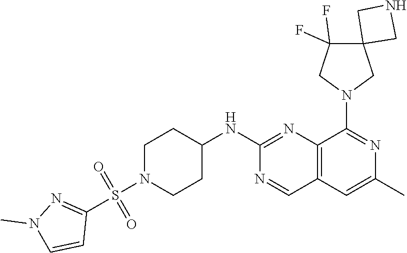 | 8-(8,8-difluoro-2,6- diazaspiro[3.4]octan- 6-yl)-6-methyl-N- (1-((1-methyl-1H- pyrazol-3- yl)sulfonyl)piperidin- 4-yl)pyrido[3,4- d]pyrimidin-2-amine | 534 |
| 95 | 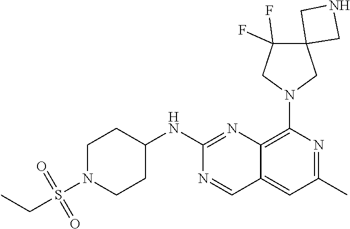 [SMILES: CCS(=O)(=O)N1CCC(Nc2ncc3cc(C)nc(N4CC(F)(F)C5(CNC5)C4)c3n2)CC1] | 8-(8,8-difluoro-2,6- diazaspiro[3.4]octan- 6-yl)-N-(1- (ethylsulfonyl) piperidin-4-yl)-6- methylpyrido[3,4- d]pyrimidin-2-amine | 482 |
| 96 | 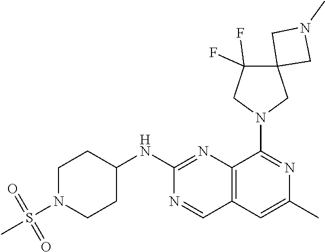 | 8-(8,8-difluoro-2- methyl-2,6- diazaspiro[3.4]octan- 6-yl)-6-methyl-N- (1-(methylsulfonyl) piperidin-4- yl)pyrido[3,4- d]pyrimidin-2-amine | 482.2 |
| 97 | 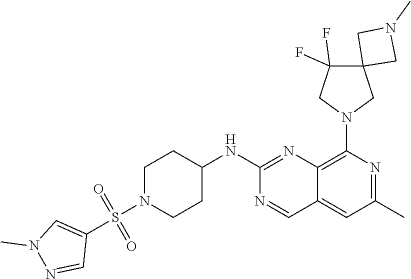 | 8-(8,8-difluoro-2- methyl-2,6- diazaspiro[3.4]octan- 6-yl)-6-methyl-N- (1-((1-methyl-1H- pyrazol-4- yl)sulfonyl)piperidin- 4-yl)pyrido[3,4- d]pyrimidin-2-amine | 548.4 |
| 98 | 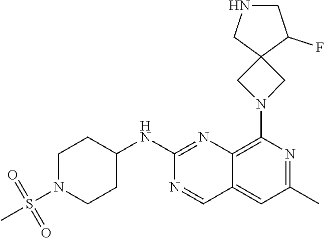 [SMILES: Cc1cc2cnc(NC3CCN(S(C)(=O)=O)CC3)nc2c(N2CC3(CNCC3F)C2)n1] | 8-(8-fluoro-2,6- diazaspiro[3.4]octan- 2-yl)-6-methyl-N- (1-(methylsulfonyl) piperidin-4- yl)pyrido[3,4- d]pyrimidin-2-amine | 450.1 |
| 99 | 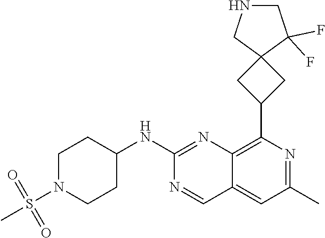 [SMILES: Cc1cc2cnc(NC3CCN(S(C)(=O)=O)CC3)nc2c(C2CC3(CNCC3(F)F)C2)n1] | 8-(8,8-difluoro-6- azaspiro[3.4]octan- 2-yl)-6-methyl-N- (1-(methylsulfonyl) piperidin-4- yl)pyrido[3,4- d]pyrimidin-2-amine | 467.3 |
| 100 | 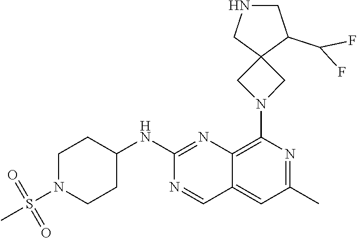 [SMILES: Cc1cc2cnc(NC3CCN(S(C)(=O)=O)CC3)nc2c(N2CC3(CNCC3C(F)F)C2)n1] | 8-(8- (difluoromethyl)- 2,6- diazaspiro[3.4]octan- 2-yl)-6-methyl-N- (1-(methylsulfonyl) piperidin-4- yl)pyrido[3,4- d]pyrimidin-2-amine | 482.4 |
| 101 | 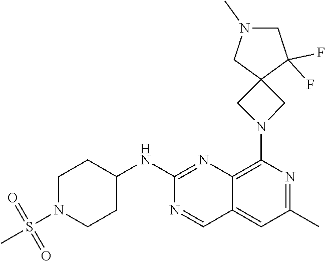 [SMILES: Cc1cc2cnc(NC3CCN(S(C)(=O)=O)CC3)nc2c(N2CC3(CN(C)CC3(F)F)C2)n1] | 8-(8,8-difluoro-6- methyl-2,6- diazaspiro[3.4]octan- 2-yl)-6-methyl-N- (1-(methylsulfonyl) piperidin-4- yl)pyrido[3,4- d]pyrimidin-2-amine | 482.3 |
| 102 | 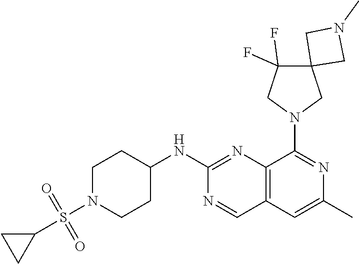 [SMILES: Cc1cc2cnc(NC3CCN(S(=O)(=O)C4CC4)CC3)nc2c(N2CC(F)(F)C3(CN(C)C3)C2)n1] | N-(1- (cyclopropylsulfonyl) piperidin-4-yl)-8- (8,8-difluoro-2- methyl-2,6- diazaspiro[3.4]octan- 6-yl)-6- methylpyrido[3,4- d]pyrimidin-2-amine | 508.2 |
| 103 | 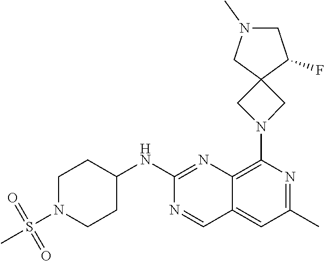 [SMILES: Cc1cc2cnc(NC3CCN(S(C)(=O)=O)CC3)nc2c(N2CC3(CN(C)C[C@@H]3F)C2)n1] | (R)-8-(8-fluoro-6- methyl-2,6- diazaspiro[3.4]octan- 2-yl)-6-methyl-N- (1-(methylsulfonyl) piperidin-4- yl)pyrido[3,4- d]pyrimidin-2-amine | 464.2 |
| 104 | 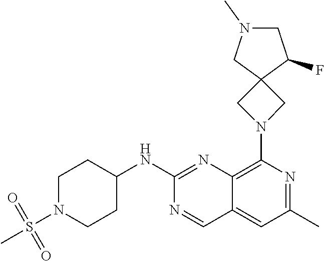 [SMILES: Cc1cc2cnc(NC3CCN(S(C)(=O)=O)CC3)nc2c(N2CC3(CN(C)C[C@H]3F)C2)n1] | (S)-8-(8-fluoro-6- methyl-2,6- diazaspiro[3.4]octan- 2-yl)-6-methyl-N- (1-(methylsulfonyl) piperidin-4- yl)pyrido[3,4- d]pyrimidin-2-amine | 464.2 |

The NMR of the compounds of Table 1 are provided in Table 1B below.

TABLE 1B

| Example | |
|---|---|
| No. | ¹H NMR (ppm) |
| 1 | ¹H NMR (400 MHz, DMSO-d₆) δ (ppm) 9.09 (s, 1H), |
| | 7.95-7.80 (m, 1H), 7.79-7.32 (m, 1H), 7.12-6.96 (m, 1H), |
| | 4.00-3.71 (m, 5H), 3.70-3.50 (m, 2H), 3.02-2.81 (m, 5H), |
| | 2.80-2.61 (m, 4H), 2.15-1.92 (m, 2H), 1.78-1.55 (m, 3H), |
| | 0.55-0.41 (m, 2H), 0.40-0.25 (m, 2H). |
| 2 | 1H NMR (400 MHz, DMSO-d6) δ 8.99 (s, 1H), 7.73 |
| | (d, J = 5.6 Hz, 1H), 7.52 (s, 1H), 6.81 (d, J = 5.2 Hz, 1H), |
| | 4.10 (S, 2H), 3.89 (S, 3H), 3.59 (d, J = 12.4 Hz, 2H), |
| | 2.93-2.86 (m, 5H), 2.75-2.56 (m, 4H), 2.13-1.96 (m, 4H), |
| | 1.69-1.56 (m, 2H). |
| 3 | ¹H NMR (400 MHz, DMSO-d₆) δ(ppm) 9.03 (s, 1H), |
| | 7.76 (d, J = 6.0 Hz, 1H), 7.57-7.51 (m, 1H), 6.87 |
| | (d, J = 6.0 Hz, 1H), 4.45-4.27 (m, 1H), 4.11-3.98 |
| | (m, 3H), 3.91-3.70 (m, 1H), 3.59-3.55 (m, 2H), 2.98-2.80 |
| | (m, 5H), 2.25-2.07 (m, 1H), 2.16-1.99 (m, 3H), 1.73-1.60 |
| | (m, 4H). |
| 4 | ¹H NMR (400 MHz, DMSO-d₆) δ (ppm) 9.48 (s, 1H), |
| | 7.90 (d, j = 5.6 Hz, 1H), 7.51 (d, j = 9.2 Hz, 2H), |
| | 7.01-6.82 (m, 3H), 4.12-3.95 (m, 2H), 3.92-3.72 |
| | (m, 2H), 3.20-3.01 (m, 4H), 2.70-2.55 (m, 4H), |
| | 2.50-2.38 (m, 4H), 2.30-2.15 (m, 3H), 2.10-1.92 (m, 2H). |
| 5 | ¹H NMR (400 MHz, DMSO-d₆) δ (ppm) 9.04 (s, 1H), |
| | 7.77 (d, j = 5.2 Hz, 1H), 7.62 (s, 1H), 6.90 (d, j = 5.6 Hz, |
| | 1H), 4.75-4.41 (m, 1H), 4.20-3.96 (m, 3H), 3.92-3.72 |
| | (m, 1H), 3.71-3.55 (m, 2H), 2.98-2.80 (m, 5H), |
| | 2.45-2.38 (m, 1H), 2.25-2.16 (m, 1H), 2.08-1.99 (m, 2H), |
| | 1.70-1.51 (m, 2H), 1.36-1.25 (m, 1H), 0.70-0.55 (m, 2H), |
| | 0.54-0.41 (m, 2H). |
| 6 | 1H NMR (400 MHz, DMSO-d6) δ (ppm) 9.13 (s, 1H), |
| | 7.94-7.62 (m, 3H), 7.14-6.97 (m, 2H), 4.39 (s, 4H), |
| | 4.12-4.18 (m, 6H), 3.98-3.92 (m, 2H), 3.27-3.25 (m, 2H), |
| | 2.67-2.61 (m, 4H), 2.16-2.13 (m, 2H), 1.42-1.34 (m, 1H), |
| | 1.31-1.232 (m, 3H). |
| 7 | ¹H NMR (400 MHz, DMSO-d₆) δ(ppm) 8.98 (s, 1H), |
| | 7.72 (d, J = 7.2 Hz, 1H), 7.46 (s, 1H), 6.78 (d, J = 7.2 Hz, |
| | 1H), 4.00-3.80 (m, 5H), 3.75-3.52 (m, 6H), 2.95-2.86 |
| | (m, 5H), 2.12-2.00 (m, 2H), 1.98-1.80 (m, 2H), |
| | 1.70-1.50 (m, 6H). |
| 8 | ¹H NMR (300 MHz, CDCl₃) δ(ppm) 8.97-8.69 (m, 1H), |
| | 7.97-7.75 (m, 1H), 6.84-6.66 (m, 1H), 5.35-5.13 (m, 1H), |
| | 4.71-4.31 (m, 4H), 4.15-3.88 (m, 1H), 3.82-3.68(m, 2H), |
| | 3.65-3.44 (m, 4H), 3.19-2.99 (m, 2H), 2.96-2.75 (m, 3H), |
| | 2.33-2.15 (m, 2H), 2.14-1.85 (m, 3H), 0.56-0.30 (m, 4H). |
| 9 | ¹H NMR (300 MHz, CDCl₃) δ(ppm) 8.94 (d, J = 3.8 Hz, |
| | 1H), 7.91 (d, J = 5.5 Hz, 1H), 7.45-7.36 (m, 2H), 7.12 |
| | (s, 1H), 6.97 (dd, J = 9.5, 2.8 Hz, 2H), 6.78 (d, J = 5.5 |
| | Hz, 1H), 4.11-3.95 (m, 3H), 3.89 (d, J = 11.7 Hz, 1H), |
| | 3.52 (dt, J = 9.9, 6.8 Hz, 1H), 3.45-3.30 (m, 3H), 3.23 |
| | (d, J = 5.7 Hz, 4H), 2.81 (d, J = 2.7 Hz, 3H), 2.69-2.57 |
| | (m, 4H), 2.39 (d, J = 5.6 Hz, 3H), 2.00 (td, J = 9.2, 7.5, |
| | 3.8 Hz, 4H). |
| 10 | ¹H NMR (400 MHz, Chloroform-d) δ (ppm) 9.18-8.91 |
| | (m, 1H), 8.08-7.80 (m, 2H), 6.90-6.69 (m, 1H), 6.44-6.24 |
| | (m, 1H), 4.48-4.32 (m, 1H), 4.28-3.99 (m, 5H), 3.75-3.55 |
| | (m, 2H), 2.94 (s, 2H), 2.63-2.45 (m, 3H), 2.30 (s, 1H), |
| | 2.05 (s, 1H), 2.05 (s, 1H), 1.52-1.36 (m, 2H). |
| 11 | ¹H NMR (400 MHz, DMSO-d₆) δ (ppm) 8.96 (s, 1H), |
| | 8.34 (s, 1H), 7.77 (s, 1H), 7.535 (s, 1H), 4.33-4.24 |
| | (m, 2H), 4.23-4.15 (m, 1H), 3.92 (s, 3H), 3.70 (m, 1H), |
| | 3.60-3.50 (m, 2H), 3.31-3.25 (m, 2H), 3.18-2.99 (m, 2H), |
| | 2.49-2.40 (m, 3H), 2.33 (s, 3H), 2.08-1.97 (m, 2H), |
| | 1.70-1.54 (m, 2H). |
| 12 | ¹H NMR (400 MHz, DMSO-d₆) δ (ppm) 8.98 (s, 1H), |
| | 7.54 (s, 1H), 6.78 (s, 1H), 4.40-4.22 (m, 4H), 3.90-3.79 |
| | (m, 1H), 3.61 (d, J = 11.6 Hz, 3H), 3.26-3.15 (m, 3H), |
| | 2.97-2.84 (m, 5H), 2.34 (s, 3H), 2.13-1.98 (m, 2H), |
| | 1.69-1.55 (m, 2h0 |
| 13 | ¹H NMR (400 MHz, DMSO-d₆) δ (ppm) 8.98 (s, 1H), |
| | 7.54 (s, 1H), 6.78 (s, 1H), 4.40-4.19 (m, 4H), 3.90-3.79 |
| | (m, 1H), 3.72-3.63 (m, 2H), 3.38-3.35 (m, 1H), 3.20 |
| | (d, J = 7.6 Hz, 2H), 3.00 (t, J = 11.6 Hz, 2H), 2.70-2.56 |
| | (m, 2H), 2.34 (s, 3H), 2.09-2.00 (m, 2H), 1.67-1.51 |
| | (m, 2H), 1.03-0.90 (m, 4H) |
| 14 | ¹H NMR (400 MHz, CDCL₃) δ (ppm) 8.92 (s, 1H), |
| | 7.92-7.90 (m, 1H), 6.81-6.79 (m, 1H), 5.70 (s, 1H), |
| | 4.82-4.75 (m, 1H), 4.32-4.08 (m, 6H), 3.96-3.91 (m, 2H), |
| | 2.93 (s, 3H), 2.36-2.27 (m, 1H), 2.17-2.01 (m, 1H), |
| | 1.65-1.60 (m, 2H). |
| 15 | ¹H NMR (400 MHz, Chloroform-d) δ (ppm) 8.83 |
| | (s, 1H), 7.87 (d, J = 5.5 Hz, 1H), 6.74 (d, J = 5.5 Hz, 1H), |
| | 5.35 (s, 1H), 4.31 (s, 2H), 4.16-3.96 (m, 2H), 3.62-3.44 |
| | (m, 2H), 3.41 (s, 2H), 3.22-3.06 (m, 3H), 2.90 (s, 3H), |
| | 2.53-2.46 (m, 2H), 2.37 (s, 3H), 2.13-2.01 (m, 4H), |
| | 1.64 (d, J = 2.6 Hz, 2H). |
| 16 | 1H NMR (400 MHz, DMSO-d6) δ (ppm) 9.04 (s, 1H), |
| | 7.85-7.71 (m, 4H), 7.69-7.53 (m, 2H), 6.96 |
| | (d, J = 5.6 Hz, 1H), 5.22-5.02 (m, 1H), 4.53-4.36 |
| | (m, 3H), 4.51-3.96 (m, 3H), 3.65-3.50 (m, 2H), 3.03-2.86 |
| | (m, 5H), 2.07-1.93 (m, 2H), 1.70-1.51 (m, 2H). |
| 17 | ¹H NMR (400 MHz, Chloroform-d) δ (ppm) 8.93-8.77 |
| | (m, 1H), 7.97-7.80 (m, 1H), 6.86-6.67 (m, 1H), |
| | 5.49-5.30 (m, 1H), 4.51-4.11 (m, 4H), 3.25-3.01 (m, 3H), |
| | 2.55-2.05 (m, 8H), 1.54-1.23 (m, 3H). |
| 18 | 1H NMR (400 MHz, DMSO-d6) δ (ppm) 9.14 (s, 1H), |
| | 7.83 (d, J = 5.2 Hz, 1H), 7.73-7.66 (m, 1H), 7.60 |
| | (d, J = 2.8 Hz, 1H), 6.97-6.91 (m, 1H), 6.87 (d, J = 5.2 |
| | Hz, 1H), 3.92 (s, 3H), 3.90-3.79 (m, 4H), 3.68-3.57 |
| | (m, 3H), 3.56-3.42 (m, 5H), 2.68-2.53 (m, 4H), 1.89-1.78 |
| | (m, 2H), 1.61-1.45 (m, 4H), 1.00-0.83 9m, 3H). |
| 19 | ¹H NMR (400 MHz, Chloroform-d) δ (ppm) 9.05-8.94 |
| | (m, 1H), 8.16-8.01 (m, 1H), 7.27-7.19 (m, 1H), 7.15-7.04 |
| | (m, 2H), 6.60 (t, J = 9.1 Hz, 1H), 5.44-5.25 (m, 1H), |
| | 4.45-4.30 (m, 2H), 3.96-3.71 (m, 3H), 2.86-2.72 |
| | (m, 5H), 2.30-2.16 (m, 4H), 1.96-1.83 m, 4H), 1.73-1.60 |
| | (m, 4H). |
| 20 | ¹H NMR (300 MHz, Chloroform-d) δ (ppm) 8.96 (s, 1H), |
| | 7.94 (d, J = 5.5 Hz, 1H), 7.75 (s, 1H), 6.85-6.75 (m, 2H), |
| | 4.29-3.93 (m, 5H), 3.12 (s, 2H), 2.45 (s, 3H), 2.35-1.98 |
| | (m, 8H), 1.48-1.41 (m, 2H). |
| 21 | ¹H NMR (300 MHz, Chloroform-d) δ (ppm) 9.04-8.98 |
| | (m, 1H), 8.09-8.01 (m, 1H), 7.99-7.88 (m, 2H), |
| | 7.69-7.60 (m, 1H), 6.91-6.76 (m, 2H), 4.40-4.29 (m, 1H), |
| | 4.26-4.07 (m, 3H), 4.04-3.95 (m, 4H), 3.43 (s, 4H), |
| | 2.59-2.41 (m, 2H), 2.36-2.27 (m, 1H), 2.11-2.00 (m, 1H), |
| | 1.48-1.38 (m, 2H), 1.02 (t, J = 7.2 Hz, 3H). |
| 22 | ¹H NMR (400 MHz, Chloroform-d) δ (ppm) 9.04-8.87 |
| | (m, 1H), 8.04-7.89 (m, 1H), 7.50-7.29 (m, 3H), 7.22-7.12 |
| | (m, 1H), 6.90-6.74 (m, 1H), 4.32-4.05 (m, 3H), 4.03-3.82 |
| | (m, 5H), 2.77-2.50 (m, 3H), 2.31-2.01 (m, 2H), 1.50-1.35 |
| | (m, 2H). |
| 23 | ¹H NMR (400 MHz, Chloroform-d) δ (ppm) 8.99-8.92 |
| | (m, 1H), 7.93 (dd, J = 5.5, 1.5 Hz, 1H), 7.46-7.36 |
| | (m, 2H), 7.35-7.29 (m, 1H), 7.24-7.15 (m, 1H), 6.80 |
| | (dd, J = 5.6, 1.5 Hz, 1H), 4.12-4.04 (m, 2H), 4.04-3.97 |
| | (m, 1H), 3.93 (s, 4H), 3.88-3.83 (m, 1H), 3.57-3.48 |
| | (m, 1H), 3.43-3.36 (m, 1H), 3.36-3.29 (m, 2H), 2.86-2.76 |
| | (m, 3H), 2.66-2.60 (m, 3H), 2.09-1.92 (m, 4H). |
| 24 | ¹H NMR (300 MHz, DMSO-d6) δ (ppm) 9.89 (s, 1H), |
| | 9.22 (s, 1H), 7.87 (d, J = 5.6 Hz, 1H), 7.72 (s, 1H), 7.45 |
| | (d, J = 8.1 Hz, 1H), 7.20 (d, J = 8.1 Hz, 1H), 7.02 |
| | (d, J = 5.5 Hz, 1H), 4.57 (s, 4H), 4.52 (s, 4H), 3.86 |
| | (s, 2H), 3.78 (s, 2H), 3.33-3.30 (s, 3H). |
| 25 | ¹H NMR (400 MHz, DMSO-d6) δ 8.93 (d, J = 2.7 Hz, 1H), |
| | 7.45 (s, 1H), 6.67 (d, J = 6.0 Hz, 1H), 4.25 (s, 4H), 3.81 |
| | (s, 1H), 3.63 (d, J = 12.1 Hz, 2H), 3.47(s, 1H), 3.07-2.78 |
| | (m, 5H), 2.68-2.54 (m, 2H), 2.30 (s, 3H), 2.17-2.07 (m, 1H), |
| | 2.06-1.92 (m, 3H), 1.66-1.48 (m, 2H), 1.07-0.86 (m, 4H). |
| 26 | ¹H NMR (300 MHz, DMSO-d6) δ (ppm) 9.47 (s, 1H), |
| | 9.04 (s, 1H), 7.94 (s, 1H), 7.53 (s, 1H), 6.77 (s, 1H), |
| | 4.82-4.67 (m, 1H), 4.48 (s, 4H), 4.10 (s, 4H), 3.21 (s, 2H), |
| | 3.12 (s, 2H), 3.03 (s, 3H), 2.65-2.56 (m, 2H), 2.56-2.54 |
| | (m, 2H), 2.34 (s, 3H), 2.17 (s, 3H). |
| 27 | ¹H NMR (300 MHz, DMSO-d6) δ (ppm) 9.88 (s, 1H), 9.11 |
| | (s, 1H), 7.87 (d, J = 1.9 Hz, 1H), 7.55-7.46 (m, 1H), 7.29 |
| | (d, J = 8.3 Hz, 1H), 6.78 (d, J = 0.9 Hz, 1H), 4.65 (d, J = |
| | 17.0 Hz, 4H), 4.38 (s, 4H), 3.30-3.25 (s, 4H), 3.00 (s, 3H), |
| | 2.38-2.32 (m, 3H), 2.19 (s, 3H). |
| 28 | 9.37(s, 1H), 9.03(s, 1H), |
| | 7.38(d, J = 8.7 Hz, 2H), 6.77 (s, 1H), 6.42 (d, J = 8.7 Hz, |
| | 2H), 4.49-4.45 (m, 1H), 3.95-3.91(m, 3H), 3.87(s, 4H), |
| | 3.24(s, 4H), 2.35-2.27(m, 4H), 2.18-2.06 (m, 4H), 1.27-1.21 |
| | (m, 1H), 0.60-0.58 (m, 2H), 0.46-0.41 (m, 2H). |
| 29 | ¹H NMR (400 MHz, Chloroform-d) δ (ppm) 8.89 (s, 1H), |
| | 7.69-7.61 (m, 1H), 7.39-7.31 (m, 1H), 7.28-7.22 (m, 2H), |
| | 6.65 (s, 1H), 4.51 (s, 4H), 4.12-4.00 (m, 8H), 3.87-3.75 |
| | (m, 1H), 3.46 (s, 4H), 2.94 (s, 3H), 2.47 (s, 3H), 2.37 (s, 3H). |
| 30 | (ppm) 9.44 (s, 1H), |
| | 9.04 (s, 1H), 7.46 (d, J = 8.7 Hz, 2H), 6.79 (d, J = 0.9 Hz, |
| | 1H), 6.50-6.39 (m, 2H), 4.56-4.40 (m, 8H), 3.82 (s, 4H), |
| | 3.25 (s, 4H), 2.35 (s, 3H), 2.19 (s, 3H). |
| 31 | ¹H NMR (400 MHz, DMSO-d₆) δ: 9.36(s, 1H), 9.17-9.09 |
| | (m, 1H), 7.85-7.50(m, 1H), 7.30-7.21(m, 1H), 6.78(s, 1H), |
| | 4.65-4.55(m, 1H), 4.15(s, 4H), 3.11-2.98(m, 6H), 2.91-2.85 |
| | (m, 4H), 2.35(s, 3H), 2.11-1.95(m, 3H) |
| 32 | ¹H NMR (400 MHz, DMSO-d₆) δ : 8.95(s, 1H), 7.45 |
| | (s, 1H), 6.68(s, 1H), 4.41(s, 4H), 3.99(s, 1H), 3.88(s, 1H), |
| | 3.75-3.61 (m, 4H), 3.60-3.50(m, 2H), 3.15-3.0(m, 2H), |
| | 2.70-2.55 (m, 1H), 2.35-2.25(m, 3H), 2.10-1.95(m, 2H), |
| | 1.65-1.55 (m, 2H), 1.08-0.91(m, 4H). |
| 33 | ¹H NMR (400 MHz, Chloroform-d) δ (ppm) 8.94-8.77 |
| | (m, 1H), 7.76-7.64 (m, 1H), 7.62-7.53 (m, 1H), 6.81-6.70 |
| | (m, 1H), 6.67-6.60 (m, 1H), 4.69-4.56 (m, 1H), 4.27-4.11 |
| | (m, 2H), 4.13-4.05 (m, 1H), 4.03-3.89 (m, 1H), 3.46-3.34 |
| | (m, 3H), 2.75-2.70 (m, 3H), 2.48-2.43 (m, 3H), 2.42-2.34 |
| | (m, 3H), 2.26-2.19 (m, 1H), 2.09-1.96 (m, 2H), 1.46-1.37 |
| | (m, 2H), 1.31-1.21 (m, 1H). |
| 34 | ¹H NMR (400 MHz, DMSO-d₆) δ (ppm) 9.31 (s, 1H), 9.01 |
| | (s, 1H), 7.40-7.30 (m, 2H), 6.74 (d, J = 0.9 Hz, 1H), |
| | 6.45-6.27 (m, 2H), 4.14-3.99 (m, 2H), 3.99-3.87 (m, 2H), |
| | 3.81 (s, 4H), 3.27 (s, 4H), 2.34 (s, 3H), 2.20 (s, 3H), |
| | 2.16-2.08 (m, 1H), 2.06-1.95 (m, 1H), 1.68-1.51 (m, 2H). |
| 35 | ¹H NMR (300 MHz, DMSO-d6) δ (ppm) 9.24 (s, 1H), 8.96 |
| | (s, 1H), 7.36 (d, J = 8.6 Hz, 2H), 6.64 (s, 1H), 6.38 (d, |
| | J = 8.7 Hz, 2H), 3.78 (d, J = 3.8 Hz, 8H), 3.63-3.43 (m, 4H), |
| | 3.23(s, 4H), 2.30 (s, 3H), 2.17 (s, 3H), 1.82-1.72 (m, 2H), |
| | 1.53-1.44 (m, 4H). |
| 36 | ¹H NMR (400 MHz, DMSO-d₆) δ (ppm) 9.42 (s, 1H), 9.06 |
| | (s, 1H), 7.85 (s, 1H), 7.52 (s, 1H), 6.79 (s, 1H), 4.18-3.87 |
| | (m, 6H), 2.36 (s, 3H), 2.18 (s, 7H), 2.12-2.04 (m, 1H), |
| | 1.69-1.60 (m, 2H), 0.69-0.64 (m, 2H), 0.59-0.54 (m, 2H). |
| 37 | ¹H NMR (400 MHz, DMSO-d₆) δ 9.35(s, 1H), 9.11(s, 1H), |
| | 7.85-7.71(m, 1H), 7.35-7.25(m, 1H), 6.78(s, 1H), 4.65(s, 4H), |
| | 4.38-4.21(m, 4H), 3.61(s, 4H), 3.05-2.95(m, 3H), 2.65(s. 3H), |
| | 2.10 (s, 1H). |
| 38 | ¹H NMR (400 MHz, Chloroform-d) δ (ppm) 8.91 (s, 1H), |
| | 7.79 (s, 1H), 7.59 (s, 1H), 6.77 (s, 1H), 6.70 (s, 1H), 4.45 |
| | (d, J = 12.0 Hz, 1H), 4.31-4.16 (m, 4H), 4.01 (d, J = 12.0 Hz, |
| | 1H), 2.48-2.34(m, 10H), 2.21-2.11 (m, 1H), 1.16-1.07 |
| | (m, 1H), 0.77 (s, 4H), 0.71-0.59 (m, 4H). |
| 39 | ¹H NMR (400 MHz, DMSO-d₆) δ 9.38 (s, 1H), 9.18-9.09 |
| | (m, 1H), 7.82-7.55 (m, 1H), 7.32-7.21 (m, 1H), 6.81-6.75 |
| | (m, 1H), 4.70-4.60 (m, 5H), 4.15 (s, 4H), 3.10-2.98 (m, 5H), |
| | 2.95-2.88 (m, 2H), 2.35 (s, 3H), 2.01-1.95 (m, 2H). |
| 40 | ¹H NMR (400 MHz, DMSO-d6) δ (ppm) 8.88 (s, 1H), 7.20 |
| | (s, 1H), 6.66 (s, 1H), 6.60 (s, 1H), 4.27 (s, 1H), 4.00 (s, 3H), |
| | 2.31 (s, 3H), 2.20-2.13 (m, 1H), 2.09 (s, 6H), 1.99 (d, J = 7.0 |
| | Hz, 4H), 1.80 (d, J = 13.2 Hz, 2H), 1.66-1.52 (m, 2H), |
| | 1.39 (s, 1H), 1.22 (s, 3H), 0.96-0.84 (m, 2H). |
| 43 | ¹H NMR (400 MHz, Chloroform-d) δ (ppm) 8.85-8.74 |
| | (m, 1H), 8.60 (s, 1H), 6.64-6.55 (m, 1H), 4.59-4.32 (m, 2H), |
| | 4.27-3.92 (m, 4H), 3.84-3.67 (m, 1H), 3.12-2.94 (m, 1H), |
| | 2.84-2.68 (m, 4H), 2.55 (d, J = 11.6 Hz, 1H), 2.43 (d, J = |
| | 3.2 Hz, 3H), 2.35-2.21 (m, 2H), 2.13-1.96 (m, 2H), 1.95-1.83 |
| | (m, 1H), 1.54-1.37 (m, 2H). |
| 45 | ¹H NMR (300 MHz, DMSO-d₆) δ (ppm) 8.76 (s, 1H), |
| | 6.66-6.48 (m, 1H), 5.33 (d, J = 7.3 Hz, 1H), 4.39-4.30 |
| | (m, 1H), 4.29-4.19 (m, 1H), 4.18-4.00 (m, 3H), 2.55-2.36 |
| | (m, 5H), 2.19 (d, J = 6.8 Hz, 2H), 2.09-2.01 (m, 1H), |
| | 1.88-1.80 (m, 2H), 1.77-1.63 (m, 6H), 1.48-1.39 (m, 3H), |
| | 1.39-1.29 (m, 3H), 1.28-1.21 (m, 2H). |
| 46 | 1H NMR (400 MHz, DMSO-d6) δ 9.12 (s, 1H), 8.80 (s, 1H), |
| | 7.88 (s, 1H), 7.19 (s, 1H), 6.75 (t, J = 55.5 Hz, 1H), 4.37 |
| | (s, 4H), 3.87 (s, 1H), 3.63 (dd, J = 12.5, 4.5 Hz, 2H), 3.36 |
| | (s, 2H), 3.22-3.14 (m, 2H), 3.11-3.01 (m, 2H), 2.60 (tt, J = |
| | 7.8, 4.9 Hz, 1H), 2.21 (t, J = 7.2 Hz, 2H), 2.02 (d, J = 12.8 |
| | Hz, 2H), 1.63 (q, J = 11.3 Hz, 2H), 1.01 (dt, J = 7.9, 2.8 Hz, |
| | 2H), 0.98-0.92 (m, 2H). |
| 47 | ¹H NMR (400 MHz, DMSO-d₆) δ (ppm) 9.71 (s, 1H), 9.08 |
| | (s, 1H), 7.76 (d, J = 8.5 Hz, 1H), 7.35 (d, J = 8.1 Hz, 1H), |
| | 7.13 (d, J = 8.1 Hz, 1H), 6.76 (d, J = 5.1 Hz, 1H), 4.24 |
| | (s, 4H), 3.95 (d, J = 8.0 Hz, 4H), 3.47 (s, 2H), 3.32 (d, J = |
| | 7.1 Hz, 1H), 2.96 (s, 1H), 2.86-2.77 (m, 1H), 2.35 (s, 3H), |
| | 2.14-2.01 (m, 2H), 1.98-1.91 (m, 1H), 0.49-0.43 (m, 2H), |
| | 0.42-0.37 (m, 2H). |
| 48 | ¹H NMR (400 MHz, DMSO-d₆) δ (ppm) 8.93 (s, 1H), 7.45 |
| | (s, 1H), 6.67 (s, 1H), 4.37 (s, 4H), 3.86 (s, 1H), 3.63 (d, J = |
| | 11.9 Hz, 2H), 3.45-3.37(m, 4H), 3.05 (t, J = 11.2 Hz, 2H), |
| | 2.68-2.56 (m, 1H), 2.31 (s, 3H), 2.06-1.98 (m, 2H), 1.87-1.80 |
| | (m, 1H), 1.70-1.55 (m, 2H), 1.06-0.91 (m, 4H), 0.39-0.29 |
| | (m, 2H), 0.24-0.17 (m, 2H). |
| 49 | ¹H NMR (400 MHz, DMSO-d₆) δ (ppm) 9.11-8.55 (m, 1H), |
| | 7.80-7.21 (m, 1H), 6.76-6.51 (m, 1H), 4.68-3.95 (m, 4H), |
| | 3.83 (s, 1H), 3.73-3.53 (m, 2H), 3.08-2.94 (m, 2H), 2.69 |
| | (s, 2H), 2.65-2.57 (m, 1H), 2.49-2.44 (m, 2H), 2.30 (s, 3H), |
| | 2.27-2.22 (m, 3H), 2.11-1.93 (m, 4H), 1.66-1.53 (m, 2H), |
| | 1.04-0.91 (m, 4H). |
| 50 | ¹H NMR (300 MHz, DMSO-d₆) δ 10.01 (s, 1H), 9.28 (s, 1H), |
| | 8.31 (s, 2H), 7.67 (d, J = 1.9 Hz, 1H), 7.44 (dd, J = 8.1, 2.0 |
| | Hz, 1H), 7.27 (s, 1H), 7.21 (d, J = 8.1 Hz, 1H), 6.80 (t, J = |
| | 55.5 Hz, 1H), 4.34 (s, 4H), 3.98 (s, 2H), 3.94 (s, 2H), 3.25 |
| | (s, 2H), 3.10 (t, J = 7.1 Hz, 2H), 2.16 (t, J = 7.1 Hz, 2H), |
| | 2.11-2.01 (m, 1H), 0.53-0.38 (m, 4H). |
| 51 | ¹H NMR (400 MHz, DMSO-d₆) δ 8.92 (s, 1H), 7.40 (s, 1H), |
| | 6.66 (d, J = 7.9 Hz, 1H), 4.24 (s, 4H), 3.82 (s, 1H), 3.63-3.55 |
| | (m, 2H), 3.06 (d, J = 6.9 Hz, 2H), 2.94 (d, J = 14.2 Hz, 4H), |
| | 2.84-2.77 (m, 2H), 2.30 (s, 3H), 2.26-2.15 (m, 1H), 2.03-1.91 |
| | (m, 4H), 1.90-1.81 (m, 2H), 1.67-1.48 (m, 6H), 1.35-1.21 |
| | (m, 3H). |
| 52 | ¹H NMR (400 MHz, Chloroform-d) δ (ppm) 8.93-8.67 |
| | (m, 1H), 6.77-6.43 (m, 1H), 5.96 (s, H), 5.25-5.03 (m, 1H), |
| | 4.70-4.42 (m, 1H), 4.16-3.72 (m, 6H), 3.01-2.89 (m, 2H), |
| | 2.83 (s, 3H), 2.86-2.36 (m, 6H), 2.28-2.36 (m, 6H), 2.28-2.18 |
| | (m, 2H), 2.06-1.82 (m, 4H). |
| 53 | ¹H NMR (400 MHz, DMSO-d₆) δ (ppm) 8.91 (s, 1H), 7.37 |
| | (s, 1H), 6.63 (s, 1H), 4.08 (s, 2H), 3.90 (s, 2H), 3.82 (d, J = |
| | 9.5 Hz, 1H), 3.59 (d, J = 12.0 Hz, 2H), 2.93-2.83 (m, 5H), |
| | 2.70-2.58 (m, 4H), 2.30 (s, 3H), 2.07-1.99 (m, 4H), 1.59 |
| | (q, J = 11.3 Hz, 2H). |
| 54 | ¹H NMR (400 MHz, DMSO-d₆) δ (ppm) 8.90 (s, 1H), 7.32 |
| | (s, 1H), 6.61 (s, 1H), 4.04 (s, 2H), 3.89-3.70 (m, 3H), 3.57 |
| | (d, J = 12.0 Hz, 2H), 2.91-2.78 (m, 5H), 2.30 (s, 3H), |
| | 2.04-1.93 (m, 2H), 1.89-1.80 (m, 2H), 1.64-1.57 (m, 2H), |
| | 0.62 (d, J = 6.4 Hz, 4H). |
| 55 | 1H NMR (400 MHz, DMSO-d6) δ 9.09 (s, 1H), 7.46 |
| | (d, J = 7.6 Hz, 1H), 7.16 (s, 1H), 6.68 (t, J = 55.7 Hz, 1H), |
| | 4.39 (q, J = 9.5 Hz, 4H), 3.94 (s, 1H), 3.64 (d, J = 12.6 Hz, |
| | 2H), 3.27 (s, 2H), 3.17 (d, J = 7.2 Hz, 2H), 3.11 (t, J = 7.1 |
| | Hz, 2H), 3.01 (ddd, J = 13.0, 10.6, 2.9 Hz, 2H), 2.75 (p, J = |
| | 7.5 Hz, 1H), 2.17 (dd, J = 8.3, 6.4 Hz, 4H), 2.02 (dd, J = 13.2, |
| | 3.7 Hz, 2H), 1.97-1.81 (m, 4H), 1.72-1.59 (m, 2H). |
| 56 | ¹H NMR (400 MHz, DMSO-d₆) δ (ppm) 8.94 (s, 1H), 7.43 |
| | (s, 1H), 6.69 (s, 1H), 5.47 (s, 1H), 4.39-4.08 (m, 3H), |
| | 3.82-3.71 (m, 1H), 3.69-3.58 (m, 2H), 3.04-2.90 (m, 2H), |
| | 2.68-2.61 (m, 1H), 2.35-2.21 (m, 3H), 2.15-1.97 (m, 2H), |
| | 1.70-1.52 (m, 2H), 1.30-1.17 (m, 2H), 1.08-0.92 (m, 4H), |
| | 0.51-0.39 (m, 4H). |
| 57 | ¹H NMR (400 MHz, DMSO-d6) δ (ppm) 8.93 (s, 1H), 7.47 |
| | (s, 1H), 6.68 (d, J = 0.9 Hz, 1H), 4.67-4.44 (m, 3H), 4.16 |
| | (s, 2H), 3.84 (s, 1H), 3.63 (d, J = 11.9 Hz, 2H), 3.39-3.35 |
| | (m, 1H), 3.06-2.95 (m, 2H), 2.64-2.53 (m, 1H), 2.31 (s, 3H), |
| | 2.02 (d, J = 12.8 Hz, 2H), 1.65-1.50 (m, 2H), 1.03-0.89 |
| | (m, 4H), 0.60-0.56 (m, 2H), 0.55-0.42 (m, 2H). |
| 58 | ¹H NMR (400 MHz, DMSO-d₆) δ (ppm) 8.93 (s, 1H), 7.41 |
| | (s, 1H), 7.09-6.67 (m, 2H), 4.93-4.89 (m, 1H), 4.28-4.12 |
| | (m, 1H), 4.04-3.79 (m, 3H), 3.67-3.58 (m, 2H), 3.02-2.91 |
| | (m, 2H), 2.59-2.56 (m, 1H), 2.59-2.56 (m, 1H), 2.32 (s, 3H), |
| | 2.23-2.11 (m, 2H), 2.09-1.90 (m, 2H), 1.70-1.53 (m, 2H), |
| | 1.23 (s, 1H), 1.02-0.98 (m, 2H), 0.97-0.90(m, 2H). |
| 59 | ¹H NMR (400 MHz, DMSO-d6) δ (ppm) 8.89 (s, 1H), 7.38 |
| | (s, 1H), 6.61 (s, 1H), 4.14 (s, 2H), 3.91-3.68 (m, 5H), 3.15 |
| | (d, J = 6.7 Hz, 2H), 3.13-3.04 (m, 4H), 2.29 (s, 3H), |
| | 2.07-1.98 (m, 4H), 1.59-1.47 (m, 2H), 1.43 (s, 3H), 1.21-1.13 |
| | (m, 2H), 0.86-0.80 (m, 2H). |
| 60 | ¹H NMR (DMSO-d₆, 499 MHz) δ 8.98 (s, 1H), 7.4-7.6 |
| | (m, 1H), 6.8-6.9 (m, 1H), 4.9-5.0 (m, 1H), 4.2-4.4 (m, 4H), |
| | 3.80 (d, 2H, J = 8.5 Hz), 3.60 (d, 2H, J = 12.3 Hz), 3.46 |
| | (d, 1H), J = 8.5 Hz), 2.8-3.0 (m, 5H), 2.04 (d, 2H, J = 12.3 |
| | Hz), 1.9-2.0 (m, 2H), 1.5-1.7 (m, 3H), 0.92 (m, 2H), |
| | 0.7-0.8 (m, 2H) |
| 61 | ¹H NMR (400 MHz, DMSO-d6) δ (ppm) 8.95 (s, 1H), 7.48 |
| | (s, 1H), 6.69 (s, 1H), 4.67-4.13 (s, 3H), 3.84 (s, 1H), 3.65 |
| | (d, J = 12.1 Hz, 2H), 2.98 (t, J = 11.3 Hz, 2H), 2.88 (t, J = |
| | 11.7 Hz, 2H), 2.71-2.63 (s, 2H), 2.61-2.54 (m, 2H), 2.31 |
| | (s, 3H), 2.07 (s, 1H), 2.04 (d, J = 12.4 Hz, 2H), 1.97 (s, 2H), |
| | 1.64-1.51 (m, 2H), 1.04-0.96 (m, 2H), 0.96-0.90 (m, 2H). |
| 62 | ¹H NMR (400 MHz, DMSO-d6) δ 9.20 (s, 1H), 9.00 (s, 1H), |
| | 7.60 (s, 1H), 6.82 (s, 1H), 4.58-4.41 (m, 2H), 4.38-4.32 |
| | (m, 2H), 4.31-4.21 (m, 2H), 4.19-4.09 (m, 2H), 3.87 (s, 1H), |
| | 3.72-3.57 (m, 2H), 3.09-2.97 (m, 2H), 2.63 (s, 3H), 2.10-1.97 |
| | (m, 2H), 1.69-1.52 (m, 2H), 1.07-0.91 (m, 4H). |
| 63 | ¹H NMR (400 MHz, Chloroform-d) δ (ppm) 9.00 (d, J = 14.1 |
| | Hz, 1H), 7.25-7.13 (m, 1H), 4.98-4.84 (m, 1H), 4.61-4.46 |
| | (m, 1H), 4.01 (d, J = 12.0 Hz, 2H), 3.21-3.16 (m, 3H), |
| | 3.09-2.98 (m, 2H), 2.76-2.56 (m, 5H), 2.48-2.39 (m, 1H), |
| | 2.38-2.29 (m, 1H), 2.07-1.94(m, 2H), 1.92-1.82 (m, 2H), |
| | 1.51 (s, 2H), 1.42 (s, 1H), 1.35-1.26 (m, 2H), 1.25-1.20 |
| | (m, 2H), 1.08-1.00 (m, 2H). |
| 64 | 1H NMR (400 MHz, Chloroform-d) δ 8.79 (s, 1H), 6.66 |
| | (s, 1H), 5.18 (d, J = 7.6 Hz, 1H), 4.52 (s, 4H), 3.95 (s, 5H), |
| | 3.78 (d, J = 12.6 Hz, 2H), 3.57 (t, J = 5.1 Hz, 2H), 3.38 |
| | (s, 3H), 3.12 (dd, J = 18.5, 9.5 Hz, 4H), 2.99 (s, 2H), |
| | 2.89 (p, J = 7.6 Hz, 1H), 2.32-2.20 (m, 2H), 2.23-2.13 |
| | (m, 2H), 2.04-1.85 (m, 5H), 1.75 (t, J = 11.5 Hz, 2H), |
| | 0.99 (dt, J = 4.8, 2.9 Hz, 2H), 0.92-0.81 (m, 2H). |
| 65 | 1H NMR (300 MHz, Chloroform-d) δ 8.92 (s, 1H), 7.09 |
| | (s, 1H), 6.52 (t, J = 56.1 Hz, 1H), 5.36-5.20 (m, 1H), |
| | 4.78-4.13 (m, 4H), 4.09-3.95 (m, 1H), 3.88-3.64 (m, 2H), |
| | 3.22 (s, 2H), 3.14-2.95 (m, 3H), 2.86 (s, 3H), 2.32-2.18 |
| | (m, 2H), 1.98 (m, 2H), 1.83-1.54 (m, 4H). |
| 67 | ¹H NMR (DMSO-d₆, 499 MHz) δ 8.99 (d, 1H), J = 8.2 Hz), |
| | 7.5-7.8 (m, 1H), 6.78 (s, 1H), 4.50 (br d, 1H, J = 7.4 Hz), |
| | 4.2-4.4 (m, 2H), 4.0-4.1 (m, 1H), 3.8-3.9 (m, 1H), 3.80 |
| | (br d, 1H, J = 8.8 Hz), 3.72 (br d, 1H, J = 8.2 Hz), 3.65 |
| | (br d, 2H, J = 11.0 Hz), 3.49 (br d, 1H, J = 8.2 Hz), 3.09 |
| | (td, 2H, J = 4.2, 8.1 Hz), 3.0-3.0 (m, 2H), 2.35 (d, 4H, J = |
| | 2.7 Hz), 2.0-2.1 (m, 2H), 1.5-1.6 (m, 5H), 1.23 (s, 3H), |
| | 1.04 (d, 4H, J = 2.5 Hz) |
| 68 | ¹H NMR (METHANOL-d4, 499 MHz) δ 8.9-9.0 (m, 1H), |
| | 6.8-6.9 (m, 1H), 5.1-5.3 (m, 2H), 4.9-4.7 (m, 2H), 3.8-3.8 |
| | (m, 2H), 3.0-3.1 (m, 2H), 2.60 (br d, 2H, J = 3.6 Hz), 2.5-2.5 |
| | (m, 1H), 2.46 (s, 2H), 2.1-2.2 (m, 2H), 1.6-1.7 (m, 2H), 1.51 |
| | (d, 1H, J = 4.7 Hz), 1.0-1.1 (m, 4H) |
| 69 | ¹H NMR (400 MHz, CDCl₃) δ 8.95 (s, 1H), 7.22-7.11 |
| | (m, 1H), 5.31 (s, 1H), 4.65-4.51 (m, 1H), 4.41-4.26 (m, 1H), |
| | 4.18-3.99 (m, 1H), 3.87-3.68 (m, 3H), 3.52 (s, 1H), 3.11-2.93 |
| | (m, 2H), 2.93-2.78 (m, 4H), 2.76-2.68 (m, 2H), 2.44-2.16 |
| | (m, 5H), 1.87-1.60 (m, 5H), 1.19 (s, 1H). |
| | ¹H NMR (400 MHz, CDCl₃) δ 8.93 (s, 1H), 7.16 (s, 1H), |
| | 5.30-5.20 (m, 1H), 4.12-3.91 (m, 2H), 3.87-3.67 (m, 4H), |
| | 3.19-2.98 (m, 5H), 2.61-2.49 (m, 4H), 2.34-2.20 (m, 3H), |
| | 2.17-2.00 (m, 1H), 1.83-1.65 (m, 2H), 1.15-1.05 (m, 2H), |
| | 1.02-0.93 (m, 2H). |
| 80 | 1H NMR (400 MHz, DMSO-d6) δ 9.80 (s, 1H), 9.15 (s, 1H), |
| | 7.42 (s, 1H), 4.17 (dd, J = 10.4, 6.4 Hz, 2H), 4.07 (d, J = |
| | 6.3 Hz, 2H), 3.92 (s, 1H), 3.60 (d, J = 12.3 Hz, 2H), 3.51 |
| | (t, J = 4.8 Hz, 2H), 3.35 (t, J = 5.6 Hz, 2H), 3.29 (s, 3H), |
| | 3.18 (d, J = 7.3 Hz, 2H), 3.00 (s, 2H), 2.73 (q, J = 8.0, 7.0 |
| | Hz, 2H), 2.60 (d, J = 8.6 Hz, 2H), 2.24-2.07 (m, 4H), 2.01 |
| | (s, 2H), 1.95-1.77 (m, 5H), 1.2 (m, 2H), 1.00-0.91 (m, 4H). |
| 83 | ¹H NMR (400 MHz, DMSO-d6) δ (ppm) 9.88 (s, 1H), 9.13 |
| | (s, 1H), 7.77 (d, J = 8.3 Hz, 2H), 7.35 (d, J = 8.2 Hz, 2H), |
| | 6.79 (s, 1H), 5.52 (s, 1H), 4.44 (s, 2H), 4.21 (s, 2H), 4.10 |
| | (d, J = 9.5 Hz, 2H), 2.90 (s, 3H), 2.36 (s, 3H), 1.29-1.18 |
| | (m, 1H), 0.50-0.42 (m, 2H), 0.41-0.35 (m, 2H). |
| 84 | ¹H NMR (400 MHz, DMSO-d₆) δ (ppm) 8.93 (s, 1H), 7.14 |
| | (s, 1H), 5.25 (s, 1H), 4.20-3.90 (m, 2H), 3.87-3.64 (m, 5H), |
| | 3.14-2.97 (m, 2H), 2.85 (s, 3H), 2.76-2.54 (m, 9H), 2.53-2.38 |
| | (m, 4H), 2.33-2.22 (m, 2H), 1.81-1.68 (m, 2H). |
| 85 | 8.96 (s, 1H), 7.48 |
| | (s, 1H), 6.73 (s, 1H), 4.57-4.36 (m, 2H), 4.28-4.07 (m, 2H), |
| | 3.92-3.76 (m, 1H), 3.66-3.52 (m, 2H), 3.25 (s, 2H), 3.16 |
| | (t, J = 14.3 Hz, 2H), 3.02-2.81 (m, 6H), 2.33 (s, 3H), |
| | 2.08-1.95 (m, 2H), 1.70-1.53 (m, 2H). |
| 86 | 8.69 (s, 1H), 6.48 |
| | (s, 1H), 5.68-5.61 (m, 1H), 4.62-4.50 (m, 2H), 4.42-4.38 |
| | (m, 2H), 4.31-4.20 (m, 2H), 4.14-4.08 (m, 1H), 4.04-3.96 |
| | (m, 2H), 3.72-3.65 (m, 1H), 3.52-3.46 (m, 1H), 3.30-3.22 |
| | (m, 1H), 2.42 (s, 3H), 2.13-2.08 (m, 1H), 1.75-1.61 (m, 1H), |
| | 1.33-1.27 (m, 1H), 0.64-0.52 (m, 2H), 0.50-0.44 (m, 2H). |
| 87 | ¹H NMR (400 MHz, DMSO-d₆) δ (ppm) 8.92 (s, 1H), 8.30 |
| | (s, 1H), 7.77 (s, 1H), 7.46 (s, 1H), 6.67 (s, 1H), 4.37-4.24 |
| | (m, 2H), 4.19-4.15 (m, 1H), 3.93 (s, 3H), 3.67-3.50 (m, 3H), |
| | 2.83-2.78(m, 2H), 2.67-2.61 (m, 2H), 2.50-2.41 (m, 4H), |
| | 2.33-2.30 (m, 3H), 2.04-2.01 (m, 2H), 1.92 (s, 2H), 1.65-1.57 |
| | (m, 2H). |
| 88 | ¹H NMR (400 MHz, Chloroform-d) δ 8.97-8.95(m, 2H), |
| | 7.29-7.26 (m, 1H), 4.63-4.60 (m, 1H), 4.10-3.83 (m, 3H), |
| | 2.99-2.93 (m, 4H), 2.89-2.87 (m, 4H), 2.72-2.70 (m, 3H), |
| | 2.54-2.20 (m, 4H), 1.84-1.78 (m, 3H), 0.47-0.41(m, 4H). |
| 89 | ¹H NMR (400 MHz, DMSO-d6) δ 9.09(s, 2H), 8.98 (s, 1H), |
| | 8.34 (s, 1H), 7.78 (s, 1H), 7.56 (s, 1H), 6.81 (s, 1H), 4.42 |
| | (s, 2H), 4.29 (s, 2H), 4.22 (d, J = 11.6 Hz, 2H), 4.09 (d, J = |
| | 11.5 Hz, 2H), 3.92 (s, 3H), 3.72 (s, 1H), 3.49 (d, J = 11.3 Hz, |
| | 2H), 2.53 (s, 2H), 2.35 (s, 3H), 2.02 (d, J = 12.2Hz, 2H), |
| | 1.69-1.57 (m, 2H). |
| 90 | ¹H NMR (400 MHz, Chloroform-d) δ (ppm) 8.92 (s, 1H), |
| | 7.09 (s, 1H), 6.52 (t, J = 56.1 Hz, 1H), 5.46-5.20 (m, 1H), |
| | 5.01-4.45 (m, 2H), 4.40-4.13 (m, 2H), 4.06-3.94 (m, 1H), |
| | 3.90-3.75 (m, 2H), 3.10-2.79 (m, 10H), 2.32-2.21 (m, 2H), |
| | 2.18-2.04 (m, 2H), 1.85-1.74 (m, 2H). |
| 91 | ¹H NMR (400 MHz, DMSO-d6) δ (ppm) 8.97 (s, 1H), 7.49 |
| | (s, 1H), 6.73 (s, 1H), 6.58 (s, 1H), 6.16 (s, 1H), 4.49 (s, 2H), |
| | 4.19 (s, 2H), 3.82 (s, 1H), 3.58 (d, J = 12.2 Hz, 2H), 2.95-2.84 |
| | (m, 5H), 2.33 (s, 3H), 2.02 (d, J = 12.0 Hz, 2H), 1.66-1.54 |
| | (m, 2H). |
| 92 | ¹H NMR (400 MHz, CDCL₃) δ (ppm) 8.83 (s, 1H), 6.68 |
| | (s, 1H), 5.25-5.24 (m, 1H), 4.47-4.41 (m, 2H), 4.37-4.34 |
| | (m, 2H), 4.12-4.09 (m, 2H), 3.84-3.81 (m, 2H), 3.62-3.51 |
| | (m, 2H), 3.03-2.97 (m, 2H), 2.86 (s, 3H), 2.45 (s, 4H), |
| | 2.26-2.24 (m, 2H), 1.78-1.70 (m, 2H), 1.28 (s, 1H). |
| 93 | ¹H NMR (400 MHz, DMSO-d₆) δ 9.16 (s, 1H), 7.70 (s, 1H), |
| | 7.37 (s, 1H), 6.06-5.85 (m, 2H), 4.08-3.86 (m, 2H), |
| | 3.60-3.50 (m, 2H), 2.96-2.83 (m, 5H), 2.72-2.62 (m, 2H), |
| | 2.59-2.52 (m, 4H), 2.14-2.01 (m, 3H), 1.67-1.55 (m, 2H). |
| 94 | ¹H NMR (400 MHz, DMSO-d₆) δ 8.96 (s, 1H), 7.96 (s, 1H), |
| | 7.52 (s, 1H), 8.76 (s, 1H), 8.67 (s, 1H), 4.40-4.11 (m, 4H), |
| | 3.96 (s, 3H), 3.82-3.59 (m, 5H), 3.38-3.32 (m, 2H), |
| | 2.73-2.57 (m, 3H), 2.33 (s, 3H), 2.09-1.95 (m, 2H), |
| | 1.70-1.46 (m, 2H). |
| 95 | ¹H NMR (400 MHz, DMSO-d₆) δ (ppm) 8.99 (s, 1H), 7.56 |
| | (s, 1H), 6.81 (s, 1H), 4.40-4.37 (m, 4H), 4.32-4.06 (m, 2H), |
| | 4.03-3.85 (m, 3H), 3.66-3.63 (m, 2H), 3.10-2.97 (m, 4H), |
| | 2.35 (s, 3H), 2.03-2.01 (m, 2H), 1.61-1.53 (m, 2H), 1.25-1.21 |
| | (m, 4H). |
| 96 | ¹H NMR (400 MHz, DMSO-d₆) δ (ppm) 8.98 (s, 1H), 7.54 |
| | (s, 1H), 6.78 (s, 1H), 4.40-4.20 (m, 4H), 4.10 (q, J = 5.2 Hz, |
| | 1H), 3.84 (s, 1H), 3.66-3.55 (m, 2H), 3.21 (d, J = 7.7 Hz, |
| | 2H), 3.17 (d, J = 5.2 Hz, 3H), 2.88 (s, 3H), 2.34 (s, 3H), |
| | 2.25 (s, 3H), 2.10-1.98 (m, 2H), 1.67-1.52 (m, 2H). |
| 97 | ¹H NMR (400 MHz, DMSO-d₆) δ 8.96 (s, 1H), 8.34 (s, 1H), |
| | 7.77 (d, J = 0.6 Hz, 1H), 7.53 (s, 1H), 6.76 (d, J = 0.9 Hz, |
| | 1H), 4.28 (s, 2H), 4.23 (s, 1H), 3.92 (s, 3H), 3.70 (s, 1H), |
| | 3.55 (d, J = 11.6 Hz, 2H), 3.39 (d, J = 7.8 Hz, 2H), 3.18 |
| | (s, 2H), 2.45 (d, J = 11.4 Hz, 1H), 2.33 (s, 3H), 2.26 (s, 3H), |
| | 2.08 (s, 2H), 2.06-1.99 (m, 2H), 1.69-1.59 (m, 2H). |
| 98 | ¹H NMR (400 MHz, DMSO-d₆) δ (ppm) 8.97-8.95 (m, 1H), |
| | 7.54-7.44 (m, 1H), 6.76-6.72 (m, 1H), 5.75-5.33 (m, 4H), |
| | 4.38-4.20 (m, 4H), 3.84-3.80 (m, 1H), 3.58-3.49 (m, 4H), |
| | 2.88-2.86 (m, 5H), 2.33 (s, 3H), 2.03-2.00 (m, 2H), 1.60-1.57 |
| | (m, 2H). |
| 99 | ¹H NMR (400 MHz, CD₃OD) δ 8.04 (s, 1H), 7.32 (s, 1H), |
| | 4.10-3.99 (m, 1H), 3.80-3.70 (m, 2H), 3.24-3.13 (m, 2H), |
| | 3.09-2.96 (m, 2H), 2.91-2.88 (m, 4H), 2.84-2.77 (m, 2H), |
| | 2.61 (s, 3H), 2.52-2.44 (m, 1H), 2.36-2.15 (m, 4H), |
| | 1.84-1.66 (m, 2H), 0.94-0.83 (m, 2H). |
| 100 | ¹H NMR (400 MHz, Chloroform-d) δ 9.04-8.68 (m, 1H), |
| | 7.45-7.11 (m, 1H), 6.64 (d, J = 4.1 Hz, 1H), 6.03 (t, J = 55.8 |
| | Hz, 1H), 5.32-5.06 (m, 1H), 4.92-4.68 (m, 1H), 4.34 (s, 3H), |
| | 4.10-3.91 (m, 1H), 3.89-3.73 (m, 2H), 3.51-3.07 (m, 4H), |
| | 3.05-2.92 (m, 2H), 2.91-2.78 (m, 3H), 2.73-2.56 (m, 3H), |
| | 2.22 (s, 2H), 1.83-1.65 (m, 2H). |
| 101 | ¹H NMR (400 MHz, DMSO-d₆) δ (ppm) 8.97 (s, 1H), 7.49 |
| | (s, 1H), 6.74 (s, 1H), 4.48 (s, 2H), 4.19 (s, 2H), 3.83 (s, 1H), |
| | 3.61-3.52 (m, 2H), 3.03-2.92 (m, 4H), 2.91-2.83 (m, 5H), |
| | 2.33 (s, 3H), 2.29 (s, 3H), 2.06-1.97 (m, 2H), 1.67-1.54 |
| | (m, 2H). |
| 102 | ¹H NMR (400 MHz, DMSO-d₆) δ (ppm) 9.03-8.92 (m, 1H), |
| | 7.55 (s, 1H), 6.81-6.72 (m, 1H), 4.44-4.15 (m, 4H), 3.84 |
| | (s, 1H), 3.67 (d, J = 12.0 Hz, 2H), 3.34-3.30 (m, 2H), 3.20 |
| | (d, J = 7.6 Hz, 2H), 3.05-2.93 (m, 2H), 2.63-2.54 (m, 1H), |
| | 2.34 (s, 3H), 2.25 (s, 3H), 2.12-1.98 (m, 2H), 1.67-1.50 |
| | (m, 2H), 1.04-0.91 (m, 4H). |
| 103 | ¹H NMR (400 MHz, DMSO-d₆) δ 8.94 (s, 1H), 7.45-7.41 |
| | (m, 1H), 6.70 (s, 1H), 5.30-5.15 (m, 1H), 4.54-4.17 (m, 3H), |
| | 3.82 (s, 1H), 3.57-3.54 (m, 2H), 2.96-2.88 (m, 5H), |
| | 2.84-2.82 (m, 2H), 2.67-2.50 (m, 3H), 2.49-2.28 (m, 6H), |
| | 2.12-1.95 (m, 2H), 1.71-1.52 (m, 2H). |
| 104 | ¹H NMR (400 MHz, DMSO-d₆) δ 8.94 (s, 1H), 7.49-7.43 |
| | (m, 1H), 6.70 (s, 1H), 5.30-5.15 (m, 1H), 4.53-4.13 (m, 3H), |
| | 3.82 (s, 1H), 3.58-3.56 (m, 2H), 3.01-2.82 (m, 7H), |
| | 2.67-2.63 (m, 3H), 2.50-2.28 (m, 6H), 2.02-1.99 (m, 2H), |
| | 1.71-1.59 (m, 2H). |

In some embodiments, the compounds of this disclosure are in Table 2.

TABLE 2

| Cmpd | | |
|---|---|---|
| No. | Structure | Name |
| 8A | 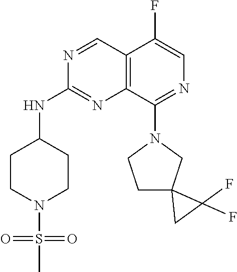 [SMILES: CS(=O)(=O)N1CCC(Nc2ncc3c(F)cnc(N4CCC5(C4)CC5(F)F)c3n2)CC1] | 8-(1,1-difluoro-5- azaspiro[2.4]heptan-5- yl)-5-fluoro-N-(1- (methylsulfonyl) piperidin-4-yl)pyrido[3,4- d]pyrimidin-2-amine |
| 9A | 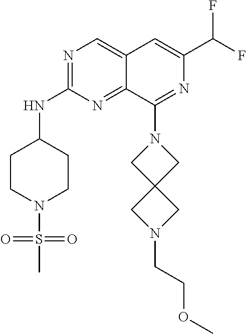 [SMILES: COCCN1CC2(C1)CN(c1nc(C(F)F)cc3cnc(NC4CCN(S(C)(=O)=O)CC4)nc13)C2] | 6-(difluoromethyl)-8-(6- (2-methoxyethyl)-2,6- diazaspiro[3.3]heptan-2- yl)-N-(1- (methylsulfonyl) piperidin-4-yl)pyrido[3,4- d]pyrimidin-2-amine |
| 10A | 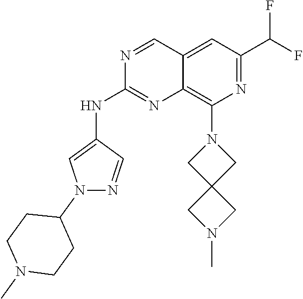 [SMILES: CN1CCC(n2cc(Nc3ncc4cc(C(F)F)nc(N5CC6(CN(C)C6)C5)c4n3)cn2)CC1] | 6-(difluoromethyl)-8-(6- methyl-2,6- diazaspiro[3.3]heptan-2- yl)-N-(1-(1- methylpiperidin-4-yl)- 1H-pyrazol-4- yl)pyrido[3,4- d]pyrimidin-2-amine |
| 11A | 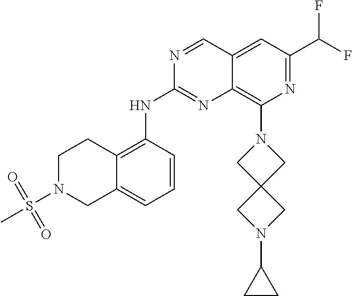 [SMILES: CS(=O)(=O)N1CCc2c(cccc2Nc2ncc3cc(C(F)F)nc(N4CC5(C4)CN(C4CC4)C5)c3n2)C1] | 8-(6-cyclopropyl-2,6- diazaspiro[3.3]heptan-2- yl)-6-(difluoromethyl)- N-(2-(methylsulfonyl)- 1,2,3,4- tetrahydroisoquinolin-5- yl)pyrido[3,4- d]pyrimidin-2-amine |
| 12A | 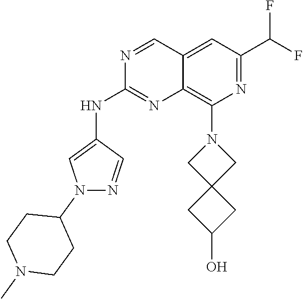 [SMILES: CN1CCC(n2cc(Nc3ncc4cc(C(F)F)nc(N5CC6(CC(O)C6)C5)c4n3)cn2)CC1] | 2-(6-(difluoromethyl)-2- ((1-(1-methylpiperidin- 4-yl)-1H-pyrazol-4- yl)amino)pyrido[3,4- d]pyrimidin-8-yl)-2- azaspiro[3.3]heptan-6-ol |
| 13A | 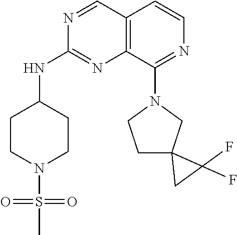 [SMILES: CS(=O)(=O)N1CCC(Nc2ncc3ccnc(N4CCC5(C4)CC5(F)F)c3n2)CC1] | 8-(1,1-difluoro-5- azaspiro[2.4]heptan-5- yl)-N-(1- (methylsulfonyl) piperidin-4-yl)pyrido[3,4- d]pyrimidin-2-amine |
| 14A | 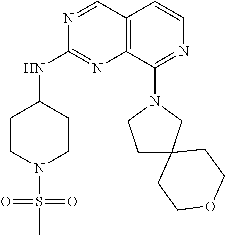 [SMILES: CS(=O)(=O)N1CCC(Nc2ncc3ccnc(N4CCC5(CCOCC5)C4)c3n2)CC1] | N-(1- (methylsulfonyl) piperidin-4-yl)-8-(8-oxa- 2-azaspiro[4.5]decan-2- yl)pyrido[3,4- d]pyrimidin-2-amine |
| 15A | 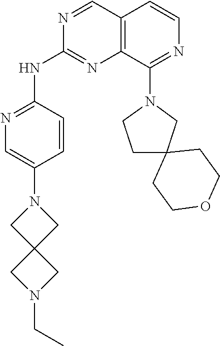 [SMILES: CCN1CC2(C1)CN(c1ccc(Nc3ncc4ccnc(N5CCC6(CCOCC6)C5)c4n3)nc1)C2] | N-(5-(6-ethyl-2,6- diazaspiro[3.3]heptan-2- yl)pyridin-2-yl)-8-(8- oxa-2- azaspiro[4.5]decan-2- yl)pyrido[3,4- d]pyrimidin-2-amine |
| 16A | 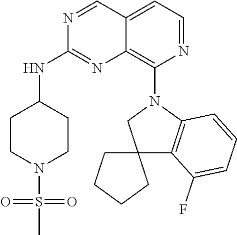 [SMILES: CS(=O)(=O)N1CCC(Nc2ncc3ccnc(N4CC5(CCCC5)c5c(F)cccc54)c3n2)CC1] | 8-(4′- fluorospiro[cyclopentane- 1,3′-indolin]-1′-yl)-N- (1- (methylsulfonyl) piperidin-4-yl)pyrido[3,4- d]pyrimidin-2-amine |
| 17A | 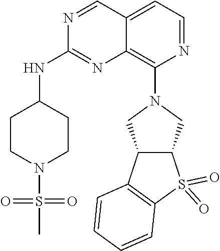 [SMILES: CS(=O)(=O)N1CCC(Nc2ncc3ccnc(N4C[C@H]5c6ccccc6S(=O)(=O)[C@H]5C4)c3n2)CC1] | (3aR,8bR)-2-(2-((1- (methylsulfonyl) piperidin-4- yl)amino)pyrido[3,4- d]pyrimidin-8-yl)- 2,3,3a,8b-tetrahydro-1H- benzo[4,5]thieno[2,3- c]pyrrole 4,4-dioxide |
| 18A | 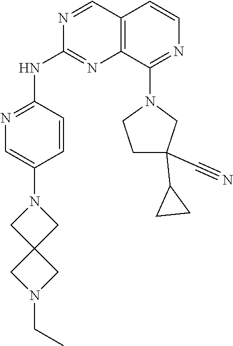 [SMILES: CCN1CC2(C1)CN(c1ccc(Nc3ncc4ccnc(N5CCC(C#N)(C6CC6)C5)c4n3)nc1)C2] | 3-cyclopropyl-1-(2-((5- (6-ethyl-2,6- diazaspiro[3.3]heptan-2- yl)pyridin-2- yl)amino)pyrido[3,4- d]pyrimidin-8- yl)pyrrolidine-3- carbonitrile |
| 19A | 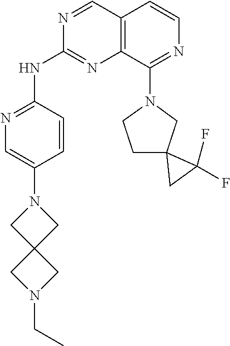 [SMILES: CCN1CC2(C1)CN(c1ccc(Nc3ncc4ccnc(N5CCC6(C5)CC6(F)F)c4n3)nc1)C2] | 8-(1,1-difluoro-5- azaspiro[2.4]heptan-5- yl)-N-(5-(6-ethyl-2,6- diazaspiro[3.3]heptan-2- yl)pyridin-2- yl)pyrido[3,4- d]pyrimidin-2-amine |
| 20A | 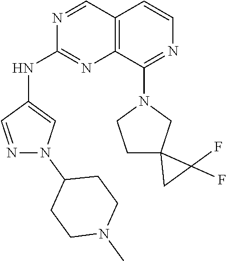 [SMILES: CN1CCC(n2cc(Nc3ncc4ccnc(N5CCC6(C5)CC6(F)F)c4n3)cn2)CC1] | 8-(1,1-difluoro-5- azaspiro[2.4]heptan-5- yl)-N-(1-(1- methylpiperidin-4-yl)- 1H-pyrazol-4- yl)pyrido[3,4- d]pyrimidin-2-amine |
| 21A | 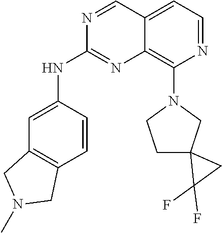 [SMILES: CN1Cc2ccc(Nc3ncc4ccnc(N5CCC6(C5)CC6(F)F)c4n3)cc2C1] | 8-(1,1-difluoro-5- azaspiro[2.4]heptan-5- yl)-N-(2- methylisoindolin-5- yl)pyrido[3,4- d]pyrimidin-2-amine |
| 22A | 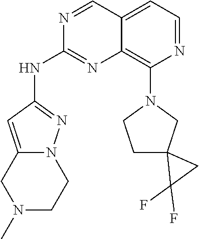 [SMILES: CN1CCn2nc(Nc3ncc4ccnc(N5CCC6(C5)CC6(F)F)c4n3)cc2C1] | 8-(1,1-difluoro-5- azaspiro[2.4]heptan-5- yl)-N-(5-methyl-4,5,6,7- tetrahydropyrazolo[1,5- a]pyrazin-2- yl)pyrido[3,4- d]pyrimidin-2-amine |
| 23A | 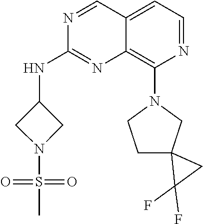 [SMILES: CS(=O)(=O)N1CC(Nc2ncc3ccnc(N4CCC5(C4)CC5(F)F)c3n2)C1] | 8-(1,1-difluoro-5- azaspiro[2.4]heptan-5- yl)-N-(1- (methylsulfonyl)azetidin- 3-yl)pyrido[3,4- d]pyrimidin-2-amine |
| 24A | 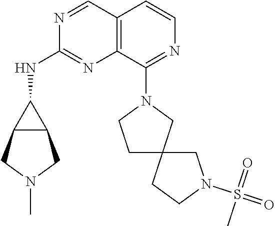 [SMILES: CN1C[C@@H]2[C@H](C1)[C@H]2Nc1ncc2ccnc(N3CCC4(CCN(S(C)(=O)=O)C4)C3)c2n1] | N-((1R,5S,6s)-3-methyl- 3- azabicyclo[3.1.0]hexan- 6-yl)-8-(7- (methylsulfonyl)-2,7- diazaspiro[4.4]nonan-2- yl)pyrido[3,4- d]pyrimidin-2-amine |
| 25A | 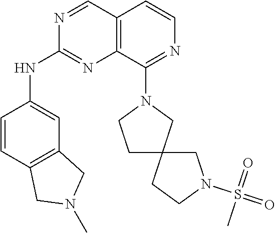 [SMILES: CN1Cc2ccc(Nc3ncc4ccnc(N5CCC6(CCN(S(C)(=O)=O)C6)C5)c4n3)cc2C1] | N-(2-methylisoindolin- 5-yl)-8-(7- (methylsulfonyl)-2,7- diazaspiro[4.4]nonan-2- yl)pyrido[3,4- d]pyrimidin-2-amine |
| 26A | 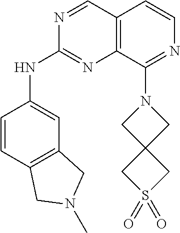 [SMILES: CN1Cc2ccc(Nc3ncc4ccnc(N5CC6(C5)CS(=O)(=O)C6)c4n3)cc2C1] | 6-(2-((2- methylisoindolin-5- yl)amino)pyrido[3,4- d]pyrimidin-8-yl)-2-thia- 6-azaspiro[3.3]heptane 2,2-dioxide |
| 27A | 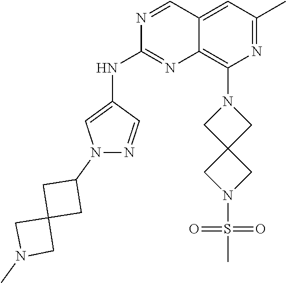 | 6-methyl-N-(1-(2- methyl-2- azaspiro[3.3]heptan-6- yl)-1H-pyrazol-4-yl)-8- (6-(methylsulfonyl)-2,6- diazaspiro[3.3]heptan-2- yl)pyrido[3,4- d]pyrimidin-2-amine |
| 28A | 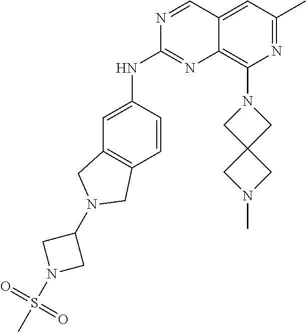 [SMILES: Cc1cc2cnc(Nc3ccc4c(c3)CN(C3CN(S(C)(=O)=O)C3)C4)nc2c(N2CC3(CN(C)C3)C2)n1] | 6-methyl-8-(6-methyl- 2,6- diazaspiro[3.3]heptan-2- yl)-N-(2-(1- (methylsulfonyl) azetidin-3-yl)isoindolin- 5-yl)pyrido[3,4- d]pyrimidin-2-amine |
| 29A | 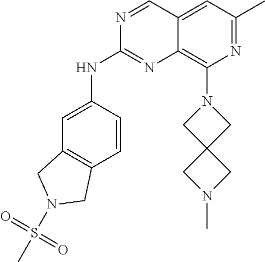 [SMILES: Cc1cc2cnc(Nc3ccc4c(c3)CN(S(C)(=O)=O)C4)nc2c(N2CC3(CN(C)C3)C2)n1] | 6-methyl-8-(6-methyl- 2,6- diazaspiro[3.3]heptan-2- yl)-N-(2- (methylsulfonyl) isoindolin- 5-yl)pyrido[3,4- d]pyrimidin-2-amine |
| 30A | 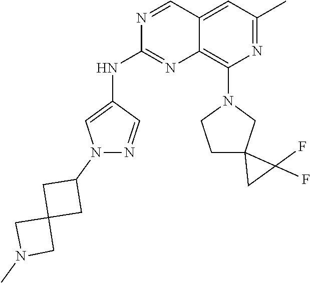 [SMILES: Cc1cc2cnc(Nc3cnn(C4CC5(C4)CN(C)C5)c3)nc2c(N2CCC3(C2)CC3(F)F)n1] | 8-(1,1-difluoro-5- azaspiro[2.4]heptan-5- yl)-6-methyl-N-(1-(2- methyl-2- azaspiro[3.3]heptan-6- yl)-1H-pyrazol-4- yl)pyrido[3,4- d]pyrimidin-2-amine |
| 31A | 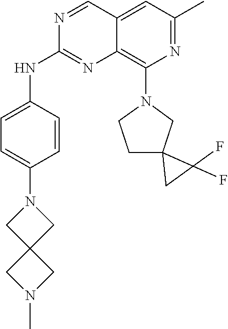 [SMILES: Cc1cc2cnc(Nc3ccc(N4CC5(CN(C)C5)C4)cc3)nc2c(N2CCC3(C2)CC3(F)F)n1] | 8-(1,1-difluoro-5- azaspiro[2.4]heptan-5- yl)-6-methyl-N-(4-(6- methyl-2,6- diazaspiro[3.3]heptan-2- yl)phenyl)pyrido[3,4- d]pyrimidin-2-amine |
| 32A | 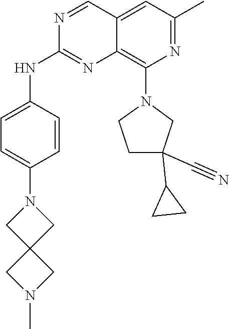 [SMILES: Cc1cc2cnc(Nc3ccc(N4CC5(CN(C)C5)C4)cc3)nc2c(N2CCC(C#N)(C3CC3)C2)n1] | 3-cyclopropyl-1-(6- methyl-2-((4-(6-methyl- 2,6- diazaspiro[3.3]heptan-2- yl)phenyl)amino)pyrido [3,4-d]pyrimidin-8- yl)pyrrolidine-3- carbonitrile |
| 33A | 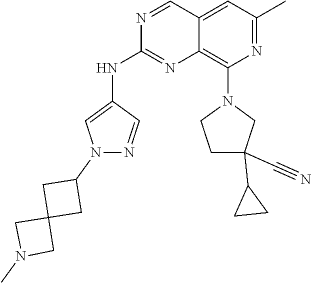 [SMILES: Cc1cc2cnc(Nc3cnn(C4CC5(C4)CN(C)C5)c3)nc2c(N2CCC(C#N)(C3CC3)C2)n1] | 3-cyclopropyl-1-(6- methyl-2-((1-(2-methyl- 2-azaspiro[3.3]heptan-6- yl)-1H-pyrazol-4- yl)amino)pyrido[3,4- d]pyrimidin-8- yl)pyrrolidine-3- carbonitrile |
| 34A | 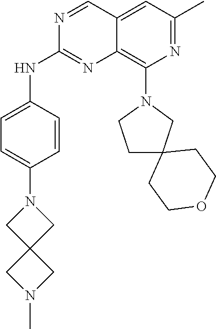 [SMILES: Cc1cc2cnc(Nc3ccc(N4CC5(CN(C)C5)C4)cc3)nc2c(N2CCC3(CCOCC3)C2)n1] | 6-methyl-N-(4-(6- methyl-2,6- diazaspiro[3.3]heptan-2- yl)phenyl)-8-(8-oxa-2- azaspiro[4.5]decan-2- yl)pyrido[3,4- d]pyrimidin-2-amine |
| 35A | 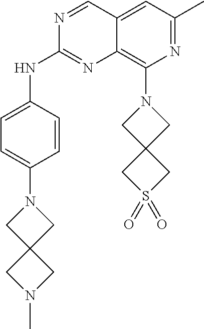 [SMILES: Cc1cc2cnc(Nc3ccc(N4CC5(CN(C)C5)C4)cc3)nc2c(N2CC3(C2)CS(=O)(=O)C3)n1] | 6-(6-methyl-2-((4-(6- methyl-2,6- diazaspiro[3.3]heptan-2- yl)phenyl)amino)pyrido [3,4-d]pyrimidin-8-yl)-2- thia-6- azaspiro[3.3]heptane 2,2-dioxide |

### Example 5: Biochemical Assays

### CDK1/Cyclin B1 ADP-Glo Kinase Assay

The purpose of CDK1/Cyclin Bi assay is to evaluate the inhibition (% inhibition and IC₅₀ values) of small molecule inhibitors by using a Luminescent based ADP-Glo assay. CDK1/Cyclin B1 catalyzes the production of ADP from ATP. ADP-Glo assay monitors ADP producing biochemical reactions. ADP-Glo is performed in 2 steps upon completion of kinase reaction: a combined termination of kinase reaction and depletion of remaining ATP in the first step, and conversion of generated ADP to ATP and the newly produced ATP to light output using luciferase/luciferin reaction in the second step. The luminescent signal generated is proportional to the ADP concentration produced and is correlated with the kinase activity. CDK1/Cyclin B1 was purchased from Carna (Cat 04-102). Typical reaction solutions (10 uL final reaction volume) contained 2% DMSO (±inhibitor), 10 mM MgCI2, 1 mM EGTA, 0.05% BSA, 2 mM DTT, 80 uM ATP (ATP Km=78.6 uM), 0.01% Brig-35, 0.75 uM substrate, and 4.917 nM CDK1/Cyclin Bl enzyme complex in 50 mM HEPES buffer at pH 7.5. The assay was initiated with the addition of ATP-containing substrate solution, following a 30-minute pre-incubation of enzyme and inhibitor at room temperature in the reaction mixture. The reaction was stopped after 90 minutes at room temperature by the addition of 10 μL of ADP-GLO Reagent. After a 90 minute incubation, 20 μL of Kinase Detection Reagent was added. Samples were incubated for 40 minutes, after which plate well luminescence was measured on a Envision microplate reader. The IC₅₀ determinations were made from a plot of the fractional velocity as a function of inhibitor concentration fit to the 4 parameters IC₅₀ equation.

CDK2/Cyclin E1 Full length ADP-Glo Kinase Assay

The purpose of CDK2/Cyclin El assay is to evaluate the inhibition (% inhibition and IC₅₀ values) of small molecule inhibitors by using a Luminescent based ADP-Glo assay. CDK2/Cyclin El full length catalyzes the production of ADP from ATP. ADP-Glo assay monitors ADP producing biochemical reactions. ADP-Glo is performed in 2 steps upon completion of kinase reaction: a combined termination of kinase reaction and depletion of remaining ATP in the first step, and conversion of generated ADP to ATP and the newly produced ATP to light output using luciferase/luciferin reaction in the second step. The luminescent signal generated is proportional to the ADP concentration produced and is correlated with the kinase activity. CDK2/Cyclin E1 was purchased from Eurofins (Cat 14-475M). Typical reaction solutions (10 uL final reaction volume) contained 2% DMSO (±inhibitor), 10 mM MgCI2, 1 mM EGTA, 0.05% BSA, 2 mM DTT, 20 uM ATP (ATP Km=64.78 uM), 0.01% Brig-35, 0.75 uM substrate, and 0.328 nM wild-type full length CDK2/Cyclin El enzyme complex in 50 mM HEPES buffer at pH 7.5. The assay was initiated with the addition of ATP-containing substrate solution, following a 30-minute pre-incubation of enzyme and inhibitor at room temperature in the reaction mixture. The reaction was stopped after 90 minutes at room temperature by the addition of 10 μL of ADP-GLO Reagent. After a 90 minute incubation, 20 uL of Kinase Detection Reagent was added. Samples were incubated for 40 minutes, after which plate well luminescence was measured on a Envision microplate reader. The IC₅₀ determinations were made from a plot of the fractional velocity as a function of inhibitor concentration fit to the 4 parameters IC₅₀ equation.

### CDK4/Cyclin D1 CHEF Assay

The purpose of CDK4/Cyclin Dl assay is to evaluate the inhibition (% inhibition and IC₅₀ values) of small molecule inhibitors by using a Chelation-Enhance Fluorescence (CHEF) assay. In a CHEF assay, phosphorylation of a peptide substrate results in proportional increase in fluorescence. CHEF kinase assay use peptide substrates containing a synthetic alpha-amino acid with a side chain bearing an 8-hydroxyquinoline derivative (sulfonamido-oxide, Sox). Upon phosphorylation of a nearby serine, threonine or tyrosine and in the presence of Mg(II), the spectral properties of the Sox residue are altered, emitting 485 nm wavelength light when excited with a 360 nm wavelength light source. CDK4/Cyclin D1 catalyzes the phosphoryl transfer to the SOX-labeled substrate peptide AQT0258 from Assayquant Technologies. Typical reaction solutions contained 2% DMSO (+/−inhibitor), 10 mM MgCl2, 1 mM DTT, 200 uM ATP (ATP Km=195.2 uM), 0.012% Brig-35, 10 uM AQT0258 peptide, 0.02% BSA, 1% Glycerol, 0.55 mM EGTA, 2.5 nM CDK4/Cyclin D1 in 54 mM HEPES buffer at pH 7.5. The reaction was initiated with the addition of substrate solution, following a 30-minute pre-incubation of enzyme and inhibitor at 22° C. in the reaction mix. Reactions were allowed to proceed for 3 hrs at 22° C., followed by fluorescence read of the reaction. The IC₅₀ determinations were made from a plot of the fractional velocity as a function of inhibitor concentration fit to the 4 parameters IC₅₀ equation.

### CDK4/Cyclin D1 Mobility Shift Assay (MSA)

The purpose CDK4/Cyclin D1 assay is to evaluate the inhibition (% inhibition and IC₅₀ values) in the presence of small molecule inhibitors by using a fluorescence based microfluidic mobility shift assay. CDK4/Cyclin D1 catalyzes the production of ADP from ATP that accompanies the phosphoryl transfer to the substrate peptide 5-FAM-Dyrktide (5-FAM-RRRFRPASPLRGPPK) (Perkin Elmer Peptide 34). The mobility shift assay (MSA) electrophoretically separates the fluorescently labelled peptides (substrate and phosphorylated product) following the kinase reaction. Both substrate and product are measured, and the ratio of these values is used to generate % conversion of substrate to product by the LabChip EZ Reader. Typical reaction solutions contained 2% DMSO (+/−inhibitor), 10 mM MgCl2, 1 mM EGTA, 0.05% BSA, 2 mM DTT, 0.2 mM ATP, 0.01% Brig-35, 1.5 uM 5-FAM-Dyrktide, 2.5 nM CDK4/Cyclin D1 in 50 mM HEPES buffer at pH 7.5. The reaction was initiated with the addition of substrate solution, following a 30-minute pre-incubation of enzyme and inhibitor at 22° C. in the reaction mix. The reaction was stopped after 180 minutes by the addition of 75 μL of 500 mM EDTA and measured on a Perkin Elmer EZ reader instrument. IC₅₀ determinations were made from a plot of the fractional velocity as a function of inhibitor concentration fit to the 4 parameters IC₅₀ equation.

### CDK6/Cyclin D3 ADP-Glo Kinase Assay

The purpose of the CDK6/Cyclin D3 assay is to evaluate the inhibition (% inhibition and IC₅₀ values) in the presence of small molecule inhibitors by using a Luminescent based ADP-Glo assay. CDK6/Cyclin D3 catalyzes the production of ADP from ATP. ADP-Glo assay monitors ADP producing biochemical reactions. ADP-Glo is performed in 2 steps upon completion of kinase reaction: a combined termination of kinase reaction and depletion of remaining ATP in the first step, and conversion of generated ADP to ATP and the newly produced ATP to light output using luciferase/luciferin reaction in the second step. The luminescent signal generated is proportional to the ADP concentration produced and is correlated with the kinase activity. CDK6/Cychin D3 was purchased from Carna. Typical reaction solutions (10 uL final reaction volume) contained 2% DMSO (±inhibitor), 10 mM MgCI2, 1 mM EGTA, 0.05% BSA, 2 mM DTT, 100 uM ATP (ATP Km=291.7 uM), 0.01% Brig-35, 0.75 uM substrate, and 5 nM wild-type CDK6/Cyclin D3 enzyme complex in 50 mM HEPES buffer at pH 7.5. The assay was initiated with the addition of ATP-containing substrate solution, following a 30-minute pre-incubation of enzyme and inhibitor at room temperature in the reaction mixture. The reaction was stopped after 90 minutes at room temperature by the addition of 10 μL of ADP-GLO Reagent. After a 90-minute incubation, 20 μL of Kinase Detection Reagent was added. Samples were incubated for 40 minutes, after which plate well luminescence was measured on a Envision microplate reader. The IC₅₀ determinations were made from a plot of the fractional velocity as a function of inhibitor concentration fit to the 4 parameters IC₅₀ equation.

### Cell Growth Inhibition

MCF-7 and OVCAR-3 cells were used to evaluate the anti-proliferation activity of the CDK inhibitors. MCF-7 (ATCC, HTB-22) cells are epithelial cells from a female patient with ER+ metastatic adenocarcinoma. OVCAR-3 (ATCC, HTB-161) cells were derived from malignant ascites of a patient with ovarian cancer and are known to have CCNE1 amplification. Both cell lines were maintained in RPMI media supplemented with 10% fetal bovine serum. For cell growth inhibition assay, CDK inhibitors in DMSO solution were dispensed with either Echo 655 (Beckman Coulter) or Tecan D300e (TIP) into 384-well plates (Corning #3765) and the 384-well plates were UV-sterilized prior to the assay. The inhibitors were typically tested in the 10-10,000 nM concentration range with half-log serial dilutions. MCF-7 or OVCAR-3 (500 cells/30 μL/well) were added to each well using Multidrop Combi (ThermoFisher) using standard cassettes. The assay plates with cells were cultured at 37° C., 5% CO₂ for 6 days. At the end of the 6-day treatment, 30 μL of CellTiterGlo 2.0 (Promega) was added to each well and the luminescent signal was read using CLARIOstar plus (BMG). The percentage of cell growth inhibition (% CGI) was calculated using the following formula % CGI=100-100×luminescenceₛₐₘₚₗₑ/luminescene_{control}. The half maximal inhibitory concentration (IC50) was determined by nonlinear curve fitting (four parameters, variable slope).

Certain compounds of the disclosure have IC₅₀ values as in Table 3.

All IC₅₀ values in Table 3 are reported as the following: ++++=IC₅₀<200 nM; +++=200 nM<IC50<500 nM; ++=500 nM<IC50<2000 nM; +=IC50>2000 nM

TABLE 3

| | | | CDK4/ | CDK4/ | | MCF-7 | |
|---|---|---|---|---|---|---|---|
| | CDK1 | CDK2/ | CyclinD1 | CyclinD1 | CDK6/ | Cell | OVCAR- |
| Cmpd | Cyclin B1 | CyclinE1 | MSA | AQT | CyclinD3 | IC₅₀ | 3 Cell |
| # | IC₅₀ nM | IC₅₀ nM | IC₅₀ nM | IC₅₀ nM | IC₅₀ nM | nM | IC₅₀ nM |
| 1 | ++ | ++++ | ++++ | | +++ | ++ | + |
| 2 | ++++ | ++++ | ++++ | | ++++ | ++ | ++ |
| 3 | ++++ | ++++ | ++++ | | ++++ | ++ | ++++ |
| 4 | +++ | ++++ | ++++ | | ++++ | +++ | ++ |
| 5 | ++++ | ++++ | ++++ | | ++++ | ++ | ++ |
| 6 | + | ++ | ++++ | | ++++ | ++ | + |
| 7 | +++ | ++++ | ++++ | | ++++ | ++ | ++ |
| 8 | +++ | | ++++ | | | ++ | ++++ |
| 9 | +++ | | ++++ | | | ++ | ++ |
| 10 | + | ++++ | ++++ | | ++++ | ++ | ++ |
| 11 | ++++ | ++++ | | ++++ | ++++ | ++ | +++ |
| 12 | ++++ | ++++ | | ++++ | ++++ | | |
| 13 | ++++ | ++++ | | ++++ | ++++ | | |
| 14 | +++ | ++++ | ++++ | | ++++ | + | + |
| 15 | + | ++ | ++ | | + | + | + |
| 16 | ++++ | ++++ | ++++ | | ++++ | ++ | ++ |
| 17 | + | +++ | ++++ | | +++ | ++ | ++ |
| 18 | + | ++ | ++++ | | ++++ | +++ | + |
| 19 | + | ++++ | ++ | | + | + | + |
| 20 | ++ | ++++ | ++++ | | ++++ | ++ | +++ |
| 21 | + | +++ | ++++ | | ++++ | | |
| 22 | ++++ | ++++ | ++++ | | ++++ | +++ | +++ |
| 23 | ++ | ++++ | | ++++ | ++++ | ++++ | ++++ |
| 24 | ++ | ++++ | | ++++ | ++++ | ++ | ++ |
| 25 | +++ | ++++ | | ++++ | ++++ | +++ | +++ |
| 26 | + | +++ | | +++ | ++ | + | + |
| 27 | ++ | ++++ | | ++ | ++ | + | ++ |
| 28 | ++ | ++++ | | ++++ | ++++ | ++ | ++ |
| 29 | + | +++ | | ++++ | ++ | + | + |
| 30 | + | +++ | | +++ | +++ | + | + |
| 31 | + | ++++ | | + | + | + | + |
| 32 | ++ | ++++ | | ++++ | ++++ | ++ | ++ |
| 33 | ++ | +++ | | ++++ | ++++ | ++ | ++ |
| 34 | ++ | ++++ | | ++++ | ++++ | ++ | ++ |
| 35 | + | +++ | | ++++ | ++++ | ++ | ++ |
| 36 | + | +++ | | +++ | ++ | + | + |
| 37 | + | +++ | | + | + | + | + |
| 38 | + | ++++ | | ++ | + | + | ++ |
| 39 | + | ++++ | | +++ | + | ++ | + |
| 40 | + | ++++ | | ++++ | ++++ | ++ | ++ |
| 41 | ++++ | ++++ | | ++++ | ++++ | + | + |
| 42 | + | ++++ | | ++++ | ++ | ++ | ++ |
| 43 | + | ++ | | ++++ | +++ | ++ | ++ |
| 44 | + | + | | +++ | ++ | + | + |
| 45 | + | + | | ++++ | ++ | + | + |
| 46 | ++++ | ++++ | | ++++ | ++++ | ++ | ++ |
| 47 | ++ | ++++ | | ++++ | ++++ | ++ | ++ |
| 48 | ++ | ++++ | | ++++ | +++ | + | + |
| 49 | ++ | ++++ | | ++++ | +++ | ++ | ++ |
| 50 | ++ | ++++ | | ++++ | ++++ | ++ | ++ |
| 51 | ++ | ++++ | | ++++ | +++ | ++ | ++ |
| 52 | ++++ | ++++ | | ++++ | ++++ | + | ++ |
| 53 | + | ++++ | | ++++ | ++++ | + | + |
| 54 | ++++ | ++++ | | ++++ | ++++ | + | ++ |
| 55 | ++++ | ++++ | | ++++ | ++++ | +++ | ++ |
| 56 | ++++ | ++++ | | ++++ | ++++ | ++ | ++ |
| 57 | +++ | ++++ | | ++++ | ++++ | + | + |
| 58 | ++++ | ++++ | | ++++ | ++++ | ++ | ++ |
| 59 | ++ | ++++ | | ++++ | ++++ | ++ | ++ |
| 60 | ++++ | ++++ | | ++++ | ++++ | ++ | + |
| 61 | ++++ | ++++ | | ++++ | ++++ | ++ | ++ |
| 62 | ++++ | ++++ | | ++++ | ++++ | ++ | ++ |
| 63 | + | +++ | | ++ | + | + | + |
| 64 | ++ | ++++ | | ++++ | +++ | + | + |
| 65 | ++++ | ++++ | | ++++ | ++++ | +++ | +++ |
| 66 | ++ | ++++ | | ++++ | ++++ | ++ | ++ |
| 67 | +++ | ++++ | | ++++ | +++ | + | + |
| 68 | + | ++++ | | +++ | ++ | | |
| 69 | ++++ | ++++ | | ++++ | ++++ | | |
| 70 | ++++ | ++++ | | ++++ | ++++ | ++ | ++ |
| 71 | +++ | ++++ | | ++++ | ++++ | + | + |
| 72 | ++ | ++++ | | +++ | +++ | + | + |
| 73 | ++ | ++++ | | ++++ | +++ | + | + |
| 74 | ++ | ++++ | | +++ | +++ | + | + |
| 75 | + | ++++ | | ++++ | ++++ | + | + |
| 76 | +++ | ++++ | | ++++ | ++++ | ++ | + |
| 77 | +++ | ++++ | | ++++ | ++++ | + | + |
| 78 | +++ | ++++ | | ++++ | +++ | ++ | ++ |
| 79 | +++ | ++++ | | ++++ | +++ | + | ++ |
| 80 | ++ | ++++ | | ++++ | ++++ | + | + |
| 81 | +++ | ++++ | | ++++ | +++ | | |
| 82 | +++ | ++++ | | +++ | +++ | | |
| 83 | +++ | ++++ | | ++++ | ++++ | + | + |
| 84 | +++ | ++++ | | ++++ | ++++ | ++ | ++ |
| 85 | ++++ | ++++ | | ++++ | ++++ | ++ | ++ |
| 86 | + | +++ | | ++++ | +++ | + | + |
| 87 | ++++ | ++++ | | ++++ | ++++ | ++ | +++ |
| 88 | +++ | ++++ | | ++++ | ++++ | | |
| 89 | ++++ | ++++ | | ++++ | ++++ | ++ | +++ |
| 90 | ++++ | ++++ | | ++++ | ++++ | ++ | ++ |
| 91 | ++++ | ++++ | | ++++ | ++++ | ++ | +++ |
| 92 | ++++ | ++++ | | ++++ | ++++ | ++ | ++ |
| 93 | ++++ | ++++ | | ++++ | ++++ | ++ | ++ |
| 94 | ++++ | ++++ | | ++++ | ++++ | ++ | +++ |
| 95 | ++++ | ++++ | | ++++ | ++++ | ++ | ++ |
| 96 | ++++ | ++++ | | ++++ | ++++ | +++ | ++ |
| 97 | ++++ | ++++ | | ++++ | ++++ | ++ | +++ |
| 98 | +++ | ++++ | | ++++ | +++ | | |
| 99 | ++++ | ++++ | | ++++ | ++++ | | |
| 100 | ++++ | ++++ | | ++++ | ++++ | | |
| 101 | +++ | ++++ | | ++++ | ++++ | | |
| 102 | ++++ | ++++ | | ++++ | ++++ | | |
| 103 | ++ | ++++ | | ++++ | ++++ | | |
| 104 | ++ | ++++ | | ++++ | +++ | | |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of treating a disease or condition comprising administering to a subject in need thereof a therapeutically effective amount of a compound having the structure of Formula (I0), or a pharmaceutically acceptable salt or solvate thereof, in combination with one or more additional therapeutic agents or therapies, wherein Formula (I0) is: 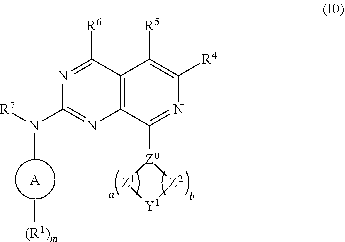
wherein,
A is a ring selected from optionally substituted carbocycle, optionally substituted 4- to 8-membered heterocycle, optionally substituted tetrahydro-triazolopyrazine, and optionally substituted isoindoline;
Z⁰ is —C(H)— or nitrogen;
each of Z¹, Z², and Y¹ are independently selected from —C(R²)₂—, —C(O)—, —NR³—, —N(C(O)R²)—, —NS(O₂)R², —O—, —S—, —S(O)—, and —S(O)₂—;
each of a and b are independently selected from 1, 2, 3, and 4;
each R¹ is independently selected from halogen, —CN, —NO₂, optionally substituted alkyl, optionally substituted alkenyl, optionally substituted alkynyl, optionally substituted carbocycle, and optionally substituted heterocycle;
m is selected from 0 to 5;
each R² is independently selected from hydrogen, halogen, —CN, —OH, optionally substituted alkoxy, optionally substituted alkyl, optionally substituted cycloalkyl, optionally substituted —O-cycloalkyl, and optionally substituted heterocycloalkyl, or two R² substituents come together to form an optionally substituted heterocycle or an optionally substituted carbocycle, or R² and R³ substituents come together to form an optionally substituted heterocycle;
each R³ is independently selected from hydrogen, optionally substituted alkyl, optionally substituted C₃₋₄ carbocycle, and optionally substituted 3- to 4-membered heterocycloalkyl;
R⁴ is selected from hydrogen, halogen, —CN, optionally substituted C₁₋₄ alkyl, optionally substituted C₃₋₄ carbocycle, and optionally substituted 3- to 4-membered heterocycloalkyl;
each of R⁵, R⁶, is independently selected from hydrogen, halogen, —CN, optionally substituted C₁₋₄ alkyl, optionally substituted C₃₋₄ carbocycle, and optionally substituted 3- to 4-membered heterocycloalkyl; and
R⁷ is selected from hydrogen and optionally substituted C₁₋₄ alkyl, and
wherein if A is optionally substituted phenyl, m is from 1 to 5 and at least one R¹ is a heterocycloalkyl,
wherein if A is optionally substituted pyridine, optionally substituted pyridazine, or optionally substituted pyrimidine, R⁴ is selected from hydrogen, halogen, and —CN,
wherein if A is an optionally substituted piperidine sulfonamide, then either (i) Y¹ is —C(R²)₂— and the two R² substituents come together to form a ring selected from an optionally substituted heterocycle and an optionally substituted carbocycle, or (ii) R⁴ is selected from hydrogen, halogen, methyl, and —CN, and
wherein if A-R¹ is 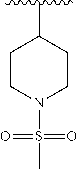 [SMILES: *C1CCN(S(C)(=O)=O)CC1]
and Z⁰ is CH, then R⁴ is methyl or cyclopropyl.

2. The method of claim 1, wherein the compound, or a pharmaceutically acceptable salt or solvate thereof, has the structure of Formula (I): 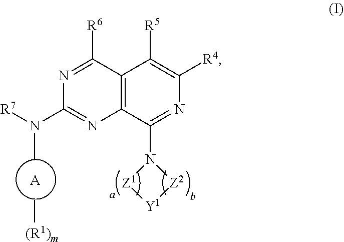
wherein, A is a ring selected from optionally substituted carbocycle, optionally substituted 4- to 6-membered heterocycle, and optionally substituted isoindoline.

3. The method of claim 1, wherein the compound, or a pharmaceutically acceptable salt or solvate thereof, has the structure of one or more of Formulae (IA), (IB), (IC), (ID), or (IE): 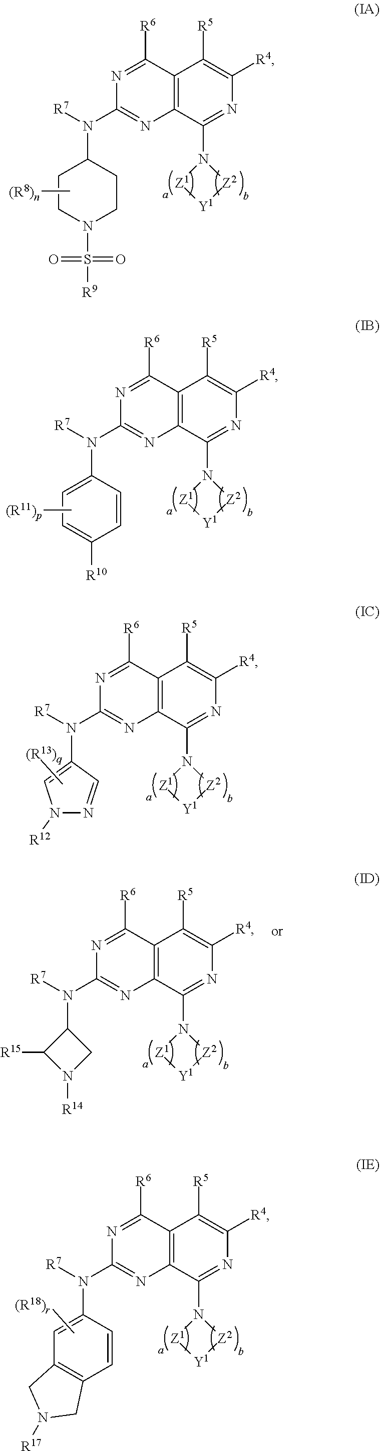
wherein,
R⁸ is selected from halogen, —CN, and optionally substituted C₁₋₄ alkyl;
R⁹ is selected from optionally substituted C₃₋₆ carbocycle, optionally substituted C₅₋₆ heteroaryl, and 3- to 6-membered heterocycloalkyl;
R¹⁰ is optionally substituted alkyl or optionally substituted heterocycloalkyl;
R¹¹ is selected from halogen, —CN, and optionally substituted C₁₋₄ alkyl;
R¹² is selected from an optionally substituted heterocycloalkyl, optionally substituted heterocycloalkylalkyl, optionally substituted cycloalkyl, and optionally substituted cycloalkylalkyl; or R¹² and R¹³ come together to form an optionally substituted heterocycle;
R¹³ is selected from halogen, —CN, and optionally substituted C₁₋₄ alkyl;
n is selected from 0 to 9;
R¹⁴ is selected from —SOR¹⁶—, and optionally substituted heterocycloalkyl;
R¹⁵ is selected from hydrogen, halogen, —CN, and optionally substituted C₁₋₄ alkyl;
R¹⁶ is selected from optionally substituted C₁₋₄ alkyl, optionally substituted C₃₋₆ carbocycle, and optionally substituted 3- to 6-membered heterocycloalkyl;
R¹⁷ is selected from —SOR¹⁹—, optionally substituted alkyl, optionally substituted carbocycle, and optionally substituted heterocycloalkyl;
R¹⁸ is selected from halogen, —CN, and optionally substituted C₁₋₄ alkyl;
R¹⁹ is selected from optionally substituted C₁₋₄ alkyl, optionally substituted C₃₋₆ carbocycle, and optionally substituted 3- to 6-membered heterocycloalkyl;
r is selected from 0 to 5.
p is selected from 0 to 4;
q is selected from 0 to 2; and
wherein when the compound is of formula (IA), either (i) Y¹ is —C(R²)₂— and the two R² substituents come together to form a ring selected from an optionally substituted heterocycle and an optionally substituted carbocycle, or (ii) R⁴ is selected from hydrogen, halogen, and —CN.

4. The method of claim 1, wherein the compound, or pharmaceutically acceptable salt thereof, has the structure of one or more of Formulae (IAA), (IBB), (ICC), (IDD), or (IEE): 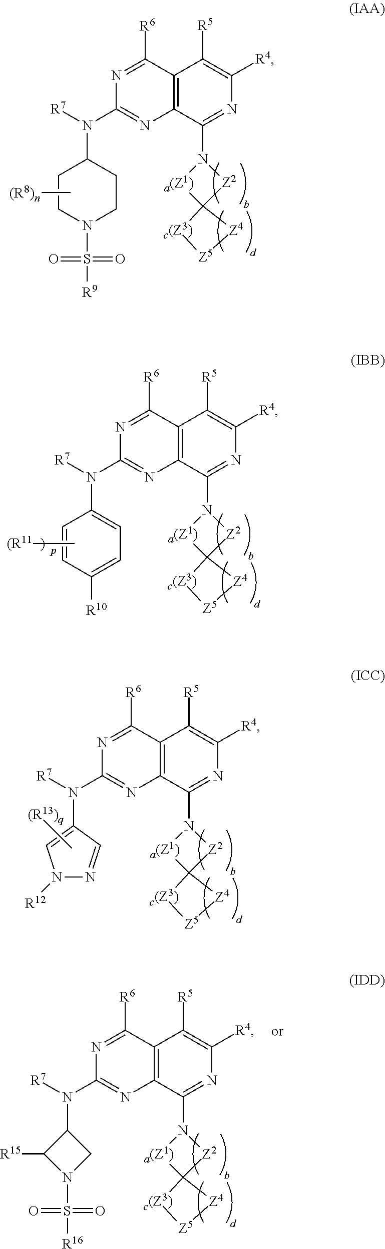 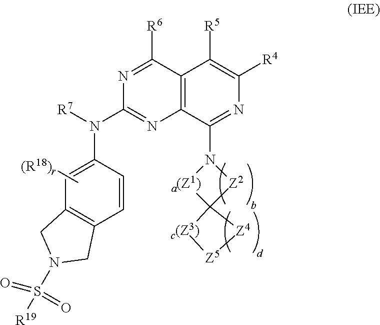
wherein,
R⁸ is selected from halogen, —CN, and optionally substituted C₁₋₄ alkyl;
R⁹ is selected from optionally substituted C₃₋₆ carbocycle, optionally substituted C₅₋₆ heteroaryl, and 3- to 6-membered heterocycloalkyl;
R¹⁰ is optionally substituted heterocycloalkyl;
R¹¹ is selected from halogen, —CN, and optionally substituted C₁₋₄ alkyl;
R¹² is optionally substituted heterocycloalkyl; or R¹² and R¹³ come together to form an optionally substituted heterocycle;
R¹³ is selected from halogen, —CN, and optionally substituted C₁₋₄ alkyl;
R¹⁵ is selected from hydrogen, halogen, —CN, and optionally substituted C₁₋₄ alkyl;
R¹⁶ is selected from optionally substituted C₁₋₄ alkyl, optionally substituted C₃₋₆ carbocycle, and optionally substituted 3- to 6-membered heterocycloalkyl;
R¹⁹ is selected from optionally substituted C₁₋₄ alkyl, optionally substituted C₃₋₆ carbocycle, and optionally substituted 3- to 6-membered heterocycloalkyl;
n is selected from 0 to 9;
p is selected from 0 to 4;
q is selected from 0 to 2;
each of Z¹, Z², Z³, Z⁴ and Z⁵ are independently selected from —C(R²)₂—, —C(O)—, —NR³—, —N(C(O)R²)—, —NS(O₂)R³, —O—, —S—, —S(O)—, and —S(O)₂—, wherein Z⁵ is additionally selected from a bond; and
each of a, b, c, and d are independently selected from 1, 2, 3, and 4.

5. The method of claim 1, wherein the compound, or a pharmaceutically acceptable salt thereof, is administered as a pharmaceutical composition that further comprises one or more pharmaceutically acceptable excipients.

6. The method of claim 1, wherein the one or more agents are selected from the group consisting of a platinum compound, a taxane, apigenin, a Wee-1 inhibitor, a PRMT5 inhibitor, an MDM2 inhibitor, a Src inhibitor, a Raf inhibitor, a MEK inhibitor, an XP01 inhibitor, a vitamin D analog, a type-I TGF-b receptor inhibitor, a TRK inhibitor, a tankyrase inhibitor, a senolytic agent, a RET inhibitor, a proteosome inhibitor, a menin inhibitor, an LC 3-KAT inhibitor, a KIT inhibitor, a KIFC inhibitor, an IGF-1R inhibitor, an HIF-2 alpha inhibitor, an HER2 antibody drug conjugate, a heat shock protein, an HDAC inhibitor, a GLI1 inhibitor, a FOXM1 inhibitor, an EZH2 inhibitor, an estrogen receptor antagonist, an estrogen receptor alpha antagonist, an elF4A inhibitor, a dihydrofolate reductase inhibitor, a CD73 inhibitor, a BTK inhibitor a BMI inhibitor, a beta-catenin inhibitor, a CBP/p300 dual inhibitor, an ALDH1A3 inhibitor, a dual c-Met/Trk inhibitor, an antrogen receptor inhibitor, a Bcl-2 inhibitor, glyoxalase 1 inhibitor, a KIFC1 inhibitor, a USP10 inhibitor, an antioxidant defense inhibitor, RANKL inhibitor, a FLT3 inhibitor, a notch inhibitor, a BRAF inhibitor, a HER2 inhibitor, an eIF4A inhibitor, SHP2 inhibitor, an ERK1/2 inhibitor, an EGFR inhibitor, an IKK beta inhibitor, a PAK inhibitor, a steroid, a steroidogenesis inhibitor, a KIT D816V inhibitor, BET inhibitor, a PI3K inhibitor, an mTOR inhibitor, an FGFR inhibitor, a pan-ERBB inhibitor, an ALK-inhibitor, an anti-PD-1 monoclonal antibody, an anti-PD-L1 monoclonal antibody, an autophagy inhibitor, a YAP-inhibitor, an androgen receptor inhibitor, a PARP inhibitor, a FOXM1 inhibitor, an aromatase inhibitor, a CDK2 inhibitor, a CDK4 inhibitor, and a CDK4/6 inhibitor.

7. The method of claim 6, wherein the one or more agents are selected from the group consisting of mTOR inhibitors, PI3K inhibitors, PARP inhibitors, Her2 antibody drug conjugates, anti-PD-1 monoclonal antibodies, anti-PD-L1 monoclonal antibodies, and Trop2 antibody drug conjugates.

8. The method of claim 1, wherein the one or more additional therapies is hormone therapy.

9. The method of claim 1, wherein the one or more additional therapeutic agents are selected from the group consisting of carboplatin, cisplatin, oxaliplatin, nedaplatin, phenanthriplatin, lobaplatin, enloplatin, paclitaxel, docetaxel, apigenin, adavosertib, pemrametosta, AZD1775, inecalcitol, SNDX-50469, NVP-CGM097, idasanutlin, nutlin-3, siremadlin, brigimadlin, saracatinib, bosutinib, dasatinib, sorafenib, trametinib, binimetinib, cobimetinib, KPT-330, SB-505124, entrectinib, MSC2504877, alectinib. bortezomib, selumetinib, PD0325901, trimetazidine, midostaurin, avapritinib, nintedanibispinesib, SR31527, ganitumab, NVP-AEW541, PT2399, T-DM1, SHetA2, tucidinostat, suberanilohydroxamic acid, vorinostat, valproate, GANT61, NB55, NB73, NB115AQB, lasofoxifene, CR-1-31-B, celastrol, gambogic acid, pralatrexate, ibrutinib, tirabrutinib, PTC-209, ICG-001, NEO2734, N,N-diethylaminobenzaldehyde, altiratinib, seviteronel, RAD140, MLN0128, temsirolimus, sapanisertib, navitoclax, venetoclax, BBGC, ispinesib, spautin-1, auranofin, neratinib, pyrotinib, tucatinib, OPG-Fc, quizartinib, CB-103, encorafenib, vemurafenib, dabrafenib, trastuzumab, CR-1-31-B, TNO155, SCH772984, LY3214996, cetuximab, PF-0647775, erlotinib, Bay 11-7082, PF03758309, progesterone, mitotane, midostaurin, avapritinib, JQ1, ZEN-3694, ARV-825, alpelisib, pictilisib, vistusertib, everolimus, infigratinib, LY2874455, rogaratinib, BLU9931, H3B-6527, FIIN-2, FIIN-3, lenvatinib, ponatinib, regorafenib. Pemigatinib, buparlisib, paxalisib, futibatinib, infigratinib, afatinib, ceritinib, pembrolizumab, nivolumab, cemiplimab, atezolizumab, avelumab, durvalumab, hydroxychloroquine, chloroquine, verteporfin, pyrvinium pamoate, enzalutamide, N-desmethyl enzalutamide, darolutamide, apalutamide, ralaniten, EPI-7170, abiraterone, bafilomycin A1, albendazole, letrozole, fulvestrant, olaparib, talazoparib, NB55, NB73, NB 115, gedatolisib, eribulin, temozolomide, cytarabine, doxorubicin, gemcitabine, pemetrexed, dexamethasone, L-asparaginase, vincristine, 5-FU, sunitinib, and irinotecan.

10. The method of claim 1, wherein the one or more agents are selected from the group consisting of a Trop2 antibody drug conjugate, a cytokine, an oncolytic virus, a bi-specific immune checkpoint inhibitor, a T-cell engager, a cancer vaccine, a cell therapy, a CD73 inhibitor, HER3 antibody drug conjugates such as patritumab or deruxtecan, or a TLR agonist.

11. The method of claim 1, wherein the one or more additional therapies is a cancer gene therapy.

12. The method of claim 1, wherein the one or more additional therapeutic agents are selected from the group consisting of vistusertib, MLN0128, alpelisib, buparlisib, paxalisib, gedatolisib, pictilisib, talazoparib, laparib, T-DM1, sacituzumab govtecan, and datopotamab deruxtecan.

13. The method of claim 1, wherein the one or more additional therapeutic agents are selected from the group consisting of carboplatin, cisplatin, oxaliplatin, nedaplatin, phenanthriplatin, lobaplatin, and enloplatin.

14. The method of claim 1, wherein the one or more additional therapeutic agents are selected from the group consisting of paclitaxel and docetaxel.

15. The method of claim 1, wherein the one or more additional therapeutic agents is apigenin.

16. The method of claim 1, wherein the one or more additional therapeutic agents are selected from the group consisting of adavosertib, pemrametosta, nutlin-3, saracatinib, and dasatinib.

17. The method of claim 1, wherein the one or more additional therapeutic agents are selected from the group consisting of sorafenib, trametinib, cobimetinib, alpelisib, pictilisib, vistusertib, and irinotecan.

18. The method of claim 1, wherein the one or more additional therapeutic agents are selected from the group consisting of FIIN-2, FIIN-3, lenvatinib, ponatinib, regorafenib, pemigatinib, futibatinib, and infigratinib.

19. The method of claim 1, wherein the one or more additional therapeutic agents are selected from the group consisting of afatinib and ceritinib.

20. The method of claim 1, wherein the one or more additional therapeutic agents are selected from the group consisting of pembrolizumab, nivolumab, cemiplimab, atezolizumab, avelumab, and durvalumab.

21. The method of claim 1, wherein the one or more additional therapeutic agents are selected from the group consisting of hydroxychloroquine, verteporfin, and olaparib.

22. The method of claim 1, wherein the one or more additional therapeutic agents are selected from the group consisting of enzalutamide, N-desmethyl enzalutamide, darolutamide, apalutamide, and abiraterone.

23. The method of claim 1, wherein the one or more additional therapies is radiotherapy or ultrasound therapy.

24. The method of claim 1, wherein the disease or condition is cancer.

25. The method of claim 24, wherein the cancer is a solid tumor.

26. The method of claim 24, wherein the cancer is ovarian cancer, pancreatic cancer, bladder cancer, brain cancer, sarcoma, melanoma, lung cancer, urothelial carcinoma mantle cell lymphoma, large B-cell lymphoma, leukemia, colorectal cancer, adenocarcinoma, adrenocortical carcinoma, breast cancer, medulloblastoma, cholangiocarcinoma, glioma, esophageal squamous cell carcinoma, meningioma, Ewing sarcoma, well-differentiated liposarcoma, dedifferentiated liposarcoma, clear cell renal cell carcinoma, lung squamous cell carcinoma, endometrial cancer, gastric cancer, pediatric astrocytoma, rare pediatric undifferentiated sarcoma, mastocytosis, glioblastoma, glioblastoma multiforme, esophageal carcinoma, thyroid cancer, neuroblastoma, colon cancer, rectal cancer, esophageal carcinoma, lung cancer, liver cancer, kidney cancer, bladder cancer, ovarian cancer, well-differentiated liposarcoma, dedifferentiated liposarcoma, advanced dedifferentiated liposarcoma, leiomyosarcoma, neuroendocrine tumor, peripheral nerve sheath tumors, pediatric cancer, mesothelioma, myeloma, chordomas, bladder cancer, nasopharyngeal carcinoma, cervical cancer, testicular germ cell tumors, brain metastasis, head and neck squamous cell carcinoma, oral squamous cell carcinoma, osteosarcoma, or prostate cancer.

27. The method of claim 24, wherein the cancer is metastatic triple-negative breast cancer, non-small cell lung cancer, small cell lung cancer, metastatic urothelial carcinoma, HR+ HER2− metastatic breast cancer, colorectal cancer, esophageal carcinoma, endometrial cancer, pancreatic ductal adenocarcinoma, castrate-resistant prostate cancer, epithelial ovarian cancer, gastric adenocarcinoma, glioblastoma multiforme, head and neck squamous cell carcinoma, hepatocellular carcinoma, cervical cancer, or renal cell carcinoma.

28. The method of claim 24, wherein the cancer is osteosarcoma, glioma, cholangiocarcinoma, glioblastoma, head and neck squamous cell carcinoma, medulloblastoma, advanced dedifferentiated liposarcoma, leiomyosarcoma, chordomas, breast cancer, or pediatric cancer.

29. The method of claim 24, wherein the cancer is medulloblastoma, head and neck squamous cell cancer, chorodomas, mesothelioma, colorectal cancer, chordomas, medulloblastoma, pancreatic ductal adenocarcinoma, thyroid cancer, or oral squamous cell carcinoma.

30. The method of claim 24, wherein the cancer is colorectal cancer, prostate cancer, castration-resistant prostate cancer, and neuroendocrine prostate cancer.

31. The method of claim 1, wherein the compound inhibits CDK 2, CDK 4, CD6, or any combination thereof.

32. The method of claim 31, wherein the CDK is selected from CDK 2/4, CDK 2/6, CDK 4/6, and CDK 2/4/6.
